(19) United States
(12) Patent Application Publication
(10) Pub. No.: US 2026/0207717 A1
(43) Pub. Date: Jul. 23, 2026

(54) METHODS FOR TREATING INFANTILE-ONSET POMPE DISEASE IN PEDIATRIC PATIENTS

(71) Applicant: Amicus Therapeutics, Inc., Philadelphia, PA (US)

(72) Inventors: Jay Barth, Teaneck, NJ (US); Sheela Sitaraman Das, Princeton, PA (US); Jeff Castelli, New Hope, PA (US)

(73) Assignee: Amicus Therapeutics, Inc., Philadelphia, PA (US)

(21) Appl. No.: 19/134,954

(22) PCT Filed: Dec. 1, 2023
(86) PCT No.: PCT/US23/82103

§ 371 Date: Jun. 2, 2025

Related U.S. Application Data
(60) Provisional application No. 63/429,969, filed on Dec. 2, 2022.
(60) Provisional application No. 63/449,810, filed on Mar. 3, 2023.

(51) Int. Cl. A61K 38/47; A61K 31/445; A61P 3/00; A61P 21/00

(52) U.S. Cl. CPC A61K 38/47; A61K 31/445; A61P 3/00; A61P 21/00; C12Y 302/0102

(57) ABSTRACT

Provided herein are methods for treating Pompe disease in pediatric patients by administering to a subject a population of recombinant human acid α-glucosidase molecules or a pharmaceutical composition or formulation thereof, and an enzyme stabilizer.

### TECHNICAL FIELD

The disclosure relates to methods for treating infantile-onset Pompe disease (IOPD) in pediatric patients by administering recombinant human α-glucosidase.

### BACKGROUND

Pompe disease (also known as glycogen storage disease type II (GSD-II) or acid maltase deficiency disease) is an inherited lysosomal storage disease that results from a deficiency in acid α-glucosidase (GAA) activity. A person having Pompe disease lacks or has reduced levels of GAA, the enzyme which breaks down glycogen to glucose, a main energy source for muscles. This enzyme deficiency causes excess glycogen accumulation in the lysosomes, which are intra-cellular organelles containing enzymes that ordinarily break down glycogen and other cellular debris or waste products.

Glycogen accumulation in certain tissues of a subject having Pompe disease, especially muscles, impairs the ability of cells to function normally. In Pompe disease, glycogen is not properly metabolized and progressively accumulates in the lysosomes, especially in skeletal muscle cells and, in the infantile-onset form of the disease, in cardiac muscle cells. The accumulation of glycogen damages the muscle and nerve cells as well as those in other affected tissues.

Current non-palliative treatment of Pompe disease involves enzyme replacement therapy (ERT) using recombinant alglucosidase alfa products sold under the trademarks LUMIZYME®, MYOZYME®, or NEXVIAZYME®. This conventional enzyme replacement therapy seeks to treat Pompe disease by replacing the missing GAA in lysosomes by administering rhGAA thus restoring the ability of cells to break down lysosomal glycogen. LUMIZYME®, MYOZYME® and NEXVIAZYME® are conventional forms of rhGAA produced or marketed as biologics by Sanofi Genzyme and approved by the U.S. Food and Drug Administration and are described by reference to the Physician's Desk Reference (2014) (which is hereby incorporated by reference).

However, the currently marketed ERT, at best, offers limited improvement in measures of muscle function, strength and respiratory function for a finite duration followed by slow decline in these parameters (Toscano and Schoser, (2013) J Neurol 260, 951-959; Wyatt et al., (2012) Health Technol Assess 16(39)). There is a need for Pompe disease treatments that offer long term improvement.

Recently, novel rhGAA formulations have been developed which provide improved efficacy in restoring the ability of cells to break down glycogen by increasing the binding efficiency of the rhGAA to lysosomal receptors. Further, the inventors have also found increased efficacy when the rhGAA is paired with an enzyme stabilizer. However, these methods of treatments have only been established for adult patient populations. There is a need for treating infantile-onset Pompe disease in pediatric (<18 years old) populations.

### SUMMARY

Various aspects of the present disclosure relate to treating Pompe disease in pediatric patients by administering a recombinant human α-glucosidase (rhGAA) in combination with an enzyme stabilizer.

### BRIEF DESCRIPTION OF THE DRAWINGS

FIG. 1A shows non-phosphorylated high mannose N-glycan, a mono-M6P N-glycan, and a bis-M6P N-glycan. FIG. 1B shows the chemical structure of the M6P group. Each square represents N-acetylglucosamine (GlcNAc), each circle represents mannose, and each P represents phosphate.

FIG. 2A describes productive targeting of rhGAA via N-glycans bearing M6P to target tissues (e.g., muscle tissues of subject with Pompe Disease). FIG. 2B describes non-productive drug clearance to non-target tissues (e.g., liver and spleen) or by binding of non-M6P N-glycans to non-target tissues.

FIG. 3 is a schematic diagram of an exemplary process for the manufacturing, capturing and purification of a recombinant lysosomal protein.

FIG. 4 shows a DNA construct for transforming CHO cells with DNA encoding rhGAA.

FIG. 5 is a graph showing the results of CIMPR affinity chromatography of ATB200 rhGAA with (Embodiment 2) and without (Embodiment 1) capture on an anion exchange (AEX) column.

FIG. 6A-FIG. 6H show the results of a site-specific N-glycosylation analysis of ATB200 rhGAA, using two different LC-MS/MS analytical techniques. FIG. 6A shows the site occupancy of the seven potential N-glycosylation sites for ATB200. FIG. 6B shows two analyses of the N-glycosylation profile of the first potential N-glycosylation site for ATB200. FIG. 6C shows two analyses of the N-glycosylation profile of the second potential N-glycosylation site for ATB200. FIG. 6D shows two analyses of the N-glycosylation profile of the third potential N-glycosylation site for ATB200. FIG. 6E shows two analyses of the N-glycosylation profile of the fourth potential N-glycosylation site for ATB200. FIG. 6F shows two analyses of the N-glycosylation profile of the fifth potential N-glycosylation site for ATB200. FIG. 6G shows two analyses of the N-glycosylation profile of the sixth potential N-glycosylation site for ATB200. FIG. 6H summarizes the relative percent mono-phosphorylated and bis-phosphorylated species for the first, second, third, fourth, fifth, and sixth potential N-glycosylation sites.

FIG. 7 is a graph showing Polywax elution profiles of LUMIZYME® (thinner line, eluting to the left) and ATB200 (thicker line, eluting to the right).

FIG. 8 is a table showing a summary of N-glycan structures of LUMIZYME® compared to three different preparations of ATB200 rhGAA, identified as BP-rhGAA, ATB200-1 and ATB200-2.

FIG. 9A and FIG. 9B are graphs showing the results of CIMPR affinity chromatography of LUMIZYME® and MYOZYME®, respectively.

FIG. 10A is a graph comparing the CIMPR binding affinity of ATB200 rhGAA (left trace) with that of LUMIZYME® (right trace). FIG. 10B is a table comparing the bis-M6P content of LUMIZYME® and ATB200 rhGAA.

FIG. 11A is a graph comparing ATB200 rhGAA activity (left trace) with LUMIZYME® rhGAA activity (right trace) inside normal fibroblasts at various GAA concentrations. FIG. 11B is a table comparing ATB200 rhGAA activity (left trace) with LUMIZYME® rhGAA activity (right trace) inside fibroblasts from a subject having Pompe Disease at various GAA concentrations. FIG. 11C is a table comparing Kᵤₚₜₐₖₑ of fibroblasts from normal subjects and subjects with Pompe disease.

FIG. 12 depicts the stability of ATB200 in acidic or neutral pH buffers evaluated in a thermostability assay using SYPRO Orange, as the fluorescence of the dye increases when proteins denature.

FIG. 13 shows tissue glycogen content of WT mice or Gaa KO mice treated with a vehicle, alglucosidase alfa, or ATB200/AT2221, determined using amyloglucosidase digestion. Bars represent Mean±SEM of 7 mice/group. *p<0.05 compared to alglucosidase alfa in multiple comparison using Dunnett's method under one-way ANOVA analysis.

FIG. 14 depicts LAMP1-positive vesicles in muscle fibers of Gaa KO mice treated with a vehicle, alglucosidase alfa, or ATB200/AT2221 or WT mice. Images were taken from vastus lateralis and were representative of 7 mice per group. Magnification=200× (1,000× in insets).

FIG. 15A shows LC3-positive aggregates in muscle fibers of Gaa KO mice treated with a vehicle, alglucosidase alfa, or ATB200/AT2221 or WT mice. Images were taken from vastus lateralis and were representative of 7 mice per group. Magnification=400×. FIG. 15B shows a western blot analysis of LC3 II protein. A total of 30 mg protein was loaded in each lane.

FIG. 16 shows Dysferlin expression in muscle fibers of Gaa KO mice treated with a vehicle, alglucosidase alfa, or ATB200/AT2221 or WT mice. Images were taken from vastus lateralis and were representative of 7 mice per group. Magnification=200×.

FIG. 17 depicts co-immunofluorescent staining of LAMP1 (green) (see for example, “B”) and LC3 (red) (see, for example, “A”) in single fibers isolated from the white gastrocnemius of Gaa KO mice treated with a vehicle, alglucosidase alfa, or ATB200. “C” depicts clearance of autophagic debris and absence of enlarged lysosome. A minimum of 30 fibers were examined from each animal.

FIG. 18 depicts stabilization of ATB200 by AT2221 at 17 μM, and 170 μM AT2221, respectively, as compared to ATB200 alone.

FIG. 19A-FIG. 19H show the results of a site-specific N-glycosylation analysis of ATB200 rhGAA, including an N-glycosylation profile for the seventh potential N-glycosylation site, using LC-MS/MS analysis of protease-digested ATB200. FIG. 19A-FIG. 19H provide average data for ten lots of ATB200 produced at different scales.

FIG. 19A shows the average site occupancy of the seven potential N-glycosylation sites for ATB200. The N-glycosylation sites are provided according to SEQ ID NO: 1. CV=coefficient of variation.

FIG. 19B-FIG. 19H show the site-specific N-glycosylation analyses of all seven potential N-glycosylation sites for ATB200, with site numbers provided according to SEQ ID NO: 5. Bars represent the maximum and minimum percentage of N-glycan species identified as a particular N-glycan group for the ten lots of ATB200 analyzed. FIG. 19B shows the N-glycosylation profile of the first potential N-glycosylation site for ATB200. FIG. 19C shows the N-glycosylation profile of the second potential N-glycosylation site for ATB200. FIG. 19D shows the N-glycosylation profile of the third potential N-glycosylation site for ATB200. FIG. 19E shows the N-glycosylation profile of the fourth potential N-glycosylation site for ATB200. FIG. 19F shows the N-glycosylation profile of the fifth potential N-glycosylation site for ATB200. FIG. 19G shows the N-glycosylation profile of the sixth potential N-glycosylation site for ATB200. FIG. 19H shows the N-glycosylation profile of the seventh potential N-glycosylation site for ATB200.

FIG. 20A-FIG. 20B further characterize and summarize the N-glycosylation profile of ATB200, as also shown in FIGS. 19A-19H. FIG. 20A shows 2-Anthranilic acid (2-AA) glycan mapping and LC/MS-MS analysis of ATB200 and summarizes the N-glycan species identified in ATB200 as a percentage of total fluorescence. Data from 2-AA glycan mapping and LC-MS/MS analysis are also depicted in Table 7. FIG. 20B summarizes the average site occupancy and average N-glycan profile, including total phosphorylation, mono-phosphorylation, bis-phosphorylation, and sialylation, for all seven potential N-glycosylation sites for ATB200. ND=not detected.

FIG. 21 shows the ATB200-03 study design schematic.

FIG. 22 shows the baseline 6-minute walk distance (6MWD) and sitting forced vital capacity (FVC) characteristics of the 122 subjects who participated in the ATB200-03 study. AT-GAA group: subjects who received the ATB200/AT2221 treatment; Alglucosidase alfa group: subjects who received the alglucosidase alfa/placebo treatment.

FIG. 23A depicts the 6MWD and FVC data, showing the baseline, change from the baseline (“CFBL”) at week 52, difference, and P-value, for the overall population (n=122). AT-GAA group: subjects who received the ATB200/miglustat treatment; Alglucosidase alfa group: subjects who received the alglucosidase alfa/placebo treatment.

FIG. 23B depicts the 6MWD and FVC data showing change from the baseline over time for the overall population (n=122). Cipaglucosidase alfa/miglustat group: subjects who received the ATB200/miglustat treatment; Alglucosidase alfa/placebo: subjects who received the alglucosidase alfa/placebo treatment.

FIG. 24 depicts the 6MWD and FVC data, showing the baseline, CFBL at week 52, difference, and P-value, for the ERT-experienced population (n=95). AT-GAA group: subjects who received the ATB200/miglustat treatment; Alglucosidase alfa group: subjects who received the alglucosidase alfa/placebo treatment.

FIG. 25 depicts the 6MWD and FVC changes relative to baseline at week 12, week 26, and week 38, and week 52, for the ERT-experienced population (n=95).

FIG. 26A depicts the 6MWD and FVC data, showing the baseline, CFBL at week 52, difference, and P-value, for the ERT-naïve population (n=27). AT-GAA group: subjects who received the ATB200/miglustat treatment; Alglucosidase alfa group: subjects who received the alglucosidase alfa/placebo treatment.

FIG. 26B depicts the 6MWD and FVC data showing change from the baseline over time for the ERT-naïve population (n=27). Cipaglucosidase alfa/miglustat group: subjects who received the ATB200/miglustat treatment; Alglucosidase alfa/placebo: subjects who received the alglucosidase alfa/placebo treatment.

FIG. 27 depicts baseline characteristics on key secondary endpoints and biomarkers for the overall and ERT-experienced populations. AT-GAA group: subjects who received the ATB200/miglustat treatment; Alglucosidase alfa group: subjects who received the alglucosidase alfa/placebo treatment.

FIG. 28 depicts the lower manual muscle testing (MMT) changes relative to baseline at week 12, week 26, week 38, and week 52, for the overall population (left) and ERT-experienced population (right).

FIG. 29 depicts the gait, stairs, gowers, chair (GSGC) changes relative to baseline at week 12, week 26, week 38, and week 52, for the overall population (left) and ERT-experienced population (right). Cipaglucosidase alfa/miglustat group: subjects who received the ATB200/miglustat treatment; Alglucosidase alfa/placebo: subjects who received the alglucosidase alfa/placebo treatment.

FIG. 30 depicts the patient-reported outcomes measurement information system (PROMIS) for physical function changes relative to baseline at week 12, week 26, week 38, and week 52, for the overall population (left) and ERT-experienced population (right).

FIG. 31 depicts the PROMIS for fatigue changes relative to baseline at week 12, week 26, week 38, and week 52, for the overall population (left) and ERT-experienced population (right).

FIG. 32 depicts the creatine kinase (CK) biomarker changes relative to baseline at week 12, week 26, week 38, and week 52, for the overall population (left) and ERT-experienced population (right).

FIG. 33 depicts the urine hexose tetrasaccharide (Hex4) biomarker changes relative to baseline at week 12, week 26, week 38, and week 52, for the overall population (left) and ERT-experienced population (right).

FIG. 34 shows the primary, secondary and biomarker endpoint heat map for the overall population (left) and ERT-experienced population (right). AT-GAA group: subjects who received the ATB200/miglustat treatment; Alglucosidase alfa group: subjects who received the alglucosidase alfa/placebo treatment.

FIG. 35 summarizes the safety data from the ATB200-03 study. AT-GAA group: subjects who received the ATB200/miglustat treatment; Alglucosidase alfa group: subjects who received the alglucosidase alfa/placebo treatment. TEAE: treatment emergent adverse event; IAR: infusion-associated reaction.

FIG. 36 summarizes results from the ATB200-03 study.

FIG. 37 describes the study objectives and statistical methods of the ATB200-03 study.

FIG. 38 describes the primary endpoint and secondary endpoints of the ATB200-03 study.

FIG. 39 summarizes the patient disposition of the ATB200-03 study.

FIG. 40 summarizes the baseline demographics of the ATB200-03 study.

FIG. 41A-FIG. 41B show subgroup analyses for the change from baseline in 6MWD and FVC by baseline status in the overall population (n=122) (FIG. 41A) and ERT-experienced patients (n=95) (FIG. 41B) in the ATB200-03 study.

FIG. 42 shows a list of treatment emergent adverse events (TEAEs) in ≥10% of patients in any group in the ATB200-03 study.

FIG. 43 shows the study design for the Phase I/II ATB200-02 study. The asterisk indicates that prior ERT was with 20 mg/kg alglucosidase alfa Q2W. Q2W, every 2 weeks

FIG. 44 shows a summary of endpoints and cohorts reported for the ATB200-02 study.

FIG. 45 shows the baseline characteristics and patient disposition for the ATB200-02 study. The asterisk indicates that 1 ERT-naïve patient had received 1 dose of alglucosidase alfa >6 months prior to study entry. M means meters; M:F means male:female ratio; N/A means not applicable; SD means standard deviation.

FIG. 46A-FIG. 46B show the mean change from baseline (CFBL) in 6-minute walk distance (6MWD) over time in ERT-experienced (FIG. 46A) and ERT-naïve (FIG. 46B) subjects in the ATB200-02 study.

FIG. 47A-FIG. 47B show the mean change from baseline (CFBL) in percentage predicted sitting forced vital capacity (FVC) over time in ERT-experienced (FIG. 47A) and ERT-naïve (FIG. 47B) subjects in the ATB200-02 study.

FIG. 48A-FIG. 48B show the mean change from baseline (CFBL) in manual muscle testing (MMT) lower extremity score over time in ERT-experienced (FIG. 48A) and ERT-naïve (FIG. 48B) subjects in the ATB200-02 study.

FIG. 49A-FIG. 49B show the mean percentage change from baseline (CFBL) in urine hexose tetrasaccharide (Hex4) levels (FIG. 49A) and plasma creatine kinase (CK) levels (FIG. 49B) in ERT-experienced and ERT-naïve subjects in the ATB200-02 study.

FIG. 50 shows a summary of treatment emergent adverse events (TEAEs) in the ATB200-02 study. Asterisk indicates diffuse large B-cell lymphoma. IAR means infusion-associated reaction; TEAE means treatment-emergent adverse event with onset date on or after first dose of study drug.

FIG. 51 shows a comparison of the long-term effects of cipaglucosidase alfa/miglustat and avalglucosidase alfa on change from baseline for 6MWD and percentage predicted FVC (sitting) in ERT-experienced subjects.

FIG. 52 shows a comparison of the long-term effects of cipaglucosidase alfa/miglustat and avalglucosidase alfa on change from baseline for 6MWD and percentage predicted FVC (sitting) in ERT-naïve subjects.

FIG. 53A-FIG. 53B show the 6-minute walk test (6MWT) percentage predicted during treatment of ERT-experienced subjects with alglucosidase alfa. FIG. 53B shows replotted data from FIG. 53A only from year 2 onward.

FIG. 54 shows the FVC percentage predicted during treatment of ERT-experienced subjects with alglucosidase alfa.

FIG. 55 shows a summary of endpoints and cohorts for Cohort 2 (non-ambulatory ERT-experienced patients) of the ATB200-02 study.

FIG. 56 shows the baseline characteristics and patient disposition for Cohort 2 (non-ambulatory ERT-experienced patients) of the ATB200-02 study. The asterisk indicates that baseline assessment is the last non-missing result on or prior to the administration of the first dose of study medication (20 mg/kg cipaglucosidase alfa+260 mg miglustat co-administration dose). M:F means male:female ratio; SD means standard deviation.

FIG. 57 show the mean change from baseline (CFBL) in percentage predicted sitting forced vital capacity (FVC) over time in Cohort 2 (non-ambulatory ERT-experienced patients).

FIG. 58 shows a summary of treatment emergent adverse events (TEAEs) for Cohort 2 (non-ambulatory ERT-experienced patients) in the ATB200-02 study. Asterisk indicates urticaria considered to be an IAR. IAR means infusion-associated reaction; TEAE means treatment-emergent adverse event with onset date on or after first dose of study drug.

FIG. 59 shows the baseline characteristics of the seven clinical studies identified by the systematic literature review (SLR)

FIG. 60 shows longitudinal efficacy results versus trial for 6MWD (m) and FVC (% predicted) as changed from baseline for each of the identified studies.

FIG. 61 shows a Network for 6MWD (m) and sitting FVC (% predicted).

FIG. 62 shows a forest plot of relative effect estimates with 95% credible intervals for 6MWD in the base-case scenario (main analysis).

FIG. 63 shows a forest plot of relative effect estimates with 95% credible intervals for FVC in the base-case scenario (main analysis).

FIG. 64 shows a forest plot of relative effect estimates with 95% credible intervals for 6MWD by ERT duration.

FIG. 65 shows a forest plot of relative effect estimates with 95% credible intervals for FVC by ERT duration.

FIG. 66 shows a forest plot of relative effect estimates with 95% credible intervals for 6MWD in the base-case scenario (sensitivity analysis).

FIG. 67 shows a forest plot of relative effect estimates with 95% credible intervals for FVC in the base-case scenario (sensitivity analysis).

FIG. 68 shows a line plot of NT-proBNP for a pediatric patient over the course of treatment with Myozyme and ATB200/AT2221.

FIG. 69 shows a line plot of echocardiography shortening fraction (fs) for a pediatric patient over the course of treatment with Myozyme and ATB200/AT2221.

FIG. 70 shows a GMFM Map for a pediatric patient over the course of treatment with Myozyme and ATB200/AT2221.

FIG. 71 shows a plot of urinary Hex4 for a pediatric patient over the course of treatment with Myozyme and ATB200/AT2221.

FIG. 72A-72C show a cine MRI of the long axis (FIG. 72A) short axis of the left ventricle (FIG. 72B) and the apex (FIG. 72C).

FIG. 73 shows the mean±SD (error bars) describing the concentration of miglustat (ng/ml) for the capsule or prototypes 1, 2, 3, or 4 over the course of 24 hours in rabbits.

FIG. 74 shows the log-transformed mean±SD (error bars) describing the concentration of miglustat (ng/mL) for the capsule or prototypes 1, 2, 3, or 4 over the course of 24 hours in rabbits.

FIG. 75 shows PK summary plots of the mean±SD (error bars) and log-transformed mean±SD (error bars) miglustat concentration over the course of 72 hours after administration to humans either prototype 3 or 4 at a dose of 260 mg.

FIG. 76 shows PK summary plots of the mean±SD (error bars) and log-transformed mean±SD (error bars) miglustat concentration over the course of 72 hours after administration to humans either prototype 3 or 4 at a dose of 1040 mg.

FIG. 77 shows a dose proportionality PK summary plot over the course of 72 hours for the mean±SD (error bars) miglustat concentration in humans following the administration of prototype 3 or 4 at a dose of either 260 mg or 1040 mg.

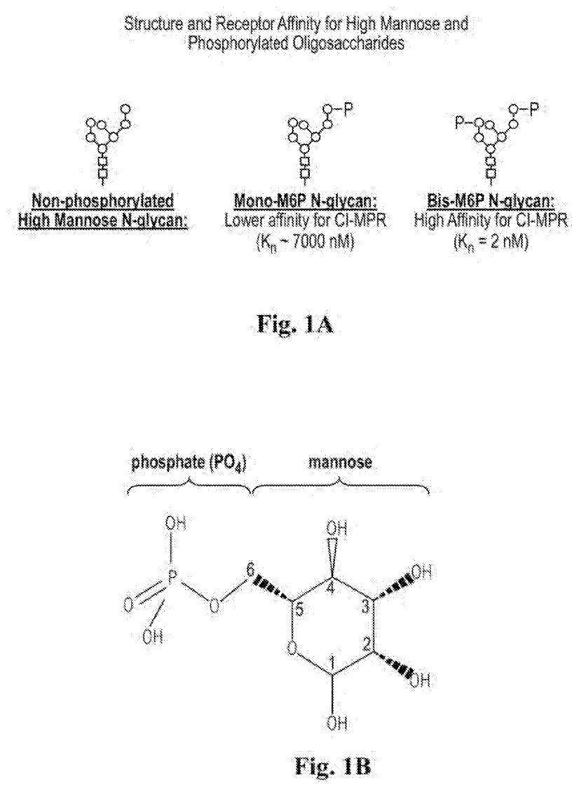

### DETAILED DESCRIPTION

Provided herein are methods for treating infantile-onset Pompe disease (IOPD) comprising administering to an individual less than 18 years of age a recombinant human α-glucosidase (rhGAA) and an enzyme stabilizer. The methods may provide benefits such as improvement in cardiac function (e.g. LVMi or ejection fraction), motor function, muscle strength and/or pulmonary function. The methods provided herein also have a favorable safety profile. The benefits of the methods provided herein are an improvement over the current enzyme replacement therapy (ERT) options for treating Pompe disease.

The current ERT options for treating Pompe disease offer improvements for a limited duration, followed by a slow decline. In 2012, a systematic review of all studies performed in subjects with late-onset Pompe disease (LOPD) was performed (Toscano and Schoser, (2013) J Neurol 260, 951-959). The review included data on 368 subjects with LOPD from published studies, including 27 juvenile subjects (age range: 2 to 17 years old) and 251 adult subjects who received alglucosidase alfa for at least 2 preceding years. Results indicated that >30% of subjects did not show an initial improvement during treatment with alglucosidase alfa and continued to experience deterioration of muscular and respiratory functions despite treatment. In the group of subjects who initially responded to alglucosidase alfa treatment, several additional longer-term studies showed that improvements usually lasted for only approximately 2 years. Thereafter, subjects generally plateaued before beginning to progressively decline.

In 2012, the United Kingdom Health Technology Assessment program, as part of the National Institute for Health Research (Wyatt et al., (2012) Health Technol Assess 16(39), issued recommendations drawn from review of longitudinal data for 81 patients with Pompe disease (including infantile- and late-onset forms (children and adults)) who received the current approved ERT standard of care, alglucosidase alfa. Key markers of Pompe disease progression (forced vital capacity, ventilation dependency, mobility, 6-Minute Walk Test, muscle strength, and body mass index) were assessed and modeled with time of treatment on alglucosidase alfa therapy. Results of this assessment indicated that improvements in FVC, 6-minute walk distance, and muscle strength by patients with LOPD occurred for the first 2 years after commencing ERT with alglucosidase alfa, and decline occurred with continuing treatment beyond this timeframe. Additionally, a 3-year study in 38 subjects with LOPD receiving alglucosidase alfa showed that the subjects demonstrated an improvement in motor function in the first year of treatment, which remained generally stable in the second year and began to decline in the third year (Regnery et al., (2012) Journal of Inherited Metabolic Disease 35:837-845).

Furthermore, a report providing a 10-year follow-up on a Phase 3 LUMIZYME® (Genzyme Corporation) study showed that after experiencing some improvement in motor and pulmonary function during the first couple of years of treatment, subjects began to slowly decline with ongoing treatment (van der Ploeg et al., (2017) European journal of neurology 24.6:768-e31). In the study, from years 3 to 6 on therapy, there was an average decline of approximately 10% in percent predicted baseline 6 minute walk distance, with approximately 80% of subjects experiencing a decline.

The most serious tolerability issue with alglucosidase alfa is the occurrence of infusion-associated reactions (IARs), which, in some instances can include life-threatening anaphylaxis or other severe allergic responses (MYOZYME® Summary of Product Characteristics, December 2018). Management of these events include dose reduction, reduced infusion rates and prolonged infusion times, and dose interruption or discontinuation. Premedication with antihistamines and steroids (prior to infusion) is also regularly used to prevent and reduce the occurrence and severity of IARs and hypersensitivities related to alglucosidase alfa infusion. Despite these measures, patients with Pompe disease may still experience IARs, and some cannot tolerate regular infusions of the currently approved ERT.

In 2017, a systematic review of the literature was undertaken by the European Pompe Consortium, a network of experts from 11 European countries in the field of Pompe disease (van der Ploeg et al., (2017) European journal of neurology 24.6:768-e31). Based on the data obtained from one clinical study and 43 observational studies, covering a total of 586 individual adult subjects, evidence of an effect of ERT at group level was assessed by the consortium. The current European Pompe Consortium consensus is to discontinue ERT therapy upon the occurrence of severe IARs or the progressive clinical worsening of disease symptoms, as well as occurrence of high-neutralizing antibody (Ab) titers, which effectively inactivate the existing ERT treatment. The European Pompe Consortium consensus recommendation also included consideration for re-initiation of ERT treatment if disease progression and clinical worsening recur after ERT has been stopped.

The cellular uptake of a rhGAA molecule is facilitated by the specialized carbohydrate, mannose-6-phosphate (M6P), which binds to the cation-independent mannose-6-phosphate receptor (CIMPR) present on target cells such as muscle cells. Upon binding, rhGAA molecule is taken up by target cells and subsequently transported into the lysosomes within the cells. Most of the conventional rhGAA products, however, lack a high total content of mono-M6P- and bis-M6P-bearing N-glycans (i.e., N-glycans bearing one M6P residue or N-glycans bearing two M6P residues, respectively), which limits their cellular uptake via CIMPR and lysosomal delivery, thus making conventional enzyme replacement therapy insufficiently effective. For example, while conventional rhGAA products at 20 mg/kg or higher doses do ameliorate some aspects of Pompe disease, they are not able to adequately, among other things, (i) treat the underlying cellular dysfunction, (ii) restore muscle structure, or (iii) reduce accumulated glycogen in many target tissues, such as skeletal muscles, to reverse disease progression. Further, higher doses may impose additional burdens on the subject as well as medical professionals treating the subject, such as lengthening the infusion time needed to administer rhGAA intravenously.

The glycosylation of GAA or rhGAA can be enzymatically modified in vitro by the phosphotransferase and uncovering enzymes described by Canfield, et al., U.S. Pat. No. 6,534,300, to generate M6P groups. However, enzymatic glycosylation cannot be adequately controlled and can produce rhGAA having undesirable immunological and pharmacological properties. Enzymatically modified rhGAA may contain only high-mannose oligosaccharide which all could be potentially enzymatically phosphorylated in vitro with a phosphotransferase or uncovering enzyme. The glycosylation patterns produced by in vitro enzymatic treatment of GAA are problematic because the additional terminal mannose residues, particularly non-phosphorylated terminal mannose residues, negatively affect the pharmacokinetics of the modified rhGAA. When such an enzymatically modified product is administered in vivo, these mannose groups increase non-productive clearance of the GAA, increase the uptake of the enzymatically-modified GAA by immune cells, and reduce rhGAA therapeutic efficacy due to less of the GAA reaching targeted tissues, such as skeletal muscle myocytes. For example, terminal non-phosphorylated mannose residues are known ligands for mannose receptors in the liver and spleen which leads to rapid clearance of the enzymatically-modified rhGAA and reduced targeting of rhGAA to target tissue. Moreover, the glycosylation pattern of enzymatically-modified GAA having high mannose N-glycans with terminal non-phosphorylated mannose residues resembles that on glycoproteins produced in yeasts and molds, and increases the risk of triggering immune or allergic responses, such as life-threatening severe allergic (anaphylactic) or hypersensitivity reactions, to the enzymatically modified rhGAA.

Compared with conventional recombinant rhGAA products and in vitro-phosphorylated rhGAA, the rhGAA used in the two-component therapy described herein has an optimized N-glycan profile for enhanced biodistribution and lysosomal uptake, thereby minimizing non-productive clearance of rhGAA once administered. These formulations provide stable or declining Pompe patients an effective therapy that reverses disease progression at the cellular level-including clearing lysosomal glycogen more efficiently than the current standard of care. Patients treated with the two-component therapy comprising rhGAA and an enzyme stabilizer (e.g., miglustat) exhibit significant health improvements, including improvements in cardiac function, muscle strength, motor function, and/or pulmonary function, and/or including a reversal in disease progression.

Moreover, a comparison of three randomized clinical trials (LOTS: alglucosidase alfa vs placebo; COMET: avalglucosidase alfa vs alglucosidase alfa; and PROPEL: cipaglucosidase alfa/miglustat vs alglucosidase alfa) share 6MWD and % predicted FVC as key primary or secondary endpoints. Using patient-level data from the PROPEL randomized clinical trial (RCT) plus aggregate published data from other RCTs, Phase I/II and open label extension trials, a multi-level network meta-regression was conducted, adjusting for various baseline covariates including previous ERT duration. The base case scenario had covariates resembling the PROPEL population (naïve and ERT-experienced) and analyzed 6MWD and FVC change from baseline at 52 weeks. Cipaglucosidase alfa/miglustat was favored vs. alglucosidase alfa and avalglucosidase alfa for 6MWD and FVC, with 6MWD relative effects of 16.3 meters (95% confidence interval: 9.6-24.3) and 29.5 meters (7.4-52.6), respectively, and FVC relative effects of 3.1% (2.4-3.8) and 2.8% (1.0-4.6), respectively. For naïve subjects, relative effects of cipaglucosidase alfa/miglustat remained significant for both endpoints, except cipaglucosidase alfa/miglustat vs. Aval for FVC: 0.4% (−0.8-1.7). These findings suggest cipaglucosidase alfa/miglustat may be clinically differentiated for LOPD patients on key motor and respiratory endpoints, particularly in the ERT-experienced population.

Some benefits of this two-component therapy are described in Gragnaniello et al., “Newborn screening for Pompe disease in Italy: Long-term results and future challenges,” Molecular Genetics and Metabolism Reports 33 (2022), which is hereby incorporated by reference in its entirety. Gragnaniello 2022 showed that an IOPD patient receiving cipaglucosidase alfa/miglustat had improvements in serum creatine phosphokinase (CPK), urine glucose tetrasaccharide (Glc4) and left ventricular mass index (LVMi) after switching from the standard-of-care ERT.

### Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them. The articles “a” and “an” refer to one or to more than one (i.e., to at least one) of the grammatical object of the article. The term “or” means, and is used interchangeably with, the term “and/or,” unless context clearly indicates otherwise. In this application, the use of the singular includes the plural unless specifically stated otherwise. Furthermore, the use of the term “including,” as well as other forms, such as “includes” and “included,” are not limiting. Any range described herein will be understood to include the endpoints and all values between the endpoints. In the present specification, except where the context requires otherwise due to express language or necessary implication, the word “comprises”, or variations such as “comprising” is used in an inclusive sense, i.e., to specify the presence of the stated features but not to preclude the presence or addition of further features in various embodiments of the disclosure.

The term “GAA” refers to human acid α-glucosidase (GAA) enzyme that catalyzes the hydrolysis of α-1,4- and α-1,6-glycosidic linkages of lysosomal glycogen as well as to insertional, relational, or substitution variants of the GAA amino acid sequence and fragments of a longer GAA sequence that exert enzymatic activity. Human acid α-glucosidase is encoded by the GAA gene (National Centre for Biotechnology Information (NCBI) Gene ID 2548), which has been mapped to the long arm of chromosome 17 (location 17q25.2-q25.3). An exemplary amino acid sequence of GAA is NP 000143.2, which is incorporated by reference. This disclosure also encompasses DNA sequences that encode the amino acid sequence of NP 000143.2. More than 500 mutations have currently been identified in the human GAA gene, many of which are associated with Pompe disease. Mutations resulting in misfolding or misprocessing of the acid α-glucosidase enzyme include T1064C (Leu355Pro) and C2104T (Arg702Cys). In addition, GAA mutations which affect maturation and processing of the enzyme include Leu405Pro and Met519Thr. The conserved hexapeptide WIDMNE (SEQ ID NO: 7) at amino acid residues 516-521 is required for activity of the acid α-glucosidase protein. As used herein, the abbreviation “GAA” is intended to refer to human acid α-glucosidase enzyme, while the italicized abbreviation “GAA” is intended to refer to the human gene coding for the human acid α-glucosidase enzyme. The italicized abbreviation “Gaa” is intended to refer to non-human genes coding for non-human acid α-glucosidase enzymes, including but not limited to rat or mouse genes, and the abbreviation “Gaa” is intended to refer to non-human acid α-glucosidase enzymes.

The term “rhGAA” is intended to refer to the recombinant human acid α-glucosidase enzyme and is used to distinguish endogenous GAA from synthetic or recombinant-produced GAA (e.g., GAA produced from CHO cells or other host cells transformed with DNA encoding GAA). The term “rhGAA” encompasses a population of individual rhGAA molecules. Characteristics of the population of rhGAA molecules are provided herein. The term “conventional rhGAA product” is intended to refer to products containing alglucosidase alfa, such as LUMIZYME®, MYOZYME®, or avalglucosidase alfa such as NEXVIAZYME®.

The term “genetically modified” or “recombinant” refers to cells, such as CHO cells, that express a particular gene product, such as rhGAA, following introduction of a nucleic acid comprising a coding sequence which encodes the gene product, along with regulatory elements that control expression of the coding sequence. Introduction of the nucleic acid may be accomplished by any method known in the art including gene targeting and homologous recombination. As used herein, the term also includes cells that have been engineered to express or overexpress an endogenous gene or gene product not normally expressed by such cell, e.g., by gene activation technology.

As used herein, the term “alglucosidase alfa” is intended to refer to a recombinant human acid α-glucosidase identified as [199-arginine,223-histidine]prepro-α-glucosidase (human); Chemical Abstracts Registry Number 420794-05-0. Alglucosidase alfa is approved for marketing in the United States by Genzyme, as the products LUMIZYME® and MYOZYME®.

As used herein, the term “ATB200” is intended to refer to a recombinant human acid α-glucosidase described in International Pat. App. No. PCT/2015/053252, U.S. Pat. Nos. 10,208,299, and 10,961,522, the disclosures of which are herein incorporated by reference. ATB200 is also referred to as “cipaglucosidase alfa” or “cipaglucosidase alfa-atga.” In some embodiments, “ATB200” refers to a rhGAA with a high content of N-glycans bearing mono-M6P and bis-M6P, which is produced from a GA-ATB200 cell line and purified using methods described herein. Cipaglucosidase alfa is currently marketed under the name POMBILITI™ in the United States and POMBILITI® in the European Union. POMBILITI is currently indicated, in combination with OPFOLDA, for the treatment of adult patients with late-onset Pompe disease. The US Prescribing Information and European Union Summary of Product Characteristics for POMBILITI are hereby incorporated by reference in their entireties.

As used herein, the term “glycan” is intended to refer to an oligosaccharide covalently bound to an amino acid residue on a protein or polypeptide. As used herein, the term “N-glycan” or “N-linked glycan” is intended to refer to a polysaccharide chain attached to an asparagine residue on a protein or polypeptide through covalent binding to a nitrogen atom of the asparagine residue. In some embodiments, the N-glycan units attached to a rhGAA are determined by liquid chromatography-tandem mass spectrometry (LC-MS/MS) utilizing an instrument such as the Thermo Scientific™ Orbitrap Velos Pro™ Mass Spectrometer, Thermo Scientific™ Orbitrap Fusion™ Lumos Tribid™ Mass Spectrometer, or Waters Xevo® G2-XS QTof Mass Spectrometer.

As used herein, forced vital capacity, or “FVC,” is the amount of air that can be forcibly exhaled from the lungs of a subject after the subject takes the deepest breath possible.

As used herein, a “six-minute walk test” (6MWT) is a test for measuring the distance an individual is able to walk over a total of six minutes on a hard, flat surface. The test is conducted by having the individual to walk as far as possible in six minutes.

As used herein, a “ten-meter walk test” (10MWT) is a test for measuring the time it takes an individual in walking shoes to walk ten meters on a flat surface.

As used herein, the compound miglustat, also known as N-butyl-1-deoxynojirimycin or NB-DNJ or (2R,3R,4R,5S)-1-butyl-2-(hydroxymethyl)piperidine-3,4,5-triol, is a compound having the following chemical formula:

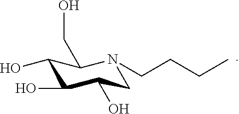

Alternatively shown as:

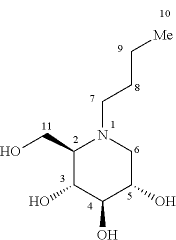 [SMILES: CCCCN1C[C@H](O)[C@@H](O)[C@H](O)[C@H]1CO]

One formulation of miglustat is marketed commercially under the trade name ZAVESCA® as monotherapy for type 1 Gaucher disease. In some embodiments, miglustat is referred to as AT2221.

Another formulation of miglustat is currently marketed under the name OPFOLDA™ in the United States and OPFOLDA® in the European Union. OPFOLDA is currently indicated, in combination with POMBILITI, for the treatment of adult patients with late-onset Pompe disease. The US Prescribing Information and European Union Summary of Product Characteristics for OPFOLDA are hereby incorporated by reference in their entireties.

As discussed below, pharmaceutically acceptable salts of miglustat may also be used in the present disclosure. When a salt of miglustat is used, the dosage of the salt will be adjusted so that the dose of miglustat received by the patient is equivalent to the amount which would have been received had the miglustat free base been used.

As used herein, the compound duvoglustat, also known as 1-deoxynojirimycin or DNJ or (2R,3R,4R,5S)-2-(hydroxymethyl)piperidine-3,4,5-triol, is a compound having the following chemical formula:

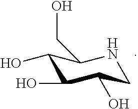

As used herein, the term “enzyme stabilizer” is intended to refer to a molecule that specifically binds to acid α-glucosidase and has one or more of the following effects:
enhances the formation of a stable molecular conformation of the protein;
enhances proper trafficking of the protein from the endoplasmic reticulum to another cellular location, preferably a native cellular location, so as to prevent endoplasmic reticulum-associated degradation of the protein;
prevents aggregation of conformationally unstable or misfolded proteins;
restores and/or enhances at least partial wild-type function, stability, and/or activity of the protein;
improves the phenotype or function of the cell harboring acid α-glucosidase; and/or
stabilizing the acid α-glucosidase in vitro and/or in vivo (e.g., in a patient's bloodstream).

Thus, an enzyme stabilizer for acid α-glucosidase is a molecule that binds to acid α-glucosidase, resulting in proper folding, trafficking, non-aggregation, and/or activity of acid α-glucosidase. In at least one embodiment, the enzyme stabilizer is miglustat. Another non-limiting example of an enzyme stabilizer for acid α-glucosidase is duvoglustat.

As used herein, the term “pharmaceutically acceptable” is intended to refer to molecular entities and compositions that are physiologically tolerable and do not typically produce untoward reactions when administered to a human. Preferably, as used herein, the term “pharmaceutically acceptable” means approved by a regulatory agency of the federal or a state government or listed in the U.S. Pharmacopeia or other generally recognized pharmacopeia for use in animals, and more particularly in humans. As used herein, the term “carrier” is intended to refer to a diluent, adjuvant, excipient, or vehicle with which a compound is administered. Suitable pharmaceutical carriers are known in the art and, in at least one embodiment, are described in “Remington's Pharmaceutical Sciences” by E. W. Martin, 18th Edition, or other editions.

The term “pharmaceutically acceptable salt” as used herein is intended to mean a salt which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Berge et al., J. Pharm. Sci., 1977, 66, pp. 1-19, herein incorporated by reference. The term “pharmaceutically-acceptable acid addition salt” as used herein is intended to mean those salts which retain the biological effectiveness and properties of the free bases and which are not biologically or otherwise undesirable, formed with inorganic acids. The term “pharmaceutically-acceptable base addition salt” as used herein is intended to mean those salts which retain the biological effectiveness and properties of the free acids and which are not biologically or otherwise undesirable, formed with inorganic bases.

As used herein, the term “buffer” refers to a solution containing a weak acid and its conjugate base or a weak base and its conjugate acid that helps to prevent changes in pH.

As used herein, the terms “therapeutically effective dose” and “effective amount” are intended to refer to an amount of acid α-glucosidase and/or of miglustat and/or of a two-component therapy thereof, which is sufficient to result in a therapeutic response in a subject.

The therapeutic response may also include molecular responses such as glycogen accumulation, lysosomal proliferation, and formation of autophagic zones. The therapeutic responses may be evaluated by comparing physiological and molecular responses of muscle biopsies before and after treatment with a rhGAA described herein. For instance, the amount of glycogen present in the biopsy samples can be used as a marker for determining the therapeutic response. Another example includes biomarkers such as LAMP-1, LC3, and Dysferlin, which can be used as an indicator of lysosomal storage dysfunction. For instance, muscle biopsies collected prior to and after treatment with a rhGAA described herein may be stained with an antibody that recognizes one or more of the biomarkers. The therapeutic response may also include a decrease in fatigue or improvement in other patient-reported outcomes (e.g., daily living activities, well-being, etc.).

As used herein, the term “enzyme replacement therapy” or “ERT” is intended to refer to the introduction of a non-native, purified enzyme into an individual having a deficiency in such enzyme. The administered protein can be obtained from natural sources or by recombinant expression. The term also refers to the introduction of a purified enzyme in an individual otherwise requiring or benefiting from administration of a purified enzyme. In at least one embodiment, such an individual suffers from enzyme insufficiency. The introduced enzyme may be a purified, recombinant enzyme produced in vitro, or a protein purified from isolated tissue or fluid, such as, for example, placenta or animal milk, or from plants.

As used herein, the term “two-component therapy” is intended to refer to any therapy wherein two or more individual therapies are administered concurrently or sequentially. In some embodiment, the results of the two-component therapy are enhanced as compared to the effect of each therapy when it is performed individually. Enhancement may include any improvement of the effect of the various therapies that may result in an advantageous result as compared to the results achieved by the therapies when performed alone. Enhanced effect or results can include a synergistic enhancement, wherein the enhanced effect is more than the additive effects of each therapy when performed by itself; an additive enhancement, wherein the enhanced effect is substantially equal to the additive effect of each therapy when performed by itself; or less than additive effect, wherein the enhanced effect is lower than the additive effect of each therapy when performed by itself, but still better than the effect of each therapy when performed by itself. Enhanced effect may be measured by any means known in the art by which treatment efficacy or outcome can be measured.

“Pompe disease” refers to an autosomal recessive LSD characterized by deficient acid alpha glucosidase (GAA) activity which impairs lysosomal glycogen metabolism. The enzyme deficiency leads to lysosomal glycogen accumulation and results in progressive skeletal muscle weakness, reduced cardiac function, respiratory insufficiency, and/or CNS impairment at late stages of disease. Genetic mutations in the GAA gene result in either lower expression or produce mutant forms of the enzyme with altered stability, and/or biological activity ultimately leading to disease, (see generally Hirschhorn R, 1995, Glycogen Storage Disease Type II: Acid a-Glucosidase (Acid Maltase) Deficiency, The Metabolic and Molecular Bases of Inherited Disease, Scriver et al., eds., McGraw-Hill, New York, 7th ed., pages 2443-2464). The three recognized clinical forms of Pompe Disease (infantile, juvenile, and adult) are correlated with the level of residual α-glucosidase activity (Reuser A J et al., 1995, Glycogenosis Type II (Acid Maltase Deficiency), Muscle & Nerve Supplement 3, S61-S69). Infantile Pompe disease (type I or A) is most common and most severe, characterized by failure to thrive, generalized hypotonic, cardiac hypertrophy, and cardiorespiratory failure within the second year of life. Juvenile Pompe disease (type II or B) is intermediate in severity and is characterized by a predominance of muscular symptoms without cardiomegaly. Juvenile Pompe individuals usually die before reaching 20 years of age due to respiratory failure. Adult Pompe disease (type III or C) often presents as a slowly progressive myopathy in the teenage years or as late as the sixth decade (Felicia K J et al., 1995, Clinical Variability in Adult-Onset Acid Maltase Deficiency: Report of Affected Sibs and Review of the Literature, Medicine 74, 131-135). In Pompe disease, it has been shown that α-glucosidase is extensively modified post-translationally by glycosylation, phosphorylation, and proteolytic processing. Conversion of the 110 kilodalton (kDa) precursor to 76 and 70 KDa mature forms by proteolysis in the lysosome is required for optimum glycogen catalysis. The formulations and dosing regimens disclosed in this application may be used to treat, for example, Type I, Type II or Type III Pompe disease.

Pompe disease is now considered to be a continuous spectrum of phenotypes, with the clinically most severe, rapidly progressive phenotypes being the classic infantile-onset Pompe disease (IOPD) and the less severe, slowly progressive phenotypes being late-onset Pompe disease (LOPD). Symptoms of Pompe disease can first appear at any point in life. The most severe form of IOPD appears in the first 3 months of life. Muscle weakness, heart dysfunction, and respiratory dysfunction characterize IOPD progression, and life expectancy is approximately 2 years. Atypical infantile, juvenile-onset, and non-classic IOPD are a few of the terms used to describe pediatric patients with Pompe disease who do not have the significant and progressive hypertrophic cardiomyopathy that is seen in classic IOPD, and hence their phenotype is more closely comparable to LOPD.

Late-onset Pompe disease can manifest in childhood or adulthood and does not present with clinically apparent cardiac involvement (Leslie and Bailey, 2017). Late-onset Pompe disease is often referred to as juvenile-onset Pompe disease when occurring in the pediatric subpopulation of the LOPD category. LOPD has a slower rate of progression compared with classic IOPD, with most patients experiencing progressive limb-girdle weakness and respiratory failure due to involvement of muscles in the proximal lower and upper limbs, paraspinal muscles, and diaphragm. Clinical manifestations include difficulty walking and climbing stairs and progressive limitations of motor activities of daily living with progression to a need for ambulatory support followed by wheelchair dependence (Reuser, et al 2001). Clinical manifestations of the disease are compounded by respiratory involvement, initially as sleep-disordered breathing and orthopnea (shortness of breath in supine position). The progressive nature of Pompe disease generally results in the use of invasive mechanically assisted ventilation.

As used herein, “significant” refers to statistical significance. The term refers to statistical evidence that there is a difference between two treatment groups. It can be defined as the probability of making a decision to reject the null hypothesis when the null hypothesis is actually true. The decision is often made using a p-value<0.05 derived from a suitable statistical analysis for the comparison. See, e.g., Example 9.

A “subject” or “patient” is preferably a human, though other mammals and non-human animals having disorders involving accumulation of glycogen may also be treated. A subject may be a fetus, a neonate, child, juvenile, or an adolescent with Pompe disease or other glycogen storage or accumulation disorder. One example of an individual being treated is an individual (fetus, neonate, child, juvenile, or adolescent human) having GSD-II (e.g., infantile GSD-II, or juvenile GSD-II). The individual can have residual GAA activity, or no measurable activity. For example, the individual having GSD-II can have GAA activity that is less than about 1% of normal GAA activity (infantile GSD-II), GAA activity that is about 1-10% of normal GAA activity (juvenile GSD-II). In some embodiments, the subject or patient is less than 18 years of age. In some embodiments, the subject or patient is an “ERT-experienced” or “ERT-switch” patient, referring to a Pompe disease patient who has previously received enzyme replacement therapy. In some embodiments, an “ERT-experienced” or “ERT-switch” patient is a Pompe disease patient who has received or is currently receiving alglucosidase alfa for greater than or equal to 24 months. In some embodiments, an “ERT-experienced” or “ERT-switch” patient is a Pompe disease patient who is declining on currently approved ERT (e.g., MYOZYME®, LUMIZYME®, or NEXVIAZYME®). In some embodiments, the subject is a patient who has previously received enzyme replacement therapy (ERT). In some embodiments, the subject or patient is an “ERT-naïve” patient, referring to a Pompe disease patient who has not previously received enzyme replacement therapy. In certain embodiments, the subject or patient is ambulatory (e.g., an ambulatory ERT-switch patient or an ambulatory ERT-naïve patient). In certain embodiments, the subject or patient is nonambulatory (e.g., a nonambulatory ERT-switch patient). Ambulatory or nonambulatory status may be determined by a six-minute walk test (6MWT). In some embodiments, an ambulatory patient is a Pompe disease patient who is able to walk at least 200 meters in the 6MWT. In some embodiments, a nonambulatory patient is a Pompe disease patient who is unable to walk unassisted or who is wheelchair bound. In some embodiments, the subject is using effective contraception. In some embodiments, the subject and/or the subject's partner are using highly effective contraception, such as one that results in a low failure rate (e.g., <1% per year) when used consistently and correctly. Examples of highly effective methods of contraception include, but are not limited to: total abstinence; combined (estrogen- and progestogen-containing) hormonal contraception associated with inhibition of ovulation; oral, intravaginal, transdermal progestogen-only hormonal contraception associated with inhibition of ovulation: oral, injectable, implantable intrauterine device; intrauterine hormone-releasing system; bilateral tubal occlusion; and vasectomy. In some embodiments, the subject is post-menopausal. In some embodiments, the subject is not of child-bearing potential. In some embodiments, the subject is permanently sterile. In some embodiments, the subject is not pregnant. In some embodiments, the subject is not breastfeeding.

In some embodiments, the patient has previously received enzyme replacement therapy (ERT). In some embodiments, the patient is declining on currently approved ERT (e.g., MYOZYME®, LUMIZYME® or NEXVIAZYME®). In some embodiments, if of reproductive potential, both male and female patients have agreed to use a highly effective method of contraception throughout the duration of the treatment and for up to 90 days after their last dose. In some embodiments, patients who are taking β2-receptor agonists or non-selective β-blockers (e.g., propranolol, nadolol, carvedilol) maintain a stable dose as appropriate and determined by the treating physician.

The terms “treat” and “treatment,” as used herein, refer to amelioration of one or more symptoms associated with the disease, delay of the onset of one or more symptoms of the disease, and/or lessening of the severity or frequency of one or more symptoms of the disease. For example, treatment can refer to improvement of cardiac status (e.g. increase of end-diastolic and/or end-systolic volumes, or reduction or amelioration of the progressive cardiomyopathy that is typically found in GSD-II) or of pulmonary function (e.g., increase in crying vital capacity over baseline capacity, and/or normalization of oxygen desaturation during crying); improvement in neurodevelopment and/or motor skills (e.g., increase in AIMS score); reduction of glycogen levels in tissue of the individual affected by the disease; or any combination of these effects. In one embodiment, treatment includes improvement of cardiac status, particularly in reduction of GSD-II-associated cardiomyopathy.

The terms “improve,” “increase,” and “reduce,” as used herein, indicate values that are relative to a baseline measurement or the corresponding values from a control treatment, such as a measurement in the same individual prior to initiation of the treatment described herein, a measurement in a control individual (or multiple control individuals) in the absence of the treatment described herein, or a measurement after a control treatment. A control individual is an individual afflicted with the same form of GSD-II (either infantile, juvenile, or adult-onset) as the individual being treated, who is about the same age as the individual being treated (to ensure that the stages of the disease in the treated individual and the control individual(s) are comparable). In some embodiments, a control treatment comprises administering alglucosidase alfa and a placebo for an enzyme stabilizer (see Example 9).

As used herein, the phrases “stabilizing motor function”, “stabilizing pulmonary function”, and similar terms refer to reducing or arresting the decline in motor and pulmonary function, and/or restoring motor and/or pulmonary function. As untreated Pompe patients are expected to have significant decreases in motor function and pulmonary function over time, enhancements in the rate of motor and/pulmonary function deterioration and/or enhancements in motor and/pulmonary function demonstrate a benefit of therapy as described herein. Moreover, as ERT-experienced Pompe patients often continue to decline in motor and/pulmonary function over time, stabilizing motor and/or pulmonary function using the therapy described herein can include reducing and/or arresting the decline in motor and/or pulmonary function compared to such patients receiving the previous ERT treatment (e.g., MYOZYME®, LUMIZYME®, or NEXVIAZYME®).

As used herein, the terms “about” and “approximately” are intended to refer to an acceptable degree of error for the quantity measured given the nature or precision of the measurements. For example, the degree of error can be indicated by the number of significant figures provided for the measurement, as is understood in the art, and includes but is not limited to a variation of ±1 in the most precise significant figure reported for the measurement. Typical exemplary degrees of error are within 20 percent (%), preferably within 10%, and more preferably within 5% of a given value or range of values. Numerical quantities given herein are approximate unless stated otherwise, meaning that the term “about” or “approximately” can be inferred when not expressly stated.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

The section headings used herein are for organizational purposes only and are not to be construed as limiting the subject matter described.

### Recombinant Human Acid A-Glucosidase (Rhgaa)

In some embodiments, the recombinant human acid α-glucosidase (rhGAA) is an enzyme having an amino acid sequence as set forth in SEQ ID NO: 1, SEQ ID NO: 3, SEQ ID NO: 4, SEQ ID NO: 5, or SEQ ID NO: 6. In some embodiments, the rhGAA is encoded by a nucleotide sequence as set forth in SEQ ID NO: 2.

TABLE 1
Nucleotide Sequences and Protein Sequences

| SEQ ID | |
|---|---|
| NO | Sequences |
| 1 | MGVRHPPCSHRLLAVCALVSLATAALLGHILLHDFLLVPRELSGSSPVLEETHPAHQQGA |
| | SRPGPRDAQAHPGRPRAVPTQCDVPPNSRFDCAPDKAITQEQCEARGCCYIPAKQGLQGA |
| | QMGQPWCFFPPSYPSYKLENLSSSEMGYTAILTRITPTFFPKDILTLRLDVMMETENRLH |
| | FTIKDPANRRYEVPLETPRVHSRAPSPLYSVEFSEEPFGVIVHRQLDGRVLLNTTVAPLF |
| | FADQFLQLSTSLPSQYITGLAEHLSPLMLSTSWTRITLWNRDLAPTPGANLYGSHPFYLA |
| | LEDGGSAHGVFLLNSNAMDVVLQPSPALSWRSTGGILDVYIFLGPEPKSVVQQYLDVVGY |
| | PFMPPYWGLGFHLCRWGYSSTAITRQVVENMTRAHFPLDVQWNDLDYMDSRRDFTFNKDG |
| | FRDFPAMVQELHQGGRRYMMIVDPAISSSGPAGSYRPYDEGLRRGVFITNETGQPLIGKV |
| | WPGSTAFPDFTNPTALAWWEDMVAEFHDQVPFDGMWIDMNEPSNFIRGSEDGCPNNELEN |
| | PPYVPGVVGGTLQAATICASSHQFLSTHYNLHNLYGLTEAIASHRALVKARGTRPFVISR |
| | STFAGHGRYAGHWTGDVWSSWEQLASSVPEILQFNLLGVPLVGADVCGFLGNTSEELCVR |
| | WTQLGAFYPFMRNHNSLLSLPQEPYSFSEPAQQAMRKALTLRYALLPHLYTLFHQAHVAG |
| | ETVARPLFLEFPKDSSTWTVDHQLLWGEALLITPVLQAGKAEVTGYFPLGTWYDLQTVPI |
| | EALGSLPPPPAAPREPAIHSEGQWVTLPAPLDTINVHLRAGYIIPLQGPGLTTTESRQQP |
| | MALAVALTKGGEARGELFWDDGESLEVLERGAYTQVIFLARNNTIVNELVRVTSEGAGLQ |
| | LQKVTVLGVATAPQQVLSNGVPVSNFTYSPDTKVLDICVSLLMGEQFLVSWC |
| 2 | cagttgggaaagctgaggttgtcgccggggccgcgggtggaggtcggggatgaggcagcaggt |
| | aggacagtgacctcggtgacgcgaaggaccccggccacctctaggttctcctcgtccgcccgt |
| | tgttcagcgagggaggctctgggcctgccgcagctgacggggaaactgaggcacggagcgggc |
| | ctgtaggagctgtccaggccatctccaaccatgggagtgaggcacccgccctgctcccaccgg |
| | ctcctggccgtctgcgccctcgtgtccttggcaaccgctgcactcctggggcacatcctactc |
| | catgatttcctgctggttccccgagagctgagtggctcctccccagtcctggaggagactcac |
| | ccagctcaccagcagggagccagcagaccagggccccgggatgcccaggcacaccccggccgt |
| | cccagagcagtgcccacacagtgcgacgtcccccccaacagccgcttcgattgcgcccctgac |
| | aaggccatcacccaggaacagtgcgaggcccgcggctgctgctacatccctgcaaagcagggg |
| | ctgcagggagcccagatggggcagccctggtgcttcttcccacccagctaccccagctacaag |
| | ctggagaacctgagctcctctgaaatgggctacacggccaccctgacccgtaccacccccacc |
| | ttcttccccaaggacatcctgaccctgcggctggacgtgatgatggagactgagaaccgcctc |
| | cacttcacgatcaaagatccagctaacaggcgctacgaggtgcccttggagaccccgcgtgtc |
| | cacagccgggcaccgtccccactctacagcgtggagttctccgaggagcccttcggggtgatc |
| | gtgcaccggcagctggacggccgcgtgctgctgaacacgacggtggcgcccctgttctttgcg |
| | gaccagttccttcagctgtccacctcgctgccctcgcagtatatcacaggcctcgccgagcac |
| | ctcagtcccctgatgctcagcaccagctggaccaggatcaccctgtggaaccgggaccttgcg |
| | cccacgcccggtgcgaacctctacgggtctcaccctttctacctggcgctggaggacggcggg |
| | tcggcacacggggtgttcctgctaaacagcaatgccatggatgtggtcctgcagccgagccct |
| | gcccttagctggaggtcgacaggtgggatcctggatgtctacatcttcctgggcccagagccc |
| | aagagcgtggtgcagcagtacctggacgttgtgggatacccgttcatgccgccatactggggc |
| | ctgggcttccacctgtgccgctggggctactcctccaccgctatcacccgccaggtggtggag |
| | aacatgaccagggcccacttccccctggacgtccaatggaacgacctggactacatggactcc |
| | cggagggacttcacgttcaacaaggatggcttccgggacttcccggccatggtgcaggagctg |
| | caccagggcggccggcgctacatgatgatcgtggatcctgccatcagcagctcgggccctgcc |
| | gggagctacaggccctacgacgagggtctgcggaggggggttttcatcaccaacgagaccggc |
| | cagccgctgattgggaaggtatggcccgggtccactgccttccccgacttcaccaaccccaca |
| | gccctggcctggtgggaggacatggtggctgagttccatgaccaggtgcccttcgacggcatg |
| | tggattgacatgaacgagccttccaacttcatcagaggctctgaggacggctgccccaacaat |
| | gagctggagaacccaccctacgtgcctggggtggttggggggaccctccaggcggccaccatc |
| | tgtgcctccagccaccagtttctctccacacactacaacctgcacaacctctacggcctgacc |
| | gaagccatcgcctcccacagggcgctggtgaaggctcgggggacacgcccatttgtgatctcc |
| | cgctcgacctttgctggccacggccgatacgccggccactggacgggggacgtgtggagctcc |
| | tgggagcagctcgcctcctccgtgccagaaatcctgcagtttaacctgctgggggtgcctctg |
| | gtcggggccgacgtctgcggcttcctgggcaacacctcagaggagctgtgtgtgcgctggacc |
| | cagctgggggccttctaccccttcatgcggaaccacaacagcctgctcagtctgccccaggag |
| | ccgtacagcttcagcgagccggcccagcaggccatgaggaaggccctcaccctgcgctacgca |
| | ctcctcccccacctctacacactgttccaccaggcccacgtcgcgggggagaccgtggcccgg |
| | cccctcttcctggagttccccaaggactctagcacctggactgtggaccaccagctcctgtgg |
| | ggggaggccctgctcatcaccccagtgctccaggccgggaaggccgaagtgactggctacttc |
| | cccttgggcacatggtacgacctgcagacggtgccaatagaggcccttggcagcctcccaccc |
| | ccacctgcagctccccgtgagccagccatccacagcgaggggcagtgggtgacgctgccggcc |
| | cccctggacaccatcaacgtccacctccgggctgggtacatcatccccctgcagggccctggc |
| | ctcacaaccacagagtcccgccagcagcccatggccctggctgtggccctgaccaagggtgga |
| | gaggcccgaggggagctgttctgggacgatggagagagcctggaagtgctggagcgaggggcc |
| | tacacacaggtcatcttcctggccaggaataacacgatcgtgaatgagctggtacgtgtgacc |
| | agtgagggagctggcctgcagctgcagaaggtgactgtcctgggcgtggccacggcgccccag |
| | caggtcctctccaacggtgtccctgtctccaacttcacctacagccccgacaccaaggtcctg |
| | gacatctgtgtctcgctgttgatgggagagcagtttctcgtcagctggtgttagccgggcgga |
| | gtgtgttagtctctccagagggaggctggttccccagggaagcagagcctgtgtgcgggcagc |
| | agctgtgtgcgggcctgggggttgcatgtgtcacctggagctgggcactaaccattccaagcc |
| | gccgcatcgcttgtttccacctcctgggccggggctctggcccccaacgtgtctaggagagct |
| | ttctccctagatcgcactgtgggccggggcctggagggctgctctgtgttaataagattgtaa |
| | ggtttgccctcctcacctgttgccggcatgcgggtagtattagccacccccctccatctgttc |
| | ccagcaccggagaagggggtgctcaggtggaggtgtggggtatgcacctgagctcctgcttcg |
| | cgcctgctgctctgccccaacgcgaccgcttcccggctgcccagagggctggatgcctgccgg |
| | tccccgagcaagcctgggaactcaggaaaattcacaggacttgggagattctaaatcttaagt |
| | gcaattattttaataaaaggggcatttggaatc |
| 3 | MGVRHPPCSHRLLAVCALVSLATAALLGHILLHDFLLVPRELSGSSPVLEETHPAHQQGA |
| | SRPGPRDAQAHPGRPRAVPTQCDVPPNSRFDCAPDKAITQEQCEARGCCYIPAKQGLQGA |
| | QMGQPWCFFPPSYPSYKLENLSSSEMGYTATLTRTTPTFFPKDILTLRLDVMMETENRLH |
| | FTIKDPANRRYEVPLETPRVHSRAPSPLYSVEFSEEPFGVIVHRQLDGRVLLNTTVAPLF |
| | FADQFLQLSTSLPSQYITGLAEHLSPLMLSTSWTRITLWNRDLAPTPGANLYGSHPFYLA |
| | LEDGGSAHGVFLLNSNAMDVVLQPSPALSWRSTGGILDVYIFLGPEPKSVVQQYLDVVGY |
| | PFMPPYWGLGFHLCRWGYSSTAITRQVVENMTRAHFPLDVQWNDLDYMDSRRDFTFNKDG |
| | FRDFPAMVQELHQGGRRYMMIVDPAISSSGPAGSYRPYDEGLRRGVFITNETGQPLIGKV |
| | WPGSTAFPDFTNPTALAWWEDMVAEFHDQVPFDGMWIDMNEPSNFIRGSEDGCPNNELEN |
| | PPYVPGVVGGTLQAATICASSHQFLSTHYNLHNLYGLTEAIASHRALVKARGIRPFVISR |
| | STFAGHGRYAGHWTGDVWSSWEQLASSVPEILQFNLLGVPLVGADVCGFLGNTSEELCVR |
| | WTQLGAFYPFMRNHNSLLSLPQEPYSFSEPAQQAMRKALTLRYALLPHLYTLFHQAHVAG |
| | ETVARPLFLEFPKDSSTWTVDHQLLWGEALLITPVLQAGKAEVTGYFPLGTWYDLQTVPI |
| | EALGSLPPPPAAPREPAIHSEGQWVTLPAPLDTINVHLRAGYIIPLQGPGLTTTESRQQP |
| | MALAVALTKGGEARGELFWDDGESLEVLERGAYTQVIFLARNNTIVNELVRVTSEGAGLQ |
| | LQKVTVLGVATAPQQVLSNGVPVSNFTYSPDTKVLDICVSLLMGEQFLVSWC |
| 4 | MGVRHPPCSHRLLAVCALVSLATAALLGHILLHDFLLVPRELSGSSPVLEETHPAHQQGA |
| | SRPGPRDAQAHPGRPRAVPTQCDVPPNSRFDCAPDKAITQEQCEARGCCYIPAKQGLQGA |
| | QMGQPWCFFPPSYPSYKLENLSSSEMGYTATLTRTTPTFFPKDILTLRLDVMMETENRLH |
| | FTIKDPANRRYEVPLETPHVHSRAPSPLYSVEFSEEPFGVIVRRQLDGRVLLNTTVAPLF |
| | FADQFLQLSTSLPSQYITGLAEHLSPLMLSTSWTRITLWNRDLAPTPGANLYGSHPFYLA |
| | LEDGGSAHGVFLLNSNAMDVVLQPSPALSWRSTGGILDVYIFLGPEPKSVVQQYLDVVGY |
| | PFMPPYWGLGFHLCRWGYSSTAITRQVVENMTRAHFPLDVQWNDLDYMDSRRDFTFNKDG |
| | FRDFPAMVQELHQGGRRYMMIVDPAISSSGPAGSYRPYDEGLRRGVFITNETGQPLIGKV |
| | WPGSTAFPDFTNPTALAWWEDMVAEFHDQVPFDGMWIDMNEPSNFIRGSEDGCPNNELEN |
| | PPYVPGVVGGTLQAATICASSHQFLSTHYNLHNLYGLTEAIASHRALVKARGTRPFVISR |
| | STFAGHGRYAGHWTGDVWSSWEQLASSVPEILQFNLLGVPLVGADVCGFLGNTSEELCVR |
| | WTQLGAFYPFMRNHNSLLSLPQEPYSFSEPAQQAMRKALTLRYALLPHLYTLFHQAHVAG |
| | ETVARPLFLEFPKDSSTWTVDHQLLWGEALLITPVLQAGKAEVTGYFPLGTWYDLQTVPV |
| | EALGSLPPPPAAPREPAIHSEGQWVTLPAPLDTINVHLRAGYIIPLQGPGLTTTESRQQP |
| | MALAVALTKGGEARGELFWDDGESLEVLERGAYTQVIFLARNNTIVNELVRVTSEGAGLQ |
| | LQKVTVLGVATAPQQVLSNGVPVSNFTYSPDTKVLDICVSLLMGEQFLVSWC |
| 5 | QQGASRPGPRDAQAHPGRPRAVPTQCDVPPNSRFDCAPDKAITQEQCEARGCCYIPAKQG |
| | LQGAQMGQPWCFFPPSYPSYKLENLSSSEMGYTAILTRTTPTFFPKDILTLRLDVMMETE |
| | NRLHFTIKDPANRRYEVPLETPRVHSRAPSPLYSVEFSEEPFGVIVHRQLDGRVLLNTTV |
| | APLFFADQFLQLSTSLPSQYITGLAEHLSPLMLSTSWTRITLWNRDLAPTPGANLYGSHP |
| | FYLALEDGGSAHGVFLLNSNAMDVVLQPSPALSWRSTGGILDVYIFLGPEPKSVVQQYLD |
| | VVGYPFMPPYWGLGFHLCRWGYSSTAITRQVVENMTRAHFPLDVQWNDLDYMDSRRDFTF |
| | NKDGFRDFPAMVQELHQGGRRYMMIVDPAISSSGPAGSYRPYDEGLRRGVFITNETGQPL |
| | IGKVWPGSTAFPDFTNPTALAWWEDMVAEFHDQVPFDGMWIDMNEPSNFIRGSEDGCPNN |
| | ELENPPYVPGVVGGTLQAATICASSHQFLSTHYNLHNLYGLTEAIASHRALVKARGTRPF |
| | VISRSTFAGHGRYAGHWTGDVWSSWEQLASSVPEILQFNLLGVPLVGADVCGFLGNTSEE |
| | LCVRWTQLGAFYPFMRNHNSLLSLPQEPYSFSEPAQQAMRKALTLRYALLPHLYTLFHQA |
| | HVAGETVARPLFLEFPKDSSTWTVDHQLLWGEALLITPVLQAGKAEVTGYFPLGTWYDLQ |
| | TVPIEALGSLPPPPAAPREPAIHSEGQWVTLPAPLDTINVHLRAGYIIPLQGPGLTTTES |
| | RQQPMALAVALTKGGEARGELFWDDGESLEVLERGAYTQVIFLARNNTIVNELVRVTSEG |
| | AGLQLQKVTVLGVATAPQQVLSNGVPVSNFTYSPDTKVLDICVSLLMGEQFLVSWC |
| 6 | QQGASRPGPRDAQAHPGRPRAVPTQCDVPPNSRFDCAPDKAITQEQCEARGCCYIPAKQGLQG |
| | AQMGQPWCFFPPSYPSYKLENLSSSEMGYTATLTRITPTFFPKDILTLRLDVMMETENRLHFT |
| | IKDPANRRYEVPLETPHVHSRAPSPLYSVEFSEEPFGVIVRRQLDGRVLLNTTVAPLFFADQF |
| | LQLSTSLPSQYITGLAEHLSPLMLSTSWTRITLWNRDLAPTPGANLYGSHPFYLALEDGGSAH |
| | GVFLLNSNAMDVVLQPSPALSWRSTGGILDVYIFLGPEPKSVVQQYLDVVGYPFMPPYWGLGF |
| | HLCRWGYSSTAITRQVVENMTRAHFPLDVQWNDLDYMDSRRDFTFNKDGFRDFPAMVQELHQG |
| | GRRYMMIVDPAISSSGPAGSYRPYDEGLRRGVFITNETGQPLIGKVWPGSTAFPDFTNPTALA |
| | WWEDMVAEFHDQVPFDGMWIDMNEPSNFIRGSEDGCPNNELENPPYVPGVVGGTLQAATICAS |
| | SHQFLSTHYNLHNLYGLTEAIASHRALVKARGTRPFVISRSTFAGHGRYAGHWTGDVWSSWEQ |
| | LASSVPEILQFNLLGVPLVGADVCGFLGNTSEELCVRWTQLGAFYPFMRNHNSLLSLPQEPYS |
| | FSEPAQQAMRKALTLRYALLPHLYTLFHQAHVAGETVARPLFLEFPKDSSTWTVDHQLLWGEA |
| | LLITPVLQAGKAEVTGYFPLGTWYDLQTVPVEALGSLPPPPAAPREPAIHSEGQWVTLPAPLD |
| | TINVHLRAGYIIPLQGPGLTTTESRQQPMALAVALTKGGEARGELFWDDGESLEVLERGAYTQ |
| | VIFLARNNTIVNELVRVTSEGAGLQLQKVTVLGVATAPQQVLSNGVPVSNFTYSPDTKVLDIC |
| | VSLLMGEQFLVSWC |

In some embodiments, the rhGAA has a GAA amino acid sequence as set forth in SEQ ID NO: 1, as described in U.S. Pat. No. 8,592,362 and has GenBank accession number AHE24104.1 (GI:568760974). In some embodiments, the rhGAA has a GAA amino acid sequence as encoded in SEQ ID NO: 2, the mRNA sequence having GenBank accession number Y00839.1. In some embodiments, the rhGAA has a GAA amino acid sequence as set forth in SEQ ID NO: 3. In at some embodiments, the rhGAA has a GAA amino acid sequence as set forth in SEQ ID NO: 4 and has National Center for Biotechnology Information (NCBI) accession number NP_000143.2 or UniProtKB Accession Number P10253.

In some embodiments, the rhGAA is initially expressed as having the full-length 952 amino acid sequence of wild-type GAA as set forth in SEQ ID NO: 1 or SEQ ID NO: 4, and the rhGAA undergoes intracellular processing that removes a portion of the amino acids, e.g., the first 56 amino acids. Accordingly, the rhGAA that is secreted by the host cell can have a shorter amino acid sequence than the rhGAA that is initially expressed within the cell. In some embodiments, the shorter protein has the amino acid sequence set forth in SEQ ID NO: 5, which only differs from SEQ ID NO: 1 in that the first 56 amino acids comprising the signal peptide and precursor peptide have been removed, thus resulting in a protein having 896 amino acids. In some embodiments, the shorter protein has the amino acid sequence set forth in SEQ ID NO: 6, which only differs from SEQ ID NO: 4 in that the first 56 amino acids comprising the signal peptide and precursor peptide have been removed, thus resulting in a protein having 896 amino acids. Other variations in the number of amino acids are also possible, such as having 1, 2, 3, 4, 5, 6, 7, 8, 9, 10, 11, 12, 13, 14, 15, or more deletions, substitutions and/or insertions relative to the amino acid sequence described by SEQ ID NO: 1, SEQ ID NO: 4, SEQ ID NO: 5, or SEQ ID NO: 6. In some embodiments, the rhGAA product includes a mixture of recombinant human acid α-glucosidase molecules having different amino acid lengths.

In some embodiments, the rhGAA comprises an amino acid sequence that is at least 70%, 75%, 80%, 85%, 90%, 95%, 98% or 99% identical to SEQ ID NO: 4 or SEQ ID NO: 6. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 75%, 80%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

In some embodiments, the rhGAA undergoes post-translational and/or chemical modifications at one or more amino acid residues in the protein. For example, methionine and tryptophan residues can undergo oxidation. As another example, the N-terminal glutamine in SEQ ID NO: 6 can be further modified to form pyro-glutamate. As another example, asparagine residues can undergo deamidation to aspartic acid. As yet another example, aspartic acid residues can undergo isomerization to iso-aspartic acid. As yet another example, unpaired cysteine residues in the protein can form disulfide bonds with free glutathione and/or cysteine. Accordingly, in some embodiments, the enzyme is initially expressed as having an amino acid sequence as set forth in SEQ ID NO: 1, SEQ ID NO: 3, SEQ ID NO: 4, or SEQ ID NO: 5, or an amino acid sequence encoded by SEQ ID NO: 2, and the enzyme undergoes one or more of these post-translational and/or chemical modifications. Such modifications are also within the scope of the present disclosure.

N-Linked Glycosylation of rhGAA

There are seven potential N-linked glycosylation sites on a single rhGAA molecule. These potential glycosylation sites are at the following positions of SEQ ID NO: 6: N84, N177, N334, N414, N596, N826, and N869. Similarly, for the full-length amino acid sequence of SEQ ID NO: 4, these potential glycosylation sites are at the following positions: N140, N233, N390, N470, N652, N882, and N925. Other variants of rhGAA can have similar glycosylation sites, depending on the location of asparagine residues. Generally, sequences of Asn-X-Ser or Asn-X-Thr in the protein amino acid sequence indicate potential glycosylation sites, with the exception that X cannot be His or Pro.

The rhGAA molecules described herein may have, on average, 1, 2, 3, or 4 mannose-6-phosphate (M6P) groups on their N-glycans. For example, only one N-glycan on a rhGAA molecule may bear M6P (mono-phosphorylated or mono-M6P), a single N-glycan may bear two M6P groups (bis-phosphorylated or bis-M6P), or two different N-glycans on the same rhGAA molecule may each bear single M6P groups. In some embodiments, the rhGAA molecules described herein on average have 3-4 mol M6P groups on their N-glycans per mol rhGAA. Recombinant human acid α-glucosidase molecules may also have N-glycans bearing no M6P groups. In another embodiment, on average the rhGAA comprises greater than 2.5 mol M6P per mol rhGAA and greater than 4 mol sialic acid per mol rhGAA. In some embodiments, on average the rhGAA comprises about 3-3.5 mol M6P per mol rhGAA. In some embodiments, on average the rhGAA comprises about 4-5.4 mol sialic acid per mol rhGAA. On average at least about 3, 4, 5, 6, 7, 8, 9, 10%, or 20% of the total N-glycans on the rhGAA may be in the form of a mono-M6P N-glycan, for example, about 6.25% of the total N-glycans may carry a single M6P group and on average, at least about 0.5, 1, 1.5, 2.0, 2.5, 3.0% of the total N-glycans on the rhGAA are in the form of a bis-M6P N-glycan and on average less than 25% of total rhGAA contains no phosphorylated N-glycan binding to CIMPR. In some embodiments, on average about 10% to about 14% of the total N-glycans on the rhGAA are mono-phosphorylated. In some embodiments, on average about 7% to about 25% of the total N-glycans on the rhGAA are bis-phosphorylated. In some embodiments, on average the rhGAA comprises about 1.3 mol bis-M6P per mol rhGAA.

The rhGAA described herein may have on average from 0.5 to 7.0 mol M6P per mol rhGAA or any intermediate value or subrange thereof including 0.5, 1.0, 1.5, 2.0, 2.5, 3.0, 3.5, 4.0, 4.5, 5.0, 5.5, 6.0, 6.5, or 7.0 mol M6P per mol rhGAA. The rhGAA can be fractionated to provide rhGAA preparations with different average numbers of mono-M6P-bearing or bis-M6P-bearing N-glycans, thus permitting further customization of rhGAA targeting to the lysosomes in target tissues by selecting a particular fraction or by selectively combining different fractions.

In some embodiments, up to 60% of the N-glycans on the rhGAA may be fully sialylated, for example, up to 10%, 20%, 30%, 40%, 50% or 60% of the N-glycans may be fully sialylated. In some embodiments, no more than 50% of the N-glycans on the rhGAA are fully sialylated. In some embodiments, from 4% to 20% of the total N-glycans are fully sialylated. In other embodiments, no more than 5%, 10%, 20% or 30% of N-glycans on the rhGAA carry sialic acid and a terminal galactose residue (Gal). This range includes all intermediate values and subranges, for example, 7% to 30% of the total N-glycans on the rhGAA can carry sialic acid and terminal galactose. In yet other embodiments, no more than 5%, 10%, 15%, 16%, 17%, 18%, 19%, or 20% of the N-glycans on the rhGAA have a terminal galactose only and do not contain sialic acid. This range includes all intermediate values and subranges, for example, from 8% to 19% of the total N-glycans on the rhGAA in the composition may have terminal galactose only and do not contain sialic acid.

In some embodiments, 40% to 60%, 45% to 60%, 50% to 60%, or 55% to 60% of the total N-glycans on the rhGAA are complex type N-glycans; or no more than 1%, 2%, 3%, 4%, 5%, 6,%, or 7% of total N-glycans on the rhGAA are hybrid-type N-glycans; no more than 5%, 10%, 15%, 20%, or 25% of the high mannose-type N-glycans on the rhGAA are non-phosphorylated; at least 5% or 10% of the high mannose-type N-glycans on the rhGAA are mono-phosphorylated; and/or at least 1% or 2% of the high mannose-type N-glycans on the rhGAA are bis-phosphorylated. These values include all intermediate values and subranges. A rhGAA may meet one or more of the content ranges described above.

In some embodiments, the rhGAA may bear, on average, 2.0 to 8.0 moles of sialic acid residues per mole of rhGAA. This range includes all intermediate values and subranges thereof, including 2.0, 2.5, 3.0, 3.5, 4.0, 4.5, 5.0, 5.5, 6.0, 6.5, 7.0, 7.5, and 8.0 mol sialic acid residues per mol rhGAA. Without being bound by theory, it is believed that the presence of N-glycan units bearing sialic acid residues may prevent non-productive clearance of the rhGAA by asialoglycoprotein receptors.

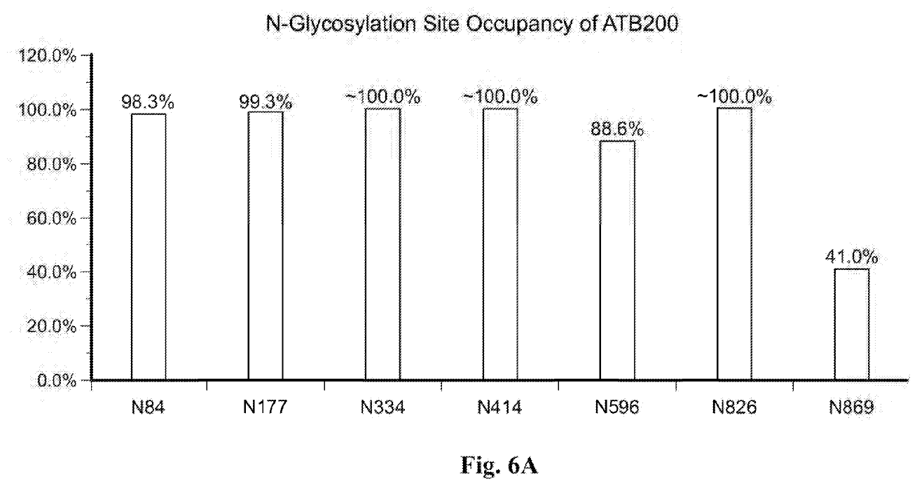

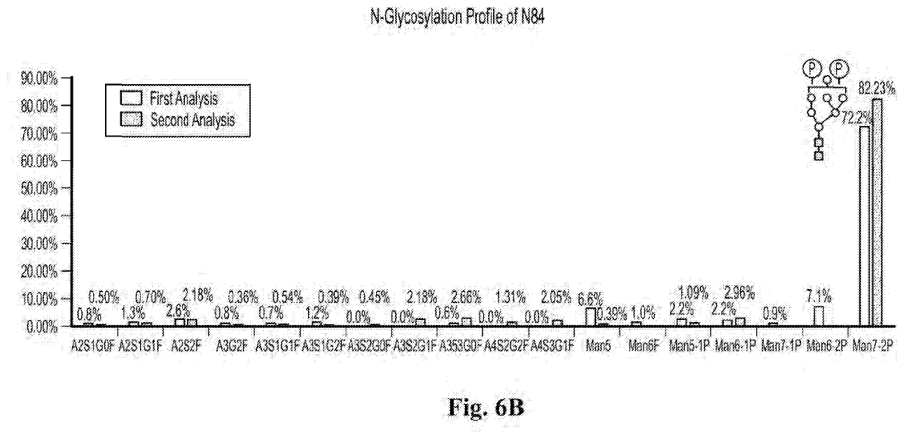

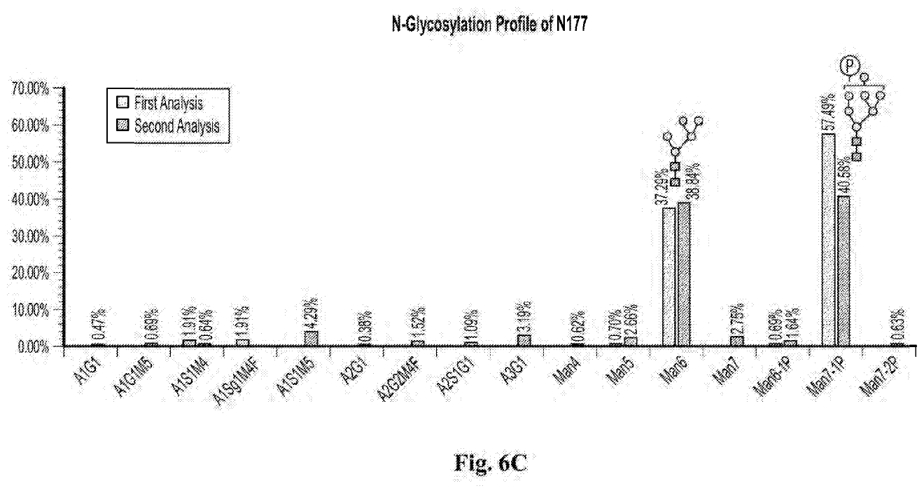

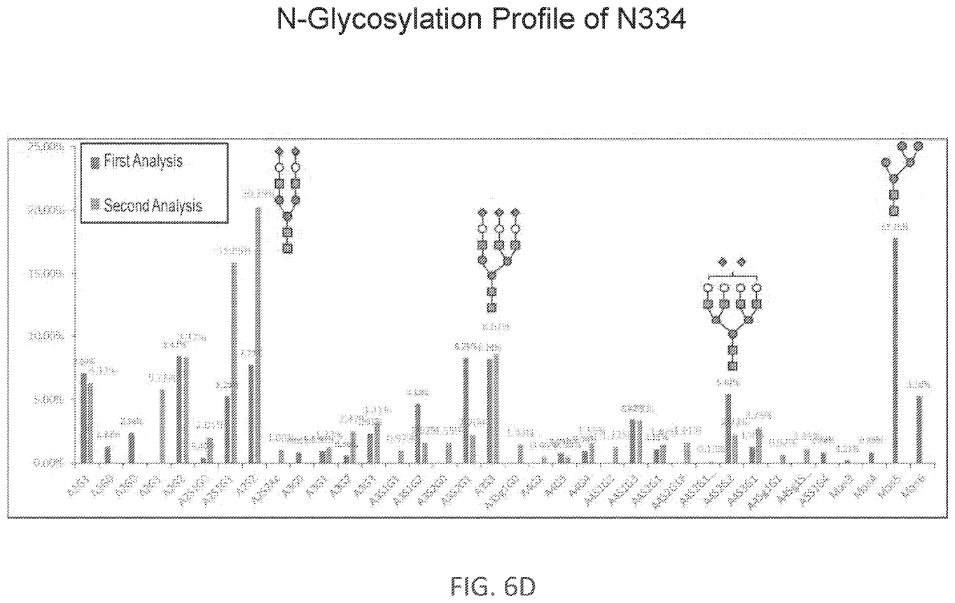

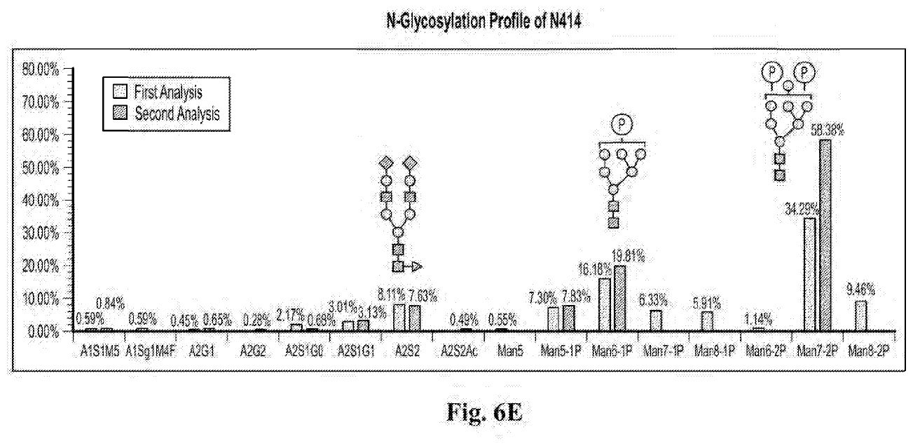

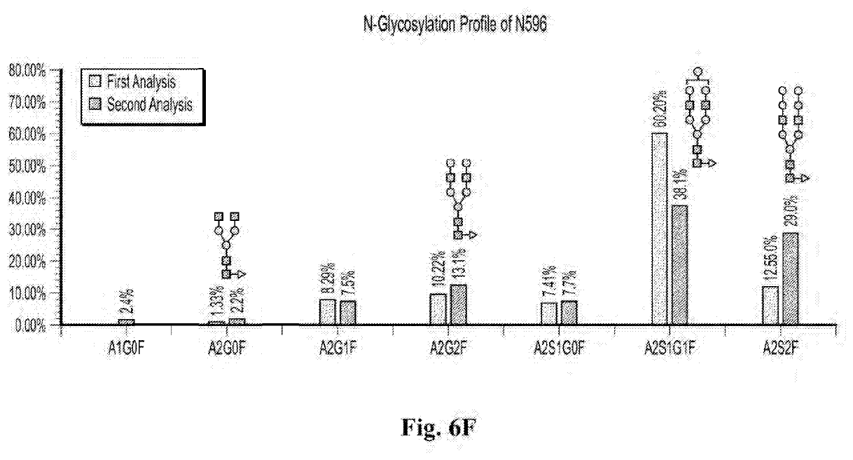

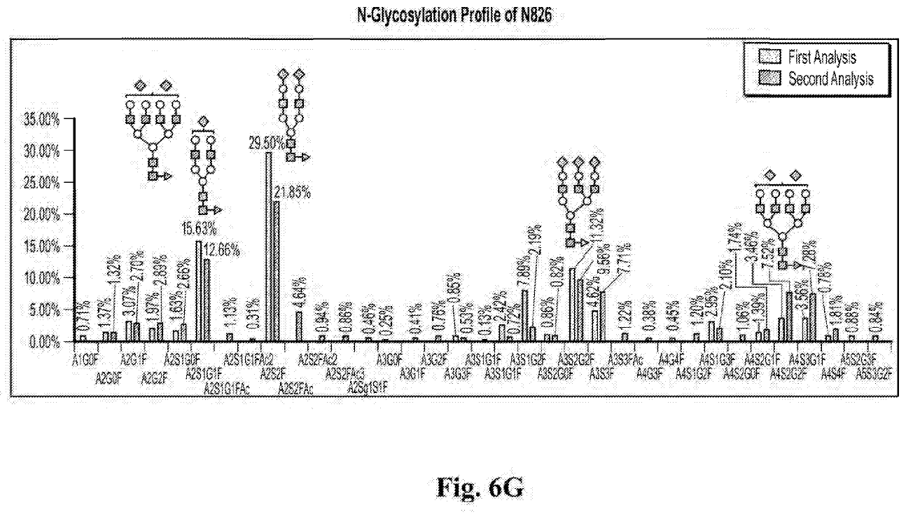

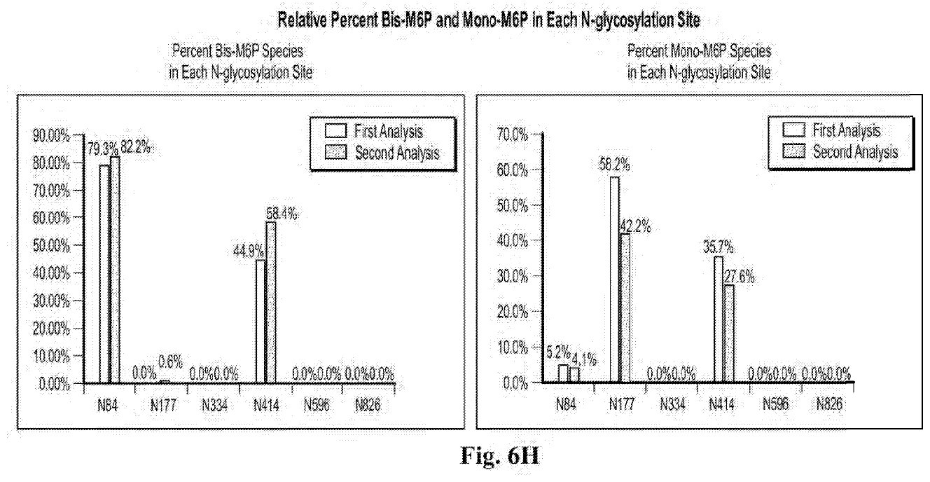

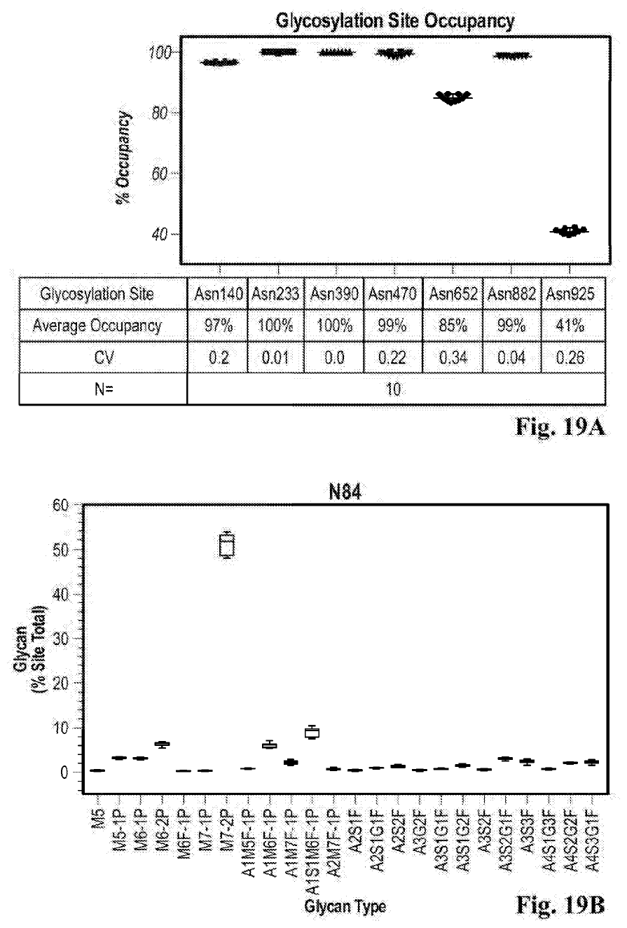

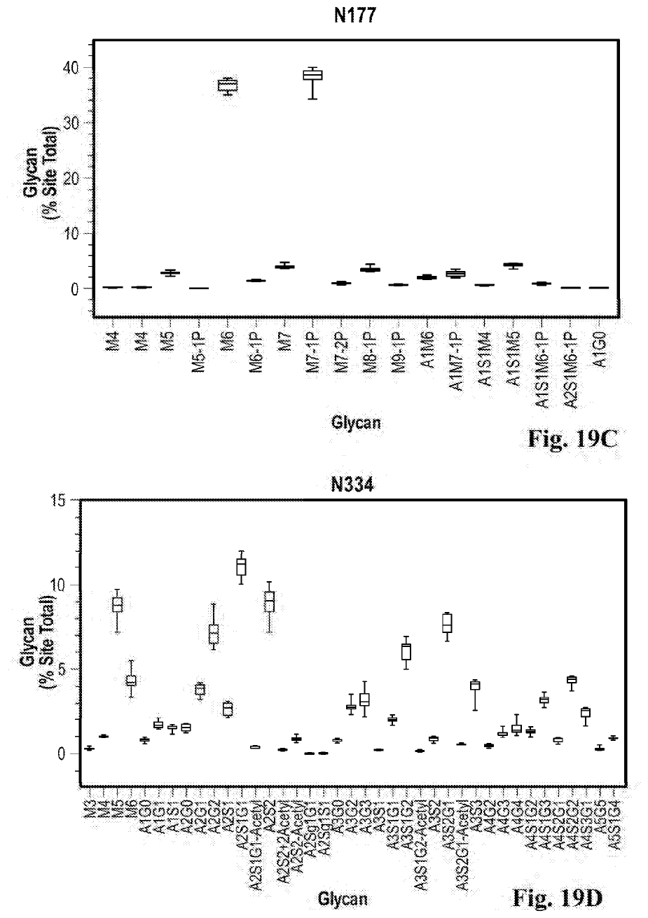

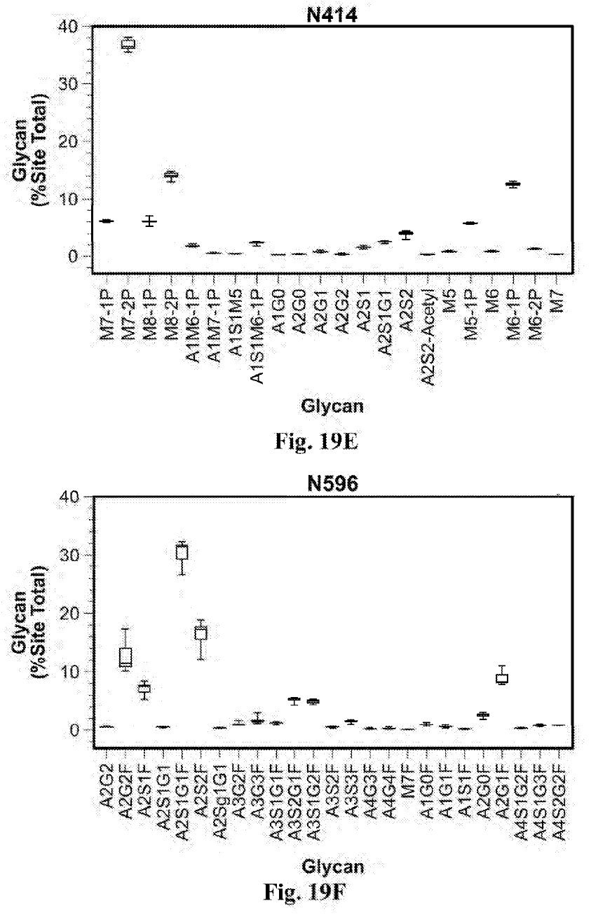

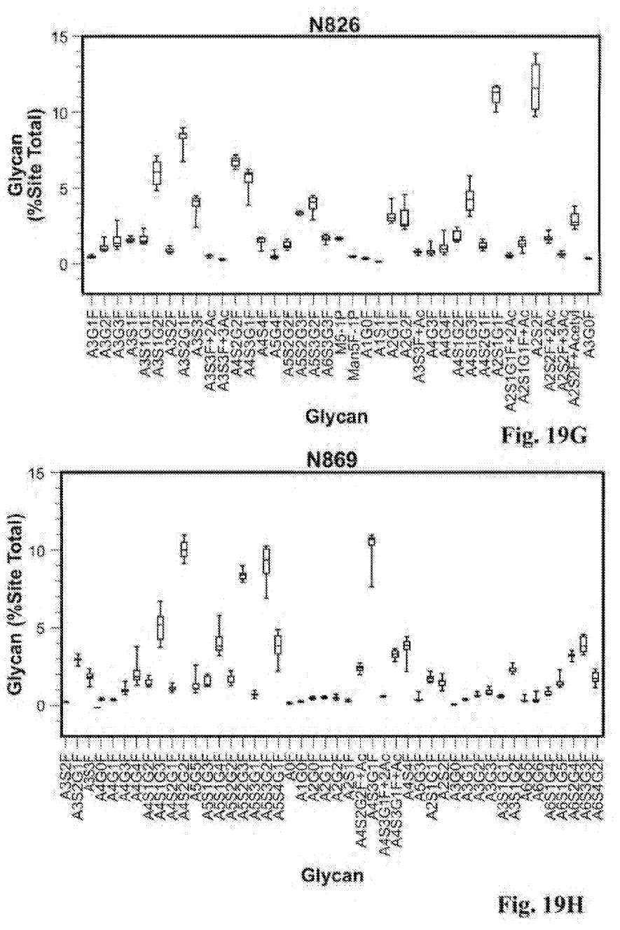

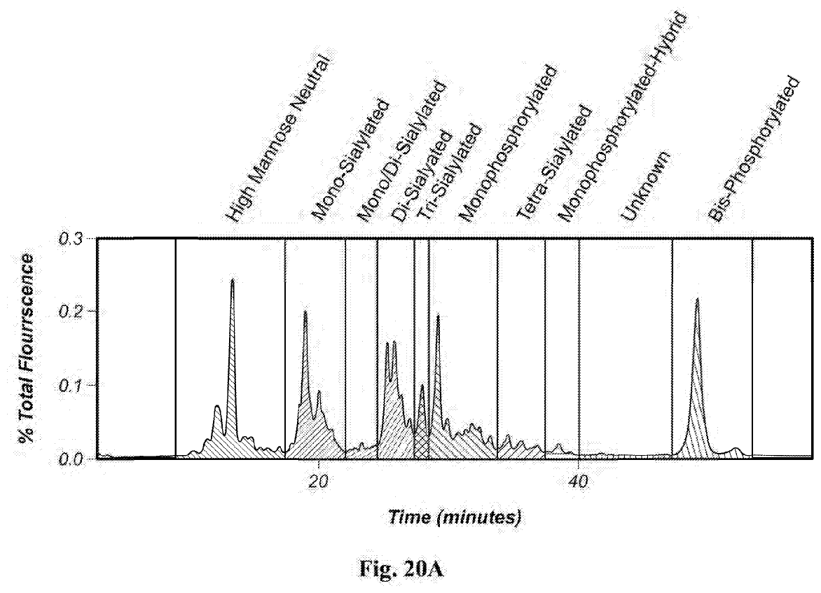

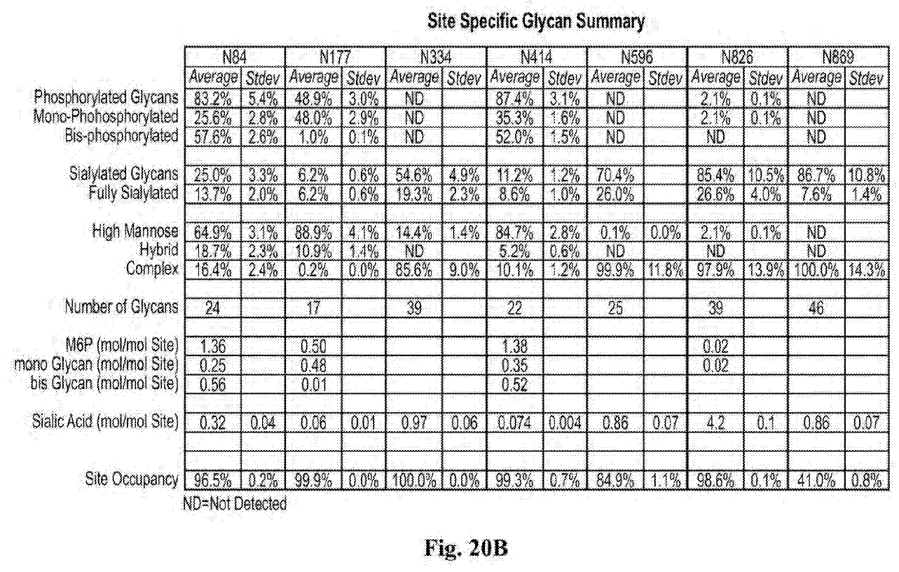

In one or more embodiments, the rhGAA has a certain N-glycosylation profile at certain potential N-glycosylation sites. In some embodiments, the rhGAA has seven potential N-glycosylation sites. In some embodiments, at least 20% of the rhGAA is phosphorylated at the first potential N-glycosylation site (e.g., N84 for SEQ ID NO: 6 and N140 for SEQ ID NO: 4). For example, at least 20%, 25%, 30%, 35%, 40%, 45%, 50%, 55%, 60%, 65%, 70%, 75%, 80%, 85%, 90%, or 95% of the rhGAA can be phosphorylated at the first potential N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a mono-M6P unit at the first potential N-glycosylation site. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a bis-M6P unit at the first potential N-glycosylation site. In some embodiments, the rhGAA comprises on average about 1.4 mol M6P (mono-M6P and bis-M6P) per mol rhGAA at the first potential N-glycosylation site. In some embodiments, the rhGAA comprises on average about at least 0.5 mol bis-M6P per mol rhGAA at the first potential N-glycosylation site. In some embodiments, the rhGAA comprises on average about 0.25 mol mono-M6P per mol rhGAA at the first potential N-glycosylation site. In some embodiments, the rhGAA comprises on average about 0.2 mol to about 0.3 mol sialic acid per mol rhGAA at the first potential N-glycosylation site. In at least one embodiment, the rhGAA comprises a first potential N-glycosylation site occupancy as depicted in FIG. 6A and an N-glycosylation profile as depicted in FIG. 6B. In at least one embodiment, the rhGAA comprises a first potential N-glycosylation site occupancy as depicted in FIG. 19A and an N-glycosylation profile as depicted in FIG. 19B or FIG. 20B.

In some embodiments, at least 20% of the rhGAA is phosphorylated at the second potential N-glycosylation site (e.g., N177 for SEQ ID NO: 6 and N223 for SEQ ID NO: 4). For example, at least 20%, 25%, 30%, 35%, 40%, 45%, 50%, 55%, 60%, 65%, 70%, 75%, 80%, 85%, 90%, or 95% of the rhGAA can be phosphorylated at the second N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a mono-M6P unit at the second N-glycosylation site. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a bis-M6P unit at the second N-glycosylation site. In some embodiments, the rhGAA comprises on average about 0.5 mol M6P (mono-M6P and bis-M6P) per mol rhGAA at the second potential N-glycosylation site. In some embodiments, the rhGAA comprises on average about 0.4 to about 0.6 mol mono-M6P per mol rhGAA at the second potential N-glycosylation site. In at least one embodiment, the rhGAA comprises a second potential N-glycosylation site occupancy as depicted in FIG. 6A and an N-glycosylation profile as depicted in FIG. 6C. In at least one embodiment, the rhGAA comprises a second potential N-glycosylation site occupancy as depicted in FIG. 19A and an N-glycosylation profile as depicted in FIG. 19C or FIG. 20B.

In one or more embodiments, at least 5% of the rhGAA is phosphorylated at the third potential N-glycosylation site (e.g., N334 for SEQ ID NO: 6 and N390 for SEQ ID NO: 4). In other embodiments, less than 5%, 10%, 15%, 20%, or 25% of the rhGAA is phosphorylated at the third potential N-glycosylation site. For example, the third potential N-glycosylation site can have a mixture of non-phosphorylated high mannose N-glycans, di-, tri-, and tetra-antennary complex N-glycans, and hybrid N-glycans as the major species. In some embodiments, at least 3%, 5%, 8%, 10%, 15%, 20%, 25%, 30%, 35%, 40%, 45%, or 50% of the rhGAA is sialylated at the third potential N-glycosylation site. In some embodiments, the rhGAA comprises on average about 0.9 to about 1.2 mol sialic acid per mol rhGAA at the third potential N-glycosylation site. In at least one embodiment, the rhGAA comprises a third potential N-glycosylation site occupancy as depicted in FIG. 6A and an N-glycosylation profile as depicted in FIG. 6D. In at least one embodiment, the rhGAA comprises a third potential N-glycosylation site occupancy as depicted in FIG. 19A and an N-glycosylation profile as depicted in FIG. 19D or FIG. 20B.

In some embodiments, at least 20% of the rhGAA is phosphorylated at the fourth potential N-glycosylation site (e.g., N414 for SEQ ID NO: 6 and N470 for SEQ ID NO: 4). For example, at least 20%, 25%, 30%, 35%, 40%, 45%, 50%, 55%, 60%, 65%, 70%, 75%, 80%, 85%, 90%, or 95% of the rhGAA can be phosphorylated at the fourth potential N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a mono-M6P unit at the fourth potential N-glycosylation site. In some embodiments, at least 10%, 15%, 20%, 25%, 30%, 35%, 40%, 45%, 50%, 55%, 60%, 65%, 70%, 75%, 80%, 85%, 90%, or 95% of the rhGAA bears a bis-M6P unit at the fourth potential N-glycosylation site. In some embodiments, at least 3%, 5%, 8%, 10%, 15%, 20%, or 25% of the rhGAA is sialylated at the fourth potential N-glycosylation site. In some embodiments, the rhGAA comprises on average about 1.4 mol M6P (mono-M6P and bis-M6P) per mol rhGAA at the fourth potential N-glycosylation site. In some embodiments, the rhGAA comprises on average about 0.4 to about 0.6 mol bis-M6P per mol rhGAA at the fourth potential N-glycosylation site. In some embodiments, the rhGAA comprises on average about 0.3 to about 0.4 mol mono-M6P per mol rhGAA at the fourth potential N-glycosylation site. In at least one embodiment, the rhGAA comprises a fourth potential N-glycosylation site occupancy as depicted in FIG. 6A and an N-glycosylation profile as depicted in FIG. 6E. In at least one embodiment, the rhGAA comprises a fourth potential N-glycosylation site occupancy as depicted in FIG. 19A and an N-glycosylation profile as depicted in FIG. 19E or FIG. 20B.

In some embodiments, at least 5% of the rhGAA is phosphorylated at the fifth potential N-glycosylation site (e.g., N596 for SEQ ID NO: 6 and N692 for SEQ ID NO: 4). In other embodiments, less than 5%, 10%, 15%, 20%, or 25% of the rhGAA is phosphorylated at the fifth potential N-glycosylation site. For example, the fifth potential N-glycosylation site can have fucosylated di-antennary complex N-glycans as the major species. In some embodiments, at least 3%, 5%, 8%, 10%, 15%, 20%, 25%, 30%, 35%, 40%, 45%, 50%, 55%, 60%, 65%, 70%, 75%, 80%, 85%, 90%, or 95% of the rhGAA is sialylated at the fifth potential N-glycosylation site. In some embodiments, the rhGAA comprises on average about 0.8 to about 0.9 mol sialic acid per mol rhGAA at the fifth potential N-glycosylation site. In at least one embodiment, the rhGAA comprises a fifth potential N-glycosylation site occupancy as depicted in FIG. 6A and an N-glycosylation profile as depicted in FIG. 6F. In at least one embodiment, the rhGAA comprises a fifth potential N-glycosylation site occupancy as depicted in FIG. 19A and an N-glycosylation profile as depicted in FIG. 19F or FIG. 20B.

In some embodiments, at least 5% of the rhGAA is phosphorylated at the sixth N-glycosylation site (e.g., N826 for SEQ ID NO: 6 and N882 for SEQ ID NO: 4). In other embodiments, less than 5%, 10%, 15%, 20% or 25% of the rhGAA is phosphorylated at the sixth N-glycosylation site. For example, the sixth N-glycosylation site can have a mixture of di-, tri-, and tetra-antennary complex N-glycans as the major species. In some embodiments, at least 3%, 5%, 8%, 10%, 15%, 20%, 25%, 30%, 35%, 40%, 45%, 50%, 55%, 60%, 65%, 70%, 75%, 80%, 85%, 90% or 95% of the rhGAA is sialylated at the sixth N-glycosylation site. In some embodiments, the rhGAA comprises on average about 1.5 to about 4.2 mol sialic acid per mol rhGAA at the sixth potential N-glycosylation site. In some embodiments, the rhGAA comprises on average about 0.9 mol acetylated sialic acid per mol rhGAA at the sixth potential N-glycosylation site. In some embodiments, the rhGAA comprises an average of at least 0.05 mol glycan species with poly-N-Acetyl-D-lactosamine (poly-LacNAc) residues per mol rhGAA at the sixth potential N-glycosylation site. In some embodiments, over 10% of the rhGAA comprises a glycan bearing a poly-LacNAc residue at the sixth potential N-glycosylation site. In at least one embodiment, the rhGAA comprises a sixth potential N-glycosylation site occupancy as depicted in FIG. 6A and an N-glycosylation profile as depicted in FIG. 6G. In at least one embodiment, the rhGAA comprises a sixth potential N-glycosylation site occupancy as depicted in FIG. 19A and an N-glycosylation profile as depicted in FIG. 19G or FIG. 20B.

In some embodiments, at least 5% of the rhGAA is phosphorylated at the seventh potential N-glycosylation site (e.g., N869 for SEQ ID NO: 6 and N925 for SEQ ID NO: 4). In other embodiments, less than 5%, 10%, 15%, 20%, or 25% of the rhGAA is phosphorylated at the seventh potential N-glycosylation site. In some embodiments, less than 40%, 45%, 50%, 55%, 60%, or 65% of the rhGAA has any N-glycan at the seventh potential N-glycosylation site. In some embodiments, at least 30%, 35%, or 40% of the rhGAA has an N-glycan at the seventh potential N-glycosylation site. In some embodiments, the rhGAA comprises on average at least 0.5 mol sialic acid per mol rhGAA at the seventh potential N-glycosylation site. In some embodiments, the rhGAA comprises on average at least 0.8 mol sialic acid per mol rhGAA at the seventh potential N-glycosylation site. In some embodiments, the rhGAA comprises on average about 0.86 mol sialic acid per mol rhGAA at the seventh potential N-glycosylation site. In some embodiments, the rhGAA comprises an average of at least 0.3 mol glycan species bearing poly-LacNAc residues per mol rhGAA at the seventh potential N-glycosylation site. In some embodiments, nearly half of the rhGAA comprises a glycan bearing a poly-LacNAc residue at the seventh potential N-glycosylation site. In at least one embodiment, all N-glycans identified at the seventh potential N-glycosylation site are complex N-glycans. In at least one embodiment, the rhGAA comprises a seventh potential N-glycosylation site occupancy as depicted in FIG. 6A or as depicted in FIG. 19A and an N-glycosylation profile as depicted in FIG. 19H or FIG. 20B.

In some embodiments, the rhGAA comprises on average 3-4 mol M6P residues per mol rhGAA and about 4 to about 7.3 mol sialic acid per mol rhGAA. In some embodiments, the rhGAA further comprises on average at least about 0.5 mol bis-M6P per mol rhGAA at the first potential N-glycosylation site, about 0.4 to about 0.6 mol mono-M6P per mol rhGAA at the second potential N-glycosylation site, about 0.9 to about 1.2 mol sialic acid per mol rhGAA at the third potential N-glycosylation site, about 0.4 to about 0.6 mol bis-M6P per mol rhGAA at the fourth potential N-glycosylation site, about 0.3 to about 0.4 mol mono-M6P per mol rhGAA at the fourth potential N-glycosylation site, about 0.8 to about 0.9 mol sialic acid per mol rhGAA at the fifth potential N-glycosylation site, and about 1.5 to about 4.2 mol sialic acid per mol rhGAA at the sixth potential N-glycosylation site. In some embodiments, the rhGAA further comprises on average at least 0.5 mol sialic acid per mol rhGAA at the seventh potential N-glycosylation site. In some embodiments, the rhGAA comprises on average at least 0.8 mol sialic acid per mol rhGAA at the seventh potential N-glycosylation site. In at least one embodiment, the rhGAA further comprises on average about 0.86 mol sialic acid per mol rhGAA at the seventh potential N-glycosylation site. In at least one embodiment, the rhGAA comprises seven potential N-glycosylation sites with occupancy and N-glycosylation profiles as depicted in FIGS. 6A-6H. In at least one embodiment, the rhGAA comprises seven potential N-glycosylation sites with occupancy and N-glycosylation profiles as depicted in FIGS. 19A-19H and FIGS. 20A-20B.

Methods of making rhGAA are disclosed, for example, in International Pat. App. No. PCT/2015/053252, U.S. Pat. Nos. 10,208,299, 10,961,522, and U.S. Provisional Patent Application No. 62/057,842, filed Sep. 30, 2014, the entire disclosures of which are incorporated herein by reference.

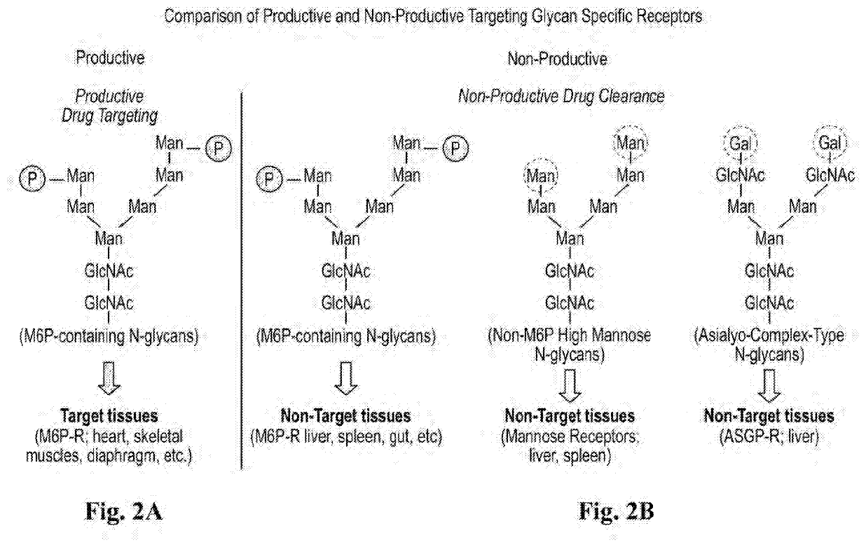

Once inside the lysosome, rhGAA can enzymatically degrade accumulated glycogen. However, conventional rhGAA products have low total levels of mono-M6P- and bis-M6P bearing N-glycans and, thus, target muscle cells poorly, resulting in inferior delivery of rhGAA to the lysosomes. The majority of rhGAA molecules in these conventional products do not have phosphorylated N-glycans, thereby lacking affinity for the CIMPR. Non-phosphorylated high mannose N-glycans can also be cleared by the mannose receptor, which results in non-productive clearance of the ERT (FIG. 2B). In contrast, as shown in FIG. 2A, a rhGAA described herein may contains a higher amount of mono-M6P- and bis-M6P bearing N-glycans, leading to productive uptake of rhGAA into specific tissues such as muscle.

Production and Purification of N-Linked Glycosylated rhGAA

As described in International Pat. App. No. PCT/2015/053252, U.S. Pat. Nos. 10,208,299, and 10,961,522, the entireties of which are incorporated herein by reference, cells such as Chinese hamster ovary (CHO) cells may be used to produce the rhGAA described therein. Expressing high M6P rhGAA in CHO cells is advantageous over modifying the glycan profile of an rhGAA post-translationally at least in part because only the former may be converted by glycan degradation to a form of rhGAA with optimal glycogen hydrolysis, thus enhancing therapeutic efficacy.

In some embodiments, the rhGAA is preferably produced by one or more CHO cell lines that are transformed with a DNA construct encoding the rhGAA described herein. Such CHO cell lines may contain multiple copies of a gene, such as 5, 10, 15, or 20 or more copies, of a polynucleotide encoding GAA. DNA constructs, which express allelic variants of acid α-glucosidase or other variant acid α-glucosidase amino acid sequences such as those that are at least 90%, 95%, 98%, or 99% identical to SEQ ID NO: 4 or SEQ ID NO: 6, may be constructed and expressed in CHO cells. Those of skill in the art may select alternative vectors suitable for transforming CHO cells for production of such DNA constructs.

Methods for making such CHO cell lines are described in International Pat. App. No. PCT/2015/053252, U.S. Pat. Nos. 10,208,299, and 10,961,522, the entireties of which are incorporated herein by reference. Briefly, these methods involve transforming a CHO cell with DNA encoding GAA or a GAA variant, selecting a CHO cell that stably integrates the DNA encoding GAA into its chromosome(s) and that stably expresses GAA, and selecting a CHO cell that expresses GAA having a high content of N-glycans bearing mono-M6P or bis-M6P, and, optionally, selecting a CHO cell having N-glycans with high sialic acid content and/or having N-glycans with a low non-phosphorylated high-mannose content. The selected CHO cell lines may be used to produce rhGAA and rhGAA compositions by culturing the CHO cell line and recovering said composition from the culture of CHO cells. In some embodiments, a rhGAA produced from the selected CHO cell lines contains a high content of N-glycans bearing mono-M6P or bis-M6P that target the CIMPR. In some embodiments, a rhGAA produced as described herein has low levels of complex N-glycans with terminal galactose. In some embodiments, the selected CHO cell lines are referred to as GA-ATB200 or ATB200-X5-14. In some embodiments, the selected CHO cell lines encompass a subculture or derivative of such a CHO cell culture. In some embodiments, a rhGAA produced from the selected CHO cell lines is referred to as ATB200.

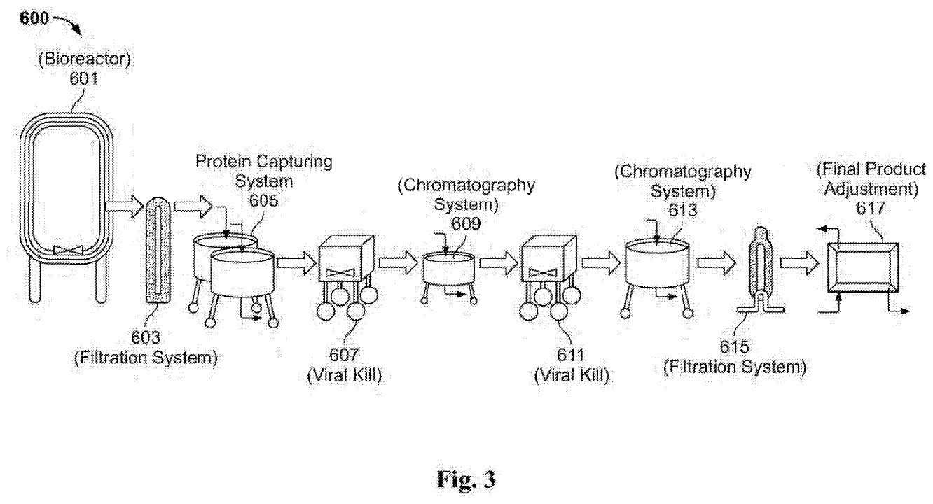

A rhGAA produced as described herein may be purified by following methods described in U.S. Pat. No. 10,227,577 and in U.S. Provisional Application No. 62/506,569, both of which are incorporated herein by reference in their entirety. An exemplary process for producing, capturing, and purifying a rhGAA produced from CHO cell lines is shown in FIG. 3.

Briefly, bioreactor 601 contains a culture of cells, such as CHO cells, that express and secrete rhGAA into the surrounding liquid culture media. The bioreactor 601 may be any appropriate bioreactor for culturing the cells, such as a perfusion, batch or fed-batch bioreactor. The culture media is removed from the bioreactor after a sufficient period of time for cells to produce rhGAA. Such media removal may be continuous for a perfusion bioreactor or may be batch-wise for a batch or fed-batch reactor. The media may be filtered by filtration system 603 to remove cells. Filtration system 603 may be any suitable filtration system, including an alternating tangential flow filtration (ATF) system, a tangential flow filtration (TFF) system, and/or centrifugal filtration system. In various embodiments, the filtration system utilizes a filter having a pore size between about 10 nanometers and about 2 micrometers.

After filtration, the filtrate is loaded onto a protein capturing system 605. The protein capturing system 605 may include one or more chromatography columns. If more than one chromatography column is used, then the columns may be placed in series so that the next column can begin loading once the first column is loaded. Alternatively, the media removal process can be stopped during the time that the columns are switched.

In various embodiments, the protein capturing system 605 includes one or more anion exchange (AEX) columns for the direct product capture of rhGAA, particularly rhGAA having a high M6P content. The rhGAA captured by the protein capturing system 605 is eluted from the column(s) by changing the pH and/or salt content in the column. Exemplary conditions for an AEX column are provided in Table 2.

TABLE 2
Exemplary conditions for an AEX column

| | | Flow rate | | Temperature |
|---|---|---|---|---|
| Procedure | Buffer | (cm/h) | Volume (CV) | (° C.) |
| Pre-used | 0.1-10M NaOH | ≤25-2500 | ≥1-3 | 15-25 |
| Sanitization | | | (≥10-120 min) | |
| Pre- | 20-2000 mM phosphate | ≤25-2500 | ≥1-5 | 15-25 |
| Equilibration | buffer (PB), pH 6.9-7.3 | | | |
| Equilibration | 4-400 mM PB, pH 6.9-7.3 | ≤25-2500 | ≥1-5 | 2-15 |
| Load | NA | ≤10-1000 | NA | 2-15 |
| Wash1 | 4-400 mM PB, pH 6.9-7.3 | ≤25-2500 | ≥2-10 | 2-15 |
| Wash2 | 4-400 mM PB, pH 6.9-7.3 | ≤25-2500 | ≥2-10 | 15-25 |
| Elution | 4-400 mM PB, 20-2000 mM | ≤25-2500 | NA | 15-25 |
| | NaCl, pH 6.1-6.5 | | | |
| Strip | 4-400 mM PB, 0.1-10M | ≤25-2500 | ≥1-5 | 15-25 |
| | NaCl, pH 6.1-6.5 | | | |
| Post-use | 0.1-10M NaOH | ≤25-2500 | ≥1-3 | 15-25 |
| Sanitization | | | (≥10-120 min) | |
| Storage | 0.01-1.0M NaOH | ≤25-2500 | ≥1-5 | 15-25 |

The eluted rhGAA can be subjected to further purification steps and/or quality assurance steps. For example, the eluted rhGAA may be subjected to a virus kill step 607. Such a virus kill step 607 may include one or more of a low pH kill, a detergent kill, or other technique known in the art. The rhGAA from the virus kill step 607 may be introduced into a second chromatography system 609 to further purify the rhGAA product. Alternatively, the eluted rhGAA from the protein capturing system 605 may be fed directly to the second chromatography system 609. In various embodiments, the second chromatography system 609 includes one or more immobilized metal affinity chromatography (IMAC) columns for further removal of impurities. Exemplary conditions for an IMAC column are provided in Table 3 below.

TABLE 3
Exemplary conditions for an IMAC column

| | | Flow rate | Vol |
|---|---|---|---|
| Procedure | Buffer | (cm/h) | (CV) |
| Rinse | 4-400 mM PB, pH 6.3-6.7 | ≤25-2500 | ≥1-5 |
| Pre-use | 0.01-1.0M NaOH | ≤25-2500 | ≥1-3 |
| Sanitization | | | (10-30 min) |
| Equilibration | 4-400 mM PB, pH 6.5 | ≤25-2500 | ≥1-5 |
| Wash with WFI | Water For Injection (WFI) | ≤25-2500 | ≥1-3 |
| Chelating | 0.01-1.0M Copper Acetate | ≤25-2500 | ≥1-5 |
| Wash with WFI | WFI | ≤25-2500 | ≥2-10 |
| Wash with acidic | 2-200 mM Sodium Acetate, 0.05-5M | ≤25-2500 | ≥2-10 |
| buffer | NaCl, pH 3.5-4.5 | | |
| Equilibration | 4-400 mM PB, pH 6.3-6.7 | ≤25-2500 | ≥1-5 |
| Blank run with | 4-400 mM PB, 15-1500 mM Glycine, | ≤25-2500 | ≥2-20 |
| elution buffer | pH 6.1-6.5 | | |
| Equilibration | 4-400 mM PB, pH 6.3-6.7 | ≤25-2500 | ≥1-5 |
| Load | NA | ≤25-2500 | ≥1-5 |
| Wash1 | 4-400 mM PB, pH 6.3-6.7 | ≤25-2500 | ≥2-10 |
| Wash2 | 4-400 mM PB, 0.1-10M NaCl, 5- | ≤25-2500 | ≥2-10 |
| | 30% propylene glycol, pH 6.3-6.7 | | |
| Wash3 | 4-400 mM PB, pH 6.3-6.7 | ≤25-2500 | ≥2-10 |
| Elution | 4-400 mM PB, 15-1500 mM Glycine, | ≤25-2500 | NA |
| | pH 6.1-6.5 | | |
| Strip | 4-400 mM PB, 50-5000 mM | ≤25-2500 | ≥1-5 |
| | imidazole, pH 6.3-6.7 | | |
| Post-use | 0.01-1M NaOH | ≤25-2500 | ≥1-3 |
| Sanitization | | | (10-30 min) |
| Rinse | 4-400 mM PB, pH 6.3-6.7 | ≤25-2500 | ≥1-5 |
| Storage | 5-30% ethanol | ≤25-2500 | ≥1-5 |

After the rhGAA is loaded onto the second chromatography system 609, the recombinant protein is eluted from the column(s). The eluted rhGAA can be subjected to a virus kill step 611. As with virus kill 607, virus kill 611 may include one or more of a low pH kill, a detergent kill, or other technique known in the art. In some embodiments, only one of virus kill 607 or 611 is used, or the virus kills are performed at the same stage in the purification process.

The rhGAA from the virus kill step 611 may be introduced into a third chromatography system 613 to further purify the recombinant protein product. Alternatively, the eluted recombinant protein from the second chromatography system 609 may be fed directly to the third chromatography system 613. In various embodiments, the third chromatography system 613 includes one or more cation exchange chromatography (CEX) columns and/or size exclusion chromatography (SEC) columns for further removal of impurities. The rhGAA product is then eluted from the third chromatography system 613. Exemplary conditions for a CEX column are provided in Table 4 below.

TABLE 4
Exemplary conditions for a CEX column

| | | Flow rate | Vol |
|---|---|---|---|
| Procedure | Buffer | (cm/h) | (CV) |
| Pre-used | 0.1-10M NaOH | ≤25-2500 | ≥1-3 |
| Sanitization | | | (≥10-120 min) |
| Equilibration | 2-200 mM Sodium citrate, pH 4.0-5.0 | ≤30-3000 | ≥2-10 |
| Load | NA | ≤30-3000 | NA |
| Wash | 2-200 mM Sodium citrate, pH 4.0-5.0 | ≤30-3000 | ≥2-10 |
| Elution | 2-200 mM Sodium citrate, 15-1500 mM | ≤30-3000 | ≥2-10 |
| | NaCl, pH 4.0-5.0 | | |
| Strip | 2-200 mM Sodium citrate, 0.1-10M NaCl, | ≤30-3000 | ≥1-5 |
| | pH 4.0-5.0 | | |
| Post-use | 0.1-10M NaOH | ≤25-2500 | ≥1-3 |
| Sanitization | | | (≥10-120 min) |
| Storage | 0.01-1.0M NaOH | ≤30-3000 | ≥1-5 |

The rhGAA product may also be subjected to further processing. For example, another filtration system 615 may be used to remove viruses. In some embodiments, such filtration can utilize filters with pore sizes between 5 and 50 μm. Other product processing can include a product adjustment step 617, in which the recombinant protein product may be sterilized, filtered, concentrated, stored, and/or have additional components for added for the final product formulation.

### Pharmaceutical Compositions

In various embodiments, a pharmaceutical composition comprising the rhGAA described herein, either alone or in combination with other therapeutic agents, and/or a pharmaceutically acceptable carrier, is provided.

In one or more embodiments, a pharmaceutical composition described herein comprises a pharmaceutically acceptable salt.

In some embodiments, the pharmaceutically acceptable salt used herein is a pharmaceutically-acceptable acid addition salt. The pharmaceutically-acceptable acid addition salt may include, but is not limited to, hydrochloric acid, hydrobromic acid, sulfuric acid, sulfamic acid, nitric acid, phosphoric acid, and the like, and organic acids including but not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like.

In some embodiments, the pharmaceutically acceptable salt used herein is a pharmaceutically-acceptable base addition salt. The pharmaceutically-acceptable base addition salt may include, but is not limited to, ammonia or the hydroxide, carbonate, or bicarbonate of ammonium or a metal cation such as sodium, potassium, lithium, calcium, magnesium, iron, zinc, copper, manganese, aluminum, and the like. Salts derived from pharmaceutically-acceptable organic nontoxic bases include, but are not limited to, salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like.

In some embodiments, the rhGAA or a pharmaceutically acceptable salt thereof may be formulated as a pharmaceutical composition adapted for intravenous administration. In some embodiments, the pharmaceutical composition is a solution in sterile isotonic aqueous buffer. Where necessary, the composition may also include a solubilizing agent and a local anesthetic to ease pain at the site of the injection. The ingredients of the pharmaceutical composition may be supplied either separately or mixed together in unit dosage form, for example, as a dry lyophilized powder or water free concentrate in a hermetically sealed container such as an ampule or sachet indicating the quantity of active agent. Where the composition is to be administered by infusion, it may be dispensed with an infusion bottle containing sterile pharmaceutical grade water, saline or dextrose/water. In some embodiments, the infusion may occur at a hospital or clinic. In some embodiments, the infusion may occur outside the hospital or clinic setting, for example, at a subject's residence. Where the composition is administered by injection, an ampule of sterile water for injection or saline may be provided so that the ingredients may be mixed prior to administration.

In some embodiments, the rhGAA or a pharmaceutically acceptable salt thereof is administered every 2 weeks as an IV infusion lasting about 4 hours. In some embodiments, the total volume of infusion is determined by the patient's body weight. Infusion rate can be lowered and infusion duration increased if patient experiences an IAR.

In some embodiments, the initial infusion rate is 1.5 mg/kg/hour. In some embodiments, the infusion rate is gradually increased by 3 mg/kg/hour every 30 minutes if there are no signs of infusion-associated reactions (IARs) until a maximum rate of 10.5 mg/kg/hour is reached; then, the infusion rate is maintained at 10.5 mg/kg/hour until the infusion is complete. In some embodiments, the approximate total infusion duration is 4 hours

Infusions should be administered in a step-wise manner using an infusion pump. Infusion rates can be increased from initial rate every 30 minutes+/−5 minutes until the maximum rate is reached as shown in the table below based on dosage and patient weight.

TABLE 5
Recommended Infusion Volumes and Rates for 30 mg/kg Dose

| Subject | Total | Step 1 | Step 2 | Step 3 | Step 4 |
|---|---|---|---|---|---|
| Weight | Infusion | 1.5 | 4.5 | 7.5 | 10.5 |
| Range | Volume | mg/kg/hr | mg/kg/hr | mg/kg/hr | mg/kg/hr |
| (kg) | (mL) | (mL/hr) | (mL/hr) | (mL/hr) | (mL/hr) |
| 1.25-10 | 50 | 3 | 8 | 13 | 18 |
| 10.1-20 | 100 | 5 | 15 | 25 | 35 |
| 20.1-30 | 150 | 8 | 23 | 38 | 53 |
| 30.1-35 | 200 | 10 | 30 | 50 | 70 |
| 35.1-50 | 250 | 13 | 38 | 63 | 88 |
| 50.1-60 | 300 | 15 | 45 | 75 | 105 |
| 60.1-100 | 500 | 25 | 75 | 125 | 175 |
| 100.1-120 | 600 | 30 | 90 | 150 | 210 |

The most serious tolerability issue with the rhGAA or a pharmaceutically acceptable salt thereof is the occurrence of infusion-associated reactions (IARs), which, in some instances can include life-threatening anaphylaxis or other severe allergic responses. In some embodiments, prior to administration of the rhGAA or a pharmaceutically acceptable salt thereof, pretreatments with antihistamines, antipyretics, and/or corticosteroids are administered. If pretreatment was used with previous enzyme replacement therapy (ERT), prior to administration of the rhGAA or a pharmaceutically acceptable salt thereof, pretreatments with antihistamines, antipyretics, and/or corticosteroids are administered.

In some embodiments, the rhGAA or a pharmaceutically acceptable salt thereof may be formulated for oral administration. Orally administrable compositions may be formulated in a form of tablets, capsules, ovules, elixirs, solutions or suspensions, gels, syrups, mouth washes, or a dry powder for reconstitution with water or other suitable vehicle before use, optionally with flavoring and coloring agents for immediate-, delayed-, modified-, sustained-, pulsed-, or controlled-release applications. Solid compositions such as tablets, capsules, lozenges, pastilles, pills, boluses, powder, pastes, granules, bullets, dragees, or premix preparations can also be used. Solid and liquid compositions for oral use may be prepared according to methods well known in the art. Such compositions can also contain one or more pharmaceutically acceptable carriers and excipients which can be in solid or liquid form. Tablets or capsules can be prepared by conventional means with pharmaceutically acceptable excipients, including but not limited to binding agents, fillers, lubricants, disintegrants, or wetting agents. Suitable pharmaceutically acceptable excipients are known in the art and include but are not limited to pregelatinized starch, polyvinylpyrrolidone, povidone, hydroxypropyl methylcellulose (HPMC), hydroxypropyl ethylcellulose (HPEC), hydroxypropyl cellulose (HPC), sucrose, gelatin, acacia, lactose, microcrystalline cellulose, calcium hydrogen phosphate, magnesium stearate, stearic acid, glyceryl behenate, talc, silica, corn, potato or tapioca starch, sodium starch glycolate, sodium lauryl sulfate, sodium citrate, calcium carbonate, dibasic calcium phosphate, glycine croscarmellose sodium, and complex silicates. Tablets can be coated by methods well known in the art.

In some embodiments, a pharmaceutical composition described herein may be formulated according to U.S. Pat. No. 10,512,676 and U.S. Provisional Application No. 62/506,574, both incorporated herein by reference in their entirety. For instance, in some embodiments, the pH of a pharmaceutical composition described herein is from about 5.0 to about 7.0 or about 5.0 to about 6.0. In some embodiments, the pH ranges from about 5.5 to about 6.0. In some embodiments, the pH of the pharmaceutical composition is 6.0. In some embodiments, the pH may be adjusted to a target pH by using pH adjusters (e.g., alkalizing agents and acidifying agents) such as sodium hydroxide and/or hydrochloric acid.

The pharmaceutical composition described herein may comprise a buffer system such as a citrate system, a phosphate system, and a combination thereof. The citrate and/or phosphate may be a sodium citrate or sodium phosphate. Other salts include potassium and ammonium salts. In one or more embodiments, the buffer comprises a citrate. In further embodiments, the buffer comprises sodium citrate (e.g., a mixture of sodium citrate dihydrate and citric acid monohydrate). In one or more embodiments, buffer solutions comprising a citrate may comprise sodium citrate and citric acid. In some embodiments, both a citrate and phosphate buffer are present.

In some embodiments, a pharmaceutical composition described herein comprises at least one excipient. The excipient may function as a tonicity agent, bulking agent, and/or stabilizer. Tonicity agents are components which help to ensure the formulation has an osmotic pressure similar to or the same as human blood. Bulking agents are ingredients which add mass to the formulations (e.g., lyophilized) and provide an adequate structure to the cake. Stabilizers are compounds that can prevent or minimize the aggregate formation at the hydrophobic air-water interfacial surfaces. One excipient may function as a tonicity agent and bulking agent at the same time. For instance, mannitol may function as a tonicity agent and also provide benefits as a bulking agent.

Examples of tonicity agents include sodium chloride, mannitol, sucrose, and trehalose. In some embodiments, the tonicity agent comprises mannitol. In some embodiments, the total amount of tonicity agent(s) ranges in an amount of from about 10 mg/mL to about 50 mg/mL. In further embodiments, the total amount of tonicity agent(s) ranges in an amount of from about 10, 11, 12, 13, 14, or 15 mg/mL to about 16, 20, 25, 30, 35, 40, 45, or 50 mg/mL.

In some embodiments, the excipient comprises a stabilizer. In some embodiments, the stabilizer is a surfactant. In some embodiments, the stabilizer is polysorbate 80. In one or more embodiments, the total amount of stabilizer ranges from about 0.1 mg/mL to about 1.0 mg/mL. In further embodiments, the total amount of stabilizer ranges from about 0.1, 0.2, 0.3, 0.4, or 0.5 mg/mL to about 0.5, 0.6, 0.7, 0.8, 0.9, or 1.0 mg/mL. In yet further embodiments, the total amount of stabilizer is about 0.1, 0.2, 0.3, 0.4, 0.5, 0.6, 0.7, 0.8, 0.9, or 1.0 mg/mL.

In some embodiments, the pharmaceutical composition comprises one or more of the following excipients: sodium citrate dihydrate, citric acid monohydrate, mannitol or polysorbate-80.

In some embodiments, a pharmaceutical composition comprises (a) a rhGAA (such as ATB200 or cipaglucosidase alfa), (b) at least one buffer selected from the group consisting of a citrate, a phosphate, and a combination thereof, and (c) at least one excipient selected from the group consisting of mannitol, polysorbate 80, and a combination thereof, and has a pH of (i) from about 5.0 to about 6.0, or (ii) from about 5.0 to about 7.0. In some embodiments, the composition further comprises water. In some embodiments, the composition may further comprise an acidifying agent and/or alkalizing agent.

In some embodiments, the pharmaceutical composition comprises (a) a rhGAA (such as ATB200 or cipaglucosidase alfa) at a concentration of about 5-50 mg/mL, about 5-30 mg/mL, or about 15 mg/mL, (b) sodium citrate buffer at a concentration of about 10-100 mM or about 25 mM, (c) mannitol at a concentration of about 10-50 mg/mL, or about 20 mg/mL, (d) polysorbate 80, present at a concentration of about 0.1-1 mg/mL, about 0.2-0.5 mg/mL, or about 0.5 mg/mL, and (e) water, and has a pH of about 6.0. In at least one embodiment, the pharmaceutical composition comprises (a) 15 mg/mL rhGAA (such as ATB200 or cipaglucosidase alfa) (b) 25 mM sodium citrate buffer, (c) 20 mg/mL mannitol (d) 0.5 mg/mL polysorbate 80, and (e) water, and has a pH of about 6.0. In some embodiments, the composition may further comprise an acidifying agent and/or alkalizing agent.

In some embodiments, the pharmaceutical composition comprising rhGAA (e.g., ATB200 or cipaglucosidase alfa) is diluted prior to administration to a subject in need thereof. In some embodiments, the pharmaceutical composition described herein may undergo lyophilization (freeze-drying) process to provide a cake or powder. Accordingly, in some embodiments, the pharmaceutical composition described herein pertains to a rhGAA composition after lyophilization. The lyophilized mixture may comprise the rhGAA described herein (e.g., ATB200), buffer selected from the group consisting of a citrate, a phosphate, and combinations thereof, and at least one excipient selected from the group consisting of trehalose, mannitol, polysorbate 80, and a combination thereof. In some embodiments, other ingredients (e.g., other excipients) may be added to the lyophilized mixture. The pharmaceutical composition comprising the lyophilized formulation may be provided in a vial, which then can be stored, transported, reconstituted and/or administered to a patient.

In some embodiments, the pharmaceutical composition comprising a rhGAA (e.g., ATB200 or cipaglucosidase alfa) as described herein is a lyophilized powder in glass vials. In some embodiments, each vial may contain about 105 mg of lyophilized rhGAA (e.g., ATB200 or cipaglucosidase alfa). The powder may be reconstituted in sterile water and then followed by dilution with 0.9% sodium chloride prior to administration by IV infusion. In some embodiments, after reconstitution, the concentrate obtained contains 15 mg of rhGAA (e.g., ATB200 or cipaglucosidase alfa) per mL. In some embodiments, after reconstitution with 7.2 mL of diluent, the vial contains a usable volume of 7.0 mL of concentrate containing 15 mg/mL of rhGAA (e.g., ATB200 or cipaglucosidase alfa). In some embodiments, the diluent is sterile water and/or 0.9% sodium chloride. In some embodiments, each vial may include an overfill to make up for fluid loss during preparation. In some embodiments, the instant disclosure provide a vial (e.g., a glass vial) containing 105 mg lyophilized rhGAA (e.g., ATB200 or cipaglucosidase alfa) composition comprising rhGAA (e.g., ATB200 or cipaglucosidase alfa), sodium citrate dihydrate, citric acid monohydrate, mannitol, polysorbate 80, wherein the amount/concentration of each ingredient may be selected from those described herein.

In some embodiments, the number of vials to be reconstituted are based on patient's body weight and the fact that each vial contains 105 mg of rhGAA (e.g., ATB200 or cipaglucosidase alfa). Accordingly, in some embodiments, patient dose (mg)=Subject weight (kg)×dose (mg/kg); and Number of vials required=patient dose (in mg)/105 (mg per vial). In some embodiments, if the number of vials includes a fraction, round up to the next whole number.

For example, in a 10 kg patient dosed at 30 mg/kg: Patient dose (mg)=10 kg×30 mg/kg=300 mg total dose; Number of vials required=300/105 mg per vial=2.857 rounded up to 3 vials for the purpose of dispensation. The patient dose is 30 mg/kg, therefore the full volume of the first 2 vials will be extracted but the 3^{rd} vial will have 6.0 mL extracted and added to the infusion bag.

In some embodiments, the method of reconstitution comprises or consists essentially of the follow processes: (1) reconstitute each vial by slowly injecting 7.2 mL of Sterile Water for Injection, to the inside wall of each vial and not directly onto the lyophilized cake; (2) roll each vial gently, do not invert, swirl, or shake; (3) dilute an amount of reconstituted rhGAA based on the patient's body weight in 0.9% Sodium Chloride for Injection, immediately after reconstitution to the total infusion volume for 30 mg/kg dose based on patient weight; (4) prior to adding the reconstituted rhGAA, remove air and total amount equal to reconstituted volume 0.9% Sodium Chloride for Injection bag; (5) slowly withdraw the reconstituted solution from each vial avoiding foaming in the syringe; (6) Slowly add the reconstituted cipaglucosidase alfa solution directly into the 0.9% Sodium Chloride for Injection bag (do not add directly into the airspace that may remain within the infusion bag) avoiding foaming in the infusion bag (Alternatively, product dilution can be accomplished using an appropriately sized empty infusion bag to add the required volume of 0.9% Sodium Chloride for Injection and the reconstituted rhGAA based on patient weight. It is recommended to use pre-filled 0.9% Sodium Chloride for Injection bag); (7) gently invert or massage the infusion bag to mix; and (8) cover the infusion bag and tubing to protect the medication from light. After reconstitution, each vial will yield a concentration of 15 mg/mL. The total extractable dose per vial is 105 mg per 7 mL.

In some embodiments, only the exact amount of reconstituted rhGAA based on the patient's body weight is diluted. In some embodiments, for partial withdrawal of the reconstituted vial, amounts are rounded up or down to 1 decimal place. In some embodiments, the infusion bag is not shaken to mix or a pneumatic tube used to transport the infusion bag. In some embodiments, the reconstituted and diluted solutions may contain particles in the form of thin white strands or translucent fibers after initial preparation and increase over time.

The present disclosure also provides a pharmaceutical composition comprising an enzyme stabilizer. In some embodiments, a pharmaceutical composition described herein comprises an enzyme stabilizer. In some embodiments, the enzyme stabilizer is miglustat or a pharmaceutically acceptable salt thereof. In another embodiment, the enzyme stabilizer is duvoglustat or a pharmaceutically acceptable salt thereof.

In some embodiments, a rhGAA described herein is formulated in one pharmaceutical composition while an enzyme stabilizer such as miglustat is formulated in another pharmaceutical composition. In some embodiments, the pharmaceutical composition comprising miglustat is based on a formulation available commercially as ZAVESCA® (Actelion Pharmaceuticals). In some embodiments, the pharmaceutical composition comprising miglustat comprises microcrystalline cellulose, pregelatinized starch, Emprove® sucralose powder, magnesium stearate, and/or colloidal silicon dioxide. In some embodiments, the pharmaceutical composition is a hard gelatin capsule for oral administration, comprising about 65 mg of miglustat, microcrystalline cellulose, pregelatinized starch, Emprove® sucralose powder, magnesium stearate, and/or colloidal silicon dioxide.

In some embodiments, a pharmaceutical composition comprising miglustat comprises 20%-40% by weight miglustat, such as 30-35% by weight miglustat. In some embodiments, a pharmaceutical composition comprising miglustat further comprises 40%-60% by weight microcrystalline cellulose, such as 45-55% by weight microcrystalline cellulose. In some embodiments, a pharmaceutical composition comprising miglustat further comprises 5%-25% by weight pregelatinized starch, such as 10-20% by weight pregelatinized starch. In some embodiments, a pharmaceutical composition comprising miglustat further comprises 0.1%-5% by weight sucralose, such as 0.2-1% by weight sucralose. In some embodiments, a pharmaceutical composition comprising miglustat further comprises 0.1%-5% by weight magnesium stearate, such as 0.2-1% by weight magnesium stearate. In some embodiments, a pharmaceutical composition comprising miglustat further comprises 0.1%-5% by weight colloidal silicon dioxide, such as 0.2-1% by weight colloidal silicon dioxide. In some embodiments, a pharmaceutical composition comprising miglustat is provided in a hard gelatin capsule for oral administration. In some embodiments, the pharmaceutical composition is a hard gelatin capsule for oral administration, comprising about 20%-40% (e.g., 30-35%) by weight of miglustat, 40%-60% (e.g., 45-55%) by weight of microcrystalline cellulose, 5%-25% (e.g., 10-20%) by weight of pregelatinized starch, 0.1%-5% (e.g., 0.2%-1%) by weight of sucralose, 0.1%-5% (e.g., 0.2%-1%) by weight of magnesium stearate, and/or 0.1%-5% (e.g., 0.2%-1%) by weight of colloidal silicon dioxide. In some embodiments, a pharmaceutical composition comprising miglustat is provided in an oral liquid dosage form, such as an oral solution, dispersion, or suspension. In some embodiments, the pharmaceutical composition is an oral liquid dosage form, comprising about 20%-40% (e.g., 30-35%) by weight of miglustat, 40%-60% (e.g., 45-55%) by weight of microcrystalline cellulose, 5%-25% (e.g., 10-20%) by weight of pregelatinized starch, 0.1%-5% (e.g., 0.2%-1%) by weight of sucralose, 0.1%-5% (e.g., 0.2%-1%) by weight of magnesium stearate, and/or 0.1%-5% (e.g., 0.2%-1%) by weight of colloidal silicon dioxide.

In some embodiments, a pharmaceutical composition comprising miglustat comprises about 50 to about 100 mg miglustat, such as about 65 mg miglustat. In some embodiments, a pharmaceutical composition comprising miglustat comprises about 50 to about 150 mg microcrystalline cellulose, such as about 75 to about 125 mg microcrystalline cellulose. In some embodiments, a pharmaceutical composition comprising miglustat comprises about 20 to about 50 mg pregelatinized starch, such as about 30 to about 40 mg pregelatinized starch. In some embodiments, a pharmaceutical composition comprising miglustat comprises about 0.1 to about 5 mg sucralose, such as about 0.5 to about 2 mg sucralose. In some embodiments, a pharmaceutical composition comprising miglustat comprises about 0.1 to about 5 mg magnesium stearate, such as about 0.5 to about 2 mg magnesium stearate. In some embodiments, a pharmaceutical composition comprising miglustat comprises about 0.1 to about 5 mg colloidal silicon dioxide, such as about 0.2 mg to about 1 mg colloidal silicon dioxide. In some embodiments, a pharmaceutical composition comprising miglustat is provided in a hard gelatin capsule for oral administration. In some embodiments, the pharmaceutical composition is a hard gelatin capsule for oral administration, comprising about 50 to about 100 mg (e.g., 65 mg) of miglustat, about 50 to about 150 mg (e.g., 75 mg to about 125 mg) of microcrystalline cellulose, about 20 to about 50 mg (e.g., 30 mg to about 40 mg) of pregelatinized starch, about 0.1 to about 5 mg (e.g., 0.5 mg to about 2 mg) of sucralose, about 0.1 to about 5 mg (e.g., 0.5 mg to about 2 mg) of magnesium stearate, and/or about 0.1 to about 5 mg (e.g., 0.5 mg to about 2 mg) of colloidal silicon dioxide. In some embodiments, a pharmaceutical composition comprising miglustat is provided in an oral liquid dosage form, such as an oral solution, dispersion or suspension. In some embodiments, the pharmaceutical composition is an oral liquid dosage form, comprising about 50 to about 100 mg (e.g., 65 mg) of miglustat, about 50 to about 150 mg (e.g., 75 mg to about 125 mg) of microcrystalline cellulose, about 20 to about 50 mg (e.g., 30 mg to about 40 mg) of pregelatinized starch, about 0.1 to about 5 mg (e.g., 0.5 mg to about 2 mg) of sucralose, about 0.1 to about 5 mg (e.g., 0.5 mg to about 2 mg) of magnesium stearate, and/or about 0.1 to about 5 mg (e.g., 0.5 mg to about 2 mg) of colloidal silicon dioxide.

In some embodiments, a pharmaceutical composition comprising miglustat is provided in a hard gelatin capsule for oral administration comprising about 65 mg miglustat, about 100 mg microcrystalline cellulose, about 32.6 mg pregelatinized starch, about 1 mg sucralose powder, about 1 mg magnesium stearate, and about 0.4 mg colloidal silicon dioxide.

In some embodiments, a pharmaceutical composition comprising miglustat is provided in an oral solution, dispersion or suspension, comprising about 65 mg miglustat, about 100 mg microcrystalline cellulose, about 32.6 mg pregelatinized starch, about 1 mg sucralose powder, about 1 mg magnesium stearate, and about 0.4 mg colloidal silicon dioxide.

In some embodiments, a pharmaceutical composition comprising miglustat is provided in an oral solution dispersion or suspension, comprising about 130 mg miglustat, about 200 mg microcrystalline cellulose, about 65.2 mg pregelatinized starch, about 2 mg sucralose powder, about 2 mg magnesium stearate, and about 0.8 mg colloidal silicon dioxide.

In some embodiments, a pharmaceutical composition comprising miglustat is provided in an oral solution, dispersion or suspension, comprising about 195 mg miglustat, about 300 mg microcrystalline cellulose, about 97.8 mg pregelatinized starch, about 3 mg sucralose powder, about 3 mg magnesium stearate, and about 1.2 mg colloidal silicon dioxide.

In some embodiments, a pharmaceutical composition comprising miglustat is provided in an oral solution, dispersion or suspension, comprising about 260 mg miglustat, about 400 mg microcrystalline cellulose, about 130.4 mg pregelatinized starch, about 4 mg sucralose powder, about 4 mg magnesium stearate, and about 1.6 mg colloidal silicon dioxide.

In some embodiments, a patient may have difficulty swallowing a hard gelatin capsule. Accordingly, in some embodiments, a pharmaceutical composition comprising miglustat is provided in an oral solution. In some embodiments, the oral solution is flavored.

In some embodiments, a pharmaceutical composition comprising miglustat is provided as an oral solution comprising one or more excipients. In some embodiments, the pharmaceutical composition comprising miglustat comprises a preservative, a buffering agent, a thickener (or a thickening agent), a sweetener, a flavor, and/or a vehicle.

In some embodiments, a pharmaceutical composition comprising miglustat is an oral solution comprising 1.0-10.0%, 1.5-8%, 2.0-5.0%, 2.0-6.5%, 2-7%, 2.5-5.0%, 2.5-6.5%, 2.5-7%, 3.0-5.0%, 3-6.5%, 3-7%, 3.5-5.0%, 3.5-6%, 3.5-7%, 4.0-5.0%, 4-6.5%, 4-7%, 4.5-5.0%, 4.5-6%, 4.5-7% 2.0-4.5%, 2.5-4.5%, 3.0-4.5%, 3.5-4.5%, 4.0-4.5%, 2.0-4.0%, 2.5-4.0%, 3.0-4.0%, or 3.5-4.0% miglustat by weight. In some embodiments, a pharmaceutical composition comprising miglustat is an oral solution comprising less than or equal to about 5% miglustat by weight. In some embodiments, a pharmaceutical composition comprising miglustat is an oral solution comprising 1 to 200 mg/mL, such as 10 to 100 mg/mL, 10 to 50 mg/mL or 20 to 60 mg/mL. Exemplary miglustat concentrations include about 5 mg/mL, 10 mg/mL, 15 mg/mL, 20 mg/mL, 30 mg/mL, 40 mg/mL, 50 mg/mL, 60 mg/mL, 70 mg/mL, 80 mg/mL, 90 mg/mL or 100 mg/mL.

In some embodiments, a pharmaceutical composition comprising miglustat is an oral solution comprising a buffer system such as a citrate system, a phosphate system, or a combination thereof. The citrate and/or phosphate may be a sodium salt (e.g., sodium citrate or sodium phosphate). Other salts include potassium and ammonium salts. In one or more embodiments, the buffer comprises a citrate. In further embodiments, the buffer comprises sodium citrate (e.g., a mixture of sodium citrate dihydrate and citric acid monohydrate). In one or more embodiments, buffer solutions comprising a citrate may comprise sodium citrate and citric acid. In some embodiments, both a citrate and phosphate buffer are present.

In some embodiments, a pharmaceutical composition comprising miglustat is an oral solution comprising a thickener or a thickening agent. In some embodiments, the thickener is selected from hydroxypropyl methylcellulose (HPMC), hydroxypropyl ethylcellulose (HPEC), hydroxypropyl cellulose (HPC), gelatin, or microcrystalline cellulose. In some embodiments, the pharmaceutical composition comprising miglustat is an oral solution comprising 0.5-5%, 1.0-3.0% or 1.0-2.0% of a thickener by weight. In some embodiments, the thickener is hydroxypropyl methylcellulose (HPMC).

In some embodiments, a pharmaceutical composition comprising miglustat is an oral solution comprising a sweetener. In some embodiments, the sweetener is selected from sucrose or sucralose. In some embodiments, the pharmaceutical composition comprising miglustat is an oral solution comprising 0.1-1%, 0.1-0.5%, 0.1-0.3% or 0.15-0.25% sweetener by weight. In some embodiments, the sweetener is sucralose.

In some embodiments, a pharmaceutical composition comprising miglustat is an oral solution comprising a preservative. In some embodiments, the preservative is potassium sorbate. In some embodiments, the pharmaceutical composition comprising miglustat is an oral solution comprising 0.1-1%, 0.1-0.5%, 0.1-0.3% or 0.15-0.25% preservative by weight.

In some embodiments, a pharmaceutical composition comprising miglustat is an oral solution comprising a flavoring agent. In some embodiments, the pharmaceutical composition comprising miglustat is an oral solution comprising 0.1-0.5%, 0.1-0.3% or 0.15-0.25% of a flavoring agent by weight.

In some embodiments, the balance of the oral solution comprising miglustat is water. In some embodiments, the water comprises greater than or equal to 80%, 90%, 91%, 92%, 93%, 94% or 95% water.

In some embodiments, the pH of the oral solution comprising miglustat is adjusted with a strong acid (e.g., hydrochloric acid) and/or a strong base (e.g., sodium hydroxide). In some embodiments, the pH of the oral solution comprising miglustat is adjusted to 5.0+/−0.5. Stated differently, in some embodiments, the pH of the oral solution comprising miglustat is in a range of 4.5-5.5, 4.7-5.3, 4.9-5.1, or is about 5.0.

In some embodiments, an oral solution comprising miglustat is an oral solution comprising 1.0-10.0%, 2.0-5.0%, 2.5-5.0%, 3.0-5.0%, 3.5-5.0%, 4.0-5.0%, 4.5-5.0%, 2.0-4.5%, 2.5-4.5%, 3.0-4.5%, 3.5-4.5%, 4.0-4.5%, 2.0-4.0%, 2.5-4.0%, 3.0-4.0%, or 3.5-4.0% miglustat by weight. In some embodiments an oral solution comprises less than or equal to about 4% miglustat by weight.

In some embodiments, an oral solution comprising miglustat is an oral solution comprising 1 to 200 mg/mL, such as 10 to 100 mg/mL, 10 to 50 mg/mL or 20 to 60 mg/mL. Exemplary miglustat concentrations include about 5 mg/mL, 10 mg/mL, 15 mg/mL, 20 mg/mL, 30 mg/mL, 40 mg/mL, 50 mg/mL, 60 mg/mL, 70 mg/mL, 80 mg/mL, 90 mg/mL or 100 mg/mL.

In some embodiments, an oral solution comprising miglustat contains a buffering agent. In some embodiments the buffering agent is sodium citrate. In some embodiments, an oral solution comprising miglustat is an oral solution comprising, 0.5-1%, 0.5-0.55%, 0.55-0.6%, 0.6-0.65%, 0.65-0.7%, 0.7-0.8%, 0.8-0.85%, 0.85-0.9%, or 0.9-0.95%. of sodium citrate by weight. In some embodiments an oral solution comprises less than, greater than, or equal to about 0.65% sodium citrate by weight. In some embodiments an oral solution comprises less than, greater than, or equal to about 0.84% sodium citrate by weight. In some embodiments an oral solution comprises a sodium citrate concentration of 0.1 to 100 mg/mL, such as 0.5 to 50 mg/mL, 1 to 20 mg/mL or 5 to 15 mg/mL. Exemplary sodium citrate concentrations include 1 mg/mL, 2 mg/mL, 3 mg/mL, 4 mg/mL, 5 mg/mL, 6 mg/ml, 7 mg/mL, 8 mg/mL, 9 mg/mL, 10 mg/mL, 11 mg/mL, 12 mg/mL, 13 mg/mL, 14 mg/mL, 15 mg/mL, 20 mg/mL, 25 mg/mL, 30 mg/mL, 35 mg/mL, 40 mg/mL or 50 mg/mL,

In some embodiments, an oral solution comprising miglustat contains a buffering agent. In some embodiments the buffering agent is citric acid. In some embodiments an oral solution comprising miglustat is an oral solution comprising 0.1-0.5%, 0.1-0.2%, 0.2-0.3%, 0.25-0.3%, 0.3-0.35%, 0.35-0.4%, 0.4-0.5%, of citric acid by weight. In some embodiments an oral solution comprises less than, greater than, or equal to about 0.27% citric acid by weight. In some embodiments an oral solution comprises less than, greater than, or equal to about 0.35% citric acid by weight. In some embodiments an oral solution comprises a citric acid concentration of 0.1 to 100 mg/mL, such as 0.5 to 50 mg/mL, 1 to 20 mg/mL or 5 to 15 mg/mL. Exemplary citric acid concentrations include 0.5 mg/mL, 1 mg/mL, 1.5 mg/mL, 2 mg/mL, 2.5 mg/mL, 3 mg/mL, 3.5 mg/mL, 4 mg/mL, 4.5 mg/mL, 5 mg/mL, 6 mg/ml, 7 mg/mL, 8 mg/mL, 9 mg/mL, 10 mg/mL, 15 mg/mL, 20 mg/mL, 25 mg/mL, 30 mg/mL, 35 mg/mL, 40 mg/mL or 50 mg/mL,

In some embodiments, an oral solution comprising miglustat contains a thickening agent. In some embodiments the thickening agent is Hypromellose. In some embodiments an oral solution comprising miglustat is an oral solution comprising the thickening agent at 1-10 mg/mL, 10-20 mg/mL, 1-5 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL. In some embodiments the oral solution comprises the thickening agent at less than, greater than, or equal to about 15 mg/mL. In some embodiments, an oral solution comprising miglustat is an oral solution comprising 1-5%, 1.5-2%, 1-1.5%, 1-2%, 2.5-3%, 3.5-4%, 4.5-5%, or 5.5-6% of the thickening agent by weight. In some embodiments, an oral solution comprises less than, greater than, or equal to about 1.5% thickening agent by weight.

In some embodiments, an oral solution comprising miglustat contains a sweetener. In some embodiments, the sweetener is sucralose. In some embodiments, an oral solution comprising miglustat is an oral solution comprising the sweetener at 1-5 mg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL. In some embodiments the oral solution comprises the sweetener at less than, greater than, or equal to about 2 mg/mL. In some embodiments, an oral solution comprising miglustat is an oral solution comprising 0.1-0.2%, 0.15-0.2%, 0.1-0.5%, 0.1-0.25%, 0.15-0.25%, 0.2-0.25%, 0.25-0.3%, 0.25-0.35%, 0.3-0.4%, 0.4-0.5% of the sweetener by weight. In some embodiments, an oral solution comprises less than, greater than, or equal to about 0.2% sweetener by weight.

In some embodiments, an oral solution comprising miglustat contains a preservative. In some embodiments, the preservative is potassium sorbate. In some embodiments, an oral solution comprising miglustat is an oral solution comprising the preservative at 1-5 mg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL. In some embodiments the oral solution comprises the preservative at less than, greater than, or equal to about 2 mg/mL. In some embodiments, an oral solution comprising miglustat is an oral solution comprising 0.1-0.2%, 0.15-0.2%, 0.1-0.5%, 0.1-0.25%, 0.15-0.25%, 0.2-0.25%, 0.25-0.3%, 0.25-0.35%, 0.3-0.4%, 0.4-0.5% of the preservative by weight. In some embodiments, an oral solution comprises less than, greater than, or equal to about 0.2% preservative by weight.

In some embodiments, an oral solution comprising miglustat contains a flavor. In some embodiments, the flavor is mixed berry. In some embodiments, an oral solution comprising miglustat is an oral solution comprising the flavor at 1-5 mg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL. In some embodiments the oral solution comprises the flavor at less than, greater than, or equal to about 2 mg/mL. In some embodiments, an oral solution comprising miglustat is an oral solution comprising 0.1-0.2%, 0.15-0.2%, 0.1-0.5%, 0.1-0.25%, 0.15-0.25%, 0.2-0.25%, 0.25-0.3%, 0.25-0.35%, 0.3-0.4%, 0.4-0.5% of the flavor by weight. In some embodiments, an oral solution comprises less than, greater than, or equal to about 0.2% flavor by weight.

Several exemplary pharmaceutical compositions comprising miglustat in an oral solution are provided below.

| | | Prototype 1 | | Prototype 2 | | Prototype 3 | | Prototype 4 | |
|---|---|---|---|---|---|---|---|---|---|
| | | | % | | % | | % | | % |
| Component | Functionality | mg/mL | (w/v) | mg/mL | (w/v) | mg/mL | (w/v) | mg/mL | (w/v) |
| Miglustat | Drug | 40 | 4.00 | 40 | 4.00 | 40 | 4.00 | 40 | 4.00 |
| | substance | | | | | | | | |
| Sodium Citrate | Buffering | 6.524 | 0.65 | 6.524 | 0.65 | 6.524 | 0.65 | 8.388 | 0.84 |
| USP/Ph. Eur. | agent | | | | | | | | |
| (Dihydrate) | | | | | | | | | |
| Citric Acid | Buffering | 2.693 | 0.27 | 2.693 | 0.27 | 2.693 | 0.27 | 3.463 | 0.35 |
| Monohydrate | agent | | | | | | | | |
| USP/Ph. Eur. | | | | | | | | | |
| Hypromellose | Thickening | — | — | 15 | 1.5 | 15 | 1.5 | — | — |
| USP/EP/JP | agent | | | | | | | | |
| Sucralose | Sweetener | 2 | 0.20 | 2 | 0.20 | 2 | 0.20 | 2 | 0.20 |
| NF/Ph. Eur. | | | | | | | | | |
| Potassium Sorbate | Preservative | 2 | 0.20 | 2 | 0.20 | 2 | 0.20 | 2 | 0.20 |
| NF | | | | | | | | | |
| Flavor (Mixed | Flavor | — | — | — | — | 2 | 0.20 | — | — |
| Berry) | | | | | | | | | |
| Purified Water | Vehicle | q.s. | — | q.s. | — | q.s. | — | q.s. | — |
| USP/Ph. Eur. | | | | | | | | | |

| | | |
|---|---|---|
| Hydrochloric Acid | pH modifier | For pH adjustment to 5.0 ± 0.5 |
| Sodium Hydroxide | pH modifier | For pH adjustment to 5.0 ± 0.5 |

### Methods of Treatment

### A. Treatment of Diseases

Another aspect of the disclosure pertains to a method of treatment of a disease or disorder related to glycogen storage dysregulation by administering the rhGAA or pharmaceutical composition described herein. In some embodiments, the disease is Pompe disease (also known as acid maltase deficiency (AMD) and glycogen storage disease type II (GSD II)). In some embodiments, the rhGAA is ATB200 or cipaglucosidase alfa. In some embodiments, the pharmaceutical composition comprises rhGAA (e.g., ATB200 or cipaglucosidase alfa). Also provided herein are uses of rhGAA or ATB200 or cipaglucosidase alfa) to treat Pompe disease.

In some embodiments, the subject treated by the methods disclosed herein is a pediatric patient. In some embodiments, the subject may have an age from 0 to 18 years of age or any intermediate value or subrange thereof including 0, 1, 2, 3, 4, 5, 6, 7, 8, 9, 10, 11 or 12 months of age or 1, 2, 3, 4, 5, 6, 7, 8, 9, 10, 11, 12, 13, 14, 15, 16, 17 or 18 years of age.

In some embodiments, the subject treated by the methods disclosed herein is an ERT-experienced patient. In some embodiments, the ERT-experienced patient is currently receiving an approved ERT (e.g., MYOZYME®, LUMIZYME® or NEXVIAZYME®). In some embodiments, the ERT-experienced patient is declining on their current treatment. In some embodiments, the methods disclosed herein are begun approximately 2 weeks after the last ERT dose. In some embodiments, the subject treated by the methods disclosed herein is an ERT-naive patient.

In some embodiments, the patient may be expected to be seen again every 3 months to ensure the clinical benefit provided to the patient and thus continue the treatment. This visit frequency may be required until the medicinal product is commercially available. In some embodiments, a patient's response to treatment is regularly assessed based on an assessment of the main clinical and laboratory parameters of the disease. In some embodiments, an rhGAA as described herein (such as cipaglucosidase alfa) and miglustat are administered every two weeks. In some embodiments, a dosage of rhGAA such as cipaglucosidase alfa is about 30 mg/kg of body weight given as a 4-hour infusion. In some embodiments, if the infusion is delayed, it should not be started more than 3 hours after oral administration of miglustat.

In some embodiments, subjects receive capsules of miglustat (or liquid dispersion prepared from miglustat capsule) approximately 1 hour (i.e., 1 hour±10 minutes) prior to cipaglucosidase alfa infusion. The number of capsules, amount of water to be used and amount of dispersion to be dosed are summarized in the table below. For example, for subjects who receive 260 mg or 195 mg of miglustat, 4 capsules or 3 capsules of miglustat will be administered orally, respectively.

In some embodiments, embodiments, if the subject cannot swallow the miglustat capsules, the capsule can be opened, and the contents transferred into water. In some embodiments, subjects receive a liquid dispersion administration prepared from miglustat capsule approximately 1 hour (i.e., 1 hour±10 minutes) prior to cipaglucosidase alfa infusion. The number of capsules, the amount of water to be used, and the amount of dispersion to be dosed are summarized in the table below. For example, for subjects who receive 260 mg of miglustat, 20 mL of water will be used to disperse 4 capsules of miglustat and will be given 20 mL.

TABLE
Number of Capsules and Amount of Water
to be Used to Prepare the Dispersion

| | | | Amount of |
|---|---|---|---|
| | # of capsules | Amount of Water | Dispersion |
| Miglustat | to be dispersed | used to | Withdrawn and |
| Dose (mg) | (capsules) | disperse (ml) | Administered (mL) |
| 260 | 4 | 20 | 20 |
| 195 | 3 | 20 | 20 |
| 175 | 3 | 20 | 18 |
| 130 | 2 | 10 | 10 |
| 115 | 2 | 10 | 8.8 |
| 85 | 2 | 10 | 6.5 |
| 65 | 1 | 5 | 5 |
| 40 | 1 | 5 | 3.1 |
| 20 | 1 | 5 | 1.5 |

In some of the above embodiments, rhGAA (such as cipaglucosidase alfa) is administered via infusion every two weeks.

In some embodiments, the miglustat is administered as an oral solution as described herein. As described above, exemplary oral solutions may comprise miglustat with one or more excipients such as a preservative, a buffering agent, a thickener (or a thickening agent), a sweetener, a flavor, and/or a vehicle. In some embodiments, the oral solution comprises miglustat and one or more of sodium citrate, citric acid, hypromellose, sucralose, potassium sorbate, flavoring and/or water.

The rhGAA or pharmaceutical composition described herein is administered by an appropriate route. In one embodiment, the rhGAA or pharmaceutical composition is administered intravenously. In some embodiments, the rhGAA or pharmaceutical composition is administered intravenously using an infusion pump. In some embodiments, when administered intravenously, the infusion bag and tubing are covered to protect from light. In other embodiments, the rhGAA or pharmaceutical composition is administered by direct administration to a target tissue, such as to heart or skeletal muscle (e.g., intramuscular), or nervous system (e.g., direct injection into the brain; intraventricularly; intrathecally). In some embodiments, the rhGAA or pharmaceutical composition is administered orally. More than one route can be used concurrently, if desired.

In some embodiments, the therapeutic effects of the rhGAA or pharmaceutical composition described herein may be assessed based on one or more of the following criteria: (1) cardiac status (e.g., increase of end-diastolic and/or end-systolic volumes, or reduction, amelioration or prevention of the progressive cardiomyopathy that is typically found in GSD-II), (2) pulmonary function (e.g., increase in crying vital capacity over baseline capacity, and/or normalization of oxygen desaturation during crying), (3) neurodevelopment and/or motor skills (e.g., increase in AIMS score), and (4) reduction of glycogen levels in tissue of the individual affected by the disease.

In some embodiments, the cardiac status of a subject is improved by 10%, 20%, 30%, 40%, or 50% (or any percentage in-between) after administration of one or more dosages of the rhGAA or pharmaceutical composition described herein, as compared to that of a subject treated with a vehicle or that of a subject prior to treatment. The cardiac status of a subject may be assessed by measuring end-diastolic and/or end-systolic volumes and/or by clinically evaluating cardiomyopathy. In some embodiments, the pulmonary function of a subject is improved by 10%, 20%, 30%, 40%, or 50% (or any percentage in-between) after administration of one or more dosages of ATB200 or cipaglucosidase alfa) or pharmaceutical composition comprising ATB200 or cipaglucosidase alfa), as compared to that of a subject treated with a vehicle or that of a subject prior to treatment. In certain embodiments, the improvement is achieved after 1 week, 2 weeks, 3 weeks, 1 month, 2 months, or more from administration (or any time period in between). In certain embodiments, rhGAA (e.g., ATB200 or cipaglucosidase alfa) or pharmaceutical composition comprising rhGAA (e.g., ATB200 or cipaglucosidase alfa) improves and/or stabilizes the pulmonary function of a subject after 1 week, 2 weeks, 3 weeks, 1 month, 2 months, or more from administration (or any time period in between).

In some embodiments, the pulmonary function of a subject is improved by 10%, 20%, 30%, 40%, or 50% (or any percentage in-between) after administration of one or more dosages of the rhGAA or pharmaceutical composition described herein, as compared to that of a subject treated with a vehicle or that of a subject prior to treatment. The pulmonary function of a subject may be assessed by crying vital capacity over baseline capacity, and/or normalization of oxygen desaturation during crying. In some embodiments, the pulmonary function of a subject is improved by 10%, 20%, 30%, 40%, or 50% (or any percentage in-between) after administration of one or more dosages of rhGAA (e.g., ATB200 or cipaglucosidase alfa) or pharmaceutical composition comprising rhGAA (e.g., ATB200 or cipaglucosidase alfa), as compared to that of a subject treated with a vehicle or that of a subject prior to treatment. In certain embodiments, the improvement is achieved after 1 week, 2 weeks, 3 weeks, 1 month, 2 months, or more from administration (or any time period in between). In certain embodiments, rhGAA (e.g., ATB200 or cipaglucosidase alfa) or pharmaceutical composition comprising rhGAA (e.g., ATB200 or cipaglucosidase alfa) improves the pulmonary function of a subject after 1 week, 2 weeks, 3 weeks, 1 month, 2 months, or more from administration (or any time period in between).

In some embodiments, the neurodevelopment and/or motor skills of a subject is improved by 10%, 20%, 30%, 40%, or 50% (or any percentage in-between) after administration of one or more dosages of the rhGAA (e.g., ATB200 or cipaglucosidase alfa) or pharmaceutical composition rhGAA (e.g., ATB200 or cipaglucosidase alfa) described herein, as compared to that of a subject treated with a vehicle or that of a subject prior to treatment. The neurodevelopment and/or motor skills of a subject may be assessed by determining an AIMS score. The AIMS is a 12-item anchored scale that is clinician-administered and scored (see Rush J A Jr., Handbook of Psychiatric Measures, American Psychiatric Association, 2000, 166-168). Items 1-10 are rated on a 5-point anchored scale. Items 1-4 assess orofacial movements. Items 5-7 deal with extremity and truncal dyskinesia. Items 8-10 deal with global severity as judged by the examiner, and the patient's awareness of the movements and the distress associated with them. Items 11-12 are yes/no questions concerning problems with teeth and/or dentures (such problems can lead to a mistaken diagnosis of dyskinesia). In some embodiments, the neurodevelopment and/or motor skills of a subject is improved by 10%, 20%, 30%, 40%, or 50% (or any percentage in-between) after administration of one or more dosages of rhGAA (e.g., ATB200 or cipaglucosidase alfa) or pharmaceutical composition comprising rhGAA (e.g., ATB200 or cipaglucosidase alfa), as compared to that of a subject treated with a vehicle or that of a subject prior to treatment. In certain embodiments, the improvement is achieved after 1 week, 2 weeks, 3 weeks, 1 month, 2 months, or more from administration (or any time period in between). In certain embodiments, rhGAA (e.g., ATB200 or cipaglucosidase alfa) or pharmaceutical composition comprising rhGAA (e.g., ATB200 or cipaglucosidase alfa) improves and/or stabilizes the neurodevelopment and/or motor skills of a subject after 1 week, 2 weeks, 3 weeks, 1 month, 2 months, or more from administration (or any time period in between).

In some embodiments, the glycogen level of a certain tissue of a subject is reduced by 10%, 20%, 30%, 40%, or 50% (or any percentage in-between) after administration of one or more dosages of the rhGAA or pharmaceutical composition described herein, as compared to that of a subject treated with a vehicle or that of a subject prior to treatment. In some embodiment, the tissue is muscle such as quadriceps, triceps, and gastrocnemius. The glycogen level of a tissue can be analyzed using methods known in the art. The determination of glycogen levels is well known based on amyloglucosidase digestion, and is described in publications such as: Amalfitano et al. (1999), Proc Natl Acad Sci USA, 96:8861-8866. In some embodiments, the glycogen level in muscle of a subject is reduced by 10%, 20%, 30%, 40%, or 50% (or any percentage in between) after administration of one or more dosages of rhGAA (e.g., ATB200 or cipaglucosidase alfa) or pharmaceutical composition comprising rhGAA (e.g., ATB200 or cipaglucosidase alfa), as compared to that of a subject treated with a vehicle or that of a subject prior to treatment. In certain embodiments, the reduction is achieved after 1 week, 2 weeks, 3 weeks, 1 month, 2 months, or more from administration (or any time period in between). In certain embodiments, rhGAA (e.g., ATB200 or cipaglucosidase alfa) or pharmaceutical composition comprising rhGAA (e.g., ATB200 or cipaglucosidase alfa) reduces the glycogen level in muscle of a subject after 1 week, 2 weeks, 3 weeks, 1 month, 2 months, or more from administration (or any time period in between).

### B. Biomarkers

Biomarkers of glycogen accumulation in a subject, such as urine hexose tetrasaccharide (Hex4), may be used to assess and compare the therapeutic effects of enzyme replacement therapy in a subject with Pompe disease. In some embodiments, the therapeutic effect of the rhGAA or a pharmaceutical composition comprising rhGAA on glycogen accumulation is assessed by measuring the levels of urinary Hex4 in a subject.

Biomarkers of muscle injury or damage such as creatine kinase (CK), alanine aminotransferase (ALT), and aspartate aminotransferase (AST) may be used to assess and compare the therapeutic effects of enzyme replacement therapy in a subject with Pompe disease. In some embodiments, the therapeutic effect of the rhGAA or a pharmaceutical composition comprising rhGAA on muscle damage is assessed by measuring the levels of CK, ALT, and/or AST in a subject. In at least one embodiment, the therapeutic effect of the rhGAA or a pharmaceutical composition comprising rhGAA on muscle damage is assessed by measuring the levels of CK in a subject.

Biomarkers such as LAMP-1, LC3, and Dysferlin may also be used to assess and compare the therapeutic effects of the rhGAA or pharmaceutical composition described herein. In Pompe disease, the failure of GAA to hydrolyze lysosomal glycogen leads to the abnormal accumulation of large lysosomes filled with glycogen in some tissues. (Raben et al., JBC 273:19086-19092, 1998.) Studies in a mouse model of Pompe disease have shown that the enlarged lysosomes in skeletal muscle cannot adequately account for the reduction in mechanical performance, and that the presence of large inclusions containing degraded myofibrils (i.e., autophagic buildup) contributes to the impairment of muscle function. (Raben et al., Human Mol Genet 17:3897-3908, 2008.) Reports also suggest that impaired autophagy flux is associated with poor therapeutic outcome in Pompe patients. (Nascimbeni et al., Neuropathology and Applied Neurobiology doi: 10.1111/nan.12214, 2015; Fukuda et al., Mol Ther 14:831-839, 2006.) In addition, late-onset Pompe disease is prevalent in unclassified limb-girdle muscular dystrophies (LGMDs) (Preisler et al., Mol Genet Metab 110:287-289, 2013), which is a group of genetically heterogeneous neuromuscular diseases with more than 30 genetically defined subtypes of varying severity. IHC examination revealed substantially elevated sarcoplasmic presence of dysferlin in the skeletal muscle fibers of Gaa KO mice.

Various known methods can be used to measure the gene expression level and/or protein level of such biomarkers. For instance, a sample from a subject treated with the rhGAA or pharmaceutical composition described herein can be obtained, such as biopsy of tissues, in particular muscle. In some embodiments, the sample is a biopsy of muscle in a subject. In some embodiments, the muscle is selected from quadriceps, triceps, and gastrocnemius. The sample obtained from a subject may be stained with one or more antibodies or other detection agents that detect such biomarkers or be identified and quantified by mass spectrometry. The samples may also or alternatively be processed for detecting the presence of nucleic acids, such as mRNAs, encoding the biomarkers via, e.g., RT-qPCR methods.

In some embodiments, the gene expression level and/or protein level of one or more biomarkers is measured in a muscle biopsy obtained from an individual prior to and post treatment with the rhGAA or pharmaceutical composition described herein. In some embodiments, the gene expression level and/or protein level of one or more biomarkers is measured in a muscle biopsy obtained from an individual treated with a vehicle. In some embodiments, the gene expression level and/or protein level of one or more biomarkers is reduced by 10%, 20%, 30%, 40%, or 50% (or any percentage in-between) after administration of one or more dosages of the rhGAA or pharmaceutical composition described herein, as compared to that of a subject treated with a vehicle or that of a subject prior to treatment. In some embodiments, the gene expression level and/or protein level of one or more biomarkers is reduced by 10%, 20%, 30%, 40%, or 50% (or any percentage in-between) after administration of one or more dosages of rhGAA (e.g., ATB200 or cipaglucosidase alfa) or pharmaceutical composition comprising rhGAA (e.g., ATB200 or cipaglucosidase alfa), as compared to that of a subject treated with a vehicle or that of a subject prior to treatment. In certain embodiments, the reduction is achieved after 1 week, 2 weeks, 3 weeks, 1 month, 2 months, or more from administration (or any time period in between). In certain embodiments, rhGAA (e.g., ATB200 or cipaglucosidase alfa) or pharmaceutical composition comprising rhGAA (e.g., ATB200 or cipaglucosidase alfa) reduces the gene expression level and/or protein level of one or more biomarkers after 1 week, 2 weeks, 3 weeks, 1 month, 2 months, or more from administration (or any time period in between).

C. Dosages of rhGAA

The pharmaceutical formulation or reconstituted composition is administered in a therapeutically effective amount (e.g., a dosage amount that, when administered at regular intervals, is sufficient to treat the disease, such as by ameliorating symptoms associated with the disease, delaying the onset of the disease, and/or lessening the severity or frequency of symptoms of the disease). The amount which is therapeutically effective in the treatment of the disease may depend on the nature and extent of the disease's effects. In addition, in vitro or in vivo assays may optionally be employed to help identify optimal dosage ranges. In at least one embodiment, a rhGAA described herein or pharmaceutical composition comprising the rhGAA is administered at a dose of about 1 mg/kg to about 100 mg/kg, such as about 5 mg/kg to about 50 mg/kg, typically about 20 mg/kg to about 40 mg/kg. In at least one embodiment, the rhGAA or pharmaceutical composition described herein is administered at a dose of about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, or about 100 mg/kg. In some embodiments, the rhGAA is administered at a dose of 5 mg/kg, 10 mg/kg, 20 mg/kg, 25 mg/kg, 30 mg/kg, 35 mg/kg, 40 mg/kg, 50 mg/kg, 75 mg/kg, or 100 mg/kg. In at least one embodiment, the rhGAA or pharmaceutical composition is administered at a dose of about 20, 21, 22, 23, 24, 25, 26, 27, 28, 29, 30, 31, 32, 33, 34, 35, 36, 37, 38, 39, or 40 mg/kg. In at least one embodiment, the rhGAA or pharmaceutical composition is administered at a dose of about 30 mg/kg. In at least one embodiment, the rhGAA or pharmaceutical composition is administered at a dose of about 25 mg/kg. In at least one embodiment, the rhGAA or pharmaceutical composition is administered at a dose of about 20 mg/kg.

In at least one embodiment the dosage of the rhGAA is dependent on the patient's age. In at least one embodiment, the dosage of the rhGAA is dependent on the patient's weight. In at least one embodiment, the dosage of the rhGAA is dependent on the patient's age and weight. In at least one embodiment, the dosage of the rhGAA is provided according to Table 6A below.

In at least one embodiment, the dosage of rhGAA and/or miglustat is provided in Tables 6A and 6B below. In some embodiments, the rhGAA or pharmaceutical composition is administered concurrently or sequentially with an enzyme stabilizer. In some embodiments, the enzyme stabilizer is miglustat. In at least one embodiment, the miglustat is administered as an oral dose in a range of about 20 mg to about 260 mg depending on patient age and weight or any intermediate value or subrange thereof including 20, 40, 65, 85, 115, 130, 175, 195, or 260 mg. The effective dose for a particular individual can also be varied (e.g., increased or decreased) over time, depending on the needs of the individual. For example, in times of physical illness or stress, or if anti-acid α-glucosidase antibodies become present or increase, or if disease symptoms worsen, the amount of rhGAA and/or miglustat can be adjusted.

In some embodiments, the therapeutic effect of the rhGAA or pharmaceutical composition described herein comprises an improvement and/or stabilization in motor function, an improvement and/or stabilization in muscle strength (upper-body, lower-body, or total-body), an improvement and/or stabilization in pulmonary function, decreased fatigue, reduced levels of at least one biomarker of muscle injury, reduced levels of at least one biomarker of glycogen accumulation, or a combination thereof. In some embodiments, the therapeutic effect of the rhGAA or pharmaceutical composition described herein comprises a reversal of lysosomal pathology in a muscle fiber, a faster and/or more effective reduction in glycogen content in a muscle fiber, an increase in six-minute walk test distance, a decrease in timed up and go test time, a decrease in four-stair climb test time, a decrease in ten-meter walk test time, a decrease in gait-stair-gower-chair score, an increase in upper extremity strength, an improvement and/or stabilization in shoulder adduction, an improvement and/or stabilization in shoulder abduction, an improvement and/or stabilization in elbow flexion, an improvement and/or stabilization in elbow extension, an improvement and/or stabilization in upper body strength, an improvement and/or stabilization in lower body strength, an improvement and/or stabilization in total body strength, an improvement in upright (sitting) forced vital capacity, an improvement and/or stabilization in maximum expiratory pressure, an improvement and/or stabilization in maximum inspiratory pressure, a decrease in fatigue severity scale score, a reduction in urine hexose tetrasaccharide levels, a reduction in creatine kinase levels, a reduction in alanine aminotransferase levels, a reduction in aspartate aminotransferase levels, or any combination thereof.

In some embodiments, the rhGAA or pharmaceutical composition described herein achieves desired therapeutic effects faster than conventional rhGAA products when administered at the same dose. Therapeutic effects may be assessed based on one or more criteria discussed above (e.g., cardiac status, glycogen level, or biomarker expression). For instance, if a single dose of a conventional rhGAA product decreases glycogen levels in tissue of a treated individual by 10% in a week, the same degree of reduction may be achieved in less than a week when the same dose of the rhGAA or pharmaceutical composition described herein is administered. In some embodiments, when administered at the same dose, the rhGAA or pharmaceutical composition described herein may achieve desired therapeutic effects at least about 1.25, 1.5, 1.75, 2.0, 3.0, or faster than conventional rhGAA products.

In some embodiments, the therapeutically effective amount of rhGAA (or composition or medicament comprising rhGAA) is administered more than once. In some embodiments, the rhGAA or pharmaceutical composition described herein is administered at regular intervals, depending on the nature and extent of the disease's effects, and on an ongoing basis. Administration at a “regular interval,” as used herein, indicates that the therapeutically effective amount is administered periodically (as distinguished from a one-time dose). The interval can be determined by standard clinical techniques. In certain embodiments, rhGAA is administered bimonthly, monthly, bi-weekly, weekly, twice weekly, or daily. In some embodiments, the rhGAA is administered intravenously twice weekly, weekly, or every other week. The administration interval for a single individual need not be a fixed interval, but can be varied over time, depending on the needs of the individual. For example, in times of physical illness or stress, if anti-rhGAA antibodies become present or increase, or if disease symptoms worsen, the interval between doses can be decreased.

In some embodiments, when used at the same dose, the rhGAA or pharmaceutical composition as described herein may be administered less frequently than conventional rhGAA products and yet capable of producing the same as or better therapeutic effects than conventional rhGAA products. For instance, if a conventional rhGAA product is administered at 20 mg/kg, 30 mg/kg or 40 mg/kg weekly, the rhGAA or pharmaceutical composition as described herein may produce the same as or better therapeutic effects than the conventional rhGAA product when administered at 20 mg/kg, 30 mg/kg, or 40 mg/kg, even though the rhGAA or pharmaceutical composition is administered less frequently, e.g., biweekly or monthly. Therapeutic effects may be assessed based on one or more criterion discussed above (e.g., cardiac status, glycogen level, or biomarker expression). In some embodiments, an interval between two doses of the rhGAA or pharmaceutical composition described herein is longer than that of conventional rhGAA products. In some embodiments, the interval between two doses of the rhGAA or pharmaceutical composition is at least about 1.25, 1.5, 1.75, 2.0, 3.0, or more, longer than that of conventional rhGAA products.

In some embodiments, under the same treatment condition (e.g., the same dose administered at the same interval), the rhGAA or pharmaceutical composition described herein provides therapeutic effects at a degree superior to that provided by conventional rhGAA products. Therapeutic effects may be assessed based on one or more criteria discussed above (e.g., cardiac status, glycogen level, or biomarker expression). For instance, when compared to a conventional rhGAA product administered at 20 mg/kg, 30 mg/kg, or 40 mg/kg weekly, the rhGAA or pharmaceutical composition administered at 20 mg/kg or 30 mg/kg weekly may reduce glycogen levels in tissue of a treated individual at a higher degree. In some embodiments, when administered under the same treatment condition, the rhGAA or pharmaceutical composition described herein provides therapeutic effects that are at least about 1.25, 1.5, 1.75, 2.0, 3.0, or more, greater than those of conventional rhGAA products.

### D. Two-Component Therapy

In one or more embodiments, the rhGAA or pharmaceutical composition comprising the rhGAA described herein is administered concurrently or sequentially with an enzyme stabilizer. In some embodiments, the rhGAA or pharmaceutical composition is administered via a different route as compared to the enzyme stabilizer. For instance, an enzyme stabilizer may be administered orally while the rhGAA or pharmaceutical composition is administered intravenously.

In some embodiments, the two-component therapy is administered in combination with additional therapies such as gene therapy or substrate reduction therapy. Exemplary gene therapy candidates include those that encode the GAA gene with optional tags such as insulin-like growth factor-II (IGF2) or variants thereof and/or binding immunoglobulin protein (Bip) or variants thereof. Exemplary substrate reduction therapy strategies include inhibiting glycogen synthesis using small molecules or genetic inhibition. In some embodiments, the two-component therapy comprises ERT or a pharmacological chaperon as the first component of the therapy and gene therapy or substrate reduction therapy as the second component of the therapy. Such substrate reduction therapy includes, for example, small molecule inhibition of glycogenin (GYG) or glycogen synthase (GYS) (e.g., GYS1 small molecule inhibitor currently in human trial NCT05249621) or genetic approaches (e.g., antisense oligonucleotide-mediated suppression of muscle GYS1 synthesis).

In various embodiments, the enzyme stabilizer is miglustat. Without wishing to be bound by any theory, it is believed that when co-administered, miglustat stabilizes rhGAA (e.g., ATB200 or cipaglucosidase alfa) from denaturation in systemic circulation, which enhances the delivery of the active component rhGAA (e.g., ATB200 or cipaglucosidase alfa) to lysosomes.

In some embodiments, the miglustat is administered at an oral dose of about 10 mg to about 600 mg. In at least one embodiment, the miglustat is administered at an oral dose of about 20 mg to about 300 mg, or at an oral dose of about 20 mg, about 40 mg, about 65 mg, about 85 mg, about 115 mg, about 130 mg, about 175 mg, about 195 mg, or about 260 mg. Exemplary oral doses for miglustat also include about 15 mg to about 25 mg, about 30 mg to about 50 mg, about 50 mg to about 80 mg, about 70 mg to about 100 mg, about 100 mg to about 130 mg, about 110 mg to about 150 mg, about 150 mg to about 200 mg, about 170 mg to about 220 mg, and about 200 mg to about 300 mg.

In at least one embodiment, the dosage of miglustat is dependent on the patient's age. In at least one embodiment, the dosage of miglustat is dependent on the patient's weight. In at least one embodiment, the dosage of miglustat is dependent on the patient's age and weight.

In at least one embodiment, the dosage of rhGAA is provided in the table below.

TABLE 6A
Dosages for rhGAA

| | Weight Group | Cipaglucosidase alfa Starting |
|---|---|---|
| Age Group | (kg) | Dose (mg/kg) |
| 0 to <30 days | <10 | 30 |
| 1 to <6 mo | <10 | 30 |
| 6 mo to <2 yr | <10 | 30 |
| | 10 to <20 | 30 |
| 2 to <6 yr | <10 | 30 |
| | 10 to <20 | 30 |
| | 20 to <30 | 30 |
| | 30 to <40 | 30 |
| 6 to <12 yr | 10 to <20 | 30 |
| | 20 to <30 | 30 |
| | 30 to <40 | 30 |
| | 40 to <50 | 30 |
| | ≥50 | 30 |
| 12 to <18 yr | 30 to <40 | 30 |
| | 40 to <50 | 30 |
| | ≥50 | 30 |

In at least one embodiment, the dosage of miglustat is provided in the table below.

TABLE 6B
Dosages for Miglustat

| | Weight Group | Fixed Miglustat |
|---|---|---|
| Age Group | (kg) | Dose (mg) |
| 0 to <30 days | <10 | 20 |
| 1 to <6 mo | <10 | 40 |
| 6 mo to <2 yr | <10 | 65 |
| | 10 to <20 | 85 |
| 2 to <6 yr | <10 | 85 |
| | 10 to <20 | 115 |
| | 20 to <30 | 130 |
| | 30 to <40 | 175 |
| 6 to <12 yr | 10 to <20 | 115 |
| | 20 to <30 | 130 |
| | 30 to <40 | 175 |
| | 40 to <50 | 195 |
| | ≥50 | 260 |
| 12 to <18 yr | 30 to <40 | 175 |
| | 40 to <50 | 195 |
| | ≥50 | 260 |

Miglustat exhibited linear pharmacokinetics with plasma area under the concentration-time curve (AUC) and maximum concentration (Cₘₐₓ) increased approximately proportional with increasing doses from 130 mg (0.5-fold of the recommended dose of 260 mg in patients weighing ≥50 kg) to 260 mg. At the recommended 260 mg dose, the mean Cₘₐₓ was approximately 3 mcg/mL and the mean AUC was approximately 25 mcg*hr/mL. The mean time to reach the maximum concentration ranged from 2 hours to 3 hours.

In some embodiments, the rhGAA is administered intravenously at a dose described herein, such as a dose of about 5 mg/kg to about 40 mg/kg and the miglustat is administered orally at a dose described herein, such as a dose of about 10 mg to about 600 mg. In some embodiments, the rhGAA is administered intravenously at a dose described herein, such as a dose of about 15 mg/kg to about 40 mg/kg and the miglustat is administered orally at a dose described herein, such as a dose of about 15 mg to about 300 mg. In some embodiments, the rhGAA is administered intravenously at a dose of about 30 mg/kg, wherein the total volume of infusion is determined by the subject's body weight (e.g., as set forth in Table 5). In some embodiments, the miglustat is administered orally at a dose determined by the age and/or weight of the subject (e.g., as set forth in Table 6B).

In some embodiments, the miglustat and the rhGAA are administered concurrently. For instance, the miglustat may administered within 10, 9, 8, 7, 6, 5, 4, 3, 2, or 1 minute(s) before or after administration of the rhGAA. In some embodiments, the miglustat is administered within 5, 4, 3, 2, or 1 minute(s) before or after administration of the rhGAA.

In some embodiments, the miglustat and the rhGAA are administered sequentially. In at least one embodiment, the miglustat is administered prior to administration of the rhGAA. In at least one embodiment, the miglustat is administered less than three hours prior to administration of the rhGAA. In at least one embodiment, the miglustat is administered about two hours prior to administration of the rhGAA. In at least one embodiment, the miglustat is administered in a range of 50 minutes to 90 minutes prior to administration of the rhGAA. For instance, the miglustat may be administered about 1.5 hours, about 1 hour, about 50 minutes, about 30 minutes, or about 20 minutes prior to administration of the rhGAA. In at least one embodiment, the miglustat is administered about one hour prior to administration of the rhGAA. In some embodiments, the miglustat is orally administered about one hour prior to administration of the rhGAA.

In some embodiments, the miglustat is administered after administration of the rhGAA. In at least one embodiment, the miglustat is administered within three hours after administration of the rhGAA. In at least one embodiment, the miglustat is administered within two hours after administration of the rhGAA. For instance, the miglustat may be administered within about 1.5 hours, about 1 hour, about 50 minutes, about 30 minutes, or about 20 minutes after administration of the rhGAA.

In some embodiments, the subject fasts for at least two hours before administration of miglustat. In some embodiments, the subject fasts for at least two hours after administration of miglustat. In some embodiments, the subject fasts for at least two hours before and at least two hours after administration of miglustat.

In some embodiments, the subject fasts for at least two hours before and at least two hours after administration of miglustat, and the miglustat is administered about one hour prior to administration of the rhGAA. In some embodiments, the fasting, miglustat administration and rhGAA administration follows the following dosing timeline:

In some embodiments, the two-component therapy according to this disclosure improves one or more disease symptoms in a subject with Pompe disease compared to (1) baseline, or (2) a control treatment comprising administering alglucosidase alfa and a placebo for the enzyme stabilizer. In such control treatment, a placebo was administered in place of the enzyme stabilizer.

Provided herein is a method for improving motor function and/or pulmonary function for at least 24 months or at least 36 months in a subject having Pompe disease, the method comprising administering to the subject a population of recombinant human acid α-glucosidase (rhGAA) molecules, concurrently or sequentially with an enzyme stabilizer; wherein each rhGAA molecule comprises seven potential N-glycosylation sites; wherein 40%-60% of the N-glycans on the rhGAA molecules are complex type N-glycans; wherein the rhGAA molecules comprise at least 0.5 mol bis-mannose-6-phosphate (bis-M6P) per mol of rhGAA at the first potential N-glycosylation site as determined using liquid chromatography tandem mass spectrometry (LC-MS/MS); and wherein the method improves motor function and/or pulmonary function in the subject compared to baseline.

In some embodiments, the subject treated by two-component therapy is an ERT-experienced patient. In some embodiments, the ERT-experienced subject had been previously treated with alglucosidase alfa. In some embodiments, the ERT-experienced subject had been previously treated with alglucosidase alfa for from about 2 years to about 6 years. In some embodiments, the ERT-experienced subject had been previously treated with alglucosidase alfa for at least about 7 years. In some embodiments, the ERT-experienced subject is non-ambulatory. In some embodiments, the ERT-experienced subject is ambulatory. In some embodiments, the subject is an ERT-naïve subject.

In some embodiments, the two-component therapy according to this disclosure improves and/or stabilizes the subject's motor function, as measured by a 6-minute walk test (6MWT). In some embodiments, compared to baseline, the subject's 6-minute walk distance (6MWD) is increased by at least 10, 11, 12, 13, 14, 15, 16, 17, 18, 19, 20, 21, 22, 23, 24, 25, 30, or 50 meters or at least 1%, 2%, 3%, 4%, 5%, 6%, 7%, 8%, 9%, or 10% after 12, 26, 38, or 52 weeks, or after 18, 24, 30, or 36 months of treatment. In some embodiments, the subject's 6MWD is increased by at least 20 meters or at least 5% after 52 weeks of treatment. In some embodiments, compared to the control treatment, the subject's 6MWD is improved by at least 5, 6, 7, 8, 9, 10, 12, 14, 16, 18, 20, 30, 40, or 50 meters after 12, 26, 38, or 52 weeks, or after 18, 24, 30, or 36 months of treatment. In some embodiments, compared to the control treatment, the subject's 6MWD is improved by at least 13 meters after 52 weeks of treatment. In some embodiments of methods for treating an ERT-experienced subject, the motor function is measured by a 6-minute walk test; and the improvement from baseline in 6-minute walk distance (6MWD) is at least 15, 16, 17, 18, 19, 20, or 21 meters at 24 months after initiation of treatment. In some embodiments of methods for treating an ERT-experienced subject, the motor function is measured by a 6-minute walk test; and the improvement from baseline in 6-minute walk distance (6MWD) is at least 35, 40, 41, 42, 43, 44, 45, 46, or 47 meters at 36 months after initiation of treatment. In some embodiments of methods for treating an ERT-naïve subject, the motor function is measured by a 6-minute walk test; and the improvement from baseline in 6-minute walk distance (6MWD) is at least 55, 56, 57, 58, 59, or 60 meters at 24 months after initiation of treatment. In some embodiments of methods for treating an ERT-naïve subject, the motor function is measured by a 6-minute walk test; and the improvement from baseline in 6-minute walk distance (6MWD) is at least 34, 35, 40, 41, 42, or 43 meters at 36 months after initiation of treatment. In some embodiments, the subject has a baseline 6MWD less than 300 meters. In some embodiments, the subject has a baseline 6MWD greater than or equal to 300 meters.

In some embodiments, the two-component therapy according to this disclosure stabilizes the subject's pulmonary function, as measured by a forced vital capacity (FVC) test. In some embodiments, after 12, 26, 38, or 52 weeks, or after 18, 24, 30, or 36 months of treatment, the subject's percent-predicted FVC is either increased compared to baseline, or decreased by less than 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% compared to baseline. In some embodiments, after 52 weeks of treatment, the subject's percent-predicted FVC is decreased by less than 1% compared to baseline. In some embodiments, compared to the control treatment, the subject's percent-predicted FVC is significantly improved and/or stabilized after treatment. In some embodiments, compared to the control treatment, the subject's percent-predicted FVC is significantly improved by at least 0.5%, 1%, 2%, 3%, 4%, 5%, or 6% after 12, 26, 38, or 52 weeks, or after 18, 24, 30, or 36 months of treatment. In some embodiments, compared to the control treatment, the subject's percent-predicted FVC is significantly improved by at least 3% after 52 weeks of treatment. In some embodiments of methods for treating an ERT-experienced subject, the pulmonary function is measured by a sitting forced vital capacity (FVC) test, and the subject's percent-predicted FVC is stable compared to baseline at 24 months or 36 months after initiation of treatment. In some embodiments of methods for treating an ERT-naïve subject, the pulmonary function is measured by a sitting forced vital capacity (FVC) test; and the improvement from baseline in the subject's percent-predicted FVC is at least 6.4, 6.5, 6.6, 6.7, or 6.8% at 24 months after initiation of treatment. In some embodiments of methods for treating an ERT-naïve subject, the pulmonary function is measured by a sitting forced vital capacity (FVC) test; and the improvement from baseline in the subject's percent-predicted FVC is at least 5.7, 5.8, 5.9, 6.0, 6.1, or 6.2% at 24 months after initiation of treatment. In some embodiments, the subject has a baseline FVC less than 55%. In some embodiments, the subject has a baseline FVC greater than or equal to 55%. In some embodiments, the subject has a baseline FVC less than 50%. In some embodiments, the subject has a baseline FVC greater than or equal to 50%.

In some embodiments, the two-component therapy according to this disclosure improves and/or stabilizes the subject's muscle strength, as measured by a manual muscle test (MMT). In some embodiments, compared to baseline, the subject's MMT lower extremity score is improved as indicated by an increase of at least 0.1, 0.3, 0.5, 0.7, 1.0, 1.5, 2.0, 2.5, or 3.0 points after 12, 26, 38 or 52 weeks, or after 18, 24, 30, or 36 months of treatment. In some embodiments, compared to the control treatment, the subject's MMT lower extremity score is significantly improved and/or stabilized after treatment. In some embodiments of methods for treating an ERT-experienced subject, the muscle strength is measured by a MMT; and the improvement from baseline in a MMT lower extremity score is at least 1.9, 2, 2.1, 2.2, or 2.3 points at 24 months after initiation of treatment. In some embodiments of methods for treating an ERT-experienced subject, the muscle strength is measured by a MMT; and the improvement from baseline in a MMT lower extremity score is at least 1.5, 1.6, 1.7, 1.8, or 1.9 points at 36 months after initiation of treatment. In some embodiments of methods for treating an ERT-naïve subject, the muscle strength is measured by a MMT; and the improvement from baseline in a MMT lower extremity score is at least 1.0, 1.1, 1.2, 1.3, 1.4, or 1.5 points at 24 months after initiation of treatment. In some embodiments of methods for treating an ERT-naïve subject, the muscle strength is measured by a MMT; and the improvement from baseline in a MMT lower extremity score is at least 2.8, 2.9, 3.0, 3.1, 3.2, or 3.3 points at 36 months after initiation of treatment. In some embodiments, the subject has a baseline MMT lower extremity score less than 25. In some embodiments, the subject has a baseline MMT lower extremity score greater than or equal to 25.

In some embodiments, the two-component therapy according to this disclosure improves and/or stabilizes the subject's motor function, as measured by a gait, stair, gower, chair (GSGC) test. In some embodiments, compared to baseline, the subject's GSGC score is improved as indicated by a decrease of at least 0.1, 0.3, 0.5, 0.7, 1.0, 1.5, or 2.5 points after 12, 26, 38 or 52 weeks, or after 18, 24, 30, or 36 months of treatment. In some embodiments, compared to baseline, the subject's GSGC score is improved as indicated by a decrease of at least 0.5 points after 52 weeks of treatment. In some embodiments, compared to the control treatment, the subject's GSGC score is significantly improved after treatment. In some embodiments, compared to the control treatment, the subject's GSGC score is significantly improved as indicated by a decrease of at least 0.3, 0.5, 0.7, 1.0, 1.5, 2.5, or 5 points after 12, 26, 38, or 52 weeks, or after 18, 24, 30, or 36 months of treatment. In some embodiments, compared to the control treatment, the subject's GSGC score is significantly improved as indicated by a decrease of at least 1.0 point after 52 weeks of treatment.

In some embodiments, the two-component therapy according to this disclosure reduces the level of at least one marker of muscle damage after treatment. In some embodiments, the at least one marker of muscle damage comprises creatine kinase (CK). In some embodiments, compared to baseline, the subject's CK level is reduced by at least 10%, 15%, 20%, 25%, 30%, 40%, or 50% after 12, 26, 38, or 52 weeks, or after 18, 24, 30, or 36 months of treatment. In some embodiments, compared to baseline, the subject's CK level is reduced by at least 20% after 52 weeks of treatment. In some embodiments, compared to the control treatment, the subject's CK level is significantly reduced after treatment. In some embodiments, compared to the control treatment, the subject's CK level is significantly reduced by at least 10%, 15%, 20%, 25%, 30%, 40%, or 50% after 12, 26, 38, or 52 weeks, or after 18, 24, 30, or 36 months of treatment. In some embodiments, compared to the control treatment, the subject's CK level is significantly reduced by at least 30% after 52 weeks of treatment.

In some embodiments, the two-component therapy according to this disclosure reduces the level of at least one marker of glycogen accumulation after treatment. In some embodiments, the at least one marker of glycogen accumulation comprises urine hexose tetrasaccharide (Hex4). In some embodiments, compared to baseline, the subject's urinary Hex4 level is reduced by at least 10%, 15%, 20%, 25%, 30%, 40%, 50%, or 60% after 12, 26, 38, or 52 weeks, or after 18, 24, 30, or 36 months of treatment. In some embodiments, compared to baseline, the subject's urinary Hex4 level is reduced by at least 30% after 52 weeks of treatment. In some embodiments, compared to the control treatment, the subject's urinary Hex4 level is significantly reduced after treatment. In some embodiments, compared to the control treatment, the subject's urinary Hex4 level is significantly reduced by at least 10%, 15%, 20%, 25%, 30%, 40%, 50%, or 60% after 12, 26, 38, or 52 weeks, or after 18, 24, 30, or 36 months of treatment. In some embodiments, compared to the control treatment, the subject's urinary Hex4 level is significantly reduced by at least 40% after 52 weeks of treatment.

In some embodiments, the two-component therapy according to this disclosure improves and/or stabilizes one or more disease symptoms in an ERT-experienced patient subject with Pompe disease compared to (1) baseline, or (2) a control treatment comprising administering alglucosidase alfa and a placebo for the enzyme stabilizer.

In some embodiments, the two-component therapy for an ERT-experienced subject with Pompe disease improves and/or stabilizes the subject's motor function, as measured by a 6MWT. In some embodiments, compared to baseline, the subject's 6MWD is increased by at least 10, 11, 12, 13, 14, 15, 16, 17, 18, 19, 20, 21, 22, 23, 24, 25, 30, or 50 meters or at least 1%, 2%, 3%, 4%, 5%, 6%, 7%, 8%, 9%, or 10% after 12, 26, 38, or 52 weeks, or after 18, 24, 30, or 36 months of treatment. In some embodiments, the subject's 6MWD is increased by at least 15 meters or at least 5% after 52 weeks of treatment. In some embodiments, compared to the control treatment, the subject's 6MWD is significantly improved after treatment. In some embodiments, compared to the control treatment, the subject's 6MWD is significantly improved by at least 10, 12, 14, 15, 16, 18, 20, 30, 40, or 50 meters after 12, 26, 38, or 52 weeks, or after 18, 24, 30, or 36 months of treatment. In some embodiments, compared to the control treatment, the subject's 6MWD is significantly improved by at least 15 meters after 52 weeks of treatment. In some embodiments, the subject has a baseline 6MWD less than 300 meters. In some embodiments, the subject has a baseline 6MWD greater than or equal to 300 meters.

In some embodiments, the two-component therapy for an ERT-experienced subject with Pompe disease improves and/or stabilizes the subject's pulmonary function, as measured by an FVC test. In some embodiments, after 12, 26, 38, or 52 weeks, or after 18, 24, 30, or 36 months of treatment, the subject's percent-predicted FVC is increased by at least 0.1%, 0.2%, 0.3%, 0.4%, 0.5%, 1%, 2%, 3%, 4%, or 5% compared to baseline. In some embodiments, after 52 weeks of treatment, the subject's percent-predicted FVC is increased by at least 0.1% compared to baseline. In some embodiments, compared to the control treatment, the subject's percent-predicted FVC is significantly improved and/or stabilized after treatment. In some embodiments, compared to the control treatment, the subject's percent-predicted FVC is significantly improved by at least 1%, 2%, 3%, 4%, 5%, 6%, 8%, or 10% after 12, 26, 38, or 52 weeks, or after 18, 24, 30, or 36 months of treatment. In some embodiments, compared to the control treatment, the subject's percent-predicted FVC is significantly improved by at least 4% after 52 weeks of treatment. In some embodiments, the subject has a baseline FVC less than 55%. In some embodiments, the subject has a baseline FVC greater than or equal to 55%.

In some embodiments, the two-component therapy for an ERT-experienced subject with Pompe disease improves and/or stabilizes the subject's motor function, as measured by a GSGC test. In some embodiments, compared to baseline, the subject's GSGC score is improved as indicated by a decrease of at least 0.1, 0.3, 0.5, 0.7, 1.0, 1.5, or 2.5 points after 12, 26, 38, or 52 weeks, or after 18, 24, 30, or 36 months of treatment. In some embodiments, compared to baseline, the subject's GSGC score is improved as indicated by a decrease of at least 0.5 points after 52 weeks of treatment. In some embodiments, compared to the control treatment, the subject's GSGC score is significantly improved and/or stabilized after treatment. In some embodiments, compared to the control treatment, the subject's GSGC score is significantly improved as indicated by a decrease of at least 0.3, 0.5, 0.7, 1.0, 1.5, 2.5, or 5 points after 12, 26, 38, or 52 weeks, or after 18, 24, 30, or 36 months of treatment. In some embodiments, compared to the control treatment, the subject's GSGC score is significantly improved as indicated by a decrease of at least 1.0 point after 52 weeks of treatment.

In some embodiments, the two-component therapy for an ERT-experienced subject with Pompe disease reduces the level of at least one marker of muscle damage after treatment. In some embodiments, the at least one marker of muscle damage comprises CK. In some embodiments, compared to baseline, the subject's CK level is reduced by at least 10%, 15%, 20%, 25%, 30%, 40%, or 50% after 12, 26, 38, or 52 weeks, or after 18, 24, 30, or 36 months of treatment. In some embodiments, compared to baseline, the subject's CK level is reduced by at least 15% after 52 weeks of treatment. In some embodiments, compared to the control treatment, the subject's CK level is significantly reduced after treatment. In some embodiments, compared to the control treatment, the subject's CK level is significantly reduced by at least 10%, 15%, 20%, 25%, 30%, 40%, or 50% after 12, 26, 38, or 52 weeks, or after 18, 24, 30, or 36 months of treatment. In some embodiments, compared to the control treatment, the subject's CK level is significantly reduced by at least 30% after 52 weeks of treatment.

In some embodiments, the two-component therapy for an ERT-experienced subject with Pompe disease reduces the level of at least one marker of glycogen accumulation after treatment. In some embodiments, the at least one marker of glycogen accumulation comprises urinary Hex4. In some embodiments, compared to baseline, the subject's urinary Hex4 level is reduced by at least 10%, 15%, 20%, 25%, 30%, 40%, 50%, or 60% after 12, 26, 38, or 52 weeks, or after 18, 24, 30, or 36 months of treatment. In some embodiments, compared to baseline, the subject's urinary Hex4 level is reduced by at least 25% after 52 weeks of treatment. In some embodiments, compared to the control treatment, the subject's urinary Hex4 level is significantly reduced after treatment. In some embodiments, compared to the control treatment, the subject's urinary Hex4 level is significantly reduced by at least 10%, 15%, 20%, 25%, 30%, 40%, 50%, or 60% after 12, 26, 38, or 52 weeks, or after 18, 24, 30, or 36 months of treatment. In some embodiments, compared to the control treatment, the subject's urinary Hex4 level is significantly reduced by at least 40% after 52 weeks of treatment.

### E. Kits

Another aspect of the disclosure pertains to kits suitable for performing the rhGAA therapy described herein. In one or more embodiments, the kit comprises a container (e.g., vial, tube, bag, etc.) comprising the rhGAA or pharmaceutical composition (either before or after lyophilization) and instructions for reconstitution, dilution and administration. In one or more embodiments, the kit comprises a container (e.g., vial, tube, bag, etc.) comprising an enzyme stabilizer (e.g., miglustat) and a pharmaceutical composition comprising rhGAA (either before or after lyophilization), and instructions for reconstitution, dilution, and administration of rhGAA with the enzyme stabilizer.

### Examples

The present invention is further illustrated by the following non-limiting examples.

Example 1: Preparation of CHO Cells Producing rhGAA Having a High Content of Mono- or Bis-M6P-Bearing N-Glycans.

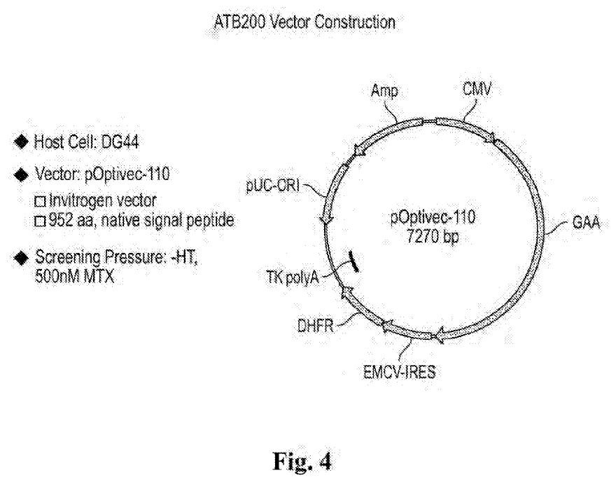

DG44 CHO (DHFR−) cells were transfected with a DNA construct that expresses rhGAA. The DNA construct is shown in FIG. 4. After transfection, CHO cells containing a stably integrated GAA gene were selected with hypoxanthine/thymidine deficient (−HT) medium). GAA expression in these cells was induced by methotrexate treatment (MTX, 500 nM).

Cell pools that expressed high amounts of GAA were identified by GAA enzyme activity assays and were used to establish individual clones producing rhGAA. Individual clones were generated on semisolid media plates, picked by ClonePix system, and were transferred to 24-deep well plates. The individual clones were assayed for GAA enzyme activity to identify clones expressing a high level of GAA. Conditioned media for determining GAA activity used a 4-MU-α-Glucosidase substrate. Clones producing higher levels of GAA as measured by GAA enzyme assays were further evaluated for viability, ability to grow, GAA productivity, N-glycan structure and stable protein expression. CHO cell lines, including CHO cell line GA-ATB200, expressing rhGAA with enhanced mono-M6P or bis-M6P N-glycans were isolated using this procedure.

Example 2: Purification of rhGAA

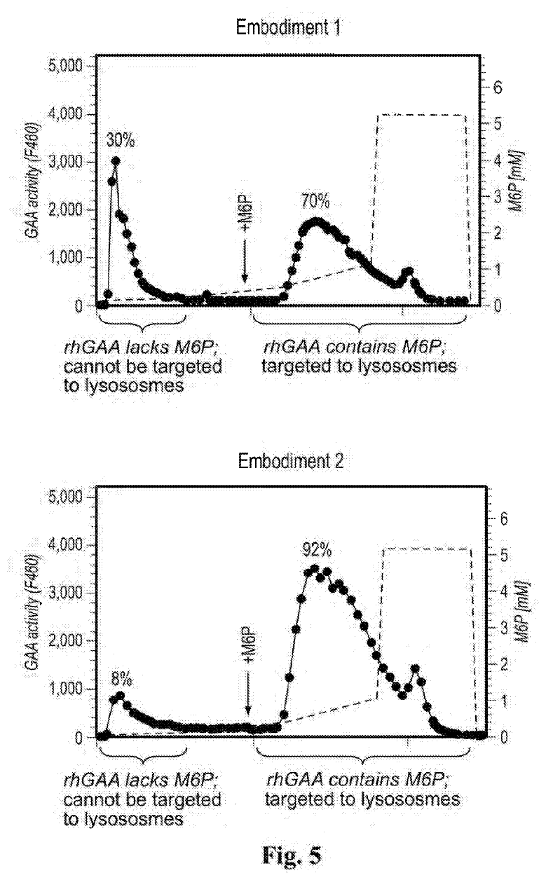

Multiple batches of the rhGAA according to the disclosure were produced in shake flasks and in perfusion bioreactors using CHO cell line GA-ATB200, the product of which is referred to as “ATB200.” Weak anion exchange (“WAX”) liquid chromatography was used to fractionate ATB200 rhGAA according to terminal phosphate and sialic acid. Elution profiles were generated by eluting the ERT with increasing amount of salt. The profiles were monitored by UV (A280 nm). Similar CIMPR receptor binding (at least ~70%) profiles were observed for purified ATB200 rhGAA from different production batches (FIG. 5), indicating that ATB200 rhGAA can be consistently produced.

Example 3: Oligosaccharide Characterization of ATB200 rhGAA

ATB200 rhGAA was analyzed for site-specific N-glycan profiles using different LC-MS/MS analytical techniques. The results of the first two LC-MS/MS methods are shown in FIGS. 6A-6H. The results of a third LC-MS/MS method with 2-AA glycan mapping are shown in FIGS. 19A-19H, FIGS. 20A-20B, and Table 7.

In the first LC-MS/MS analysis, the protein was denatured, reduced, alkylated, and digested prior to LC-MS/MS analysis. During protein denaturation and reduction, 200 μg of protein sample, 5 μL of 1 mol/L tris-HCl (final concentration 50 mM), 75 μL of 8 mol/L guanidine HCl (final concentration 6 M), 1 μL of 0.5 mol/L EDTA (final concentration 5 mM), 2 μL of 1 mol/L DTT (final concentration 20 mM), and Milli-Q® water were added to a 1.5 mL tube to provide a total volume of 100 μL. The sample was mixed and incubated at 56° C. for 30 minutes in a dry bath. During alkylation, the denatured and reduced protein sample was mixed with 5 μL of 1 mol/L iodoacetamide (IAM, final concentration 50 mM), then incubated at 10-30° C. in the dark for 30 minutes. After alkylation, 400 μL of precooled acetone was added to the sample and the mixture was frozen at −80° C. refrigeration for 4 hours. The sample was then centrifuged for 5 min at 13000 rpm at 4° C. and the supernatant was removed. 400 μL of precooled acetone was added to the pellets, which was then centrifuged for 5 min at 13000 rpm at 4° C. and the supernatant was removed. The sample was then air dried on ice in the dark to remove acetone residue. Forty microliters of 8M urea and 160 μL of 100 mM NH₄HCO₃ were added to the sample to dissolve the protein. During trypsin digestion, 50 μg of the protein was then added with trypsin digestion buffer to a final volume of 100 μL, and 5 μL of 0.5 mg/mL trypsin (protein to enzyme ratio of 20/1 w/w) was added. The solution was mixed well and incubated overnight (16±2 hours) at 37° C. Two and a half microliters of 20% TFA (final concentration 0.5%) were added to quench the reaction. The sample was then analyzed using the Thermo Scientific™ Orbitrap Velos Pro™ Mass Spectrometer.

In the second LC-MS/MS analysis, the ATB200 sample was prepared according to a similar denaturation, reduction, alkylation, and digestion procedure, except that iodoacetic acid (IAA) was used as the alkylation reagent instead of IAM, and then analyzed using the Thermo Scientific™ Orbitrap Fusion™ Lumos Tribid™ Mass Spectrometer.

The results of the first and second analyses are shown in FIGS. 6A-6H. In FIGS. 6A-6H, the results of the first analysis are represented by left bar (dark grey) and the results from the second analysis are represented by the right bar (light grey). The symbol nomenclature for glycan representation is in accordance with Varki, A., Cummings, R. D., Esko J. D., et al., Essentials of Glycobiology, 2nd edition (2009).

As can be seen from FIGS. 6A-6H, the two analyses provided similar results, although there was some variation between the results. This variation can be due to a number of factors, including the instrument used and the completeness of N-glycan analysis. For example, if some species of phosphorylated N-glycans were not identified and/or not quantified, then the total number of phosphorylated N-glycans may be underrepresented, and the percentage of rhGAA bearing the phosphorylated N-glycans at that site may be underrepresented. As another example, if some species of non-phosphorylated N-glycans were not identified and/or not quantified, then the total number of non-phosphorylated N-glycans may be underrepresented, and the percentage of rhGAA bearing the phosphorylated N-glycans at that site may be overrepresented.

FIG. 6A shows the N-glycosylation site occupancy of ATB200. As can be seen from FIG. 6A, the first, second, third, fourth, fifth, and sixth N-glycosylation sites are mostly occupied, with both analyses detecting around or over 90% and up to about 100% of the ATB200 enzyme having an N-glycan detected at each potential N-glycosylation site. However, the seventh potential N-glycosylation site is N-glycosylated about half of the time.

FIG. 6B shows the N-glycosylation profile of the first potential N-glycosylation site, N84. As can be seen from FIG. 6B, the major N-glycan species is bis-M6P N-glycans. Both the first and second analyses detected over 75% of the ATB200 having bis-M6P at the first site, corresponding to an average of about 0.8 mol bis-M6P per mol ATB200 at the first site.

FIG. 6C shows the N-glycosylation profile of the second potential N-glycosylation site, N177. As can be seen from FIG. 6C, the major N-glycan species are mono-M6P N-glycans and non-phosphorylated high mannose N-glycans. Both the first and second analyses detected over 40% of the ATB200 having mono-M6P at the second site, corresponding to an average of about 0.4 to about 0.6 mol mono-M6P per mol ATB200 at the second site.

FIG. 6D shows the N-glycosylation profile of the third potential N-glycosylation site, N334. As can be seen from FIG. 6D, the major N-glycan species are non-phosphorylated high mannose N-glycans, di-, tri-, and tetra-antennary complex N-glycans, and hybrid N-glycans. Both the first and second analyses detected over 20% of the ATB200 having a sialic acid residue at the third site, corresponding to an average of about 0.9 to about 1.2 mol sialic acid per mol ATB200 at the third site.

FIG. 6E shows the N-glycosylation profile of the fourth potential N-glycosylation site, N414. As can be seen from FIG. 6E, the major N-glycan species are bis-M6P and mono-M6P N-glycans. Both the first and second analyses detected over 40% of the ATB200 having bis-M6P at the fourth site, corresponding to an average of about 0.4 to about 0.6 mol bis-M6P per mol ATB200 at the fourth site. Both the first and second analyses also detected over 25% of the ATB200 having mono-M6P at the fourth site, corresponding to an average of about 0.3 to about 0.4 mol mono-M6P per mol ATB200 at the fourth site.

FIG. 6F shows the N-glycosylation profile of the fifth potential N-glycosylation site, N596. As can be seen from FIG. 6F, the major N-glycan species are fucosylated di-antennary complex N-glycans. Both the first and second analyses detected over 70% of the ATB200 having a sialic acid residue at the fifth site, corresponding to an average of about 0.8 to about 0.9 mol sialic acid per mol ATB200 at the fifth site.

FIG. 6G shows the N-glycosylation profile of the sixth potential N-glycosylation site, N826. As can be seen from FIG. 6G, the major N-glycan species are di-, tri-, and tetra-antennary complex N-glycans. Both the first and second analyses detected over 80% of the ATB200 having a sialic acid residue at the sixth site, corresponding to an average of about 1.5 to about 1.8 mol sialic acid per mol ATB200 at the sixth site.

An analysis of the N-glycosylation at the seventh site, N869, showed approximately 40% N-glycosylation, with the most common N-glycans being A4S3S3GF (12%), A5S3G2F (10%), A4S2G2F (8%) and A6S3G3F (8%).

FIG. 6H shows a summary of the phosphorylation at each of the seven potential N-glycosylation sites. A s can be seen from FIG. 6H, both the first and second analyses detected high phosphorylation levels at the first, second, and fourth potential N-glycosylation sites. Both analyses detected over 80% of the ATB200 was mono- or bis-phosphorylated at the first site, over 40% of the ATB200 was mono-phosphorylated at the second site, and over 80% of the ATB200 was mono- or bis-phosphorylated at the fourth site.

Another N-glycosylation analysis of ATB200 was performed according to an LC-MS/MS method as described below. This analysis yielded an average N-glycosylation profile over ten lots of ATB200 (FIGS. 19A-19H, FIGS. 20A-20B).

N-linked glycans from ATB200 were released enzymatically with PNGase-F and labeled with 2-Anthranilic acid (2-AA). The 2-AA labeled N-glycans were further processed by solid phase extraction (SPE) to remove excess salts and other contaminants. The purified 2-AA N-glycans were dissolved in acetonitrile/water (20/80; v/v), and 10 micrograms were loaded on an amino-polymer analytical column (apHera™, Supelco) for High Performance Liquid Chromatography with Fluorescence detection (HPLC-FLD) and High Resolution Mass Spectrometry (HRMS) analysis.

The liquid chromatographic (LC) separation was performed under normal phase conditions in a gradient elution mode with mobile phase A (2% acetic acid in acetonitrile) and mobile phase B (5% acetic acid; 20 millimolar ammonium acetate in water adjusted to pH 4.3 with ammonium hydroxide). The initial mobile phase composition was 70% A/30% B. For the fluorescence detection, the parameters for the detector (RF-20Axs, Shimadzu) were Excitation (Ex):320 nm; Emission (Em):420 nm. The HRMS analysis was carried out using a Quadrupole Time of Flight mass spectrometer (Sciex X500B QTOF) operating in Independent Data Acquisition (IDA) mode. The acquired datafiles were converted into mzML files using MSConvert from ProteoWizard, and then GRITS Toolbox 1.2 Morning Blend software (UGA) was utilized for glycan database searching and subsequent annotation of identified N-glycans. The N-glycans were identified using both precursor monoisotopic masses (m/z) and product ion m/z. Experimental product ions and fragmentation patterns were confirmed in-silico using the GlycoWorkbench 2 Application.

To determine the relative quantitation of N-linked glycans from ATB200, data acquired from the HPLC-FLD-QTOF MS/MS experiment was processed as follows. All of the N-glycan peaks in the FLD chromatogram were integrated, and each peak was assigned a percentage of the total area of all peaks in the FLD chromatogram. The fluorescent signal, expressed as a peak area, is a quantitative measure of the amount of each N-glycan in the sample. However, in most cases, multiple N-glycan species were contained in the same FLD peak. Therefore, the mass spectrometer data was also required to obtain relative quantitation of each N-glycan species (Table 7). The ion intensity signal for each N-glycan was “extracted” from the data to create a chromatographic peak called an extracted ion chromatogram (XIC). The XIC aligned with the FLD chromatographic peak and was specific to only one N-glycan species. The XIC peak created from the ion intensity signal was then integrated and this peak area is a relative quantitative measure of the amount of glycan present. Both the FLD peak areas and mass spectrometer XIC peak areas were used to enable relative quantitation of all the N-linked glycan species of ATB200 reported herein.

The results of this LC-MS/MS analysis are provided in Table 7 below. The symbol nomenclature for glycan representation is in accordance with Wopereis W, et al. 2006. Abnormal glycosylation with hypersialylated O-glycans in patients with Sialuria. Biochimica et Biophysica Acta. 1762:598-607; Gornik O, et al. 2007. Changes of serum glycans during sepsis and acute pancreatitis. Glycobiology. 17:1321-1332; Kattla J J, et al. 2011. Biologic protein glycosylation. In: Murray Moo-Young (ed.), Comprehensive Biotechnology, Second Edition, 3:467-486; Tharmalingam-Jaikaran T, et al. N-glycan profiling of bovine follicular fluid at key dominant follicle developmental stages. 2014. Reproduction. 148:569-580; Clerc F, et al. Human plasma protein N-glycosylation. 2015. Glycoconj J. DOI 10.1007/s10719-015-9626-2; and Blackler R J, et al. 2016. Single-chain antibody-fragment M6P-1 possesses a mannose 6-phosphate monosaccharide-specific binding pocket that distinguishes N-glycan phosphorylation in a branch-specific manner. Glycobiology. 26-2:181-192.

TABLE 7
Type and Prevalence of Oligosaccharides identified on ATB200
based on 2-AA glycan mapping and LC-MS/MS identification

| High Mannose | % | Complex | % | Complex | % | Complex | % |
|---|---|---|---|---|---|---|---|
| N-Glycans | Total | N-Glycans | Total | N-Glycans | Total | N-Glycans | Total |
| 2P-M7 | 11.39 | FA2G2S1 | 3.89 | A3G3S1 + 1Ac | 0.65 | FA2G2S1 + 1Ac | 0.29 |
| P-M7 | 7.97 | FA2G2S2 | 3.42 | A3G2S2 + 1Ac | 0.64 | A4G3 | 0.29 |
| M6 | 6.89 | A2G2S2 | 3.32 | A1G1S1 | 0.63 | A4G4 + 3KDN | 0.29 |
| P-M6 | 3.42 | FA2G2 | 2.77 | A4G3S1 | 0.61 | A4G4S3 | 0.28 |
| M5 | 2.06 | FA4G4S3 | 2.26 | FA3G3 | 0.61 | FA5G4 | 0.24 |
| P-M5 | 1.67 | A2G2S1 | 2.25 | A1G1 | 0.6 | A4G3S2 | 0.21 |
| 2P-M8 | 1.27 | FA3G3S1 | 2.12 | FA2G2S2 + 1Ac | 0.57 | FA1 | 0.21 |
| P-M8 | 1.17 | A3G3S2 | 1.8 | A3G2S1 | 0.57 | FA4G4 | 0.21 |
| BP-M6 | 0.9 | FA2G1 | 1.66 | A3G2S1 | 0.56 | A3G1 | 0.21 |
| M7 | 0.81 | A2G2 | 1.46 | A2G2S2 + 1Ac | 0.5 | FA4G3S2 | 0.21 |
| BP-M7 | 0.69 | FA3G3S1 | 1.42 | FA3G2 | 0.45 | FA3G2S2 | 0.21 |
| M4 | 0.14 | A4G4S1 | 1.28 | A3G3 + 3KDN | 0.45 | A1 | 0.2 |
| BP2-M5 | 0.04 | FA3G3S2 | 1.25 | A4G3S1 | 0.45 | A4G2 | 0.19 |
| BP2-M6 | 0.01 | FA4G4(1LN)S3 | 1.1 | A2G1S1 | 0.41 | FA4G3 | 0.19 |
| Hybrid | % | FA4G4S1 | 1.08 | A3G2 | 0.4 | FA3 | 0.18 |
| N-Glycans | Total | | | | | | |
| FA1P-M6 | 2.16 | A3G3 | 1.08 | FA4G4S1 + LN | 0.4 | A1G1S1 | 0.18 |
| M5A1G1S1 | 1.56 | FA4G4S4 | 1.07 | FA3G2S1 | 0.39 | A4G1S1 | 0.16 |
| FP-M6A1G1S1 | 0.42 | FA3G3S3 | 1.04 | FA2 | 0.38 | FA1G1 | 0.15 |
| A1M5 | 0.36 | FA4G4S2 | 0.94 | FA4G4S2 + LN | 0.38 | FA3G1 | 0.14 |
| A1G1M5 | 0.32 | A2G1 | 0.94 | A3G2S2 | 0.37 | FA5G4S2 | 0.12 |
| P-M6A1G1S1 | 0.17 | FA2G1S1 | 0.94 | A2 | 0.34 | A3G1S1 | 0.11 |
| Summary | Total | A4G4 | 0.91 | FA4G4(2LN)S3 | 0.33 | A3 | 0.11 |
| High Mannose | 38% | FA1G1S1 | 0.91 | FA2G2Sg1 | 0.32 | FA3G3S3 + 1Ac | 0.1 |
| N-Glycans | | | | | | | |
| Hybrid | 5% | FA2G2S2 + 2Ac | 0.76 | FA4G4(1LN)S4 | 0.31 | A2G2S1 + 1Ac | 0.09 |
| N-Glycans | | | | | | | |
| Complex | 57% | A4G4S2 | 0.69 | A3G3S3 | 0.29 | FA3G1S1 | 0.06 |
| N-Glycans | | | | | | | |

Based on this 2-AA and LC-MS/MS analysis, and as further summarized, the ATB200 tested has an average M6P content of 3-5 mol per mol of ATB200 (accounting for both mono-M6P and bis-M6P) and sialic acid content of 4-7 mol per mol of ATB200.

As shown in FIGS. 19A-19H and summarized in FIG. 20B, the first potential N-glycosylation site of ATB200 has an average M6P content of about 1.4 mol M6P/mol ATB200, accounting for an average mono-M6P content of about 0.25 mol mono-M6P/mol ATB200 and an average bis-M6P content of about 0.56 mol bis-M6P/mol ATB200; the second potential N-glycosylation site of ATB200 has an average M6P content of about 0.5 mol M6P/mol ATB200, with the primary phosphorylated N-glycan species being mono-M6P N-glycans; the third potential N-glycosylation site of ATB200 has an average sialic acid content of about 1 mol sialic acid/mol ATB200; the fourth potential N-glycosylation site of ATB200 has an average M6P content of about 1.4 mol M6P/mol ATB200, accounting for an average mono-M6P content of about 0.35 mol mono-M6P/mol ATB200 and an average bis-M6P content of about 0.52 mol bis-M6P/mol ATB200; the fifth potential N-glycosylation site of ATB200 has an average sialic acid content of about 0.86 mol sialic acid/mol ATB200; the sixth potential N-glycosylation site of ATB200 has an average sialic acid content of about 4.2 mol sialic acid/mol ATB200; and the seventh potential N-glycosylation site of ATB200 has an average sialic acid content of about 0.86 mol sialic acid/mol ATB200.

Also according to this 2-AA and LC-MS/MS analytical technique, an average of about 65% of the N-glycans at the first potential N-glycosylation site of ATB200 are high mannose N-glycans, about 89% of the N-glycans at the second potential N-glycosylation site of ATB200 are high mannose N-glycans, over half of the N-glycans at the third potential N-glycosylation site of ATB200 are sialylated (with nearly 20% fully sialylated) and about 85% of the N-glycans at the third potential N-glycosylation site of ATB200 are complex N-glycans, about 84% of the N-glycans at the fourth potential N-glycosylation site of ATB200 are high mannose N-glycans, about 70% of the N-glycans at the fifth potential N-glycosylation site of ATB200 are sialylated (with about 26% fully sialylated) and about 100% of the N-glycans at the fifth potential N-glycosylation site of ATB200 are complex N-glycans, about 85% of the N-glycans at the sixth potential N-glycosylation site of ATB200 are sialylated (with nearly 27% fully sialylated) and about 98% of the N-glycans at the sixth potential N-glycosylation site of ATB200 are complex N-glycans, and about 87% of the N-glycans at the seventh potential N-glycosylation site of ATB200 are sialylated (with nearly 8% fully sialylated) and about 100% of the N-glycans at the seventh potential N-glycosylation site of ATB200 are complex N-glycans.

### Example 4: Analytical Comparison of ATB200 and MYOZYME®/LUMIZYME®

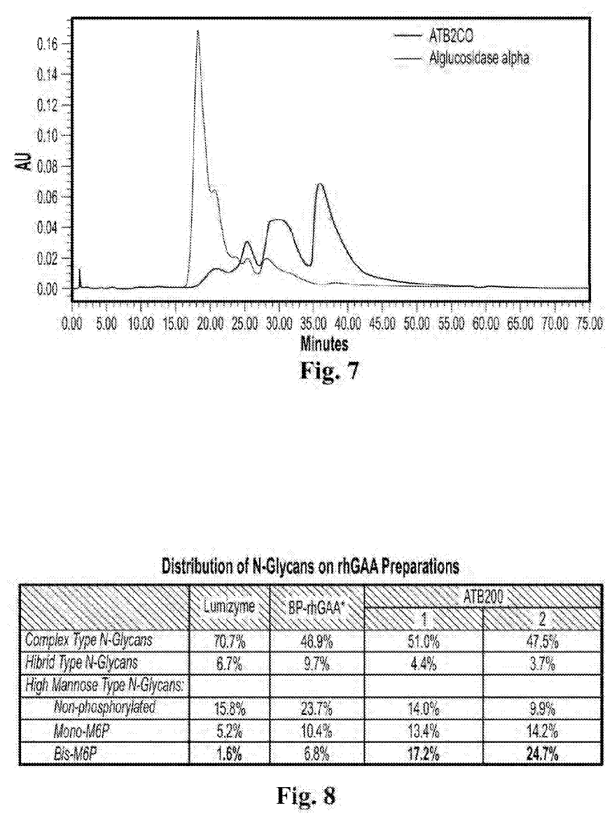

Purified ATB200 and LUMIZYME® N-glycans were evaluated by MALDI-TOF to determine the individual N-glycan structures found on each ERT. LUMIZYME® was obtained from a commercial source. As shown in FIG. 7, ATB200 exhibited four prominent peaks eluting to the right of LUMIZYME®. This confirms that ATB200 was phosphorylated to a greater extent than LUMIZYME® since this evaluation is by terminal charge rather than CIMPR affinity. As summarized in FIG. 8, ATB200 samples were found to contain lower amounts of non-phosphorylated high-mannose type N-glycans than LUMIZYME®.

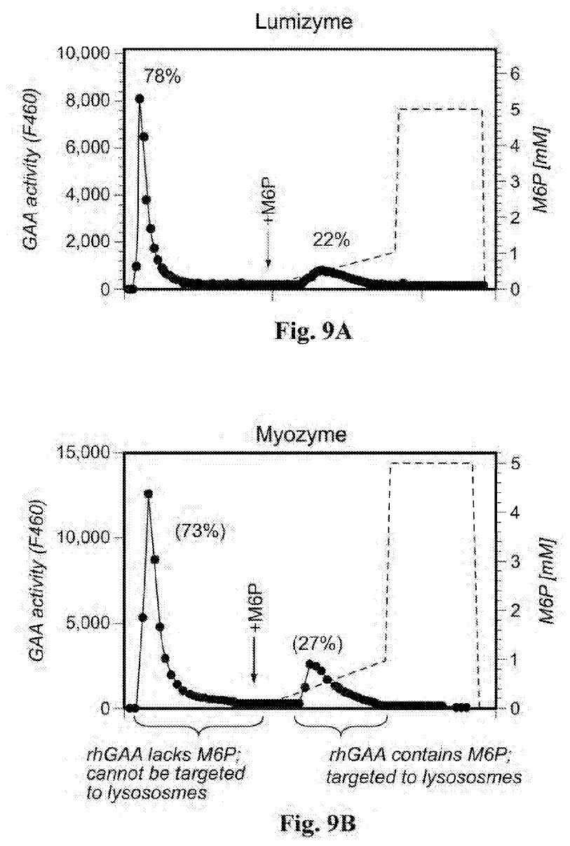

To evaluate the ability of the conventional rhGAAs in MYOZYME® and LUMIZYME® to interact with the CIMPR, the two conventional rhGAA preparations were injected onto a CIMPR affinity column (which binds rhGAA having M6P groups) and the flow through collected. The bound material was eluted with a free M6 gradient. Fractions were collected in 96-well plate and GAA activity assayed by 4MU-α-glucosidase substrate. The relative amounts of unbound (flow through) and bound (M6P eluted) rhGAA were determined based on GAA activity and reported as the fraction of total enzyme. FIGS. 9A and 9B show the binding profile of rhGAAs in MYOZYME® and LUMIZYME®: 73% of the rhGAA in MYOZYME® (FIG. 9B) and 78% of the rhGAA in LUMIZYME® (FIG. 9A) did not bind to the CIMPR. Indeed, only 27% of the rhGAA in MYOZYME® and 22% of the rhGAA in LUMIZYME® contained M6P that can be productive to target it to the CIMPR on muscle cells. In contrast, as shown in FIG. 5, under the same condition, more than 70% of the rhGAA in ATB200 was found to bind to the CIMPR.

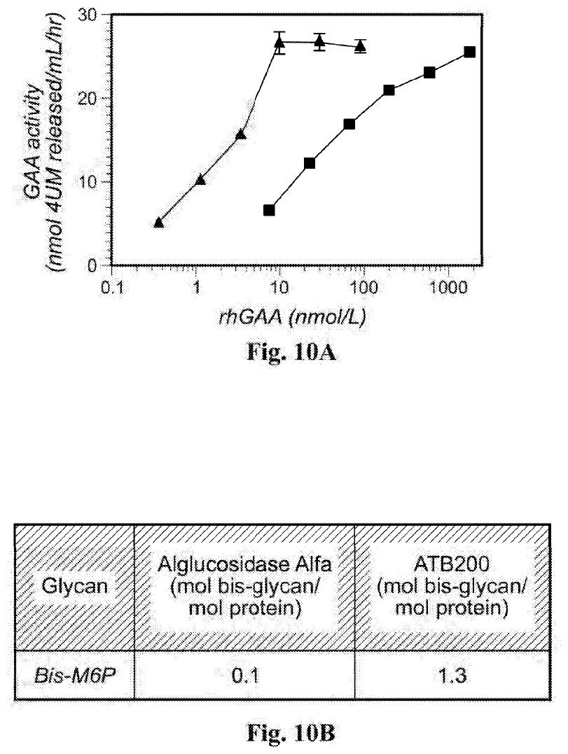

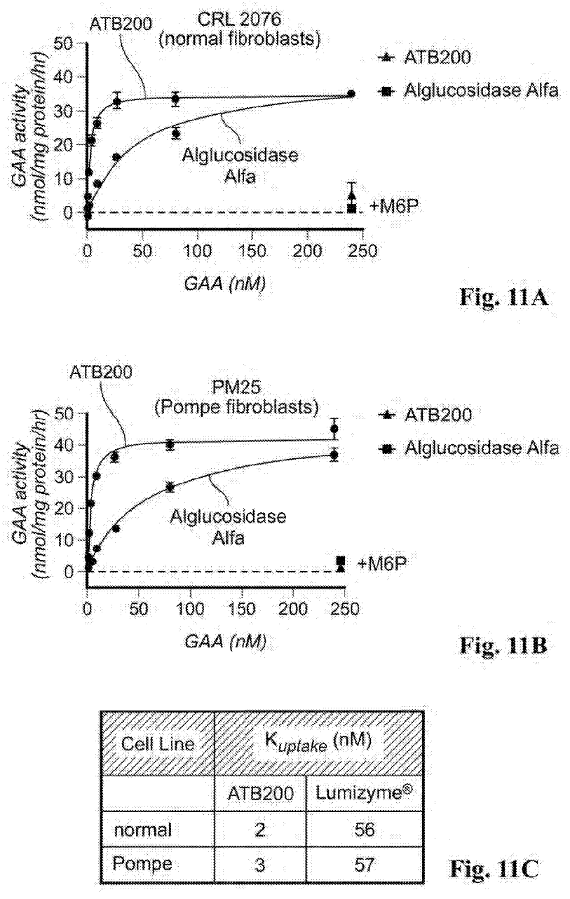

In addition to having a greater percentage of rhGAA that can bind to the CIMPR, it is important to understand the quality of that interaction. LUMIZYME® and ATB200 receptor binding was determined using a CIMPR plate binding assay. Briefly, CIMPR-coated plates were used to capture GAA. Varying concentrations of rhGAA were applied to the immobilized receptor and unbound rhGAA was washed off. The amount of remaining rhGAA was determined by GAA activity. As shown in FIG. 10A, ATB200 bound to CIMPR significantly better than LUMIZYME®. FIG. 10B shows the relative content of bis-M6P N-glycans in LUMIZYME® (a conventional rhGAA product) and ATB200 according to the invention. For LUMIZYME®, there is on average only 10% of molecules having a bis-phosphorylated N-glycan. In contrast, on average every rhGAA molecule in ATB200 has at least one bis-phosphorylated N-glycan.

Overall, the higher content of M6P N-glycans in ATB200 than in LUMIZYME® indicates that the higher portion of rhGAA molecules in ATB200 can target muscle cells. As shown above, the high percentage of mono-phosphorylated and bis-phosphorylated structures determined by MALDI agree with the CIMPR profiles which illustrated significantly greater binding of ATB200 to the CIMPR receptor. N-glycan analysis via MALDI-TOF mass spectrometry confirmed that on average each ATB200 molecule contains at least one natural bis-M6P N-glycan structure. This higher bis-M6P N-glycan content on ATB200 directly correlated with high-affinity binding to CIMPR in M6P receptor plate binding assays (K_{D} about 2-4 nM).

The relative cellular uptake of ATB200 and LUMIZYME® rhGAA were compared using normal and Pompe fibroblast cell lines. Comparisons involved 5-100 nM of ATB200 according to the disclosure with 10-500 nM conventional rhGAA product LUMIZYME®. After 16-hr incubation, external rhGAA was inactivated with TRIS base and cells were washed 3-times with PBS prior to harvest. Internalized GAA measured by 4MU-α-Glucoside hydrolysis and was graphed relative to total cellular protein and the results appear in FIGS. 11A-11C.

ATB200 was also shown to be efficiently internalized into cells. As depicted in FIGS. 11A-11B, ATB200 is internalized into both normal and Pompe fibroblast cells and is internalized to a greater degree than the conventional rhGAA product LUMIZYME®. ATB200 saturates cellular receptors at about 20 nM, while about 250 nM of LUMIZYME® is needed to saturate cellular receptors. The uptake efficiency constant (Kᵤₚₜₐₖₑ) extrapolated from these results is 2-3 nm for ATB200 and 56 nM for LUMIZYME®, as shown by FIG. 11C. These results suggest that ATB200 is a well-targeted treatment for Pompe disease.

### Example 5: ATB200 and Enzyme Stabilizer

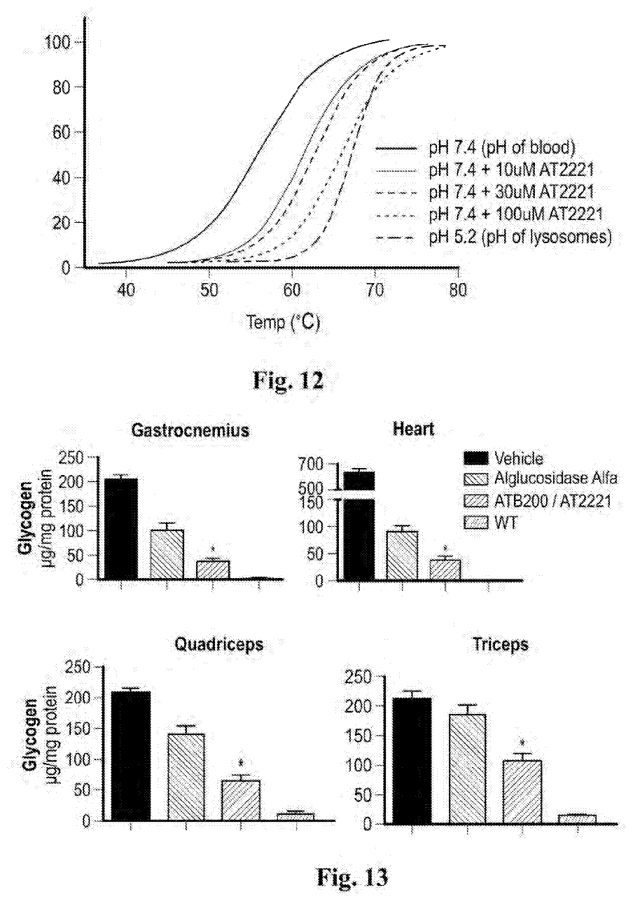

The stability of ATB200 in acidic or neutral pH buffers was evaluated in a thermostability assay using SYPRO Orange, as the fluorescence of the dye increases when proteins denature. As shown in FIG. 12, the addition of miglustat stabilized ATB200 at pH 7.4 in a concentration-dependent manner, comparable to the stability of ATB200 at pH 5.2, a condition that mimics the acidic environment of the lysosome. As summarized in Table 10, the addition of miglustat increased the melting temperature (Tₘ) of ATB200 by nearly 10° C.

TABLE 10
Stability of ATB200 with miglustat

| Test Condition | Tm (° C.) |
|---|---|
| pH 7.4 | 56.2 |
| pH 7.4 + 10 μM miglustat | 61.6 |
| pH 7.4 + 30 μM miglustat | 62.9 |
| pH 7.4 + 100 μM miglustat | 66.0 |
| pH 5.2 | 67.3 |

### Example 6: Co-Administration of ATB200 and Miglustat in Gaa KO Mice

The therapeutic effects of ATB200 and miglustat were evaluated and compared against those of Alglucosidase alfa in Gaa KO mice. For the study, male Gaa KO (3- to 4-month old) and age-matched wild-type (WT) mice were used. Alglucosidase alfa was administered via bolus tail vein intravenous (IV) injection. In the co-administration regimen, miglustat was administered via oral gavage (PO) 30 minutes prior to the IV injection of ATB200. Treatment was given biweekly. Treated mice were sacrificed after 14 days from the last administration and various tissues were collected for further analysis. Table 11 summarizes the study design:

TABLE 11
Co-administration Study Design

| | | Drug Dosage | Number of |
|---|---|---|---|
| | | per Administration | Adminis- |
| Genotype | Treatment | (bi-weekly) | tration |
| Gaa KO | Vehicle | N/A | 6 |
| Gaa KO | Alglucosidase alfa | 20 mg/kg | 6 |
| Gaa KO | ATB200/miglustat | 20 mg/kg (ATB200) | 6 |
| | | 10 mg/kg (miglustat) | |
| WT (Sve 129) | Not Treated | N/A | N/A |

Tissue glycogen content in tissues samples was determined using amyloglucosidase digestion, as discussed above. As shown in FIG. 13, a combination of 20 mg/kg ATB200 and 10 mg/kg miglustat significantly decreased the glycogen content in four different tissues (quadriceps, triceps, gastrocnemius, and heart) as compared to the same dosage of alglucosidase alfa.

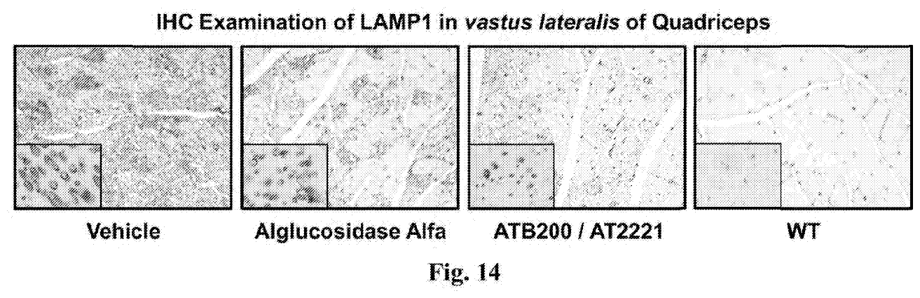

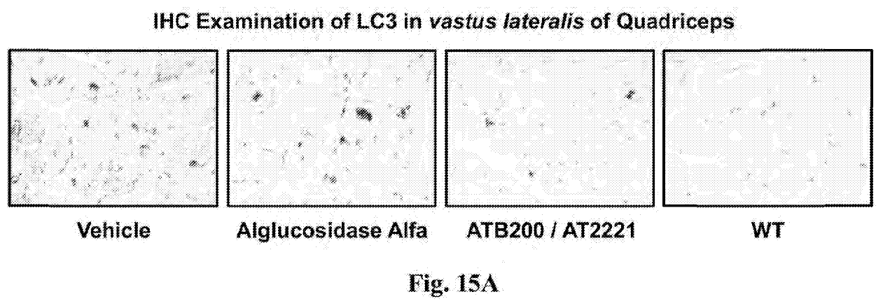

Tissue samples were also analyzed for biomarker changes following the methods discussed in: Khanna R, et al. (2012), “The pharmacological chaperone [i.e., enzyme stabilizer] AT2220 increases recombinant human acid α-glucosidase uptake and glycogen reduction in a mouse model of Pompe disease,” Plos One 7(7): e40776; and Khanna, R et al. (2014), “The Pharmacological Chaperone AT2220 Increases the Specific Activity and Lysosomal Delivery of Mutant Acid α-Glucosidase, and Promotes Glycogen Reduction in a Transgenic Mouse Model of Pompe Disease,” PLoS ONE 9(7): e102092. As shown in FIG. 14, a profound increase in and enlargement of LAMP1-positive vesicles was seen in muscle fibers of Gaa KO animals compared to WT, indicative of lysosomal proliferation. Co-administration of ATB200/miglustat led to more fibers with normalized LAMP1 level, while the remaining LAMP1-positive vesicles also reduced in size (insets).

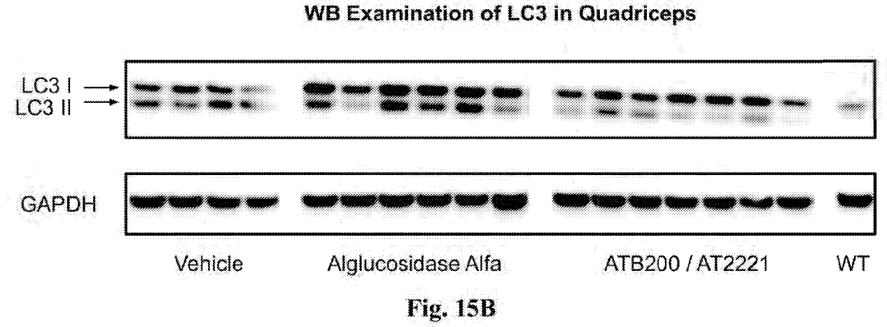

Similarly, intense LC3-positive aggregates in the muscle fibers of untreated Gaa KO mice signify the presence of autophagic zones and autophagy build-up. LC3-positive aggregates (red) were preferentially reduced in mice treated with ATB200/miglustat co-administration as compared to mice treated with alglucosidase alfa (FIG. 15A). A similar observation was made when the expression of LC3 was assessed using western blot. As shown in FIG. 15B, the majority of animals treated with ATB200/miglustat showed a significant decrease in levels of LC3 II, the lipidated form that is associated with autophagosomes, suggesting an improved autophagy flux. In comparison, the effect of alglucosidase alfa on autophagy was modest.

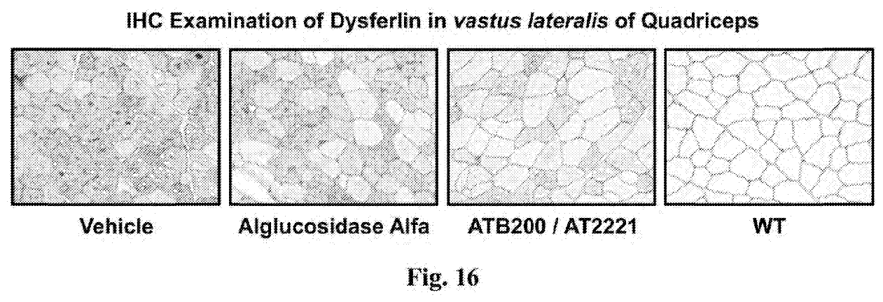

Dysferlin, a protein involved in membrane repair and whose deficiency/mistrafficking is associated with a number of muscular dystrophies, was also assessed. As shown in FIG. 16, dysferlin (brown) was heavily accumulated in the sarcoplasm of Gaa KO mice. Compared to alglucosidase alfa, ATB200/miglustat was able to restore dysferlin to the sarcolemma in a greater number of muscle fibers.

These data are consistent with improvements at the cellular level demonstrated in human Pompe disease patients treated with ATB200 and miglustat, (e.g., the patients exhibit reduced levels of biomarkers of glycogen accumulation and muscle injury), leading not only to effective treatment of Pompe disease but also a reversal in disease progression. Clinical data in human Pompe disease patients are summarized in Examples 8 and 9, below.

### Example 7: Single Fiber Analysis

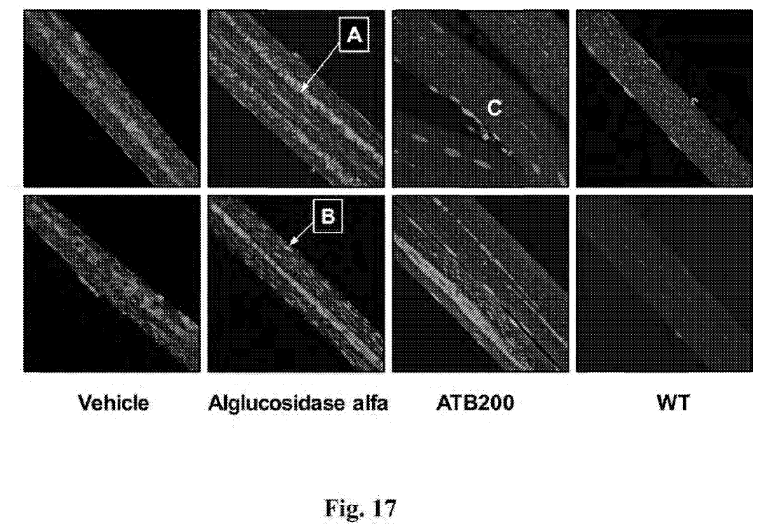

As shown in FIG. 17, majority of the vehicle-treated mice showed grossly enlarged lysosomes (green) (see, for example “B”) and the presence of massive autophagic buildup (red) (see, for example “A”). MYOZYME®-treated mice did not show any significant difference as compared to vehicle-treated mice. In contrast, most fibers isolated from mice treated with ATB200 showed dramatically decreased lysosome size (see, for example, “C”). Furthermore, the area with autophagic buildup was also reduced to various degrees (see, for example, “C”). As a result, a significant portion of muscle fibers analyzed (36-60%) from ATB200-treated mice appeared normal or near-normal. Table 12 below summarizes the single fiber analysis shown in FIG. 17.

TABLE 12
Single Fiber Analysis

| | | | | | Fibers with |
|---|---|---|---|---|---|
| | | Total Number | | Fibers with | Normal or Near- |
| | Animal | of Fibers | Lysosome | Autophagy | normal |
| Treatment | Analyzed | Analyzed (n) | Enlargement | Buildup | Appearance |
| WT | 2 | 65 | − | — | 100% |
| Vehicle | 2 | 65 | + | >90% | <10% |
| Alglucosidase | 4 | 150 | + | >90% | <10% |
| alfa | | | | | |
| ATB200 | 5 | 188 | Dramatic size | 40-64%* | 36-60% |
| | | | decrease in | | |
| | | | most fibers | | |

*This included fibers with varying degree of reduction in autophagic buildup. Overall, the extent of the buildup was smaller in ATB200-treated group compared to Vehicle-or alglucosidase alfa-treated group.

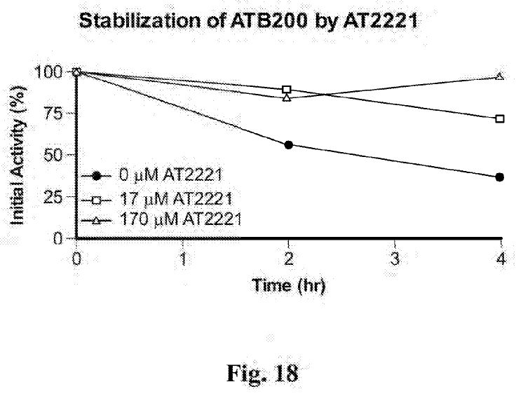

Overall, the data indicate that ATB200, with its higher M6P content, both alone and further stabilized by the enzyme stabilizer miglustat at the neutral pH of blood, is more efficient in tissue targeting and lysosomal trafficking compared to alglucosidase alfa when administered to Gaa KO mice, consistent with the stabilization of ATB200 by miglustat as depicted in FIG. 18. As a result, administration of ATB200 and co-administration of ATB200/miglustat was more effective than alglucosidase alfa in correcting some of the disease-relevant pathologies, such as glycogen accumulation, lysosomal proliferation, and formation of autophagic zones. Due to these positive therapeutic effects, administration of ATB200 and ATB200/miglustat co-administration is shown to improve the chance of muscle fiber recovery from damage and even to reverse damage by clearing glycogen that had accumulated in the cell due to lack of optimal GAA activity. As with Example 6, these data are also consistent with improvements at the cellular level demonstrated in human Pompe disease patients that lead to both effective treatment of Pompe disease and reversal in disease progression following administration of ATB200 and miglustat. Clinical data in human Pompe disease patients are summarized in Examples 8 and 9, below.

### Example 8: The ATB200-02 Trial

A phase 1/2 (ATB200-02, NCT-02675465) open-label, fixed-sequence, ascending-dose clinical study was conducted to assess safety, tolerability, pharmacokinetics, pharmacodynamics, and interim efficacy of IV infusion of ATB200 with miglustat in adult subjects with Pompe disease. The data was reported in International Publication No. WO 2020/163480, the disclosure of which is herein incorporated by reference.

Example 9: The ATB200-03 Trial: A Phase 3 in-Human Study of ATB200/Miglustat in Patients with Pompe Disease

The ATB200-03 trial was a phase 3 double-blind, randomized, multicenter, international study of ATB200/miglustat in adult subjects with late-onset Pompe disease (LOPD) who had received enzyme replacement therapy with alglucosidase alfa (i.e., ERT-experienced) or who had never received ERT (i.e., ERT naïve), compared with alglucosidase alfa/placebo.

### Study Design

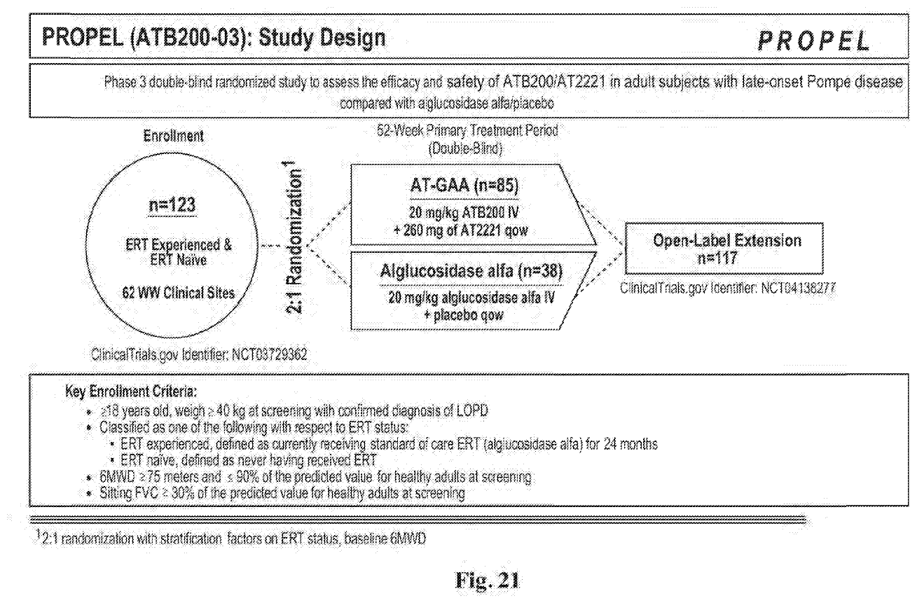

As shown in FIG. 21, the trial consisted of a screening period up to 30 days, a 12-month treatment period, and a 30-day safety follow-up period. Eligible subjects were randomly assigned in a 2:1 ratio to receive ATB200/miglustat or alglucosidase alfa/placebo and stratified by ERT status (ERT-experienced, ERT-naïve) and baseline 6-minute walk distance (6MWD) (75 to <150 meters, 150 to <400 meters, ≥400 meters).

Efficacy assessments (i.e., functional assessments) included evaluation of ambulatory function (6MWT), motor function tests (Gait, Stair, Gower, and Chair maneuver (GSGC) test and Timed Up and Go (TUG) test), muscle strength (manual muscle testing and quantitative muscle testing), and pulmonary function tests (FVC, SVC, MIP, MEP, and SNIP). Patient reported outcomes (Rasch-built Pompe-specific Activity (R Pact) Scale, EuroQol 5 Dimensions 5 Levels Instrument (EQ-5D-5L), Patient-Reported Outcomes Measurement Information System (PROMIS®) instruments for physical function, fatigue, dyspnea, and upper extremity, and Subject's Global Impression of Change) were recorded. The Physician's Global Impression of Change were also performed.

Pharmacodynamic assessments included measurement of biomarkers of muscle injury (creatine kinase (CK) and disease substrate (urinary hexose tetrasaccharide (Hex4)). Sparse blood samples were collected for determination of total GAA protein levels and miglustat concentrations in plasma for a population PK analysis in ERT-experienced subjects. Serial blood sampling for characterization of the PK profile of total GAA protein and miglustat were done in ERT-naïve subjects.

Safety assessments included monitoring of adverse events (AEs), including infusion associated reactions (IARs), clinical laboratory tests (chemistry, hematology, and urinalysis), vital signs, physical examinations including weight, electrocardiograms (ECGs), and immunogenicity. Concomitant medications and nondrug therapies were also be recorded.

### Subject Selection

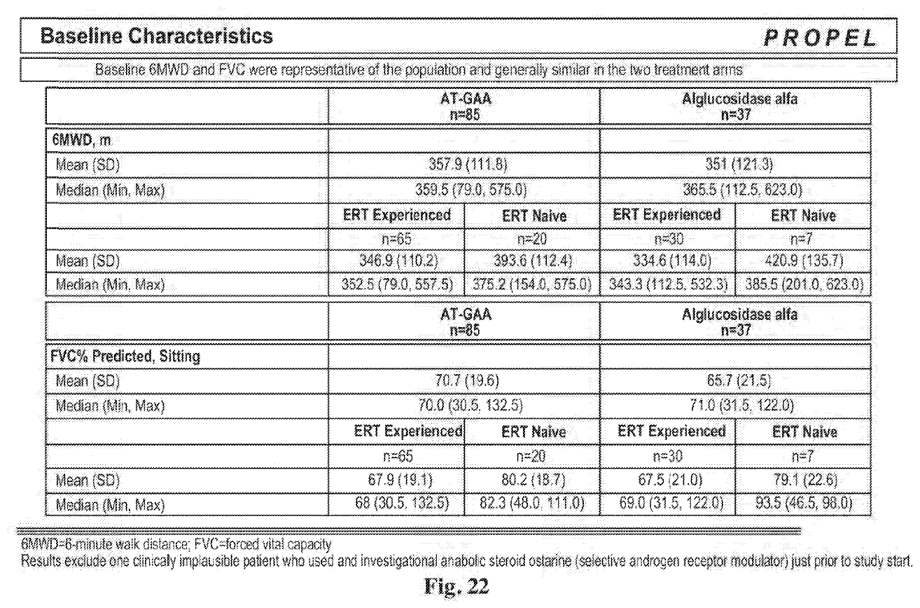

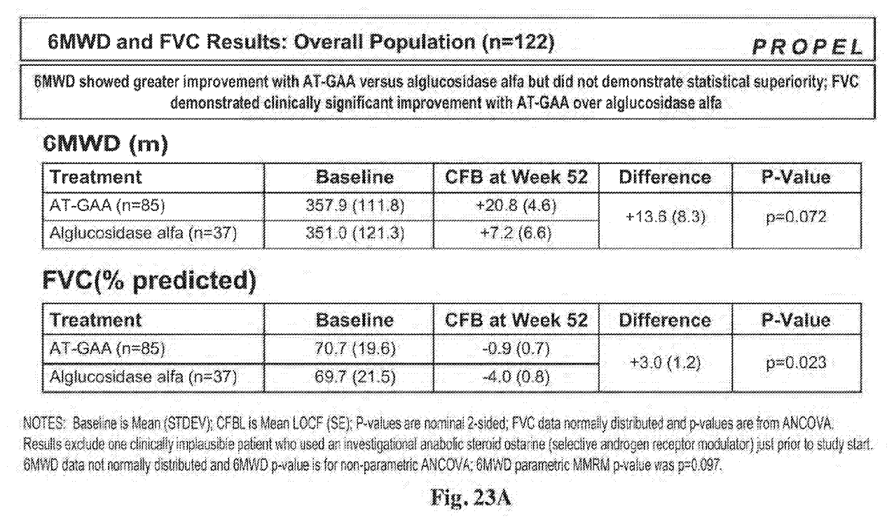

Subjects who participated in the study met all of the following inclusion criteria and none of the exclusion criteria. In total, 122 subjects participated in the ATB200-03 trial. Among them, 85 subjects (ERT-experienced: 65; ERT-naïve: 20) received the ATB200/miglustat treatment, and 37 subjects (ERT-experienced: 30; ERT-naïve: 7) received the alglucosidase alfa/placebo treatment. As shown in FIG. 22, the baseline 6MWD and FVC data was representative of the population and generally similar in the two treatment arms.

### Inclusion Criteria:

1. Subject provided signed informed consent prior to any study-related procedures being performed.
2. Male and female subjects were ≥18 years old and weighed ≥40 kg at screening.
3. Female subjects of childbearing potential and male subjects agreed to use medically accepted methods of contraception during the study and for 90 days after the last dose of study drug.
4. Subject had a diagnosis of LOPD based on documentation of one of the following:
   a. deficiency of GAA enzyme
   b. GAA genotyping
5. Subject was classified as one of the following with respect to ERT status:
   a. ERT-experienced, defined as had received standard of care ERT (alglucosidase alfa) at the recommended dose and regimen (i.e., 20 mg/kg dose every 2 weeks) for ≥24 months Specific to Australia, ERT-experienced, defined as had received standard of care ERT (alglucosidase alfa) at the recommended dose and regimen, at a dose of 20 mg/kg based on lean or ideal body weight every 2 weeks
   b. ERT-naïve, defined as never had received investigational or commercially available ERT
6. Subject had a sitting FVC≥30% of the predicted value for healthy adults (National Health and Nutrition Examination Survey III) at screening.
7. Subject performed two 6MWTs at screening that were valid, as determined by the clinical evaluator, and that met all of the following criteria:
   a. both screening values of 6MWD were ≥75 meters
   b. both screening values of 6MWD were ≤90% of the predicted value for healthy adults
   c. the lower value of 6MWD was within 20% of the higher value of 6MWD

### Exclusion Criteria:

1. Subject had received any investigational therapy or pharmacological treatment for Pompe disease, other than alglucosidase alfa, within 30 days or 5 half-lives of the therapy or treatment, whichever was longer, before Day 1 or was anticipated to do so during the study.
2. Subject had received gene therapy for Pompe disease.
3. Subject was taking any of the following prohibited medications within 30 days before Day 1:
   miglitol
   miglustat
   acarbose
   voglibose
4. Subject required the use of invasive or noninvasive ventilation support for >6 hours per day while awake.
5. Subject had a hypersensitivity to any of the excipients in ATB200, alglucosidase alfa, or miglustat.
6. Subject had a medical condition or any other extenuating circumstance that, in the opinion of the investigator or medical monitor, posed an undue safety risk to the subject or compromised his/her ability to comply with or adversely impact protocol requirements. This included clinical depression (as diagnosed by a psychiatrist or other mental health professional) with uncontrolled or poorly controlled symptoms.
7. Subject, if female, was pregnant or breastfeeding at screening.
8. Subject, whether male or female, was planning to conceive a child during the study.
9. Subject refused to undergo genetic testing.

### Investigational Product, Dosage, and Mode of Administration

Subjects were randomized with a randomization ratio of at least 2:1 to receive either ATB200/miglustat or alglucosidase alfa/placebo. Table 13 below summarizes the treatment of the enrolled subjects.

TABLE 13
Treatment Assignment and Regimen

| Treatment | | |
|---|---|---|
| Assignment | Treatment | Regimen |
| ATB200/ | miglustat^{a} | Subjects ≥50 kg: 260 mg (4 × 65-mg |
| miglustat | | oral capsules) 1 hour prior to ATB200 |
| | | infusion every 2 weeks |
| | | Subjects ≥40 kg to <50 kg: 195 mg |
| | | (3 × 65-mg oral capsules) 1 hour prior |
| | | to ATB200 infusion every 2 weeks |
| | ATB200 | 20 mg/kg IV infusion over a 4-hour |
| | | duration every 2 weeks |
| Alglucosidase | Placebo | Subjects ≥50 kg: Placebo (4 oral |
| alfa/placebo | | capsules) 1 hour prior to alglucosidase |
| | | alfa infusion every 2 weeks |
| | | Subjects ≥40 kg to <50 kg: Placebo |
| | | (3 oral capsules) 1 hour prior to |
| | | alglucosidase alfa infusion every |
| | | 2 weeks |
| | Alglucosidase | 20 mg/kg IV infusion over a 4-hour |
| | alfa | duration every 2 weeks |

Abbreviations: IV = intravenous
^{a}Note:
Subjects were required to fast at least 2 hours before and 2 hours after administration of miglustat or placebo.

### Data Evaluation and Statistical Considerations

The primary efficacy endpoint was the change from baseline to Week 52 in 6MWD. The primary endpoint was tested for superiority of ATB200/miglustat vs Alglucosidase alfa/placebo, using mixed-effect model for repeated measures (MMRM) and pre-specified nonparametric test in case of violation of normality.

Key secondary efficacy endpoints in a pre-specified hierarchical order of importance were as follows. These secondary endpoints were analyzed using analysis of covariance (ANCOVA) model with last observation carried forward (ITT LOCF).
change from baseline to Week 52 in sitting FVC (% predicted)
change from baseline to Week 52 in the manual muscle test score for the lower extremities
change from baseline to Week 52 in the total score for the PROMIS—physical function
change from baseline to Week 52 in the total score for the PROMIS—fatigue
change from baseline to Week 52 in GSGC total score

Other secondary efficacy endpoints were as follows:
change from baseline to Week 52 in the following variables related to motor function:
   time to complete the 10-meter walk (i.e., assessment of gait) of the GSGC test
   time to complete the 4-stair climb of the GSGC test
   time to complete the Gower's maneuver of the GSGC test
   time to arise from a chair as part of the GSGC test
   time to complete the TUG test
change from baseline to Week 52 in the following variables related to muscle strength:
   manual muscle test score for the upper extremities
   manual muscle test total score
   quantitative muscle test value (kg) for the upper extremities
   quantitative muscle test value (kg) for the lower extremities
   quantitative muscle test total value (kg)
change from baseline to Week 52 in the following variables from patient-reported outcome measures:
   total score for the PROMIS—dyspnea
   total score for the PROMIS—upper extremity
   R-PAct Scale total score
   EQ-5D-5L health status
actual value of the subject's functional status (improving, stable, or declining) pertaining to the effects of study drug in the following areas of life at Week 52, as measured by the Subject's Global Impression of Change
   overall physical wellbeing
   effort of breathing
   muscle strength
   muscle function
   ability to move around
   activities of daily living
   energy level
   level of muscular pain
actual value of the subject's functional status (improving, stable, or declining) at Week 52, as measured by the Physician's Global Impression of Change
change from baseline to Week 52 in the following measures of pulmonary function, as follows:
   sitting FVC (% predicted)
   MIP (cmH₂O)
   MIP (% predicted)
   MEP (cmH₂O)
   MEP (% predicted)
   SNIP (cmH₂O)

Pharmacodynamic endpoints were as follows:
change from baseline to Week 52 in serum CK level
change from baseline to Week 52 in urinary Hex4 level

For ERT-experienced subjects, pharmacokinetic endpoints from a population PK analysis of total GAA protein level and miglustat concentration were collected. For ERT-naïve subjects, PK parameters for plasma total GAA protein concentration and miglustat were calculated.

The safety profile of ATB200/miglustat was characterized using incidence of treatment emergent adverse events (TEAEs), serious adverse events (SAEs), and AEs leading to discontinuation of study drug, frequency and severity of immediate and late IARs, and any abnormalities noted in other safety assessments. The impact of immunogenicity to ATB200 and alglucosidase alfa on safety and efficacy was also assessed.

Statistical methods included the following considerations on sample randomization, sample size calculation, efficacy analyses, and safety analyses.

Randomization. The following two factors were identified as design stratification variables: 1. baseline 6MWD (75 to <150 meters, 150 to <400 meters, ≥400 meters); and 2. ERT status (ERT-experienced, ERT-naïve). These two factors formed six factorial combinations (i.e., levels, strata). A centralized block randomization procedure was used to balance the above risk factors, 1) to reduce bias and increase the precision of statistical inference, and 2) to allow various planned and unplanned subset analyses. The block randomization scheme was performed for each of the 6 strata. The randomization ratio is 2:1 ATB200/miglustat to alglucosidase alfa/placebo, fixed.

Sample Size Calculation. A 2-group t-test with a 2-sided significance level of 0.05 and a 2:1 randomization scheme (66 subjects in the ATB200/miglustat group and 33 subjects in the alglucosidase alfa/placebo group, for a total sample size of 99 subjects) was determined to have approximately 90% power to detect a standardized effect size of 0.7 between the 2 groups in a superiority test. This calculation was performed using Nquery 8©®. Assuming a 10% dropout rate, the sample size would be approximately 110 subjects.

Efficacy Analyses. The primary efficacy endpoint (i.e., change from baseline to Week 52 in 6MWD) was analyzed using a parametric analysis of covariance (ANCOVA) model to compare between the new treatment and the control. This model would typically adjust for baseline 6MWD (as a continuous covariate), and the 2 factors used to stratify randomization: ERT status (ERT naïve vs. ERT-experienced) and baseline 6MWD (75 to <150 meters, 150 to <400 meters, ≥400 meters). However, the baseline 6MWD could not be used in the model twice (both as a continuous and a categorical variable) due to the expected high point biserial correlation between them. Thus, the 6MWD continuous variable remained in the model but the categorical 6MWD was removed. The ANCOVA model then had terms for treatment, baseline 6MWD (continuous), and ERT status (categorical).

Additionally, potential treatment-by-covariate interactions (i.e., treatment by ERT status and treatment-by-baseline 6MWD continuous) were examined. If an interaction term was statistically significant (e.g., p<0.10, 2 sided), and there was logical biological interpretation, then the interaction term could potentially be added in the final ANCOVA model that would be used for the primary endpoint analysis. The data was then be analyzed based on the ANCOVA model, and all the relevant estimates (e.g., LS means for each treatment group, LS means difference, 95% confidence intervals (CIs) for the LS mean difference, and p-value for comparing between the 2 treatment groups) were provided.

To support the interpretation of clinical benefit, a composite subject-level response was defined based on the totality of the treatment outcome data. Subjects were classified by an ordinal response variable consisting of significant improvement, moderate improvement, or minor/no improvement based on treatment outcomes.

Key secondary endpoints were analyzed according to the hierarchical order, using stepwise closed testing procedure to control the type I error rate. Key secondary and other secondary endpoints were analyzed separately with a similar method used for the primary endpoint analysis.

Safety Analyses. Safety data was summarized using counts and percentages for categorical data and descriptive statistics (mean, standard deviation, median, minimum, maximum) for continuous data.
Efficacy Results from the ATB200-03 Trial

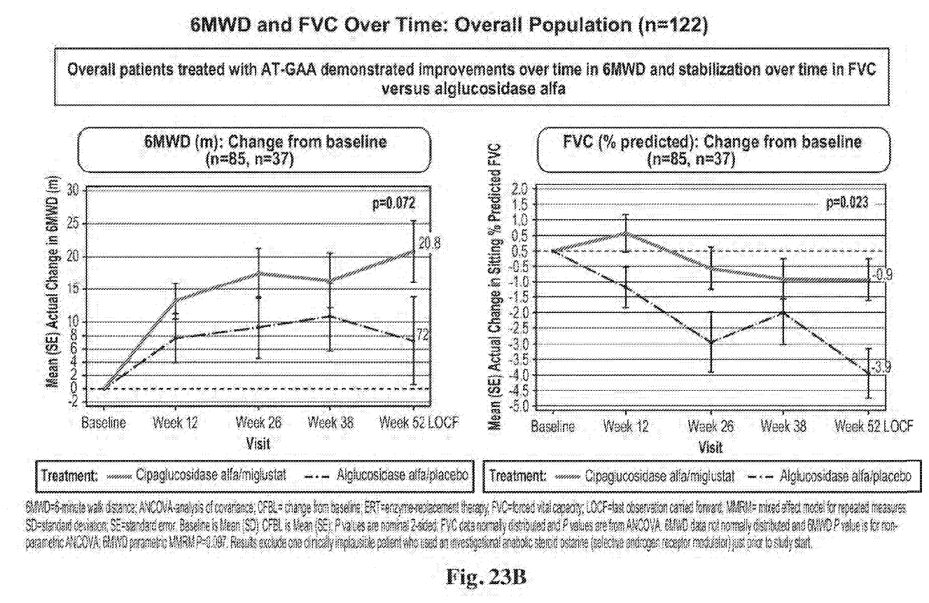

In the overall population, ATB200/miglustat treatment showed improvement in 6MWD and stabilization in percent-predicted FVC, relative to baseline at week 52 (FIG. 23A) and over time (FIG. 23B). Compared to alglucosidase alfa/placebo, ATB200/miglustat treatment showed greater improvement in 6MWD in the overall population at week 52 (FIG. 23A). Furthermore, as shown in FIG. 23A, ATB200/miglustat treatment showed clinically significant improvement in percent-predicted FVC in the overall population at week 52, compared to alglucosidase alfa/placebo.

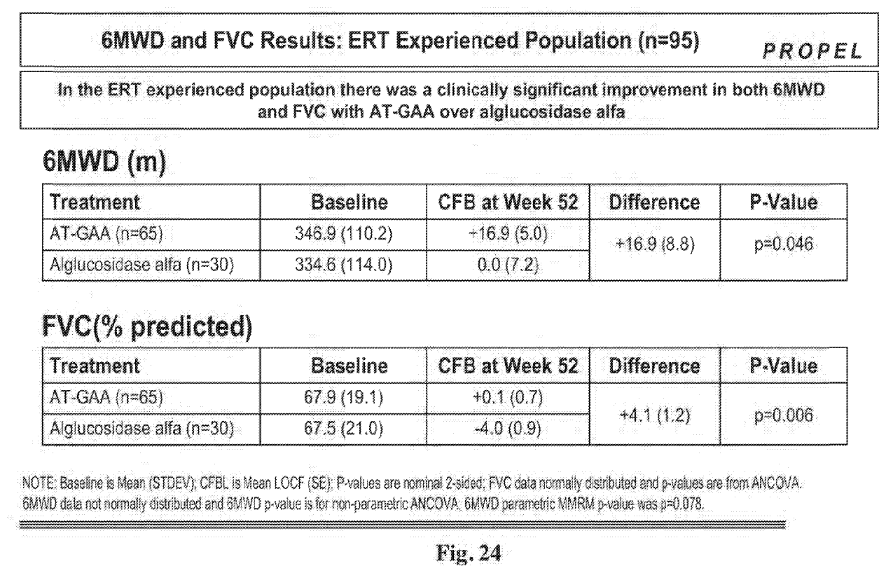

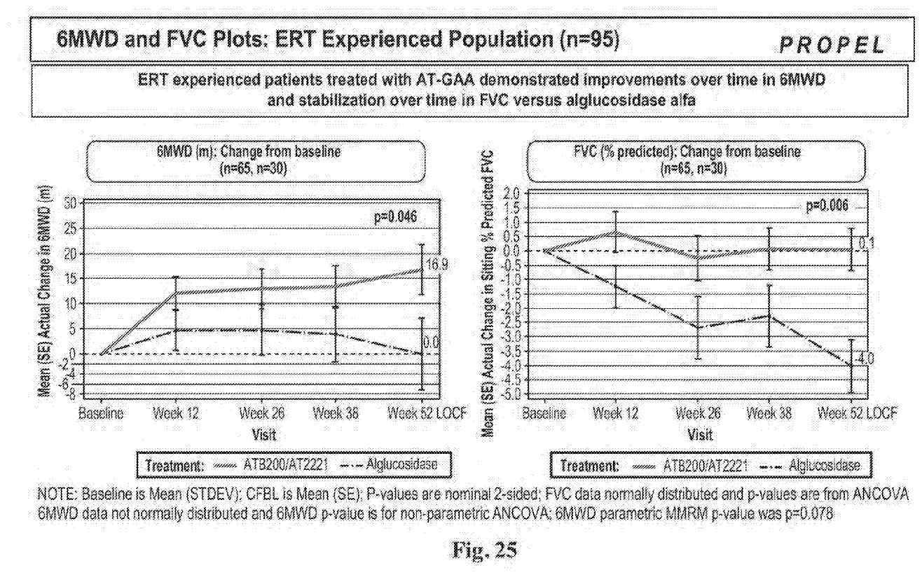

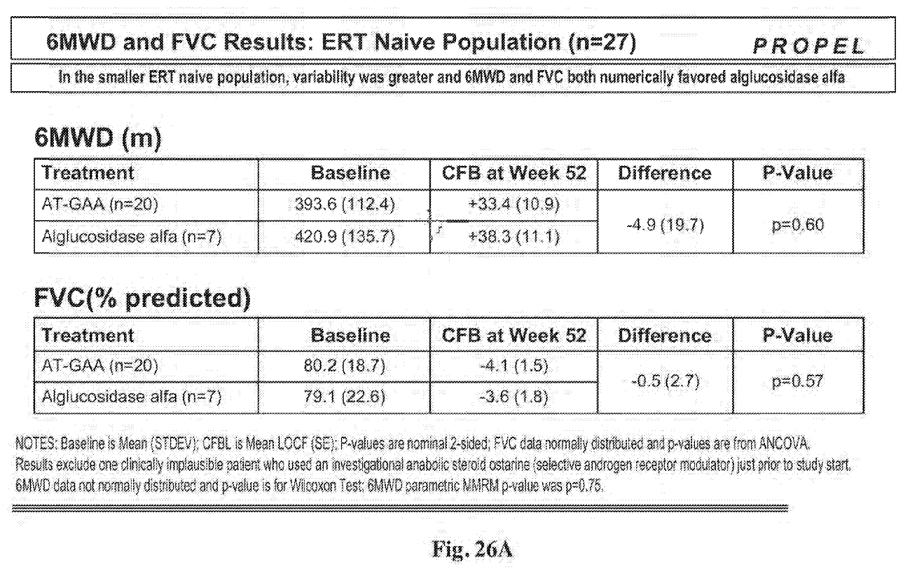

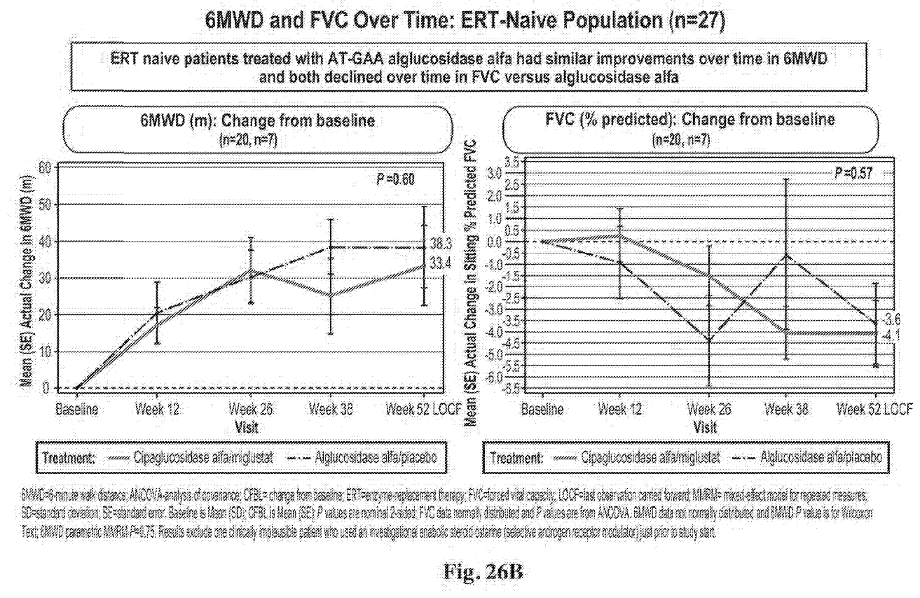

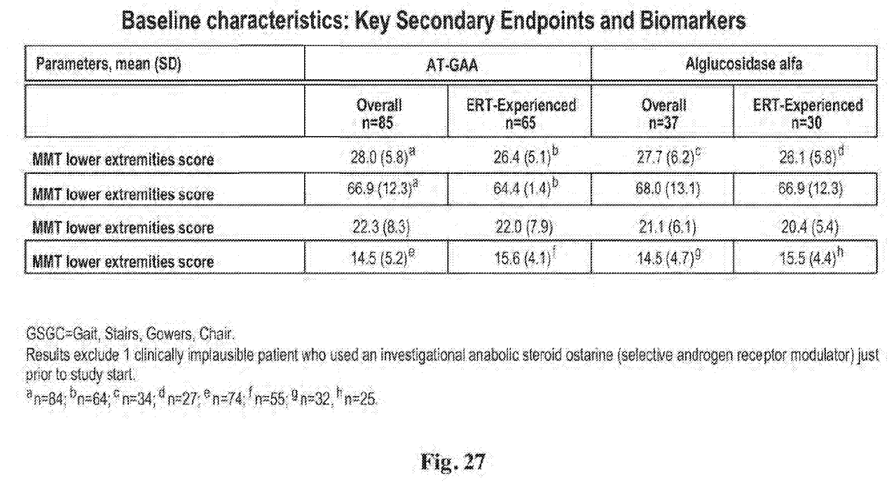

In the ERT-experienced population, ATB200/miglustat treatment showed improvement in 6MWD and stabilization in percent-predicted FVC, relative to baseline at week 52 (FIG. 24). Compared to alglucosidase alfa/placebo, ATB200/miglustat treatment showed improvements over time in 6MWD and stabilization over time in percent-predicted FVC in the ERT-experienced population (FIG. 25). Furthermore, as shown in FIG. 24, ATB200/miglustat treatment showed clinically significant improvement in both 6MWD and percent-predicted FVC in the ERT-experienced population at week 52, compared to alglucosidase alfa/placebo.

As shown in FIGS. 26A and 26B, in the smaller ERT-naive population (n=27), ATB200/miglustat treatment showed improvement in 6MWD and stabilization in percent-predicted FVC, relative to baseline at week 52 (FIG. 26A) and over time (FIG. 26B). Variability between the two treatment groups was greater and no clinically significant improvement was observed in 6MWD or percent-predicted FVC (FIG. 26A).

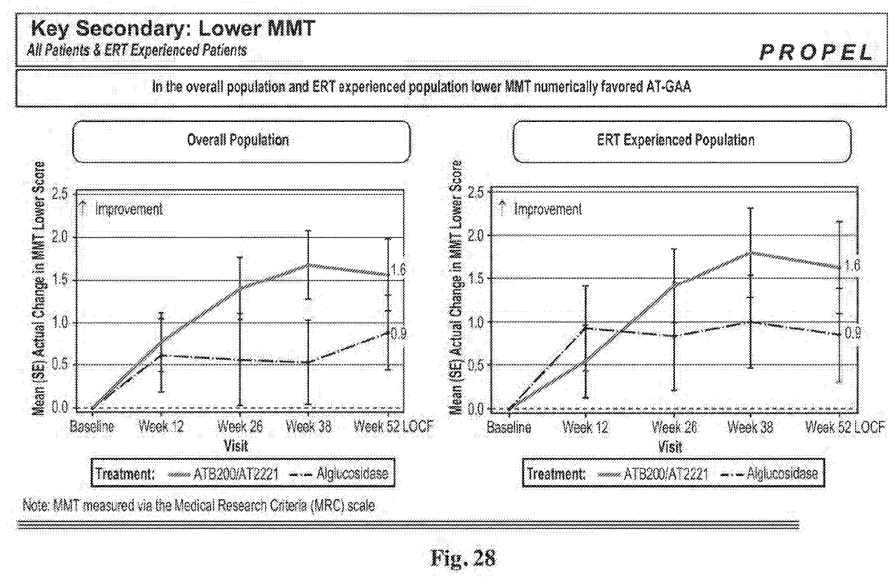

As shown in FIG. 28, in the overall population ERT-experienced populations, lower MMT numerically favored ATB200/miglustat treatment, compared to alglucosidase alfa/placebo.

The mean change (SD) in MMT score for lower extremities from baseline to week 52 showed a numerical improvement of 1.6 (3.78) in subjects treated with co-administration of cipaglucosidase alfa and miglustat compared to 0.9 (2.58) in those treated with alglucosidase alfa and placebo (p=0.191).

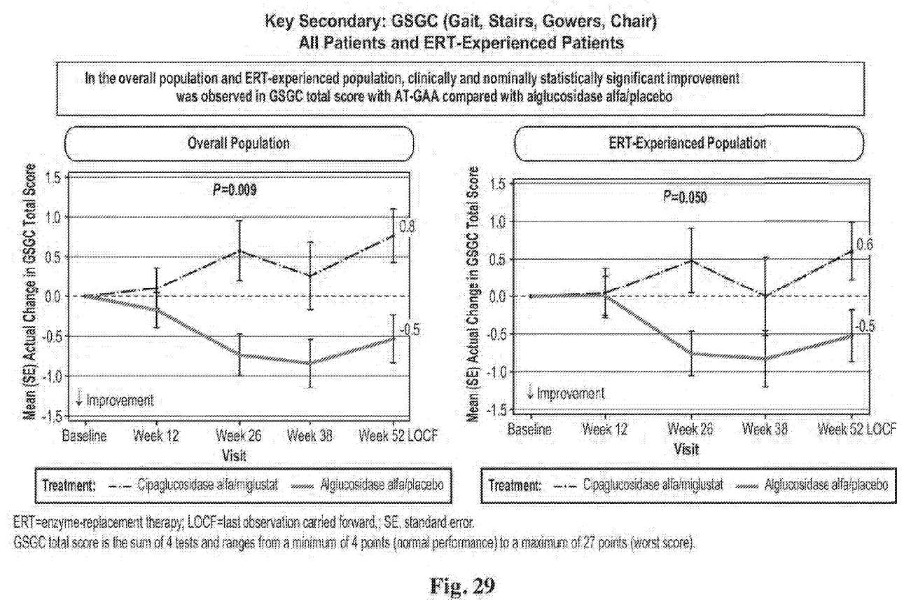

As shown in FIG. 29, in the overall and ERT-experienced populations, ATB200/miglustat treatment showed clinically significant improvement in GSGC at week 52, compared to alglucosidase alfa/placebo.

The mean change in total GSGC score from baseline to week 52 showed an improvement of −0.53 (2.5) in subjects treated with co-administration of cipaglucosidase alfa and miglustat compared to 0.77 (1.8) in those treated with alglucosidase alfa and placebo (p=0.009).

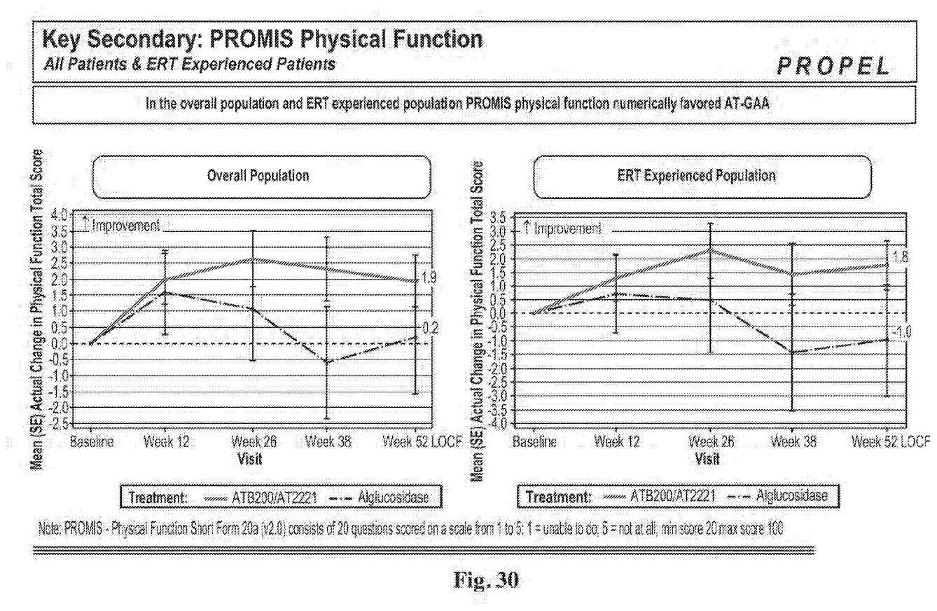

As shown in FIG. 30, in the overall and ERT-experienced populations, PROMIS physical function numerically favored ATB200/miglustat treatment, compared to alglucosidase alfa/placebo.

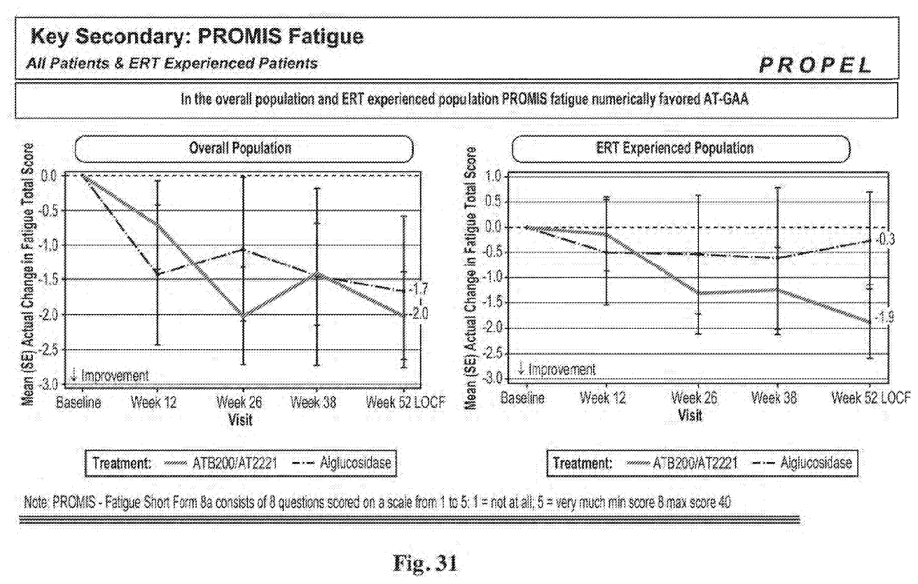

As shown in FIG. 31, in the overall and ERT experienced populations, PROMIS fatigue improved similarly between the two treatment groups.

Biomarker Results from the ATB200-03 Trial

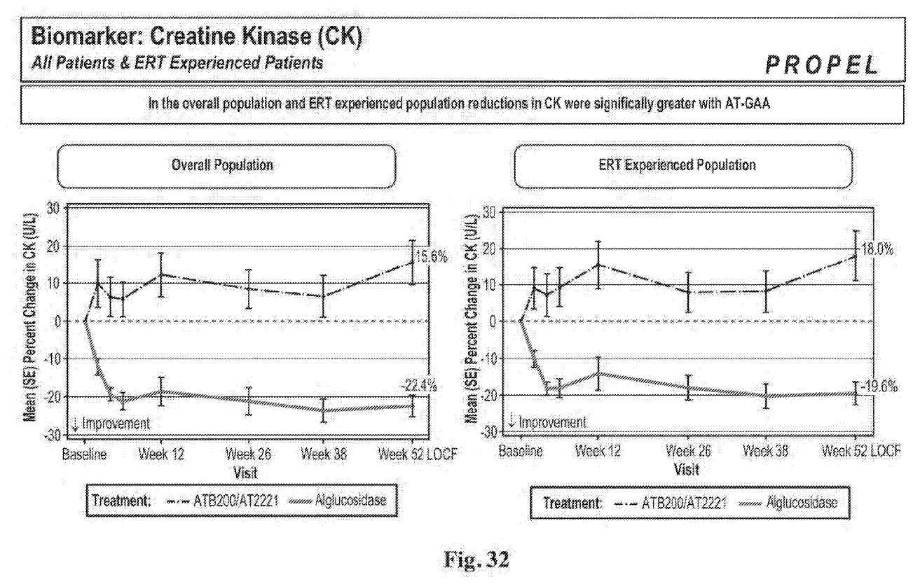

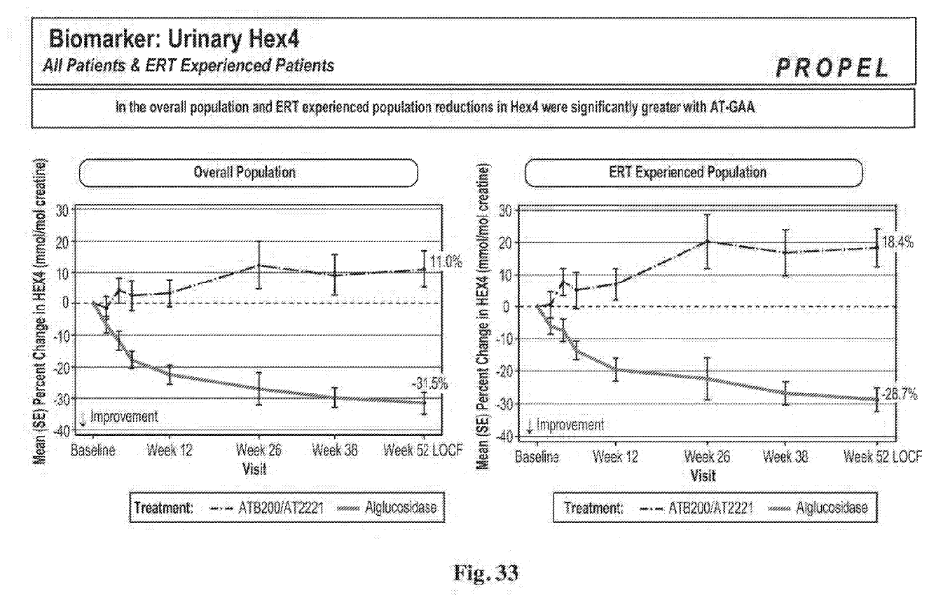

In the overall and ERT-experienced populations, ATB200/miglustat treatment showed improvement in biomarkers of muscle damage (CK) and disease substrate (Hex4) over time (FIGS. 32 and 33). Furthermore, as shown in FIGS. 32 and 33, in the overall and ERT-experienced populations, reductions in CK and urinary Hex4 were significantly greater with ATB200/miglustat treatment at week 52, compared to alglucosidase alfa/placebo.

In ERT-experienced patients, the baseline mean urinary Glc4 concentration was 4.6 mmol/mol and 7.2 mmol/mol in the ATB200/miglustat treatment and alglucosidase alfa with placebo treatment, respectively. At Week 52, the mean urinary Glc4 concentration was 2.9 mmol/mol and 9.1 mmol/mol in the ATB200/miglustat treatment and alglucosidase alfa with placebo treatment group, respectively.

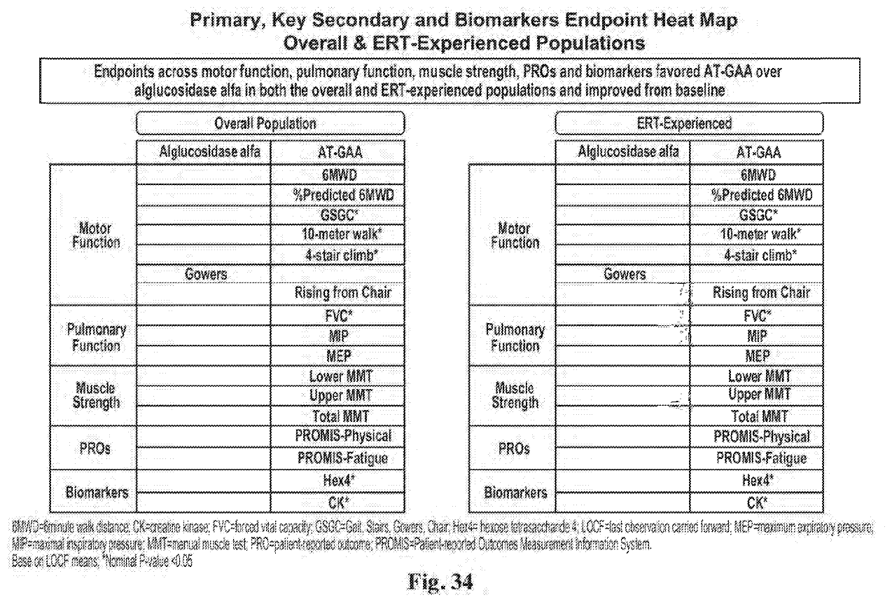

As summarized in FIG. 34, in the overall and ERT-experienced populations, endpoints across motor function, pulmonary function, muscle strength, patient-reported outcomes (PROs) and biomarkers consistently favored ATB200/miglustat treatment over alglucosidase alfa/placebo. Furthermore, of the 17 efficacy and biomarker endpoints assessed, 16 favored ATB200/miglustat treatment over alglucosidase alfa/placebo.

Safety Results from the ATB200-03 Trial

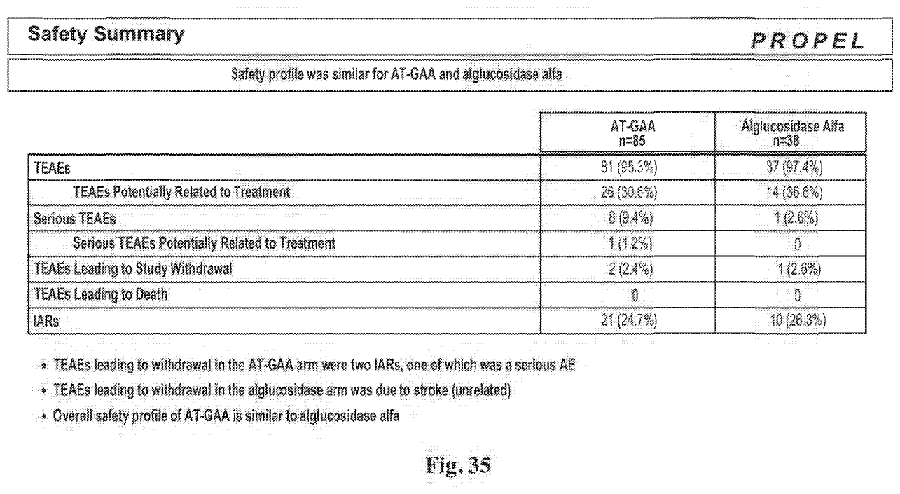

As shown in FIG. 35, the overall safety profile of ATB200/miglustat treatment group was similar to that of the alglucosidase alfa/placebo group.

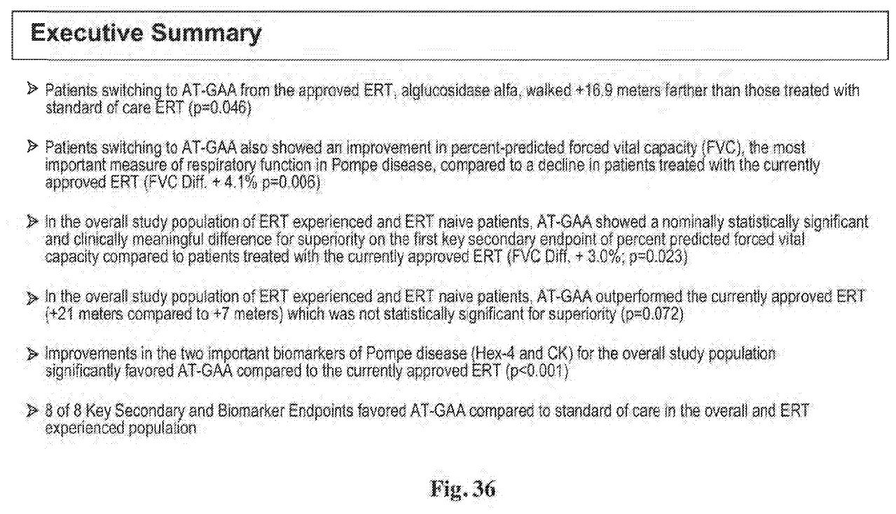

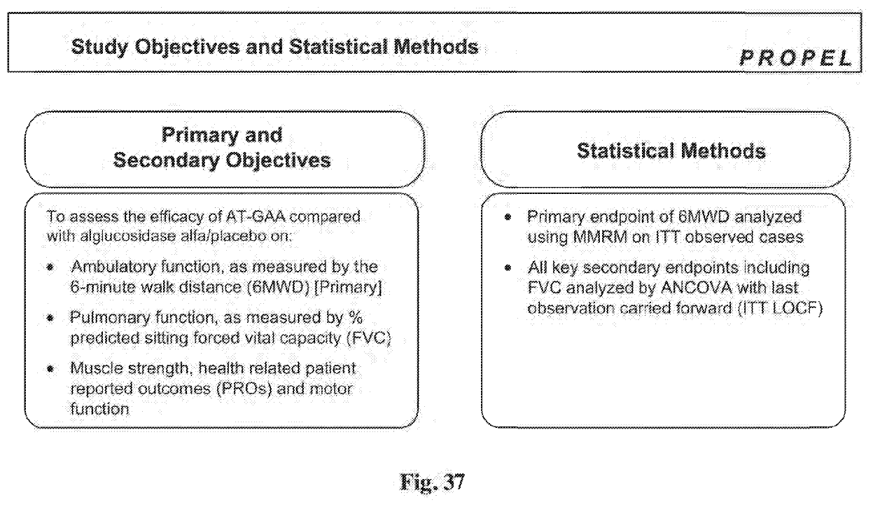

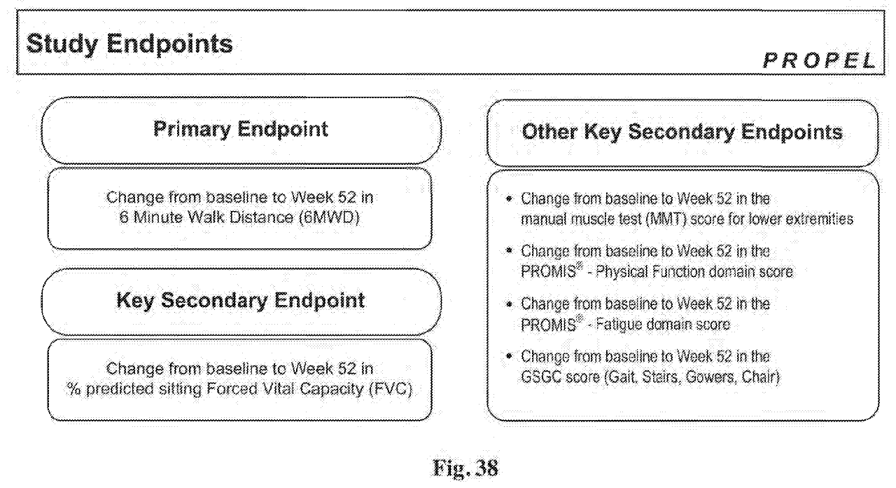

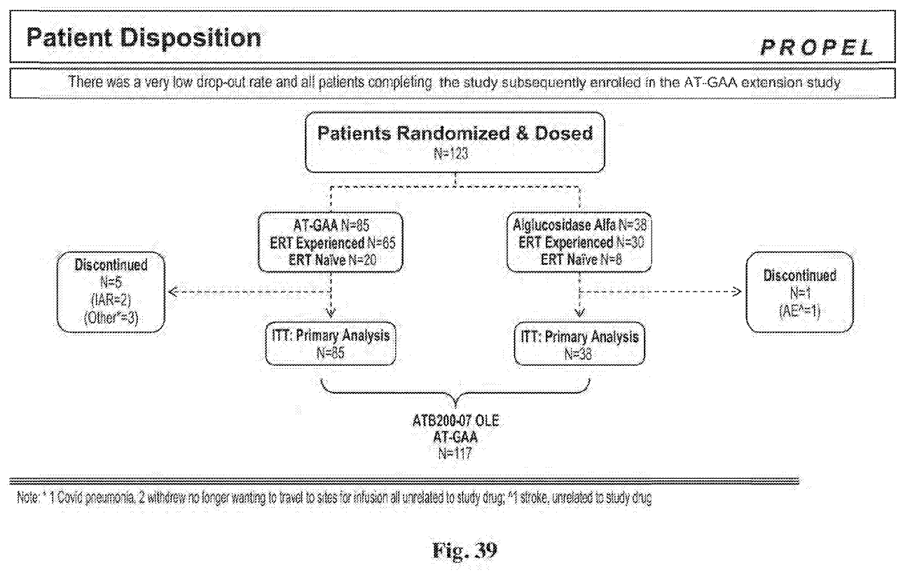

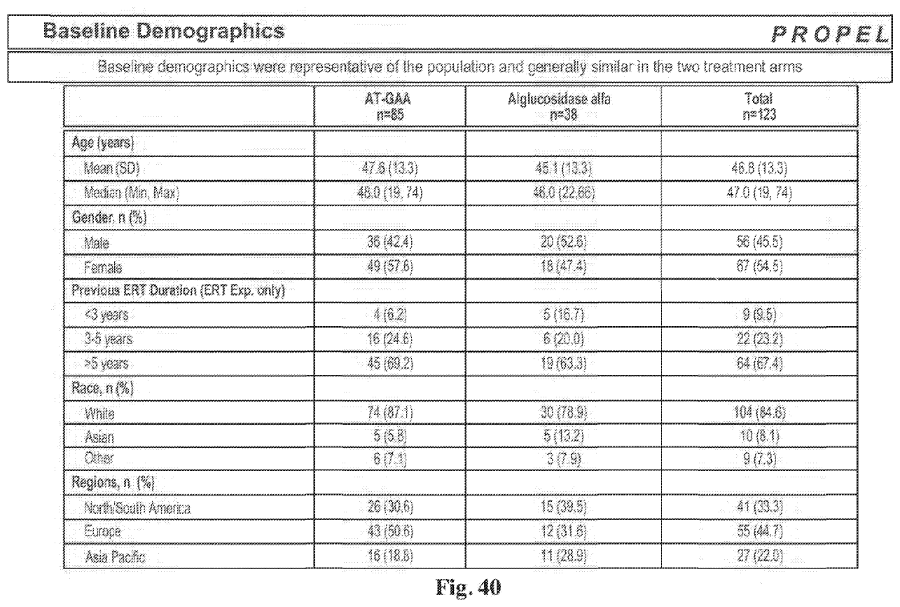

FIG. 36-FIG. 40 describe additional aspects of the ATB200-03 Trial.

### Example 10: Results of PROPEL Phase 3 Clinical Trials

AT-GAA showed clinically meaningful & significant improvements in both musculoskeletal and respiratory measures in late-onset Pompe disease compared to standard of care in pivotal phase 3 PROPEL study. PROPEL is also referred to as “ATB200-03”, see Example 9.

Patients switching to AT-GAA from the approved standard of care ERT (alglucosidase alfa) walked on average 17 meters farther (p=0.046).

Patients switching to AT-GAA also showed an improvement in percent-predicted forced vital capacity (FVC), the most important measure of respiratory function in Pompe disease, compared to a decline in patients treated with alglucosidase alfa (FVC Diff. 4.1%; p=0.006).

AT-GAA showed a nominally statistically significant and clinically meaningful difference for superiority on the first key secondary endpoint of FVC compared to patients treated with alglucosidase alfa (FVC Diff. 3.0%; p=0.023).

In the combined study population of ERT switch and ERT naïve patients, AT-GAA outperformed alglucosidase alfa by 14 meters (21 m compared to 7 m) on the primary endpoint and was not statistically significant for superiority (p=0.072).

Improvements in the two important biomarkers of Pompe disease (Hex-4 and CK) for the combined study population significantly favored AT-GAA compared to alglucosidase alfa (p<0.001).

PROPEL was a 52-week, double-blind randomized global study designed to assess the efficacy, safety and tolerability of AT-GAA compared to the current standard of care, alglucosidase alfa, an enzyme replacement therapy (ERT). The study enrolled 123 adult Pompe patients who still had the ability to walk and to breathe without mechanical ventilation and was conducted at 62 clinical sites in 24 countries on 5 continents. It was the largest controlled clinical study ever conducted in a lysosomal disorder.

Patients enrolled in PROPEL were randomized 2:1 so that for every two patients randomized to be treated with AT-GAA, one was randomized to be treated with alglucosidase alfa. Of the Pompe patients enrolled in PROPEL, 77% were being treated with alglucosidase alfa (n=95) immediately prior to enrollment and 23% had never been treated with any ERT (n=28). 117 patients completed the PROPEL study and all 117 have voluntarily enrolled in the long-term extension study and are now being treated solely with AT-GAA for their Pompe disease.

### Pre-Specified Analyses of 6-Minute Walk Distance (6MWD) and Percent-Predicted Forced Vital Capacity (FVC) in the Combined ERT Switch and ERT Naïve Study Population:

The primary endpoint of the study was the mean change in 6-minute walk distance as compared with baseline measurements at 52 weeks across the combined ERT switch and ERT naïve patient populations. In this combined population patients taking AT-GAA (n=85) walked on average 21 meters farther at 52 weeks compared to 7 meters with those treated with alglucosidase alfa (n=37) (Table 14). This primary endpoint in the combined population was assessed for superiority and while numerically greater, statistical significance for superiority on this combined population was not achieved for the AT-GAA arm as compared to the alglucosidase alfa arm (p=0.072).

Per the hierarchy of the statistical analysis plan, the first key secondary endpoint of the study was the mean change in percent-predicted FVC at 52 weeks across the combined population. In this combined population patients taking AT-GAA demonstrated a nominally statistically significant and clinically meaningful difference for superiority over those treated with alglucosidase alfa. AT-GAA significantly slowed the rate of respiratory decline in patients after 52 weeks. Patients treated with AT-GAA showed a 0.9% absolute decline in percent-predicted FVC compared to a 4.0% absolute decline in the alglucosidase alfa arm (p=0.023) (Table 15). Percent-predicted FVC is the most important measure of respiratory muscle function in Pompe disease and was the basis of approval for alglucosidase alfa.

TABLE 14
6MWD (m) in the Overall ERT Switch and ERT Naïve Study Population

| Treatment | Baseline | CFBL at Week 52 | Difference | P-Value |
|---|---|---|---|---|
| AT-GAA (n = 85) | 357.9 (111.8) | +20.8 (4.6) | +13.6 (8.3) | p = 0.072 |
| Alglucosidase alfa (n = 37) | 351.0 (121.3) | +7.2 (6.6) | | |

TABLE 15
FVC (% predicted) in the Overall ERT Switch and ERT Naïve Study Population

| Treatment | Baseline | CFBL at Week 52 | Difference | P-Value |
|---|---|---|---|---|
| AT-GAA (n = 85) | 70.7 (19.6) | −0.9 (0.7) | +3.0 (1.2) | p = 0.023 |
| Alglucosidase alfa (n = 37) | 69.7 (21.5) | −4.0 (0.8) | | |

Pre-Specified Analyses of 6-Minute Walk Distance (6MWD) and Percent-Predicted Forced Vital Capacity (FVC) in the ERT Switch Study Population (n=95):

The PROPEL switch patients entered the study having been treated with alglucosidase alfa for a minimum of two years. More than two thirds (67%+) of those patients had been on ERT treatment for more than five years prior to entering the PROPEL study (mean of 7.4 years).

A pre-specified analysis of the patients switching from alglucosidase alfa on 6-minute walk distance showed that after 52 weeks from switching, AT-GAA treated patients (n=65) walked 16.9 meters farther than their baseline, compared to 0.0 meters for those patients who were randomized to remain on alglucosidase alfa (n=30) (p=0.046) (Table 16).

A pre-specified analysis of the patients switching from alglucosidase alfa on percent-predicted FVC showed that AT-GAA treated patients stabilized and slightly improved their respiratory function on this important measure while those patients remaining on alglucosidase alfa continued to significantly decline in respiratory muscle function. AT-GAA patients showed a 0.1% absolute increase in percent-predicted FVC while the alglucosidase alfa patients showed a 4.0% absolute decline over the course of the year (p=0.006) (Table 17).

TABLE 16
6MWD (m) in the ERT Switch Study Population

| Treatment | Baseline | CFBL at Week 52 | Difference | P-Value |
|---|---|---|---|---|
| AT-GAA (n = 65) | 346.9 (110.2) | +16.9 (5.0) | +16.9 (8.8) | p = 0.046 |
| Alglucosidase alfa | 334.6 (114.0) | 0.0 (7.2) | | |
| (n = 30) | | | | |

TABLE 17
FVC (% predicted) in the ERT Switch Study Population

| Treatment | Baseline | CFBL at Week 52 | Difference | P-Value |
|---|---|---|---|---|
| AT-GAA (n = 65) | 67.9 (19.1) | +0.1 (0.7) | +4.1 (1.2) | p = 0.006 |
| Alglucosidase alfa (n = 30) | 67.5 (21.0) | −4.0 (0.9) | | |

Pre-Specified Analyses of 6-Minute Walk Distance (6MWD) and Percent-Predicted Forced Vital Capacity (FVC) in the ERT Treatment Naive Population (n=28):

A pre-specified analysis of the patients previously never treated with any ERT on 6-minute walk distance showed that after 52 weeks AT-GAA treated patients (n=20) walked 33 meters farther than their baseline. The alglucosidase alfa treated patients (n=7) walked 38 meters further than their baseline. The difference between the two groups was not statistically significant (p=0.60) (Table 18).

A pre-specified analysis of the patients never previously treated with any ERT showed similar declines in percent-predicted forced vital capacity (FVC) at 52 weeks of −4.1% for AT-GAA treated patients and −3.6% for alglucosidase alfa treated patients (Table 19). The difference between the two groups was not statistically significant (p=0.57).

TABLE 18
6MWD (m) in the ERT Treatment Naive Population

| Treatment | Baseline | CFBL at Week 52 | Difference | P-Value |
|---|---|---|---|---|
| AT-GAA (n = 20) | 393.6 (112.4) | +33.4 (10.9) | −4.9 (19.7) | p = 0.60 |
| Alglucosidase alfa | 420.9 (135.7) | +38.3 (11.1) | | |
| (n = 7) | | | | |

TABLE 19
FVC (% predicted) in the ERT Treatment Naive Population

| Treatment | Baseline | CFBL at Week 52 | Difference | P-Value |
|---|---|---|---|---|
| AT-GAA (n = 20) | 80.2 (18.7) | −4.1 (1.5) | −0.5 (2.7) | p = 0.57 |
| Alglucosidase alfa (n = 7) | 79.1 (22.6) | −3.6 (1.8) | | |

Note:
One patient in the alglucosidase alfa arm was excluded from the study analysis due to use of an investigational anabolic like steroid that impacted his baseline performance.

### Pre-Specified Analyses of Other Key Secondary and Biomarker Endpoints Across the Overall ERT Switch and ERT Naïve Study Population:

### Musculoskeletal & Other Key Secondary Endpoints:

GSGC (Gait, Stairs, Gower's Chair): GSGC is an important and commonly used endpoint in Pompe Disease capturing strength, coordination and mobility. AT-GAA treated patients demonstrated statistically significant improvements on the scores in this important assessment, compared to a worsening for alglucosidase alfa treated patients in the overall population (p<0.05).

Lower MMT (Manual Muscle Testing), PROMIS Physical Function: On both of these validated measures of muscle strength and patient reported outcomes, AT-GAA treated patients improved numerically more than alglucosidase alfa treated patients, though the results were not statistically significant.

PROMIS Fatigue: Fatigue as measured by this scale slightly favored AT-GAA treated patients over alglucosidase alfa treated patients.

### Biomarkers of Treatment Effects on Disease:

Urine Hex-4: For the combined study population of both ERT switch and ERT naïve patients, those patients receiving AT-GAA showed substantial improvements on this biomarker, with a mean reduction of Hex-4 of −31.5% after 52 weeks compared to an increase of +11.0% (i.e., worsening) in Hex-4 in the alglucosidase alfa treated patients (p=<0.001). Urine Hex-4 is a common biomarker in Pompe disease and is used as an indirect measure of the degree of skeletal glycogen clearance in Pompe patients receiving ERT. Glycogen is the substrate that accumulates in the lysosomes of muscles of Pompe patients.

CK (Creatine Kinase): After 52 weeks, AT-GAA treated patients showed substantial improvements on this biomarker as well with a mean −22.4% reduction in CK compared to an increase (i.e., worsening) of +15.6% in the alglucosidase alfa treated patients. (p<0.001). CK is an enzyme that leaks out of damaged muscle cells and is elevated in Pompe patients.

AT-GAA demonstrated a similar safety profile to alglucosidase alfa. Two patients receiving AT-GAA (2.4%) discontinued treatment due to an adverse event compared to one (2.6%) for alglucosidase alfa unrelated to treatment. Injection associated reactions (IARs) were reported in 25% of AT-GAA participants and 26% of alglucosidase alfa patients.

### Post Hoc Subgroup Analyses:

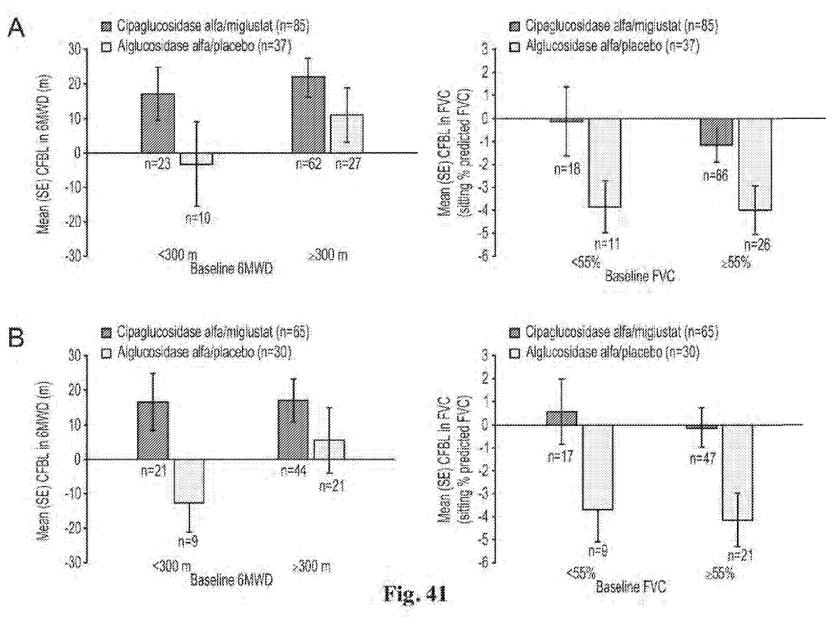

Baseline 6MWD and FVC categories: ERT-naïve population (n=27): three patients had a baseline 6MWD of <300 m and three had a baseline FVC of <55%; analyses of CFBL were not performed in these subgroups owing to the small patient numbers. Baseline 6MWD ≥300 m: both the cipaglucosidase alfa/miglustat (AT-GAA) (n=18) and alglucosidase alfa/placebo (n=6) groups had similar improvements over time (mean [SE] CFBL to week 52: +34.4 [12.1] m and +30.8 [9.6] m, respectively). Baseline FVC≥55%: both the cipaglucosidase alfa/miglustat (n=19) and alglucosidase alfa/placebo (n=5) groups declined over time (mean [SE] CFBL to week 52: −3.7 [1.5] % and −3.3 [2.6] %, respectively). Outcomes consistently favored cipaglucosidase alfa/miglustat in the overall and ERT-experienced populations in patients with baseline 6MWD of <300 m and ≥300 m, and FVC of <55% and ≥55%, as shown in FIG. 41A and FIG. 41B.

In the overall study population including ERT-naïve and ERT-experienced patients, cipaglucosidase alfa/miglustat showed positive trends or clinically meaningful improvements on motor and respiratory functions compared with approved ERT, regardless of baseline 6MWD and % FVC assessments, and across both prespecified and post hoc subgroup analyses.

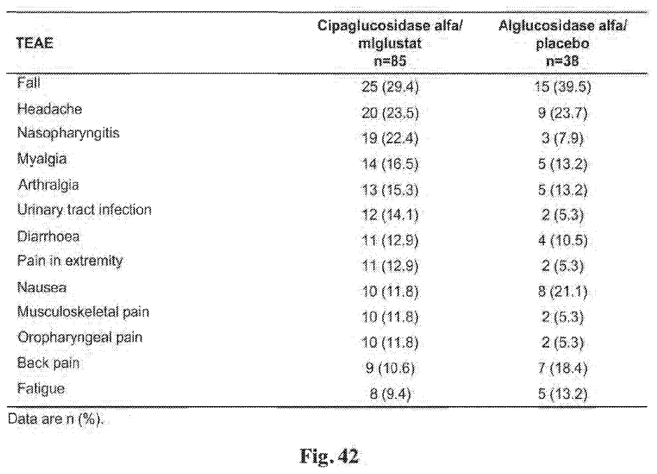

Cipaglucosidase alfa/miglustat demonstrated a similar safety profile to that of alglucosidase alfa/placebo (FIG. 42).

### About AT-GAA

AT-GAA is an investigational two-component therapy that consists of cipaglucosidase alfa (ATB200), a unique recombinant human acid alpha-glucosidase (rhGAA) enzyme with optimized carbohydrate structures, particularly bis-phosphorylated mannose-6 phosphate (bis-M6P) glycans, to enhance uptake into cells, administered in conjunction with miglustat (AT2221), a stabilizer of cipaglucosidase alfa. In preclinical studies, AT-GAA was associated with increased levels of the mature lysosomal form of GAA and reduced glycogen levels in muscle, alleviation of the autophagic defect and improvements in muscle strength.

### About Pompe Disease

Pompe disease is an inherited lysosomal disorder caused by deficiency of the enzyme acid alpha-glucosidase (GAA). Reduced or absent levels of GAA levels lead to accumulation of glycogen in cells, which is believed to result in the clinical manifestations of Pompe disease. The disease can be debilitating and is characterized by severe muscle weakness that worsens over time. Pompe disease ranges from a rapidly fatal infantile form with significant impacts to heart function to a more slowly progressive, late-onset form primarily affecting skeletal muscle. It is estimated that Pompe disease affects approximately 5,000 to 10,000 people worldwide.

### Example 11: Results of an Open-Label Phase I/II Study (ATB200-02)

ATB200-02 (NCT02675465) is an open-label, Phase I/II clinical trial that aimed to evaluate the safety, tolerability, pharmacokinetics, pharmacodynamics, and efficacy of cipaglucosidase alfa/miglustat in adults with Pompe disease. Cipaglucosidase alfa/miglustat is an investigational, two-component therapy for late-onset Pompe disease (LOPD) comprised of intravenous cipaglucosidase alfa, a rhGAA, administered in conjunction with oral miglustat, an enzyme stabilizer.

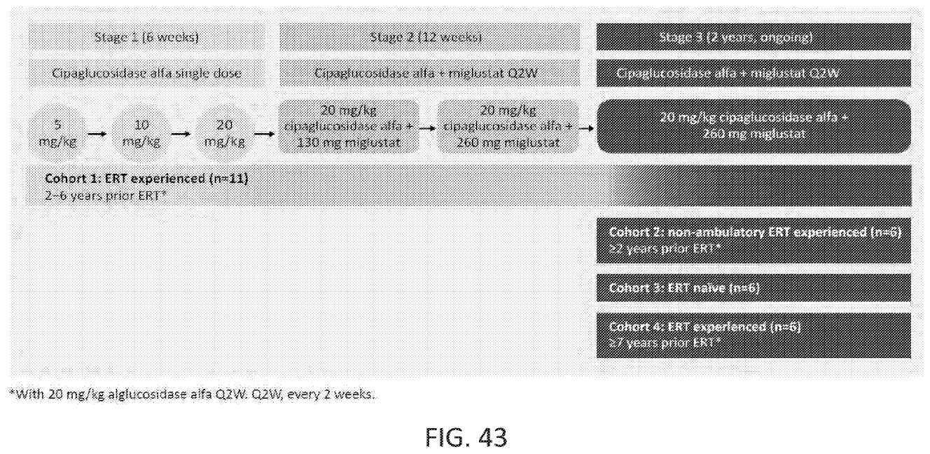

FIG. 43 shows the study design for the Phase I/II ATB200-02 study. The study is conducted in 16 centers across 5 countries. Four cohorts of patients with Pompe disease were enrolled in the ATB200-02 study:
Cohort 1: ERT-experienced, aged 18-65 years, with 2-6 years prior ERT with 20 mg/kg alglucosidase alfa every 2 weeks (n=11)
Cohort 2: Non-ambulatory ERT-experienced, aged 18-65 years, with ≥2 years prior ERT with 20 mg/kg alglucosidase alfa every 2 weeks (n=6)
Cohort 3: ERT-naïve, aged 18-65 years (n=6)
Cohort 4: ERT-experienced, aged 18-75 years, with ≥7 years prior ERT with 20 mg/kg alglucosidase alfa every 2 weeks (n=6)

Eligible ambulatory patients had a 6-minute walk distance (6MWD) of at least 200 m (cohorts 1 and 3) or 75 m (cohort 4) and upright forced vital capacity (FVC) of 30-80% of predicted normal value.

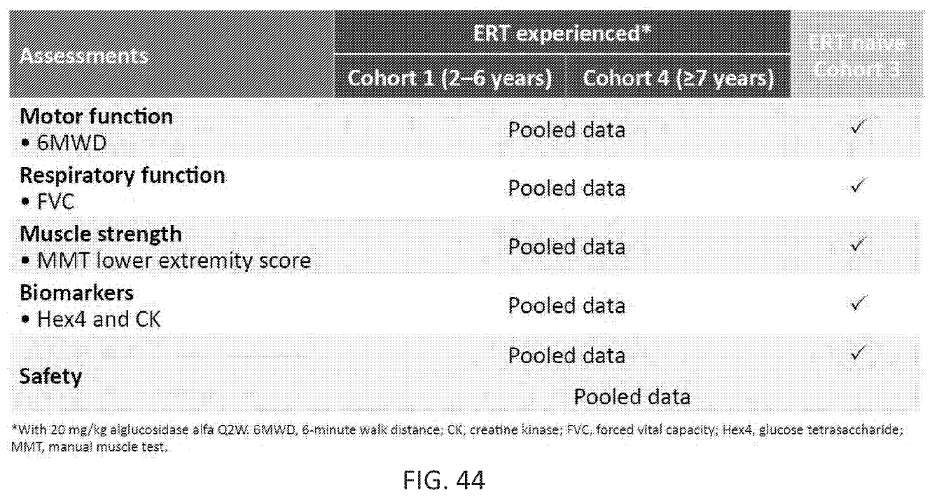

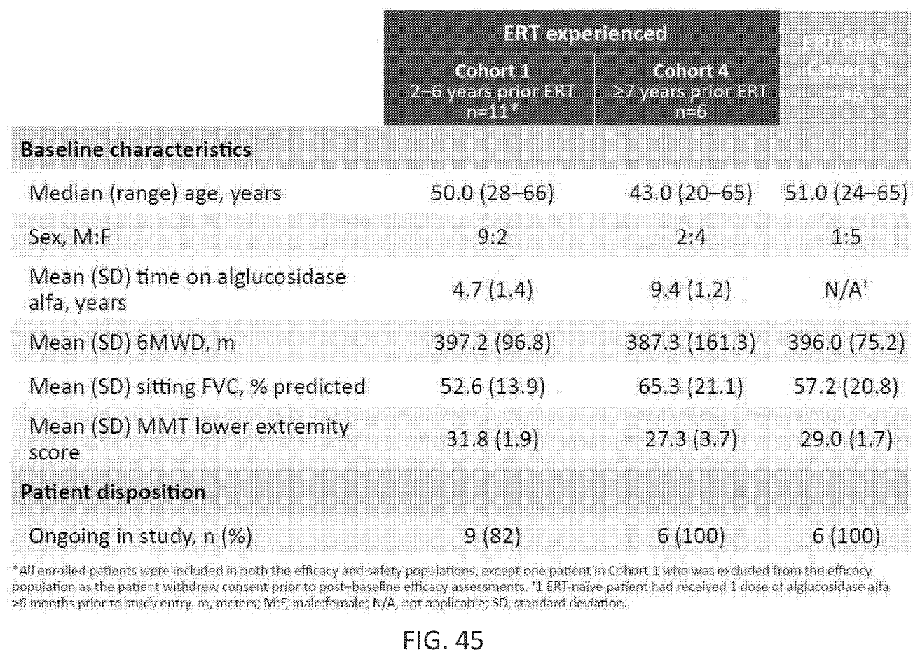

The change from baseline (CFBL) in 6MWD, % predicted sitting FVC, manual muscle test (MMT) and biomarkers—urine glucose tetrasaccharide (Hex4) and serum creatine kinase (CK)—were assessed at regular intervals. Data with up to 36 months of follow-up in ERT-experienced and ERT-naïve patients is provided herein. FIG. 44 shows a summary of endpoints and cohorts reported. FIG. 45 shows the baseline characteristics and patient disposition. Due to the staggered timing of patient enrollment, the number of patients with data currently available decreases at later time-points in this ongoing study.

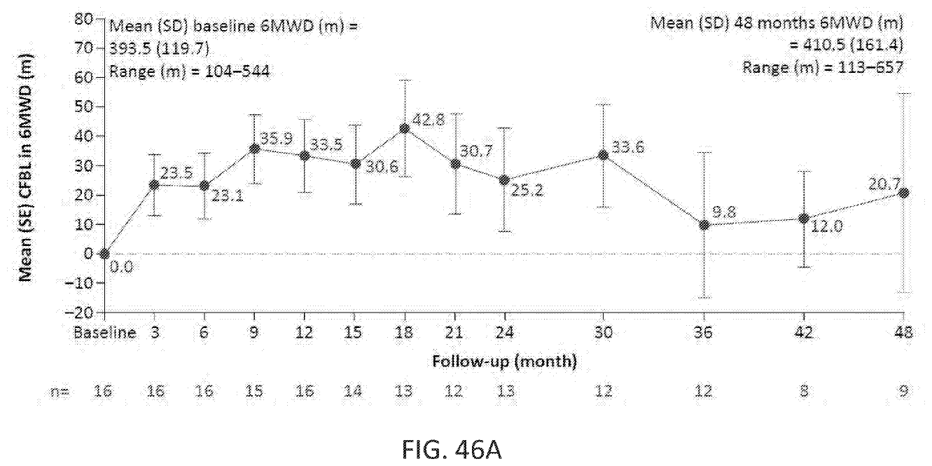

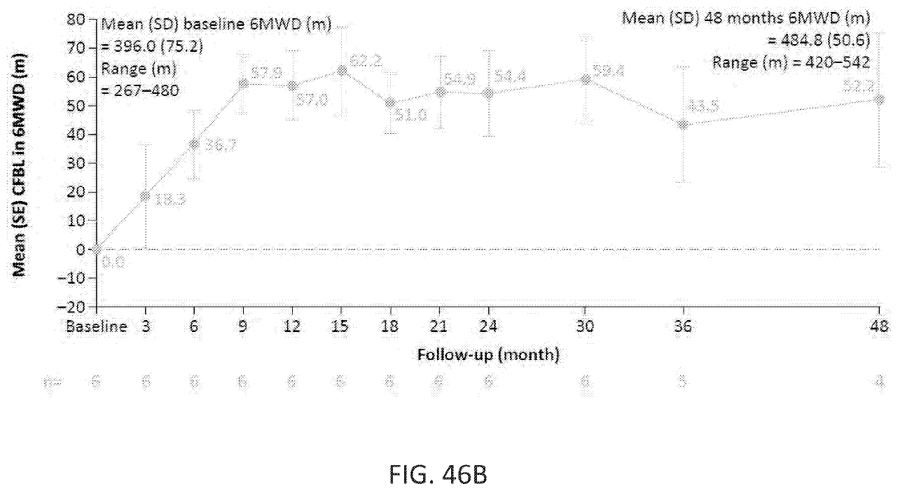

ERT-experienced patients showed durable mean improvements from baseline in 6MWD up to 48 months. After 12-, 24-, 36- and 48-months of follow-up, 6MWD improved numerically from baseline in 13/16, 9/13, 6/12 and 6/9 ERT-experienced patients, respectively (FIG. 46A). The mean increases were 33 meters (m) by month 12, 25 m by month 24, 9 m by month 36 and 20 m by month 48.

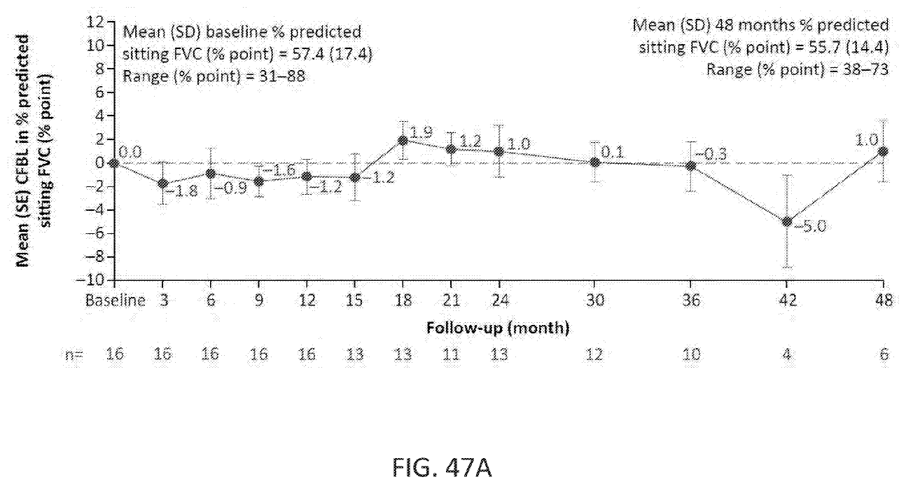

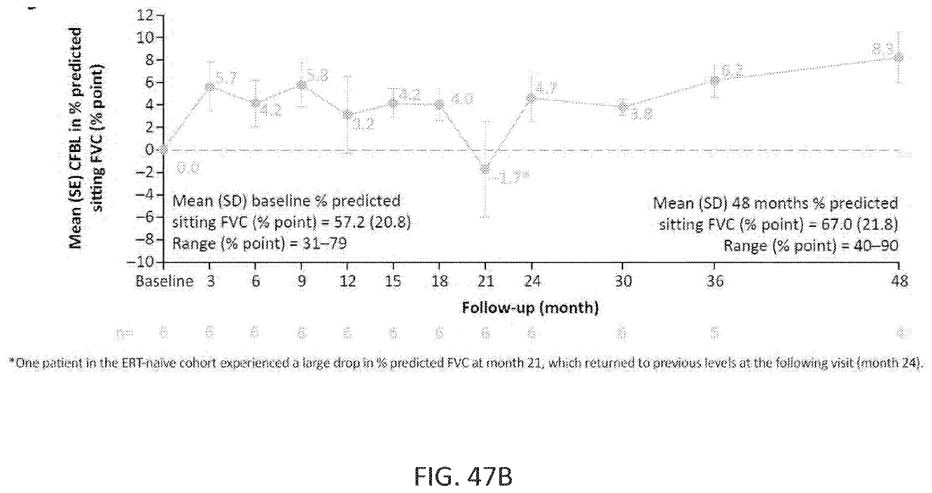

For ERT-experienced patients, change from baseline (CFBL) in FVC was generally stable for up to 48 months of follow-up. After 12-, 24-, 36- and 48-months of follow-up, FVC improved (>3%) or remained stable (±3%) from baseline in 7/16, 8/13, 4/10 and 4/6 patients, respectively (FIG. 47A). The mean changes were −1.2% by month 12, +1.0% by month 24, −0.3% by month 36 and +1.0% by month 48.

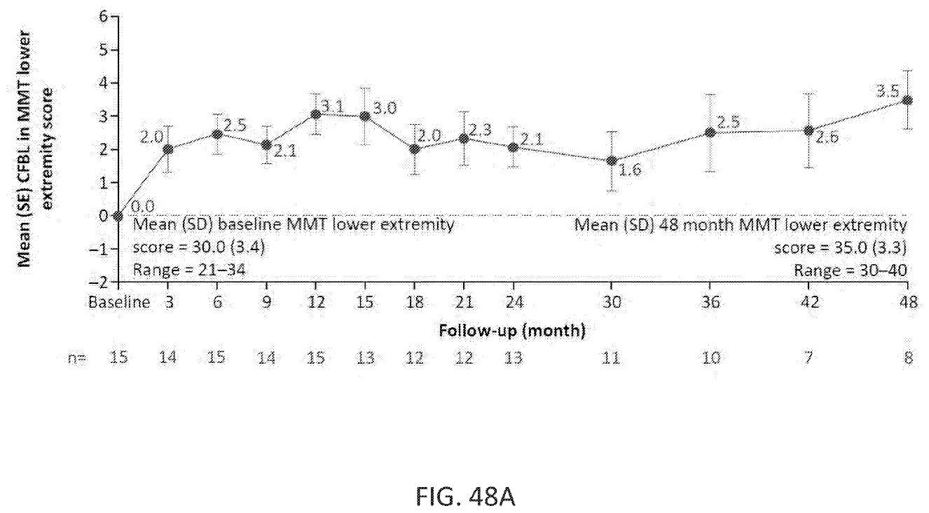

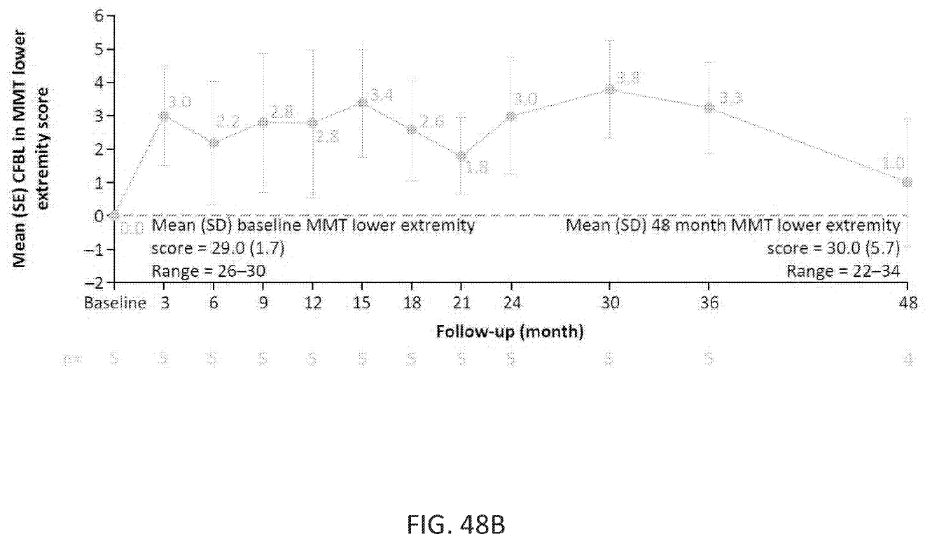

In ERT-experienced patients, mean change in MMT lower extremity score improved numerically from baseline and improvements were maintained for up to 48-months of follow-up (FIG. 48A). The mean increases were 3.1 pts by month 12, 2.1 pts by month 24, 2.5 pts by month 36 and 3.5 pts by month 48.

ERT-naïve patients showed durable mean improvements from baseline in 6MWD up to 48 months. After 12-, 24-, 36- and 48-months of follow-up, 6MWD improved numerically from baseline in 6/6, 6/6, 4/5 and 4/4 ERT-naïve patients, respectively (FIG. 46B). The mean increases were 57 m by month 12, 54 m by month 24, 43 m by month 36 and 52 by month 48.

In ERT-naïve patients, mean CFBL in FVC improved numerically from baseline for up to 48 months of follow-up. After 12-, 24-36-, and 48-months of follow-up, FVC improved (>3%) or remained stable (±3%) from baseline in 4/6, 5/6, 5/5 and 4/4 patients, respectively (FIG. 47B). As indicated by the asterisk in FIG. 47B, one patient in the ERT-naïve cohort experienced a large drop in % predicted FVC at month 21, which returned to previous levels at the following visit (month 24). The mean changes were 3.2% by month 12, 4.7% by month 24, 6.2% by month 36 and 8.3% by month 48.

In ERT-naïve patients, mean change in MMT lower extremity score improved numerically from baseline and improvements were maintained for up to 48-months of follow-up (FIG. 48B). The mean increases were 2.8 pts by month 12, 3.0 pts by month 24, 3.3 pts by month 36 and 1.0 pts by month 48.

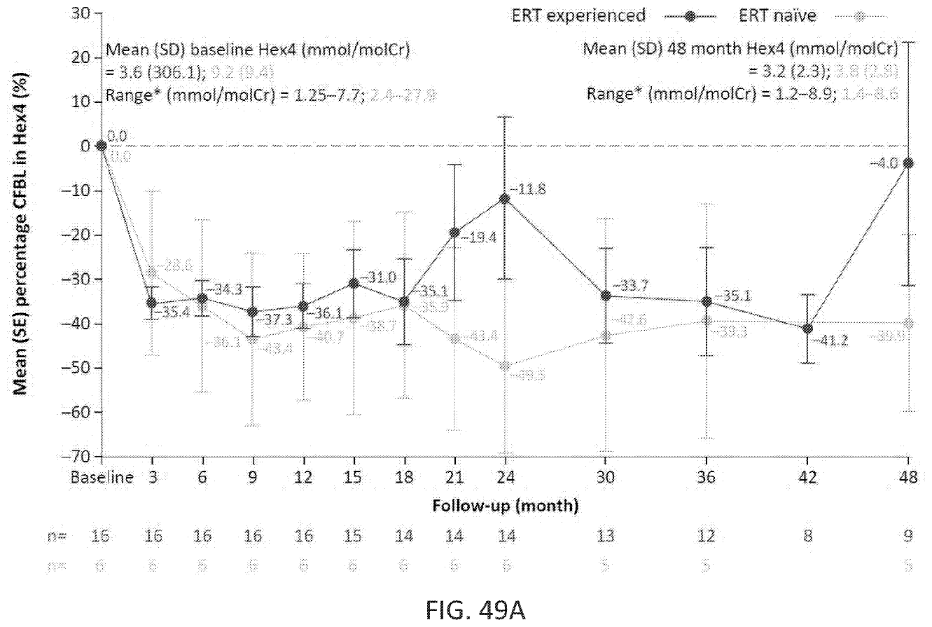

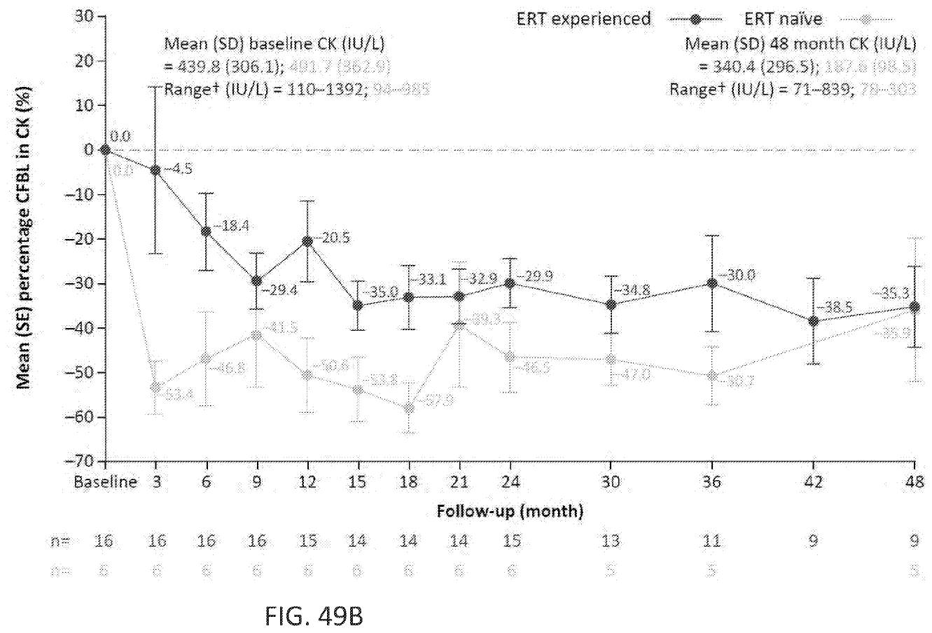

During 48 months of follow-up, cipaglucosidase alfa/miglustat was generally associated with mean reductions from baseline in urine Hex4, with greater reductions in ERT-naïve patients. After 12-, 24-, 36- and 48-months of follow-up, Hex4 levels decreased numerically from baseline in 16/16, 11/14, 11/12 and 6/9 ERT-experienced patients, and in 5/6, 5/6, 4/5 and 4/5 ERT-naïve patients, respectively (FIG. 49A).

During 48 months of follow-up, cipaglucosidase alfa/miglustat was associated with either stable levels of, or mean reductions from baseline, in plasma CK, with greater reductions in ERT-naïve patients. After 12-, 24-, 36- and 48-months of follow-up, CK levels decreased numerically from baseline in 13/15, 14/15, 9/11 and 8/9 ERT-experienced patients, and in 6/6, 6/6, 5/5 and 4/5 ERT-naïve patients, respectively (FIG. 49B).

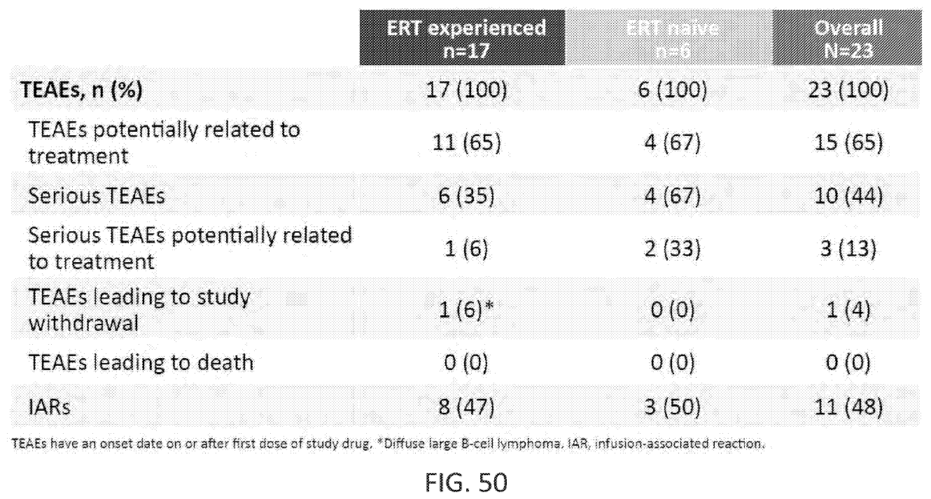

FIG. 50 shows a summary of treatment emergent adverse events (TEAEs) with onset date on or after first dose of study drug in the ATB200-02 study. Mean (SD) duration of treatment was 37.2 (14.48), 19.9 (4.13) and 36.9 (12.14) months in cohorts 1 (prior ERT 2-6 years), 4 (prior ERT≥7 years) and 3 (ERT naive), respectively. The most common TEAEs included fall, nasopharyngitis, arthralgia, headache and diarrhea; most TEAEs were mild or moderate in severity and did not lead to study withdrawal.

Results from up to 48-months of follow-up in ambulatory patients from the ATB200-02 study of cipaglucosidase alfa plus miglustat indicate the following. ERT-experienced patients had durable mean improvements from baseline in motor function that were sustained for up to 48 months of follow-up, while respiratory function was stable and maintained over the same period: an improvement relative to the expected decline in many patients receiving long-term ERT. ERT-naïve patients showed durable mean improvements from baseline in motor and respiratory function that were sustained for up to 48 months of follow-up. Mean levels of two biomarkers, Hex4 and CK, were either stable or decreased from baseline up to 48 months of follow-up, with decreases most notable in the ERT-naïve cohort. The safety profile of cipaglucosidase alfa plus miglustat was similar to that reported for alglucosidase alfa.

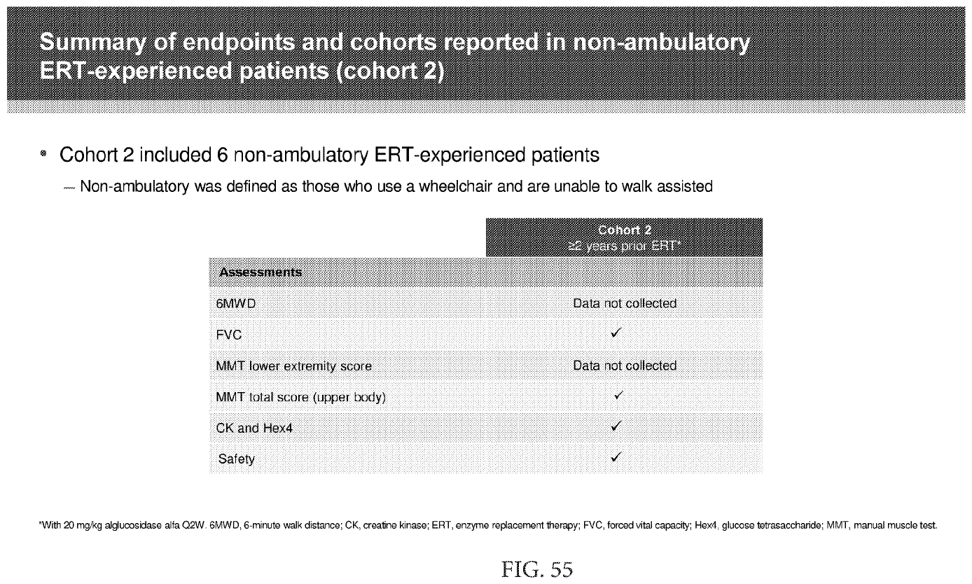

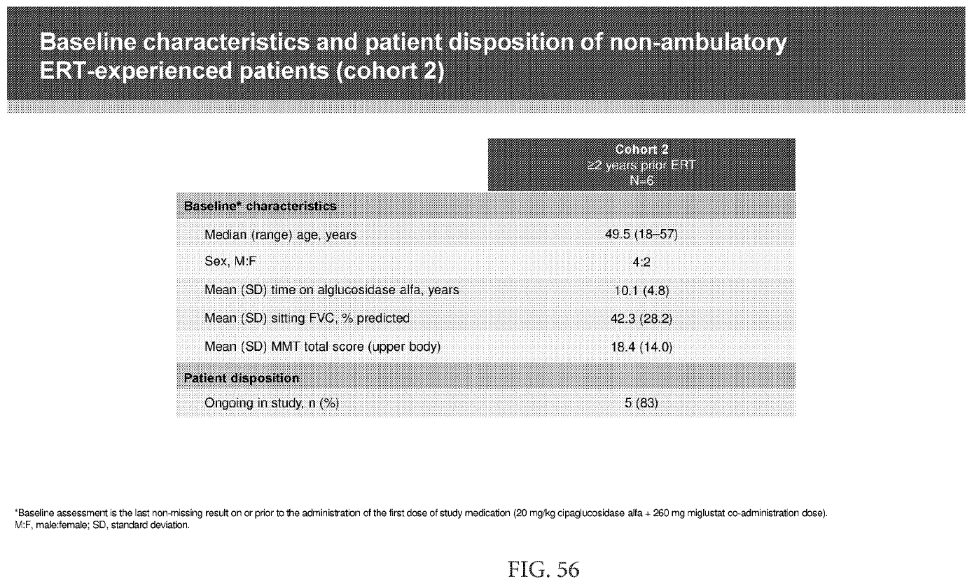

FIG. 55 shows a summary of endpoints and cohorts reported for Cohort 2 (non-ambulatory ERT-experienced patients). FIG. 56 shows the baseline characteristics and patient disposition for Cohort 2.

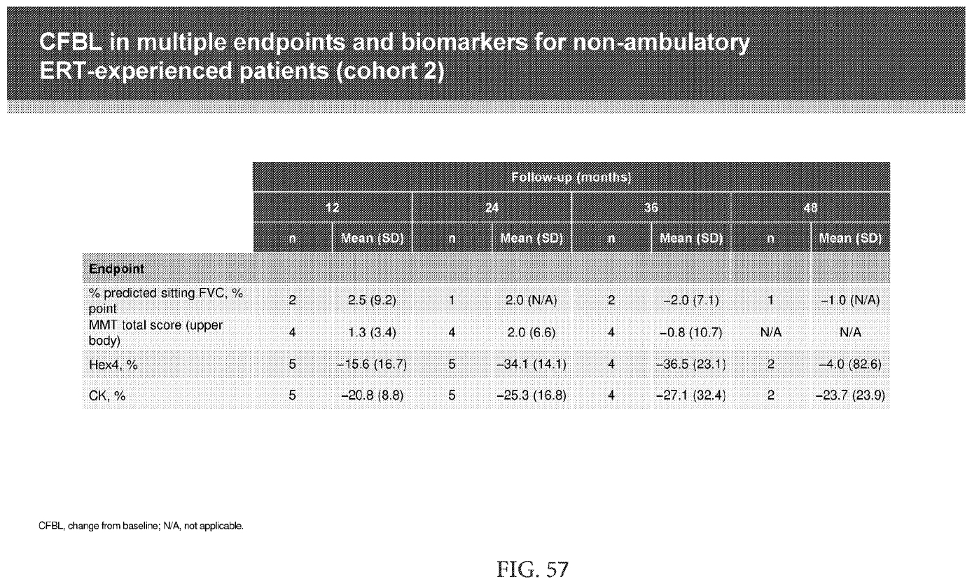

For these non-ambulatory ERT-experienced patients, change from baseline (CFBL) in FVC improved numerically from baseline and/or remained stable for up to 48 months of follow-up (FIG. 57). The mean changes were +2.5% by month 12, +2.0% by month 24, −2.0% by month 36 and −1.0% by month 48.

In these non-ambulatory ERT-experienced patients, mean change in MMT total score (upper body) improved numerically from baseline and/or remained stable for up to 36-months of follow-up (FIG. 57). The mean changes were +1.3 pts by month 12, +2.0 pts by month 24 and −0.8 pts by month 36.

During 48 months of follow-up in these these non-ambulatory ERT-experienced patients, cipaglucosidase alfa/miglustat was generally associated with mean reductions from baseline in urine Hex4 and plasma CK (FIG. 57). After 12-, 24-, 36- and 48-months of follow-up, Hex4 levels decreased numerically from baseline with mean reductions of −15.6%, −34.1%, −36.5% and −4.0%, respectively. After 12-, 24-, 36- and 48-months of follow-up, plasma CK levels decreased numerically from baseline with mean reductions of −20.8%, −25.3%, −27. % and −23.7%, respectively.

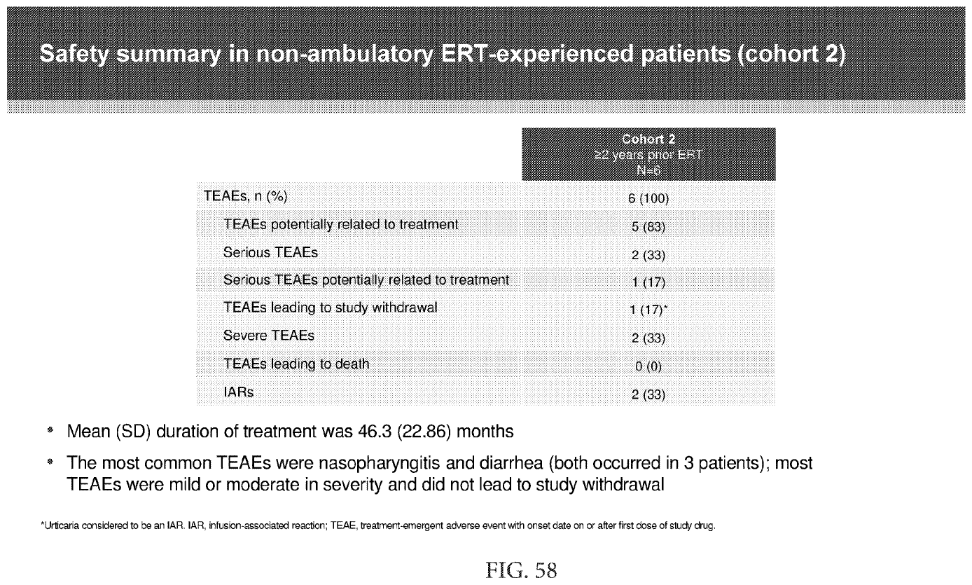

FIG. 58 shows a summary of treatment emergent adverse events (TEAEs) with onset date on or after first dose of study drug or Cohort 2 (in non-ambulatory ERT-experienced patients) in the ATB200-02 study. Mean (SD) duration of treatment was 46.3 (22.86) months. The most common TEAEs included nasopharyngitis and diarrhea (both occurred in 3 patients); most TEAEs were mild or moderate in severity and did not lead to study withdrawal.

Results from up to 48-months of follow-up in non-ambulatory ERT-experienced patients from the ATB200-02 study of cipaglucosidase alfa plus miglustat indicate the following. These patients had durable mean improvements from baseline and/or stabilization in motor function and pulmonary function that were sustained for up to 48 months of follow-up: an improvement relative to the expected decline in many patients receiving long-term ERT. Mean levels of two biomarkers, Hex4 and CK, were either stable or decreased from baseline up to 48 months of follow-up. Cipaglucosidase alfa plus miglustat was generally well-tolerated in this patient group.

### Example 12: Long-Term Effects of ERT in Pompe Disease Patients

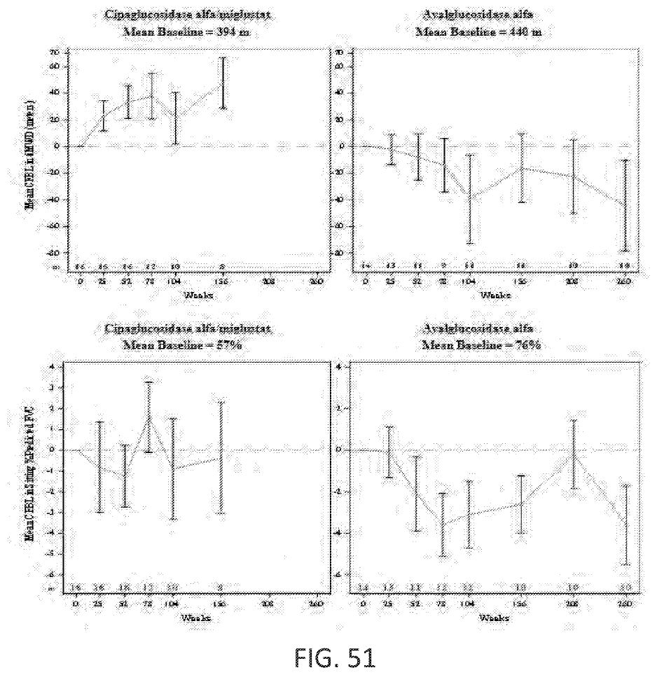

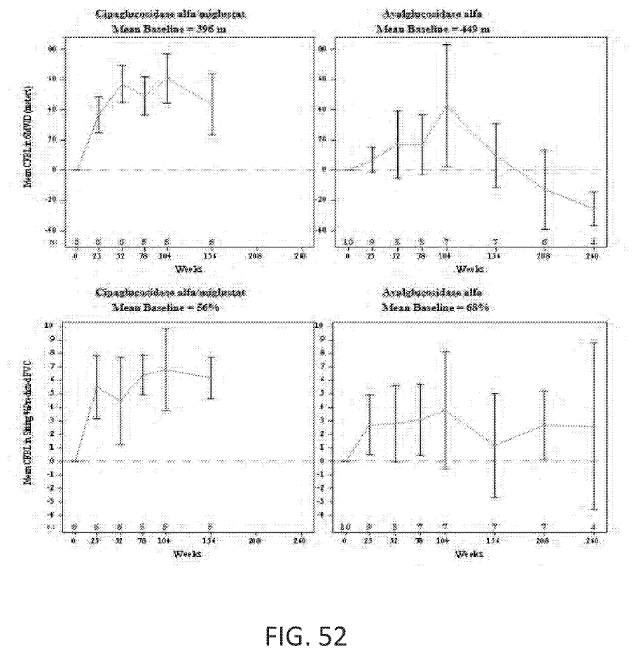

FIG. 51 shows a comparison of the long-term effects of cipaglucosidase alfa/miglustat and avalglucosidase alfa on change from baseline for 6MWD and percentage predicted FVC (sitting) in ERT-experienced subjects. FIG. 52 shows a comparison of the long-term effects of cipaglucosidase alfa/miglustat and avalglucosidase alfa on change from baseline for 6MWD and percentage predicted FVC (sitting) in ERT-naïve subjects. The comparison shows that ERT-experienced patients switching from alglucosidase alfa to avalglucosidase alfa continue to experience progressive loss of motor function as assessed by 6MWD, similar to the progression shown above for patients remaining on alglucosidase alfa long-term, whereas patients switching from alglucosidase alfa to cipaglucosidase alfa/miglustat experience a categorical change showing progressive gains in motor function as assessed by 6MWD. Similar trends are observed for FVC, where patients switching to avalglucosidase alfa experience progressive loss of pulmonary function as assessed by FVC whereas patients switching from alglucosidase alfa to cipaglucosidase alfa/miglustat experience a stability in pulmonary function as assessed by FVC.

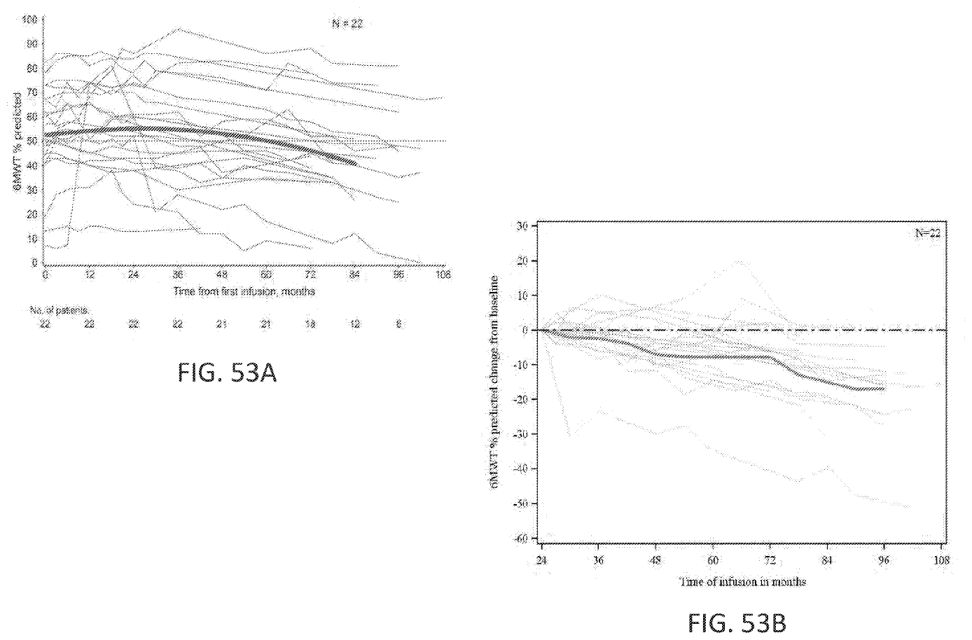

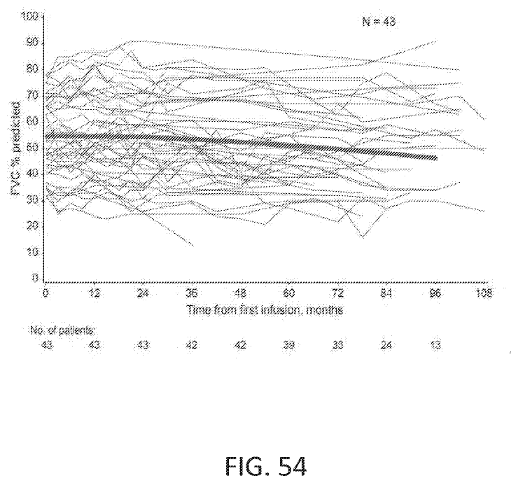

FIG. 53A-FIG. 53B show the 6-minute walk test (6MWT) percentage predicted during treatment with alglucosidase alfa. FIG. 53B shows replotted data from FIG. 53A only from year 2 onward. FIG. 54 shows the FVC percentage predicted during treatment with alglucosidase alfa. The data from year 2 onward show the decline expected in ERT-experienced patients remaining on alglucosidase alfa.

### Example 13: Comparison of Alglucosidase Alfa (Alglu), Avalglucosidase Alfa (Aval) and Cipaglucosidase Alfa+Miglustat (Cipa+mig)

In the absence of head-to-head trials comparing Aval to Cipa+mig, an indirect treatment comparison (ITC) is a suitable approach to better understand clinical differentiation of the three treatments for LOPD. ITCs are widely requested by health technology assessment agencies (HTAs) to support comparative health economic evaluation.

An ITC providing relative effect estimates in the target population of interest (i.e. an LOPD population including a mix of ERT-naïve and ERT-experienced subjects as in the pivotal Phase III trial comparing Cipa+mig with Alglu [PROPEL]) was performed.

A systematic literature review (SLR) was conducted to identify relevant published clinical studies of ERTs in LOPD. Outcomes assessed were change from baseline in 6-minute walking distance (6MWD) (m) and in forced vital capacity (FVC; % predicted) at week 52, acknowledged by clinicians, HTA agencies and payers as key LOPD trial endpoints. Aggregate results on 6MWD and FVC change from baseline over time and baseline characteristics (age, gender, ethnicity, previous ERT duration, baseline 6MWD and baseline FVC) were extracted from included studies.

A multi-level network meta regression (ML-NMR) was performed, which is an extension of standard network meta-analyses (NMAs) that take into account the effect of study-level covariates, and that can be applied to any connected network with any mixture of individual patient-level data (IPD) and aggregate data. ML-NMR is an accepted method by the National Institute for Health and Care Excellence (NICE) in support of cost effectiveness analysis. Single-arm study results were matched to appropriate comparator arms of the comparative studies to allow for inclusion into the network. Mean treatment differences with associated 95% credible intervals (CrIs) were calculated for 6MWD and FVC change from baseline at week 52.

A base-case scenario was evaluated in which all covariates were set to the target population of the PROPEL trial. To study the impact of previous ERT duration on relative effects, ERT duration value was varied, keeping remaining covariate values as in the base-case scenario. A sensitivity analysis was performed by excluding all matched single-arm evidence from the network to assess its impact on the results.

Both fixed effects (FE) and random effects (RE) ML-NMR models were applied, and the deviance information criteria (DIC) was used to assess goodness-of-fit of the models and to identify the appropriate model (FE or RE model) for the data. Models were implemented in a Bayesian framework using Stan with help of the R package multinma.

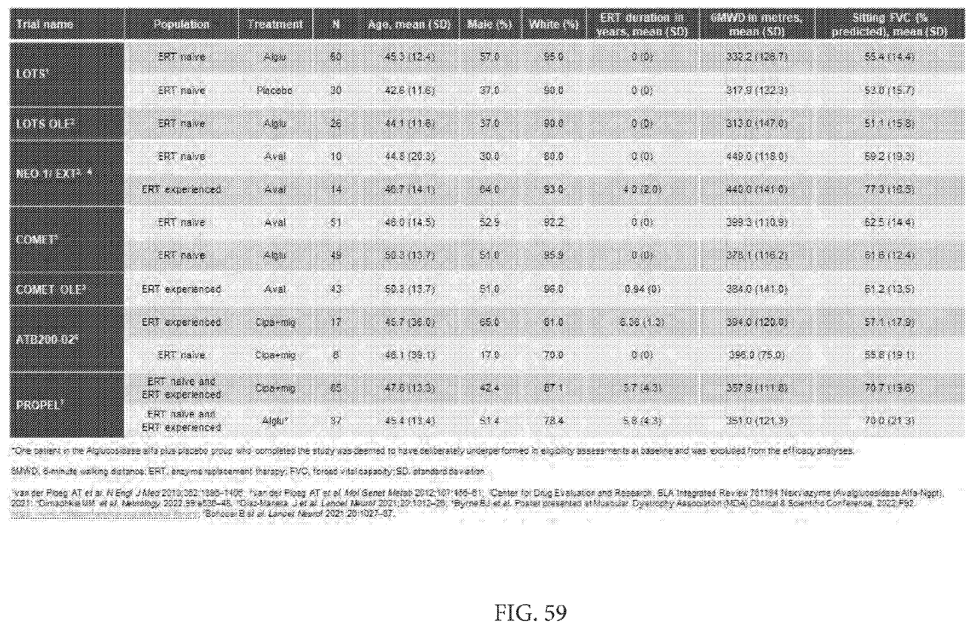

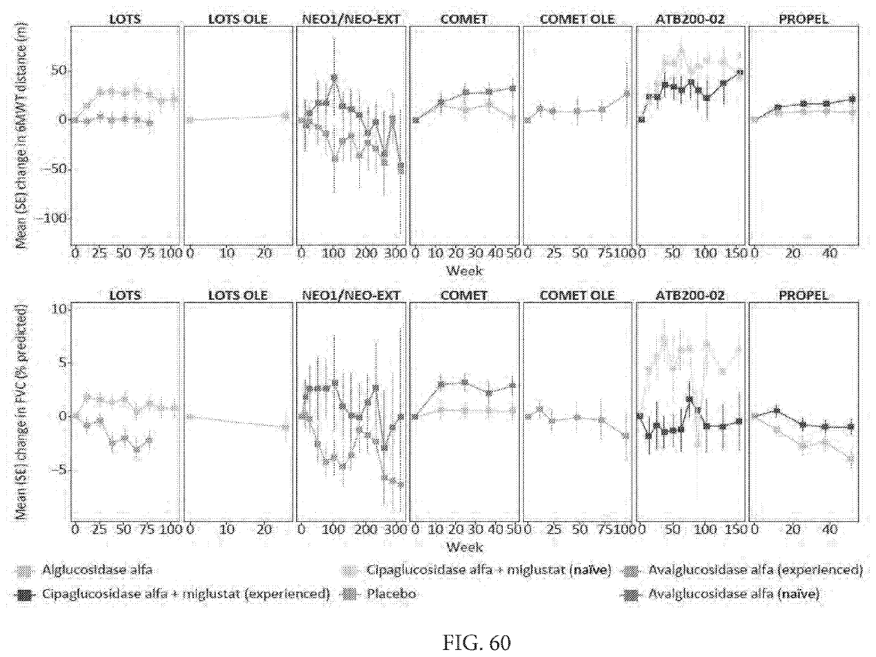

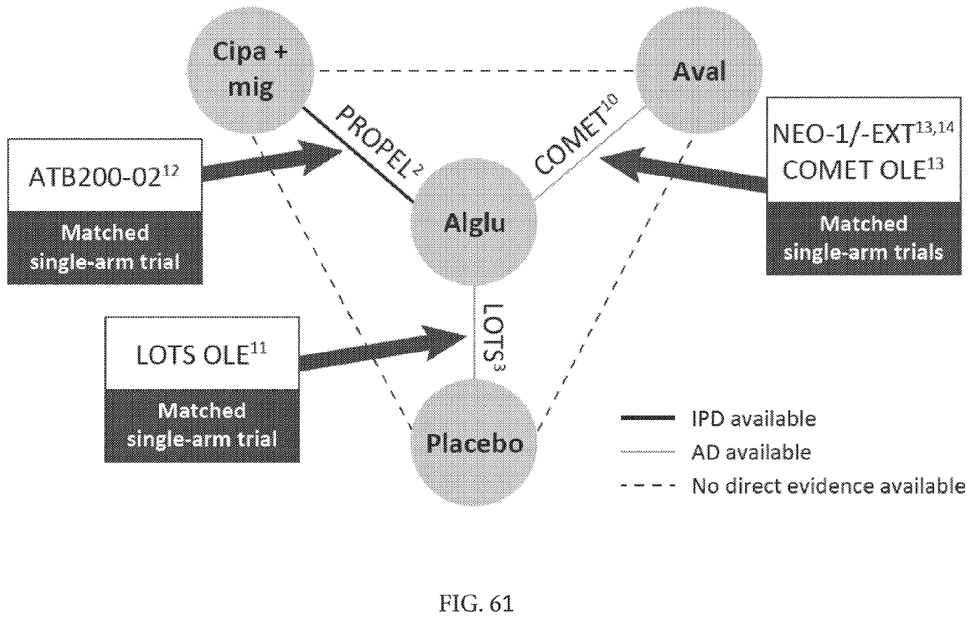

The SLR identified seven clinical studies for which baseline characteristics are shown in FIG. 59. These studies included but were not limited to three randomized clinical trials (LOTS: Alglu versus Placebo; COMET: Aval versus Alglu; PROPEL: Cipa+mig versus Alglu). Each share 6MWD and FVC as key primary or secondary endpoints (see FIGS. 60 and 61) but differ in their trial populations (PROPEL is the only randomized controlled trial [RCT] that comprised both ERT-naïve and -experienced subjects). Efficacy results of the included studies are shown in FIG. 60.

For both endpoints, the network is the same and shown in FIG. 61. Evidence from the single-arm studies LOTS OLE, NEO-1/-EXT, COMET OLE and ATB200-02 was included into the network, as shown in the blue boxes, by matching the single-arm results to appropriate comparator results from the head-to-head trials.

In the base-case scenario, the covariates were set to the baseline characteristics of the target population (i.e. the PROPEL trial; see Table 20), and time was set to 52 weeks.

TABLE 20
Base-case Scenario Covariate Setting

| Age | % | % | ERT duration | 6MWD | FVC (% |
|---|---|---|---|---|---|
| (years) | Male | White | (years) | (m) | predicted) |
| 46.95 | 45.08 | 84.43 | 5.744 | 355.8 | 70.42 |

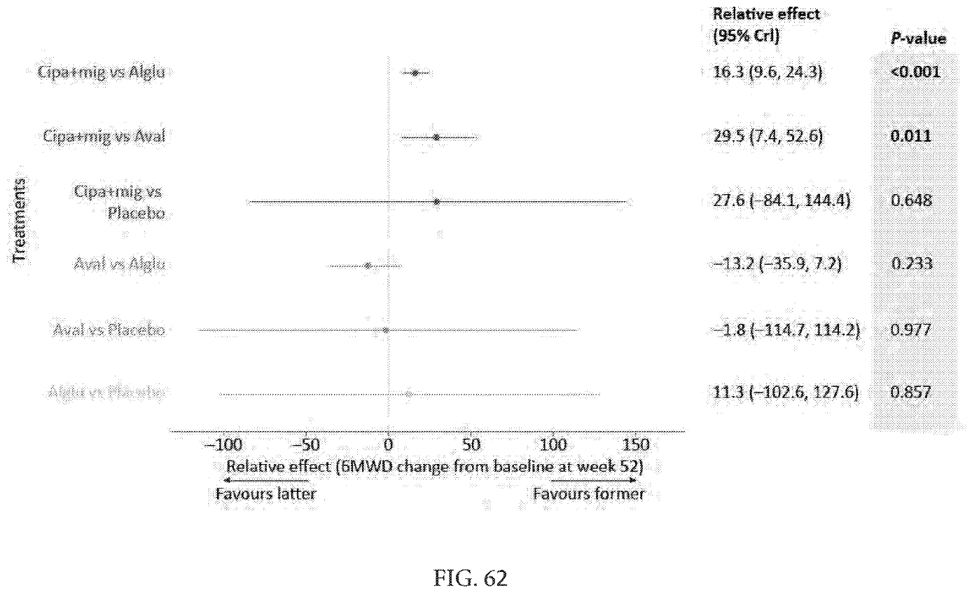

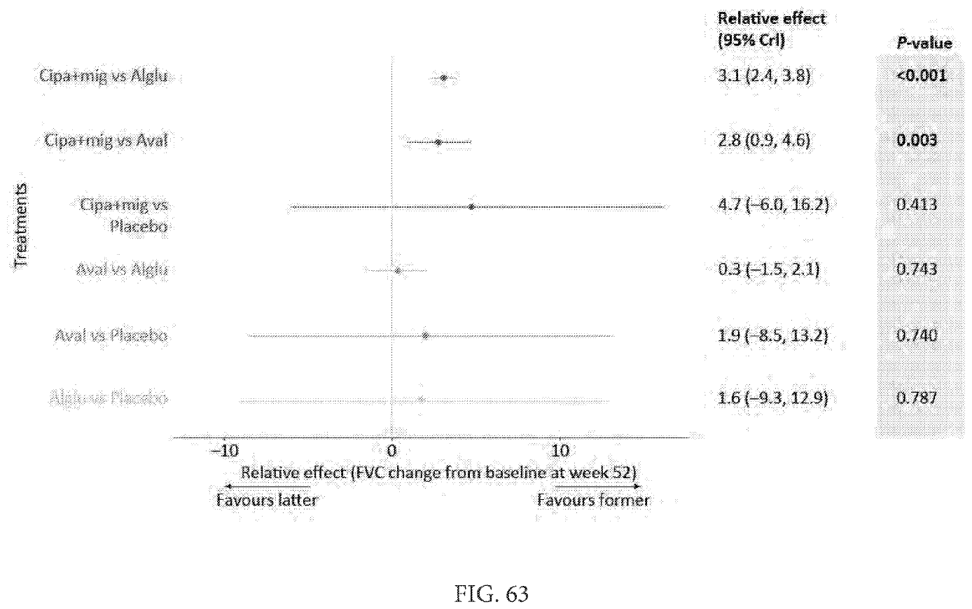

Based on the DIC, an RE model was chosen for 6MWD and an FE model for FVC. For both endpoints (FIGS. 62 and 63): Cipa+mig showed a statistically significant favorable effect versus Alglu and Aval; and Cipa+mig showed a numerically favorable effect versus placebo.

Note that the 95% CrIs of the relative effect estimates versus placebo are generally much wider than those versus Alglu or versus Aval. This reflects the larger uncertainty of those estimates, since data on placebo were only available for ERT-naïve subjects and previous ERT duration of the base-case scenario is relatively long (5.7 years).

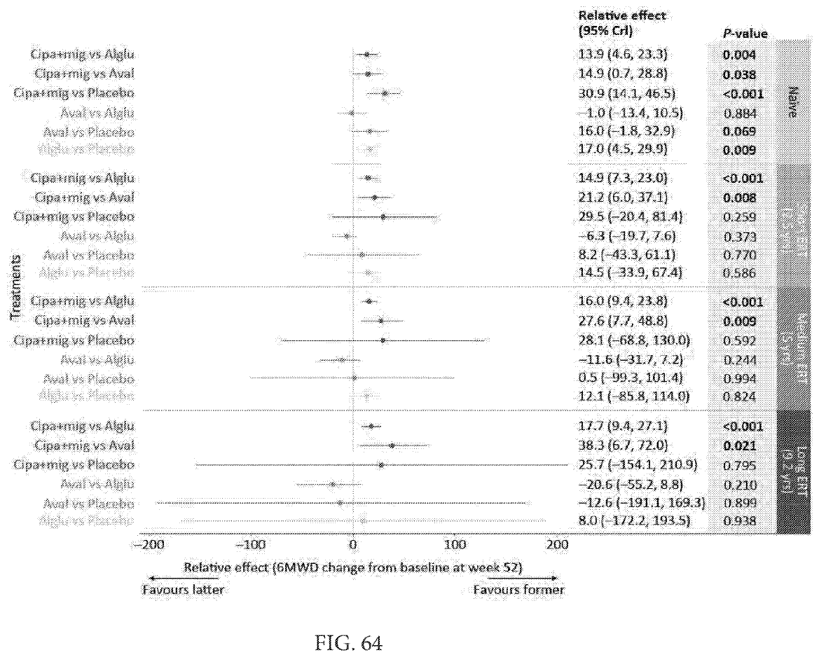

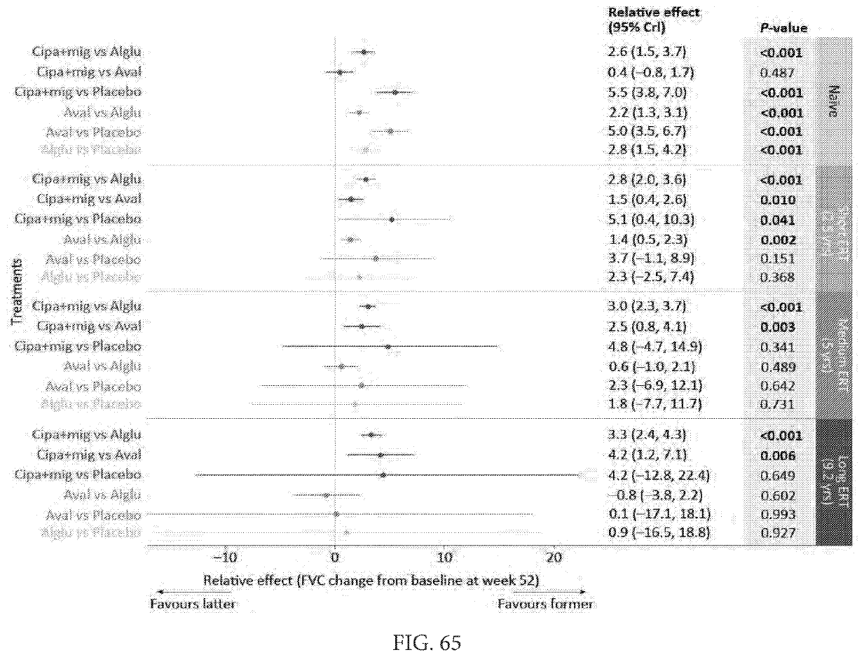

Relative effect estimates for different previous ERT durations (previous ERT duration=0 years [i.e. naïve patients], 2.5 years, 5 years, and 9.2 years) are shown in FIG. 64 (6MWD) and FIG. 65 (FVC).

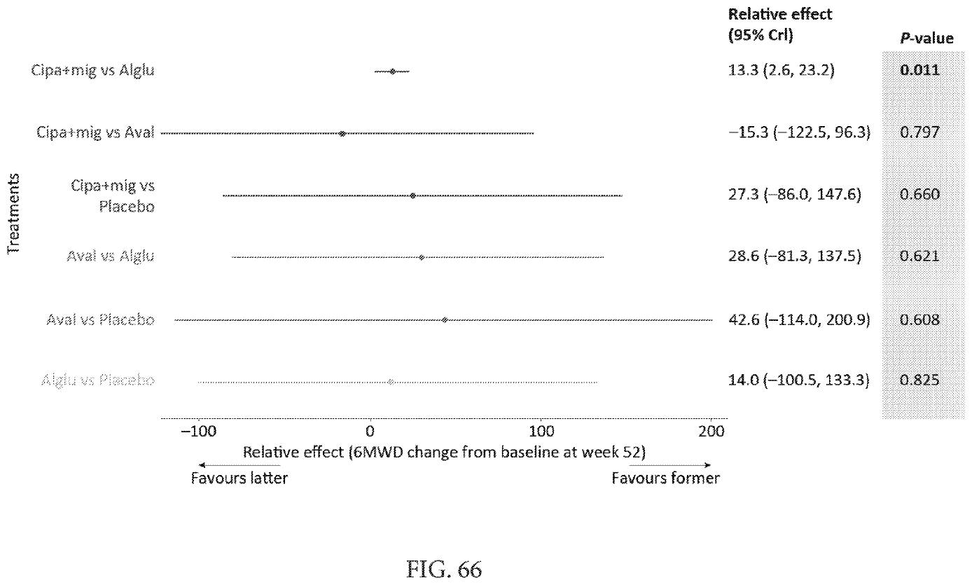

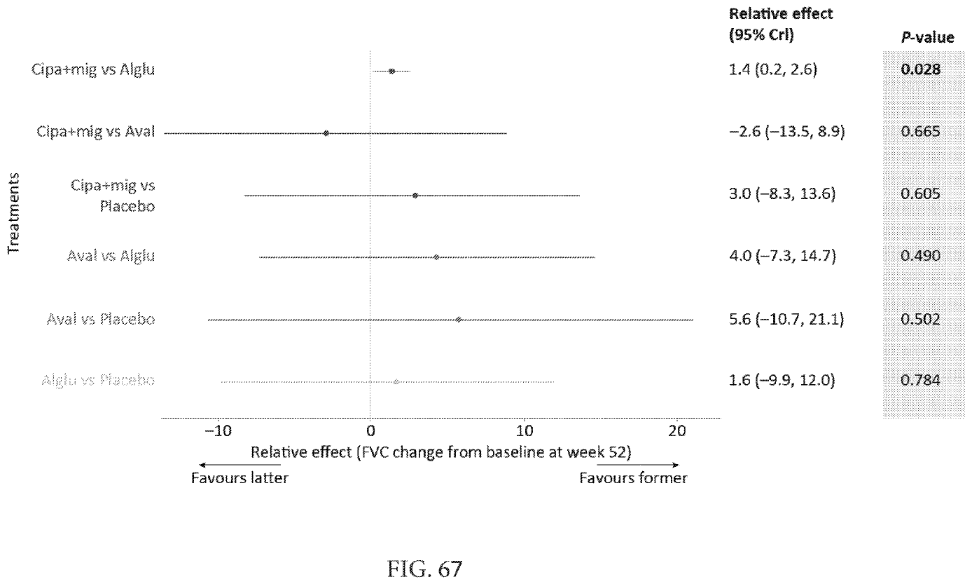

RE models were chosen for both 6MWD and FVC based on the DIC. FIGS. 66 and 67 provide an overview of the relative effect estimates with 95% CrIs for the base-case scenario using the sensitivity analysis and show:
Inclusion of matched single-arm evidence into the network for the main analysis reduces uncertainty of the relative effect estimates
Cipa+mig: statistically favorable versus Alglu; numerically unfavorable versus Aval; numerically favorable versus placebo (6MWT and FVC)
Aval: numerically favorable versus Alglu and placebo (6MWT and FVC)
Alglu: numerically favorable versus placebo

In conclusion, the ML-NMR comparison presented here showed that Cipa+mig was statistically significantly favorable versus Alglu and Aval for 6MWD and FVC in the base-case scenario of the main analysis. Cipa+mig was also statistically significantly favorable over Alglu and Aval for 6MWD and FVC for different ERT durations, with one exception: for FVC, Cipa+mig was only numerically favorable vs. Aval in the ERT-naïve setting.

The sensitivity analysis (only including RCT data) demonstrates that the inclusion of matched single-arm evidence into the network for the main analysis reduces uncertainty of the relative effect estimates. Overall, these results point to Cipa+mig potentially having a differentiated clinical profile versus the other ERTs, particularly for individuals with some level of previous ERT treatment. Further analyses are anticipated to test and refine the findings, when additional longer-term data are published.

### Example 14: Long-Term Comparison of Cipaglucosidase Alfa+Miglustat (Cipa+Mig) and Alglucosidase Alfa (Alglu)

The Phase III double-blind PROPEL study (NCT03729362; ATB200-03) described in Examples 9 and 10 above compared the investigational two-component therapy cipaglucosidase alfa/miglustat (cipa/mig) with alglucosidase alfa/placebo (alglu) in adults with late-onset Pompe disease (LOPD) over 52 weeks. The ongoing open-label extension (OLE) of PROPEL (NCT04138277; ATB200-07) evaluates long-term safety and efficacy of cipa/mig. Outcomes include 6 minute walk distance (6MWD), forced vital capacity (FVC), creatine kinase (CK) and hexose tetrasaccharide (Hex4) levels and safety. Data are reported as change from the PROPEL baseline to OLE week 52 (104 weeks after the PROPEL baseline). In the OLE (N=119; 91 enzyme replacement therapy [ERT]-experienced and 28 ERT-naïve), 82/85 (96.5%) patients previously treated with cipa/mig continued cipa/mig, and 37/38 (97.4%) switched from alglu to cipa/mig; 90.8% of patients remained in the OLE study through week 52. Mean change in % predicted 6MWD was +3.1 (8.07 standard deviation) for cipa/mig-cipa/mig and −0.5 (7.76) for alglu-cipa/mig in ERT-experienced patients and +8.6 (8.57) for cipa/mig-cipa/mig and +8.9 (11.65) for alglu-cipa/mig in ERT-naïve patients. Mean change in % predicted FVC was −0.6 (7.50) for cipa/mig-cipa/mig and −3.8 (6.23) for alglu-cipa/mig in ERT-experienced patients and −4.8 (6.48) and −3.1 (6.66) in ERT-naïve patients. Mean reduction in CK (U/L) for ERT-experienced and ERT-naïve patients was −132.1 (215.74) and −216.9 (243.66) for cipa/mig-cipa/mig and −161.0 (269.52) and −218.6 (316.47) for alglu-cipa/mig, respectively. Mean reduction in Hex4 (mmol/mol) for ERT-experienced and ERT-naïve patients was −1.9 (3.22) and −2.9 (2.45) for cipa/mig-cipa/mig and −2.6 (3.75) and −2.9 (2.22) for alglu-cipa/mig, respectively. During PROPEL through week 52 of the OLE, treatment-emergent adverse events occurred in 84 (98.8%) cipa/mig-cipa/mig and 36 (97.3%) alglu-cipa/mig patients. Three patients discontinued the OLE due to infusion-associated reactions (urticaria, urticaria and hypotension, and anaphylaxis, respectively). No new safety signals were identified. Data demonstrate treatment with cipa/mig up to 104 weeks was associated with a durable effect and was well tolerated, supporting long-term benefits of treatment for patients with LOPD.

Example 15: ATB200-08 Trial: Safety, Pharmacokinetics, Efficacy, Pharmacodynamics, and Immunogenicity in Pediatrics Subjects with IOPD

The ATB200-08 trial seeks to evaluate the safety and tolerability of co-administration of cipaglucosidase alfa/miglustat in ERT-experienced subjects with infantile-onset Pompe disease (IOPD) aged 6 months to <18 years and in ERT-naïve subjects with IOPD aged 0 to <12 months. Further, the ATB200-08 study seeks:
to evaluate the efficacy of co-administration of cipaglucosidase alfa/miglustat in ERT-experienced subjects with IOPD aged 6 months to <18 years and in ERT-naïve subjects with IOPD aged 0 to <12 months;
to characterize the pharmacokinetics (PK) of cipaglucosidase alfa and miglustat and evaluate the dose regimen in ERT-experienced subjects with IOPD aged 6 months to <18 years and in ERT-naïve subjects with IOPD aged 0 to <12 months for the co-administration of cipaglucosidase alfa/miglustat
to evaluate the effect of the co-administration of cipaglucosidase alfa/miglustat on pharmacodynamic (PD) markers in ERT-experienced subjects with IOPD aged 6 months to <18 years and in ERT-naïve subjects with IOPD aged 0 to <12 months as measured by urinary hexose tetrasaccharide (Hex4) and creatine kinase (CK)
to evaluate the effect of anti-rhGAA antibodies on cipaglucosidase alfa exposure, safety, and efficacy in ERT-experienced subjects with IOPD aged 6 months to <18 years and in ERT-naïve subjects with IOPD aged 0 to <12 months
to evaluate the effect of the co-administration of cipaglucosidase alfa/miglustat on growth in ERT-experienced subjects with IOPD aged 6 months to <18 years and in ERT-naïve subjects with IOPD aged 0 to <12 months.

This is a Phase 3, open-label, multicenter study to evaluate the safety, efficacy, PK, pharmacodynamics (PD), and immunogenicity of cipaglucosidase alfa/miglustat treatment in ERT-experienced and ERT-naïve pediatric subjects with IOPD. These subjects will be enrolled in 1 of 2 cohorts:
Cohort 1 will be treated with cipaglucosidase alfa/miglustat weekly and will include approximately 16 ambulatory ERT-experienced subjects experiencing clinical decline (as described below), aged 6 months to <18 years.
Cohort 2 will be treated with cipaglucosidase alfa/miglustat weekly and will include approximately 16 ERT-naïve subjects aged 0 to <12 months, including at least 6 subjects aged 0 to <6 months.

Subjects may be cross-reactive immunologic material (CRIM)-positive or -negative. All CRIM-negative subjects will receive, or will have received, immunotherapy before or concurrent with receiving treatment. CRIM-positive subjects may receive immunotherapy according to institution standards at the investigator's discretion. The type, dosage, and regimen of immunotherapy will be determined by the investigator or according to institution standards.

The study will be conducted in 2 Stages:
Stage 1 will consist of a 104-week treatment period with cipaglucosidase alfa/miglustat.
Stage 2 will consist of a long-term extension treatment period. This stage will last until approval or until study termination by the sponsor.

Infusion visits will be scheduled every week throughout the study. Study visits that include efficacy, additional safety, and other assessments will be scheduled and may occur over up to 4 days. All study assessments and procedures (with the exception of PK sample collection and post-dose electrocardiograms [ECGs]) are performed before administration of study drug.

All subjects will undergo the following study procedures/assessments:
Safety assessments will be performed during Stages 1 and 2 and will include monitoring of adverse events (AEs), clinical laboratory tests (chemistry, hematology, and urinalysis), vital signs, physical examinations, 12-lead ECGs, and echocardiograms.
Sparse blood samples for determination of total human acid α-glucosidase (GAA) protein levels and miglustat concentrations in plasma for a population PK analysis will be collected on Day 1, Week 26, Week 52, Week 78, and Week 104 for Cohorts 1 and 2 during Stage 1 according to a schedule to be determined based on modeling and simulation.
Samples will be collected for detection of anti-human acid α-glucosidase (GAA) total, cross-reactive, and neutralizing antibodies.
Efficacy assessments will be performed during Stages 1 and 2 and will include the following:

| | Age (years) at Time of Assessment | | | |
|---|---|---|---|---|
| Efficacy Assessments | 0 to <2 | 2 to <4 | 4 to <12 | 12 to <18 |

Motor Function

| | | | | |
|---|---|---|---|---|
| Alberta Infant Motor Scale | Y | N | N | N |
| (AIMS) | (birth to | | | |
| | walking | | | |
| | independently | | | |
| | or 18 months) | | | |
| Gross Motor Fanction | Y | Y | Y | Y |
| Measure-88 (GMFM-88) | (≥5 months) | | | |
| (total score | | | | |
| 6-minute walk test (6MWT) | N | N | Y | Y |
| (ambulatory subjects only) | | | (≥5 years) | |
| Timed Up and Go (TUG) | N | N | Y | Y |
| (ambulatory subjects only) | | | | |
| Manual muscle test (MMT) | N | N | Y | Y |
| (nonambulatory subjects will | | | | |
| perform muscle strength tests | | | | |
| for upper limbs only) | | | | |
| Use of assistive device or new | Y | Y | Y | Y |
| assistive device for | | | | |
| ambulation (ambulatory | | | | |
| subjects only) | | | | |

Respiratory Function

| | | | | |
|---|---|---|---|---|
| Pulmonary function tests | N | N | Y | Y |
| (FVC sitting) | | | (≥5 years) | |
| Ventilation (noninvasive | Y | Y | Y | Y |
| [CPAP/BiPAP] or invasive; | | | | |
| hours per day) | | | | |
| Ventilator-independence | Y | Y | Y | Y |
| (for non-ventilator dependent | | | | |
| subjects only) | | | | |

Cardiac Function

| | | | | |
|---|---|---|---|---|
| Echocardiogram^{a} | Y | Y | Y | Y |
| Electrocardiogram^{b} | Y | Y | Y | Y |

Other Assessments

| | | | | |
|---|---|---|---|---|
| Urinary Hex4 | Y | Y | Y | Y |
| CK | Y | Y | Y | Y |
| Overall survival | Y | Y | Y | Y |
| Physician's Global Impression | Y | Y | Y | Y |
| of Severity (PGIS) | | | | |
| Subject's or Caregiver's | Y | Y | Y | Y |
| Global Impression of Severity | by caregiver | by caregiver | by caregiver | by subject |
| (SGIS) | | | or subject | |
| Quality of Life in | N/A | N/A | Y | Y |
| Neurological Disorders | | | by caregiver | by subject |
| *NeuroQOL) Pediatric Lower | | | | |
| Extremity Function - Mobility | | | or subject | |
| EuroQol 5 Dimensions Youth | N/A | N/A | Y | Y |
| 5 Levels Instrument | | | by caregiver | by subject |
| (EQ-5D-Y-5L [4 to 16 years] | | | or subject | |
| or EQ-5D-5L [17 to 18 years]) | | | | |
| Growth measures: | Y | Y | Y | Y |
| height/length, weight, and | | | | |
| head circumference | | | | |
| (≤2 years) | | | | |

Abbreviations: BiPAP = bilevel positive airway pressure; CK = creatine kinase; CPAP = continuous positive airway pressure; ECG = electrocardiogram; FVC = forced vital capacity; Hex4 = hexose tetrasaccharide; N = no; N/A = not applicable; Y = yes
^{a}Echocardiogram parameters to be collected include but are not limited to left ventricular mass index (LVMI), left ventricular ejection fraction, fractional shortening, left ventricular internal diameter at end-diastole and at end systole, left ventricular mid wall fractional shortening, and left ventricular wall thickness.
^{b}ECG parameters collected include but are not limited to ECG mean heart rate, PR interval, QRS duration, QT interval, QTcB interval, QTcF interval, and RR interval.

### Diagnosis and Main Criteria for Inclusion:

### Cohort 1 (ERT-Experienced, 6 Months to <18 Years):

### Inclusion Criteria

Subjects must meet all of the following criteria to be considered for enrollment in the study:
1. Male or female subjects who are aged 6 months to <18 years on Day 1.
2. Subject's parent or legally authorized representative is willing and able to provide written informed consent and authorization for study participation and use and disclosure of personal health information or research-related health information.
3. Subject must have documentation of all of the following prior to initiation of study drug:
   a. Bi-allelic gene encoding human acid α-glucosidase (GAA) variants
   b. CRIM status
   c. No or low measurable GAA enzyme activity
4. Subject must have had hypertrophic cardiomyopathy at the time of diagnosis defined as:
   >1 standard deviation above the mean age-specific LVMI for subjects diagnosed by newborn screening or sibling screening
   >2 standard deviations above the mean age-specific LVMI for subjects diagnosed by clinical evaluation
5. Subject must have received ERT for at least 6 months immediately before enrollment. For subjects whose ERT dosage has been modified, the subject must have been on the modified dosage and regimen for at least 3 months before enrollment.
6. Subject must have experienced a clinical decline based on the protocol-specified criteria (see below) on their current approved rhGAA dose and frequency.
7. If of reproductive potential and sexually active, female and male subjects must agree to use a highly effective method of contraception throughout the duration of the study and for at least 90 days after their last dose of cipaglucosidase alfa/miglustat.
8. For subjects aged ≥12 to <18 years, subject must perform one 6-minute walk test (6MWT) (≥75 meters) at screening that is valid, as determined by the clinical evaluator; for subjects aged ≥5 to <12 years, subject must perform one 6MWT (≥40 meters) at screening that is valid, as determined by the clinical evaluator. Subjects aged 18 months to <5 years must be ambulatory and in the opinion of the investigator assessed to be likely to be able to perform 6MWT (≥40 meters) when they turn 5 years old.

### Criteria for Clinical Decline for Inclusion of Subjects in Cohort 1:

Clinical decline will be defined for a subject being on their current approved rhGAA dose and frequency for any of the following:
Decline in 6-minute walk distance (6MWD) (10%) or sitting forced vital capacity (FVC) (10%) between the screening visit and a previous assessment at least 3 months prior obtained as part of routine clinical care
Motor milestone or skills regression (e.g., losing the ability to sit without support, losing the ability to stand without support, losing hands-and-knees crawling ability, losing the ability to walk without assistance, losing the ability to stand alone, losing the ability to walk alone) as assessed by the investigator
Increase in LVMI z-score of more than 1.0 as calculated from the echocardiogram at screening compared to a previous assessment at least 3 months prior
New use of an assistive device for ambulation

### Exclusion Criteria

Subjects who meet any of the following criteria will be excluded from participating in the study:
1. Subject requires invasive ventilation (e.g., tracheostomy).
2. Subject requires respiratory assistance for ≥16 hours per day (including noninvasive ventilator support) continuously for ≥14 days in the absence of an acute reversible illness, excluding perioperative ventilation.
3. Subject received any investigational drug or any investigational biologic for Pompe disease within 30 days or 5 half-lives of the therapy or treatment, whichever is longer, before screening.
4. Subject is CRIM-negative and has not received prophylactic immunomodulation.
5. Subject has high and sustained antibody titers to previous ERT, as defined per local standard of care.
6. Subject has received any gene therapy within the past 3 years.
7. Subject has any intercurrent illness or condition at screening or baseline that may preclude the subject from fulfilling the protocol requirements or suggests to the investigator and/or the medical monitor that the potential subject may have an unacceptable risk by participating in this study.
8. Subject has any prior history of illness or condition known to affect motor function, such as, but not limited to, Guillain Barre syndrome, cerebral palsy, etc.
9. Subject has a hypersensitivity to any of the excipients in cipaglucosidase alfa, approved rhGAA, or miglustat.
10. Female subject is pregnant (or intends to get pregnant) or breastfeeding at screening.
11. In the opinion of the investigator, the parent or legally authorized representative is unlikely or unable to comply with the study requirements.

### Cohort 2 (ERT-Naïve, 0 to <12 Months):

### Inclusion Criteria

Subjects must meet all the following criteria to be considered for enrollment in the study:
1. Male or female subjects who are aged 0 to <12 months at on Day 1.
2. Subject's parent or legally authorized representative is willing and able to provide written informed consent and authorization for study participation and use and disclosure of personal health information or research-related health information.
3. Subject must have documentation of all the following prior to initiation of study drug:
   a. Bi-allelic GAA variants
   b. CRIM status
   c. No or low measurable GAA enzyme activity
4. Subject must have had hypertrophic cardiomyopathy at the time of diagnosis defined as:
   >1 standard deviation above the mean age-specific LVMI for subjects diagnosed by newborn screening or sibling screening
   >2 standard deviations above the mean age-specific LVMI for subjects diagnosed by clinical evaluation
5. Subject is ERT-naïve (having had no prior ERT-treatment).

### Exclusion Criteria

Subjects who meet any of the following criteria will be excluded from participating in the study:
1. Subject requires invasive ventilation (e.g., tracheostomy).
2. Subject requires respiratory assistance for ≥16 hours per day (including noninvasive ventilator support) continuously for ≥14 days in the absence of an acute reversible illness, excluding perioperative ventilation.
3. Subject received any investigational drug or any investigational biologic for Pompe disease within 30 days or 5 half-lives of the therapy or treatment, whichever is longer, before screening.
4. Subject is CRIM-negative and will not be receiving prophylactic immunomodulation.
5. Subject has received any gene therapy at any time.
6. Subject has any intercurrent illness or condition at screening or baseline that may preclude the subject from fulfilling the protocol requirements or suggests to the investigator and/or the medical monitor that the potential subject may have an unacceptable risk by participating in this study.
7. Subject has any prior history of illness or condition known to affect motor function, such as, but not limited to, Guillain Barre syndrome, cerebral palsy, etc.
8. Subject has a hypersensitivity to any of the excipients in cipaglucosidase alfa, approved rhGAA, or miglustat.
9. In the opinion of the investigator, the parent or legally authorized representative is unlikely or unable to comply with the study requirements.

The cipaglucosidase alfa dose of 30 mg/kg being evaluated in this protocol was determined from population PK modeling and simulation analyses that included pooled data from 96 adults and 11 pediatric subjects dosed at 20, 25, and 30 mg/kg, including 3 pediatric IOPD patients from the Expanded Access Program (EAP) (ages 1 to 3 years) at the 30 mg/kg dose level. The 30 mg/kg dose level in combination with additional effect from miglustat provided exposures that are well within the safety margin and multiples of exposure established by the juvenile toxicology no-observed-adverse effect-level (NOAEL) of 150 mg/kg. The 30 mg/kg cipaglucosidase alfa dose level in combination with miglustat has been generally well-tolerated in 3 pediatric IOPD patients in the EAP.

Estimated systemic exposures from actual plasma total GAA protein levels at doses of 20 mg/kg, 25 mg/kg, and 30 mg/kg or simulated 30 mg/kg exposures derived from actual plasma total GAA protein exposures from lower doses are presented in Table 4 for adults and for 3 pediatric IOPD subjects in the EAP program. As shown in Table 21 below, cipaglucosidase alfa dose-related increases in systemic exposures are observed for all 3 pediatric subjects. The exposure ratio for 30 mg/kg cipaglucosidase alfa in the 3 children relative to adults who received 20 mg/kg cipaglucosidase alfa approaches unity or is below levels observed in adults.

TABLE 21
Estimated Exposure Levels in 3 IOPD Subjects at 30 mg/kg Cipaglucosidase

| | | Cipaglucosidase | AUC | Cₘₐₓ (μg/mL)/ |
|---|---|---|---|---|
| | Body Weight | Alfa + Miglustat | (μg · h/mL)/Ratio | Ratio to |
| Subject | (kg) | Dose | to Adult % | Adult % |
| Adultₐ | ≥50 | 20 mg/kg + 260 mg | 1444 (1035-1949) | 295 (228-366) |
| Subject Aₐ | 14.0 | 20 mg/kg + 80 mg | 1076/74.5 | 217/73.6 |
| Subject Aₐ | 14.8 | 25 mg/kg + 80 mg | 1170/81.0 | 250/84.7 |
| Subject A_{b} | 14.8 | 30 mg/kg + 80 mg | 1504/104 | 308/104 |
| Subject Bₐ | 16.4 | 20 mg/kg + 80 mg | 779/53.9 | 190/64.4 |
| Subject Bₐ | 18.2 | 25 mg/kg + 80 mg | 1081/74.9 | 255/86.4 |
| Subject B_{b} | 18.2 | 30 mg/kg + 80 mg | 1368/94.7 | 314/106 |
| Subject Cₐ | 8.73 | 20 mg/kg + 30 mg | 516/35.7 | 121/41.0 |
| Subject Cₐ | 9.87 | 25 mg/kg + 35 mg | 728/50.4 | 167/56.6 |
| Subject Cₐ | 11.7 | 30 mg/kg + 80 mg | 997/69.0 | 223/75.6 |

Abbreviations:
AUC = area under the curve; Cmax = observed maximum concentration; GAA = human acid α-glucosidase; LOPD = late-onset Pompe disease
ₐAUC and Cmax estimated from actual observed plasma total GAA protein concentrations; summary for adults is based on an N of 93 subjects with LOPD, median (10th and 90th percentiles)
_{b}AUC and Cmax simulated from observed plasma total GAA protein concentrations at lower doses; actual data pending

This frequency of dosing is based on the systemic clearance of the cipaglucosidase alfa and miglustat, and the acuity/severity of disease in IOPD, as well as observations in 3 IOPD patients enrolled in the EAP (ERT-experienced patients in clinical decline). These patients were initiated on every-other-week regimens, and the frequency has been increased to weekly administrations with no evidence of accumulation of either drug and a well-tolerated safety profile. Additional support for a weekly dose regimen is found in a recent publication of a multicenter observational cohort study (Maartje, Huidekper et al, 2022), which showed that survival of patients with IOPD was significantly improved with a 40 mg/kg weekly dose compared with a 20 or 40 mg/kg biweekly dose.

At fixed milligram dose levels, miglustat doses will be administered based on body weight cutoffs from modeling and simulations that match exposures to adults weighing ≥50 kg from administration of a 260 mg dose. Miglustat will be administered approximately 1 hour before the start of IV infusion of cipaglucosidase alfa, which will be administered over approximately 4 hours. Subjects are required to fast at least 2 hours before and 2 hours after the oral administration of miglustat, with the exception that infants (0 to <2 years) may receive Pedialyte® or a similar dextrose-electrolyte based oral solution.

Since it is expected that subjects will gain weight as they grow, dose adjustments of miglustat based on changes in weight category may be made, but only after the weight increase is confirmed at 2 consecutive infusion visits.

A weekly frequency of 30 mg/kg cipaglucosidase alfa and miglustat administration will be evaluated in this study. A weekly 30 mg/kg cipaglucosidase alfa and miglustat dose has been administered in pediatric subjects with IOPD from the EAP. The PK disposition in these subjects indicated that both drugs were cleared with no accumulation from weekly dosing. The rationale for weekly dosing in both cohorts is based on evidence supporting improved survival for IOPD patients receiving weekly dosing of standard of care ERT compared to every other week dosing (Ditters, Huidekper et al, 2021). In addition, the ERT-experienced IOPD subjects in Cohort 1 will be in clinical decline and are expected to be transitioning from a weekly regimen of standard of care treatment, so a transition to every other week dosing may decrease the likelihood of stabilizing or improving the clinical status of these subjects.

### Criteria for Evaluation:

### Stage 1 (Baseline to Week 104)

### Safety

The primary endpoint is the proportion of subjects with infusion-associated reactions (IARs).

Additional safety endpoints from baseline to Week 104 are as follows:
incidence of treatment-emergent adverse events (TEAEs), including treatment-emergent serious adverse events (TESAEs), anaphylactic reactions, and TEAEs leading to discontinuation of study drug
changes in clinical laboratory test results
changes in vital signs
changes in 12-lead ECG results
changes in echocardiogram parameters (other than LVMI)

### Efficacy

Efficacy will be assessed by the composite and age-appropriate individual endpoints as follows:

Cohort 1 only: a composite efficacy endpoint will assess the proportion of subjects at Week 104 who experience any of the following events of clinical decline relative to entry into the study:
death due to complications of Pompe disease
new invasive ventilator dependency in the absence of intercurrent illness
increase in LVMI z-score from baseline of more than 1.0 as calculated from the most recent echocardiogram
% predicted sitting FVC decrease ≥15% from baseline in the absence of intercurrent illness
new use of an assistive device for ambulation

### Cohort 2 Only:

the proportion of subjects who are alive and free of invasive ventilation at Week 104
the proportion of subjects at Week 104 who experience a decrease in LVMI z-score from baseline of more than 1.0 as calculated from the most recent echocardiogram

### Both Cohorts:

### Motor Function:

change from baseline to Week 104 in the total score for the Alberta Infant Motor Scale (AIMS) (<18 months)
change from baseline to Week 104 in the total score for the Gross Motor Function Measure-88 (GMFM-88) items (≥5 months)
change from baseline to Week 104 in the total score for Timed Up and Go (TUG) (≥4 years)
change from baseline to Week 104 in ambulatory function, as measured by 6MWT (>5 years)
change from baseline to Week 104 in the following variables related to muscle strength (≥4 years):
   manual muscle test (MMT) score for the lower and upper extremities and neck
   MMT total score
time to use of an assistive device or new assistive device for ambulation

### Pulmonary Function:

change from baseline to Week 104 in % predicted sitting FVC (≥5 years)
change from baseline to Week 104 in hours per day on noninvasive ventilation
time from birth to either death or use of invasive ventilation (e.g., tracheostomy) or respiratory assistance for ≥16 hours per day (including noninvasive ventilator support) continuously for ≥14 days in the absence of an acute reversible illness, excluding perioperative ventilation

### Cardiac:

change from baseline to Week 104 in LVMI and LVMI z-score

### Other Measures:

overall survival
subject's disease severity (improving, stable, or declining) at Week 104, as measured by the Physician Global Impression of Severity (PGIS)
subject's disease severity (improving, stable, or declining) at Week 104, as measured by the Subject Global Impression of Severity (SGIS) reported by the subject or caregiver
change from baseline to Week 104 in the following variables from patient-reported outcome (PRO) measures:
   score for Quality of Life in Neurological Disorders (NeuroQoL) assessment of lower extremity function
   score for EuroQol 5 Dimensions Youth 5 Levels Instrument (EQ-5D-Y-5L [4 to 16 years] or EuroQol 5 Dimensions 5 Levels Instrument (EQ-5D-5L [17 to 18 years]) assessment of quality of life
change from baseline to Week 104 in the proportion of subjects requiring continuous positive airway pressure (CPAP)/bilevel positive airway pressure (BiPAP) use
change from baseline to Week 104 in body weight, height/length, and head circumference (<2 years of age) (both absolute value and percentile for all 3 parameters)

### Pharmacokinetics

Pharmacokinetic parameters include clearance (CL) and volume of distribution (V), estimated from total GAA protein concentrations and miglustat concentrations in plasma. Other PK parameters will be derived as follows:
AUC: area under the plasma drug concentration-time curve
Cₘₐₓ: maximum observed concentration obtained directly from the concentration profile

### Immunogenicity

anti-rhGAA antibodies (total, neutralizing, and cross-reactive)

### Pharmacodynamics

change from baseline to Week 104 in serum CK level
change from baseline to Week 104 in urinary Hex4 level

### Stage 2 (Baseline to End of Study)

Endpoints regarding safety, efficacy, PK, and PD (optional) for Stage 2 will be similar to those for Stage 1.
Example 16: Treatment of Pediatric IOPD Patient with ATB200/AT2221

A 6-month-old male patient with severe infantile Pompe disease presented. The patient underwent a comprehensive physical examination, measurement of GAA enzyme activity, genetic testing of the GAA gene and muscle biopsy to establish the diagnosis.

The patient received the high-dose enzyme replacement therapy with Myozyme after diagnosis. The patient responded very poorly to therapy. Due to rapidly progressing Pompe disease and inadequate response to therapy, the patient's survival was at risk. For the patient, the opportunity arose to receive the new ATB200/AT2221 ERT via a compassionate use/Expanded Access Program (EAP).

### Enzymatic Diagnostics of Acid Alpha Glucosidase

In the context of metabolic diagnostics, the activity of acid alpha glucosidase was determined in fibroblasts and lymphocytes at pH 4 and 7. To confirm the results, additional dry blood diagnostics were performed with a measurement of the activity of alpha-glucosidase at a pH of 3.8 and 7. Additional indicators were evaluated, such as the activity of GAA with and without inhibitor, as well as the ratio of the activity at pH 3.8 to pH 7.0.

### Genetic Analysis

DNA was isolated from EDTA blood. The gene encoding acidic alpha-glucosidase is located on chromosome 17q25.2-q25.3. First, polymerase chain reaction was used to amplify the 19 protein-coding exons including the adjacent intron regions. Subsequently, DNA sequencing and for the detection of deletions and duplications multiplex-ligation-dependent-product-amplification was performed. Because of the negative family history and autosomal recessive transmission of Pompe disease, it was assumed that the mutations by the patient were located on two separate chromosomes. Therefore, genetic testing of the parents was performed. Afterwards the identified mutations were compared with the mutations known to be disease-causing in the Human Gene Mutation Database.

### Muscle Biopsy

Within the first year of life a muscle biopsy from the pectoralis muscle was taken. The obtained material was examined by electron microscopy and enzyme histochemistry. For the patient PAS-, oil red-, trichrome-, NADH-, ATPase- and acid phosphatase-staining was applied to the sample.

### Glc4 Methodology

The index patient has regular measurement of the urinary Glc4-levels since the change of the ERT to ATB200. The Glc4-levels were determined by an established method from the Duke University. The analysis of Glc4 is standardized as a butyl-4-aminobenzoate derivative using [¹³C₆] Glc4 as the internal standard (IS). The measurement is performed as ultra-performance liquid chromatography-tandem mass spectrometry (UPLC-MS/MS). The Glc4-values are then normalized to creatinine.

### Clinical Treatment

The patient presented with IOPD at 6 months and was started on the standard dose of Myozyme (20 mg/kg/bi-weekly). Due to the inadequate response to therapy, the dose was doubled just one month after the start of therapy in form of a weekly administration of Myozyme. Under this dose of 20 mg/kg/weekly, the patient showed slight motor improvements. But the cardiological situation deteriorated progressively.

Therefore, just three months after initiation of therapy, the dose was again doubled to 40 mg/kg/weekly, so the patient received a total of four times the standard dose. With the intensification of ERT and cardiac therapy, the patient's condition initially stabilized. Nevertheless, over the next few months, the patient's general condition steadily deteriorated, and the patient decompensated, especially respiratory.

Despite the fourfold dose of ERT and the lack of ADA due to immunomodulation, the patient's response to therapy was very poor for unexplained reasons. At this time, it was assumed that the clinical course of patient might be fatal.

The patient was switched from Myozyme to ATB200/AT2221 after eight months of insufficient Myozyme therapy response. The patient was 14.5 months old at the time of the therapy switch. The new therapy (via EAP) was initiated with the oral administration of 30 mg premedication AT2221 (Miglustat) one hour prior to enzyme administration. ATB200 was administered at a bi-weekly interval at a dosage of 20 mg/kg. In the further course of treatment, the dosage of premedication and ERT was repeatedly adjusted and optimized (see Table 22). Some actual dosages in Table 22 (e.g., 37 mg/kg and 34.5 mg/kg at 32 and 38 months, respectively) varied from the target dose (e.g., 30 mg/kg) to avoid waste of extra material left in vials.

TABLE 22
Dosage Plan for ERT

| | | Dosage | Dosage of | |
|---|---|---|---|---|
| | | of GAA | Miglustat | |
| Age | Therapy | (mg/kg) | (mg) | Interval |
| 6 months | Myozyme | 20 | | 2-weekly |
| February 2019 | | | | |
| 7 months | Myozyme | 20 | | Weekly |
| 9 months | Myozyme | 40 | | Weekly |
| 14 months | ATB200/AT2221 | 20 | 30 | 2-weekly |
| 16 months | ATB200/AT2221 | 25 | 30 | 2-weekly |
| 18 months | ATB200/AT2221 | 25 | 35 | 2-weekly |
| 19 months | ATB200/AT2221 | 30 | 40 | Weekly |
| 24 months | ATB200/AT2221 | 30 | 60 | Weekly |
| 31 months | ATB200/AT2221 | 30 | 80 | Weekly |
| 32 months | ATB200/AT2221 | 37 | 80 | Weekly |
| 38 months | ATB200/AT2221 | 34.5 | 115 | Weekly |

### Results

The parents of the patient reported motor progress shortly after the first administration of ATB200/AT2221. The patient developed increased facial expressions and muscle strength, which resulted in new motor skills.

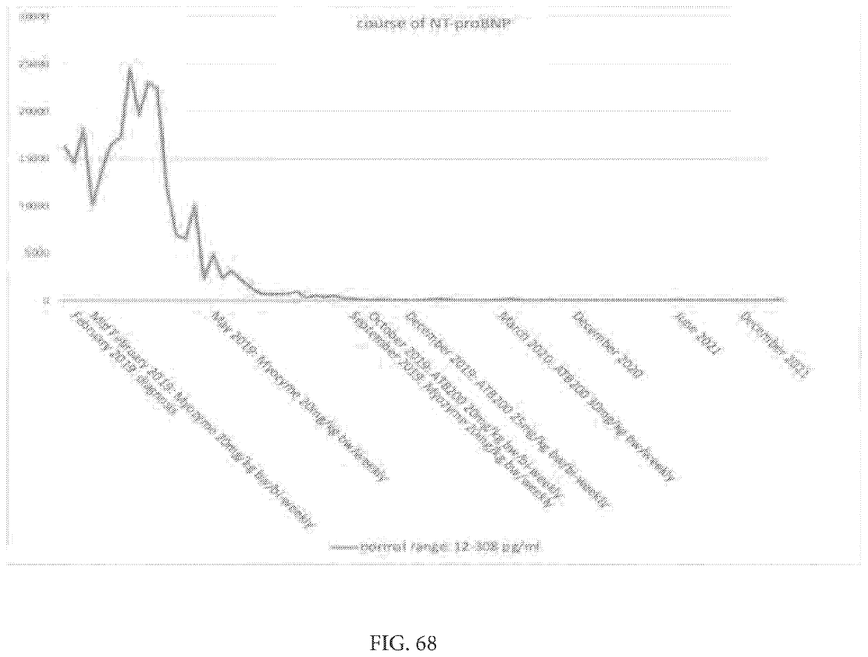

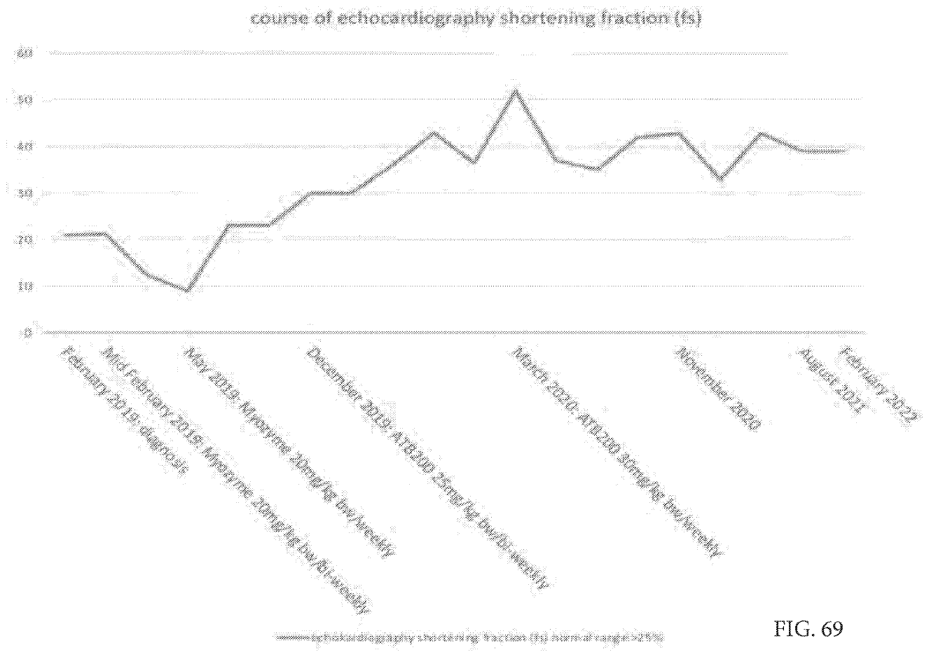

ATB200/AT2221 significantly improved the cardiac and respiratory situation. The heart failure parameters such as NT-proBNP returned to the normal range (FIG. 68). Cardiac function showed a significant improvement (FS 30-43%, FIG. 69). With time, the supportive cardiological therapy could be reduced and finally completely discontinued.

After three months with ATB200/AT2221, the patient no longer needed oxygen during the day except in the context of infections. The patient developed during the course a pulmonary infection that required invasive ventilation at times. Because of the need of mechanical ventilation, a tracheostomy was discussed. Ultimately, it was decided that intensification of the ERT would be performed first due to the patient's good response to therapy so far. Subsequently, 30 mg/kg ATB200 was administered weekly five months after the start of therapy.

After the switch to weekly enzyme administration, the doctors and parents noted clinical improvement. Only two days after the first weekly administration of ATB200, the patient regained his voice.

The patient's respiratory status improved steadily under the new therapy, so the patient did not need non-invasive ventilation even at night seven months after the switch of the ERT. After 14 months with ATB200/AT2221, the patient no longer required any oxygen at night.

Furthermore, a constantly positive motor development could be observed. The patient now shows significantly improved head control and trunk stability. The long-term motor goals include further development of head control and independent turning on the side.

The patient is recently able to sit freely cross-legged for a short time. The muscle strength of the patient is now sufficient to move independently in the wheelchair. Communication with the patient has also improved greatly, as he has already learned some words and signs. Due to improved motor skills, the patient can play and enjoys for example stacking building blocks or doing puzzles.

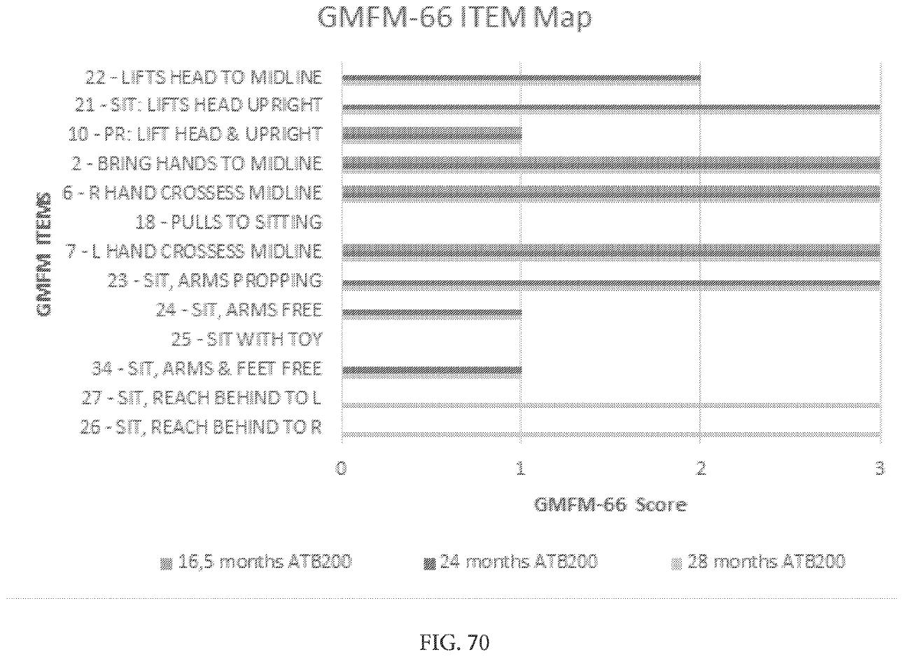

The Gross Motor Function Measure 66 (GMFM 66) is used to quantify a patients' motor skills. The GMFM 66 is a worldwide standardized test developed and validated for children with cerebral palsy to measure the development of gross motor function over time. During testing 66 items are examined and a score from 0 to 100 points is possible. The GMFM 66 has been performed three times on the patient so far at half-yearly intervals and the following test scores were achieved (FIG. 70):
16.5 months of ATB200 therapy: 17.0 points
24 months of ATB200 therapy: 24.0 points
28 months of ATB200 therapy: 29.3 points

In the context of dose adjustments, treatment efficacy according to the EAP was higher with high-dose therapy of ERT. With high-dose ERT a faster respiratory and cardiological stabilisation as well as especially a motor improvement was noticed. The treatment-response of the patient improved at doses above the recommended standard doses of the EAP. The patient did not develop any treatment-related side effects even under higher doses of ERT.

### Glc4 Analysis

In the patient, urinary Glc4 has been determined regularly since the switch to ATB200 at 14 months of age. At the start of therapy with ATB200, the urinary Glc4 value was significantly elevated. The Glc4 levels remained relatively constant under the biweekly administration of a dose of 20 mg/kg.

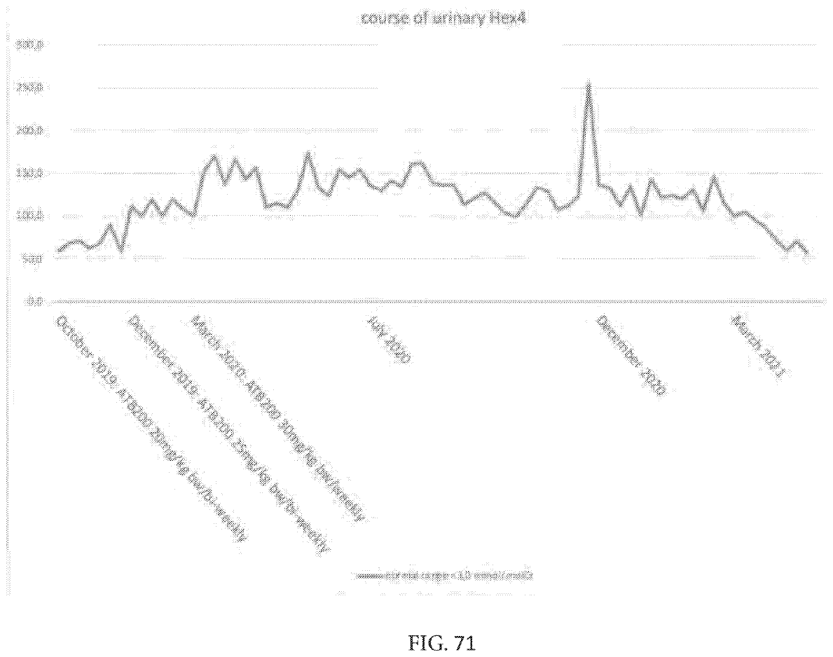

An increase in Glc4 levels was observed with the dose increase to 25 mg/kg/bi-weekly and also most notably with the switch to a weekly administration of 30 mg/kg at 19 months of age (13 months of treatment). The values stayed at a relatively high level until 32 months of age (26 months of treatment). Since then, a decrease of the values to the initial level as at the beginning of therapy can be observed (FIG. 71).

Further details regarding the treatment of this patient are described in Fiege et al., “Improved Enzyme Replacement Therapy with Cipaglucosidase Alfa/Miglustat in Infantile Pompe Disease”, Pharmaceuticals 2023; 16 (9): 1199, which is hereby incorporated by reference in its entirety.

Example 17: Treatment of Pediatric IOPD Patient with ATB200/AT2221

The patient, male, first-born of unrelated parents of African descent (Burkina Faso), was born at 37 weeks by an emergency delivery due to fetal heart failure. At birth, he was resuscitated and admitted to the NICU for invasive ventilatory assistance and circulation support.

Echocardiography showed a hypertrophic cardiomyopathy (HCM, LVMI 232 g/m2 Simpson's method) with severe biventricular systolic and diastolic dysfunction (ejection fraction EF 30%). The ECG showed a short PR interval (0.07 sec). Moreover, the patient presented with severe hypotonia, elevated serum CPK (1063 U/L) and BNP (9169 U/L, nv 0-100).

At 3 days of life, an extended newborn screening resulted positive for Pompe Disease (GAA activity in DBS 0.40 μM/L, nv>3). uGlc4 was elevated (71.2 mmol/mol creatinine, nv <16.3). The diagnosis was confirmed by GAA molecular analysis (compound heterozygous c.2560C>T (p.Arg854*)+deletion exons 4-8), and the GAA Western Blot assay on lymphocytes showed a CRIM negative status.

At 5 days of life, the patient was treated with alglucosidase alfa (Myozyme®). Simultaneously, an immunomodulation protocol with rituximab, ev immunoglobulins and methotrexate was started. In addition, an appropriate therapy for the heart failure was started (betablocker, diuretics, ACE inhibitor).

The patient progressively improved, and, at the age of 2 months, he was discharged from the NICU. At 1 year, cardiac mass (LVMI 116.6 g/m2) and function (EF 50%) were improved, and, at the same time, improvements in motor function and biomarkers (uGlc4 20.2 mmol/mol crea, nv<7.7, CPK 1877 u/L) were also recorded.

Unfortunately, the patient subsequently developed a high anti-ERT antibody titer (1:102,400), followed by clinical and biochemical worsening (psychomotor delay, elevated serum CPK, maximum 6,795 U/L, elevated uGlc4, maximum 50 mmol/mol creatinine). Cardiac mass was increased (LVMI 229 g/m2), as well as BNP levels (237 U/L) while the EF remained stable (50%).

The patient underwent a new immunomodulation cycle with bortezomib, rituximab, sirolimus and ev immunoglobulins, with a reduction of the antibody titer at 1:6,400 after 1 year, but with only partial clinical and biochemical improvement. In particular, he showed a persistence of psychomotor delay, HCM (LVMI 118 g/m2, EF 48%), increased uGlc4 (35 mmol/mol crea) and CPK (4,728 U/L).

At 2.5 years of life, he started a compassionate use/EAP therapy that combined a new ERT (cipaglucosidase 30 mg/kg/week) with an enzyme stabilizer (miglustat 115 mg) (ATB200-15 Program, Amicus Therapeutics), with clinical benefit.

After 3 months, the patient could walk independently, biomarkers were reduced (CPK 2,989 U/L, uGlc4 32.6 mmol/mol crea) cardiac mass (LVMI 82 g/m2) and function (EF 67%) improved. But an echocardiography showed deep and large recesses and marked hypertrabeculation especially in the apex.

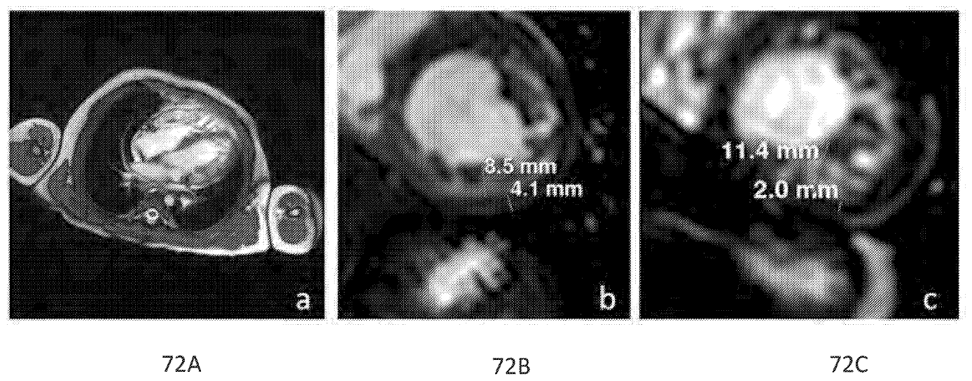

After 1 year of the new therapy (3.5 years old), a new echocardiography fulfilled the criteria of non-compaction myocardium (NC/C 2.6) with normal mass (LVMI 64 g/m2) and function (EF 75%). Cine MRI confirmed the diagnosis of non-compaction myocardium, biventricular, evident both in long axis (FIG. 72A) and in short axis, with a ratio NC/C 8.4/4.1 in medial segments of the left ventricle (FIG. 72B) with a maximum of NC/C 11.4/2 in the apex (FIG. 72C). Disease biomarkers remained stable (CPK 2,557 U/L, uGlc4 30.4 mmol/mol creatinine) and the patient had no cardiac symptoms.

Further details regarding the treatment of this patient are described in Gragnaniello et al., “Unusual Evolution of Hypertrophic Cardiomyopathy in Non-Compaction Myocardium in a Pompe Disease Patient”, Journal of Clinical Medicine 2023, 12, 2365, which is hereby incorporated by reference in its entirety.

### Example 18: Treatment of Taiwanese Female Pediatric IOPD Patient

A newborn female initially presented after abnormal newborn screening results for Pompe disease. The mother received regular prenatal examination with normal results. There was no family history of genetic disease.

Due to abnormal newborn screening of GAA, the following heterozygous mutation c.[1935C>A;1726G>A] (p.[D645E; G576S])/c.2024_2026del (p.N675del) was detected and she was referred for further study. Her chest X-ray (CXR) showed cardiomegaly with a cardiothoracic ratio (C/T) ratio 0.6. Cardiac enzymes were as following: creatine kinase (CK) 880 U/L and N-terminal pro b-type natriuretic peptide (NT-pro BNP) 10169 pg/mL. Heart echo revealed hypertrophic cardiomyopathy, without right ventricular outflow tract obstruction or left ventricular outflow tract obstruction. Intermittent bradycardia was noted, and her EKG showed sinus bradycardia with biventricular hypertrophy and a short PR interval.

Enzyme replacement therapy with Myozyme 40 mg/kg/q2wk was first provided shortly after birth for 6 months. At approximately 3 years of age, she began to use ATB200 and AT2221 via the expanded access program (EAP).

### Diagnostic Lab Data

### GAA Enzyme Activity (Whole Blood)

### Newborn

nGAA activity: 0.32 (umol/L Wb/h) (12.22±5.88)
GAN/nGAA ratio: 147.20 (<20)
Acarbose Inhibition: 91.33% (<70)

### GAA Enzyme Activity (Lymphocyte)

Newborn: α-Glucosidase: 0.84 nmol/mg Prot/hr
α-Glucosidase (+Inhibitor): 0.11 nmol/mg Prot/hr
   GAA gene: GAA c.[1935C>A;1726G>A] (p.[D645E; G576S])/c.2024_2026del (p.N675del) compound Heterozygote.

### Follow-Up Condition Notes

At 3 months of age, the patient developed well. She had a loud cry, similar to that of her 2 older brothers. Her breathing sounds increased continually, and she had not been choking. She had good activity, truncal hypotonia, and positive head control. She was able to grasp with her whole palm, had good bilateral leg kicking, and could hold the prone position for a short time. However, she was not good with stepping, had no subcostal retraction, had upper extremity spasticity of the arms and she did not like the prone position.

At 7 months old, her biweekly treatment of Myozyme was increased to 50 mg/kg for 10 months. At 8-months of age, the patient had been treated with Enzyme Replacement Therapy (ERT) every two weeks since birth. However, after being on ERT for 4 months, she developed a skin rash, dyspnea, a cough and a pale-like complexion. The symptoms could be relieved by giving diphenhydramine and solumedrol. After 7 months on ERT, the cough was noted soon after her infusion started, and a slower infusion rate was suggested. Upon follow up, she was admitted to complete ERT at lower infusion rate with diluted Myozyme.

At 1Y3M of age, she needed assistance to stand but could exchange things between two hands, clap her hands, grab a pen, was able to pronounce “mama,” “Buffy,” “monmon,” and wave to say goodbye. At 1Y4M of age, the patient began to learn to walk.

At 1Y of age, the patient displayed CK levels of 1213 U/L which increased to 2722 U/L 5 months later when the patient was 1Y5M. At 1Y6M, the patient progressively weakened, and she developed bilateral ptosis. However, she continued to learn to walk. Due to these weakening clinical results, it was recommended to increase her ERT dose to 50 mg/kg/Q2W. When the patient was 1Y6M, her biweekly Myozyme treatment was increased to 60 mg/kg for 5 months. At 1Y7M of age, the patient was able to climb stairs, and had continued her ability to exchange things between two hands, clap her hands, and grab a pen.

At 1Y8M of age, the patient started immunotherapy due to her weakness. One month later, at 1Y9M of age, the patient had good activity and no diarrhea. Her CK levels reduced in one month from 3143 U/L at 1Y9M years old to 2550 U/L at 1Y10M of age. Due to her progression in weakness, her ERT dose increased to 60 mg/kg/Q2W, as recommended, and she was continually observed for the development of an allergy.

Myozyme treatment for the patient changed to weekly at 30 mg/kg for 3 months when the patient was 1Y11M. At this age, the patient displayed CK levels of 2665 U/L. Two months later, her CK levels lowered to 2397 U/L. At 2Y0M of age, the patient had fair activity and walked quickly. Her chest displayed clear, soft, hyperactive breathing sounds, and her cardiac exam showed no murmur. Due to her continual weakness, following recommendations, her treatment was changed to an ERT dose of 40 mg/kg/QW and the patient was observed for the development of infusion-associated reactions. When the patient turned 2Y2M, her weekly treatment of Myozyme increased to 40 mg/kg for 10 months.

At 2Y10M of age, the patient displayed CK levels of 1878 U/L. Due to evidence of CO₂ retention on blood gas analysis, home non-invasive ventilation (BiPAP) was suggested. At 3 years of age, her CK levels reduced to 1444U/L and the patient began using a therapy consisting of ATB200 328 mg (20 mg/kg) and ATB2221 80 mg q2w via the expanded access program (EAP) while continuing her premedication use. At the beginning of her ATB200/AT2221 treatment, she did not develop a skin rash, dyspnea, a cough, or a pale-like complexion. The patient's mother noted that at the start of ATB200 infusion, the patient tended to fall frequently and that her activity was below what it was before infusion. From time of ATB200/AT221 treatment start when the patient was 3Y of age, the number of Anti-rhGAA antibodies reduced 1-fold after 1 month of treatment. This level remained steady after another month of treatment when the patient turned 3Y2M. After 2 months on ATB200/AT2221 treatment, the CK levels of the now 3Y2M old patient increased to 1717 U/L. Following recommendations, she was kept under observations and was given a sponsor with which to plan for an infusion dose adjustment if needed.

When the patient was 3Y3M, her CK levels had continued to reduce compared to the prior month, to 1674 U/L. At 3Y4M, the patient had been on a ATB200/AT2221 treatment for 17 weeks. She had adjusted to weekly dosing from 3Y3M, after 13 weeks of biweekly dosing, due to her frequently falling particularly before infusions. She kept her weight between 17.8 kg and 18.3 kg and had good activity. She could also walk independently with an ankle-foot orthosis. The patient's mother mentioned that she was falling less, and her activity was better after the dose regimen changed to weekly infusions. At this time, she had not developed any skin rash, dyspnea, cough or pale-like complexion.

At 3Y5M of age, after 2 months on the previous dose, the weekly treatment given to the patient was increased to 25 mg/kg of ATB200 and 80 mg of AT2221. At this age, her CK levels had continued to reduce compared to the prior month, to 992 U/L, and her NT-proBNP levels were 231.9 μg/mL. At 3Y7M, her CK levels and NT-proBNP levels remained stable following an additional month of treatment (CK: 1687 U/L; NT-proBNP: 33.24 μg/mL). The dosage was increased again after 2 months to weekly treatment of 30 mg/kg of ATB2000 and 80 mg of AT2221 in the patient when she was 3Y7M. During this time, she kept her weight between 18.3 kg and 18.8 kg. Her sponsor agreed to adjust the infusion dose to 25 mg/kg since 3Y5M (week21). After adjusting to 25 mg qw, the patient's mother reported an increase in speech and activity, but the patient still exhibited weakness and easy falling before subsequent infusions. Overall, she noted this condition to be similar to the patient's previously performance when on Myozyme 40 mg qw.

After reviewing the patient's PK and biochemical data, and her poor clinical status the cipaglucosidase alfa dosage was changed from 25 mg/kg to 30 mg/kg qw starting at age 3Y7M. At 3Y8M of age, the CK levels and NT-proBNP levels of the patient remained stable but decreased one month later, at 3Y9M of age, as the patient had been on ATB200/AT2221 for 9 months. Demographic, hematologic, and biochemical data for the patient can be found in Table 23. She kept her weight between 18.8 kg and 19.1 kg. The patient's mother reported she is in a better condition, including less falls and stronger muscle power, as well as less ptosis. At this time, she had not developed any skin rash, dyspnea, cough, or pale-like complexion.

At 3Y11M of age, her CK levels improved from prior months. At 4Y of age, the patient had been using ATB200/AT2221 for one year and her CK level had further improved while her NT-proBNP level remained stable. Demographic, hematologic, and biochemical data for the patient can be found in Table 23. Her weight remained between 19.1 kg and 19.5 kg. At this time, she had not developed any skin rash, dyspnea, cough, or pale-like complexion. The patient could not cooperate with using non-invasive ventilation during the night. She began to walk quickly, could count from 1 to 10, and had a good performance reported by her schoolteachers. She started a rehabilitation program and was eating without choking.

The urinary Glu4 levels of the patient increased between age of 4Y and 4Y2M. Between age of 4Y2M and 4Y3M, her CK levels improved, and her NT-proBNP levels remained stable. At 4Y3M the patient had been using ATB200/AT2221 for 64 weeks. Demographic, hematologic, and biochemical data for the patient can be found in Table 23. Her dose regimen remained at AT2221 130 mg and ATB200 30 mg/kg qw since 4Y3M. When the patient was using an ankle foot orthosis, she had a steady, quick walk. She presented with hypotonia, hypernasality, and very good power in her lower leg muscle. She had a good hand and palm grasp. At this time, the patient had not developed any skin rash, dyspnea, cough, or pale-like complexion.

Urinary Glu4 levels of the patient remained steady between her years of being 4Y2M and 4Y4M. Between age of 4Y3M and 4Y4M, her CK and NT-proBNP levels remained stable. The demographic, hematologic, and biochemical data for the patient can be found in Table 23. At 4Y6M of age, the patient had been using ATB200 for 78 weeks and had continued on doses of AT2221 130 mg and ATB200 30 mg/kg qw since 4Y3M. She continued to struggle with use of non-invasive ventilation throughout the night. When she walked with an ankle foot orthosis, she had a steady, quick walk. She presented with hypotonia, hypernasality, and very good power in her lower leg muscle. She had a good hand and palm grasp. At this time, the patient had not developed any skin rash, dyspnea, cough, or pale-like complexion.

After 52 weeks of the treatment, the patient at 4Y7M of age demonstrated an AUC and Cₘₐₓ for ATB200 that were both within the adult 10^{th}-90^{th} percentile. For AT2221, the AUC is below the adult 10^{th} percentile and Cₘₐₓ was within the adult 10^{th}-90^{th} percentile.

The demographic, hematologic and biochemical data for the patient across her time on either Myozyme or ATB200/AT2221 treatment is provided in Table 23.

TABLE 23
Demographic, Hematologic and Biochemical Data of the Female Taiwanese Patient.

| | | | Pro- | | | | |
|---|---|---|---|---|---|---|---|
| | | | BNP | LVEF | LVMI | Glc4 | Dose |
| Age | BW (kg) | CK (U/L) | (pg/mL) | (%, 2 D) | (g/m², 2 D) | (mmol/mol CRE) | Myozyme (M) or ATB200 (A) |
| ~1 | 3.5 | 880 | 10169 | | 147 | 41.88 | double dose 40 mg/kg q2wk (M) |
| week | | | | | (M-mode) | | |
| 0 Y 1 M | 4 | 431-689 | 545.6-1590 | | | 17.45 | double dose 40 mg/kg q2wk (M) |
| 0 Y 2 M | 5.2 | 148 | 160.7 | | 69.3 | 21.13 | double dose 40 mg/kg q2wk (M) |
| | | | | | (M-mode) | | |
| 0 Y 3 M | 6 | 102 | 249.2 | | | 30.06 | double dose 40 mg/kg q2wk (M) |
| 0 Y 4 M | 6.5-6.7 | 123 | | 71.1 | 69.1 | 14.29 | double dose 40 mg/kg q2wk (M) |
| | | | | (M-mode) | (M-mode) | | |
| 0 Y 6 M | 7.7 | | | 71.1 | 76.1 | | double dose 40 mg/kg q2wk (M) |
| | | | | (M-mode) | (M-mode) | | |
| 0 Y 7 M | 7.8 | 262 | | | | 29.2 | 50 mg/kg q2wk (M) |
| 0 Y 8 M | 8.1 | 303 | | | | 13.62 | 50 mg/kg q2wk (M) |
| 0 Y 9 M | 8 | 437 | | | 82.9 | 16.63 | 50 mg/kg q2wk (M) |
| | | | | | (M-mode) | | |
| 1 Y 0 M | 8.4 | 1213 | | | 81.5 | 8.01 | 50 mg/kg q2wk (M) |
| | | | | | (M-mode) | | |
| 1 Y 1 M | 9.1 | 1079 | | | | | 50 mg/kg q2wk (M) |
| 1 Y 2 M | 8.2 | 1167 | | | | | 50 mg/kg q2wk (M) |
| 1 Y 3 M | 9.6 | 1474-2217 | | | | | 50 mg/kg q2wk (M) |
| 1 Y 5 M | 10 | 2514-2722 | | | | 45.13 | 50 mg/kg q2wk (M) |
| 1 Y 6 M | 10 | 1792 | | 70.5 | 26.2 | 64.02 | 60 mg/kg q2wk (M) |
| 1 Y 7 M | 10.2 | 3139 | | | | 32.46 | 60 mg/kg q2wk (M) |
| 1 Y 8 M | 10.8 | 2502-2528 | | | | 24.9 | 60 mg/kg q2wk(M) |
| 1 Y 9 M | 11 | 3143-3625 | | | | 25.22 | 60 mg/kg q2wk (M) |
| 1 Y 10 M | 11.2 | 2550 | | | | 36.85 | 60 mg/kg q2wk (M) |
| 1 Y 11 M | 11.9 | 2665 | | | | 33.78 | 60 mg/kg q2wk (M) |
| 2 Y | 12.5 | 2397-2656 | | 61 | 65.9 | 42.83 | *30 mg/kg qwk (M) |
| | | | | | | | (2019 Sep. 5-December 2023) |
| | | | | | | | *40 mg/kg qwk (M) |
| | | | | | | | (2019 Oct. 24-2020 Oct. 14) |
| 2 Y 6 M | 14 | 2603 | | | | | *30 mg/kg qwk (M) |
| | | | | | | | (2019 Sep. 5-December 2023) |
| | | | | | | | *40 mg/kg qwk (M) |
| | | | | | | | (2019 Oct. 24-2020 Oct. 14) |
| 2 Y 9 M | | | | 72.2 | 45.9 | | *30 mg/kg qwk (M) |
| | | | | | | | (2019 Sep. 5-December 2023) |
| | | | | | | | *40 mg/kg qwk (M) |
| | | | | | | | (2019 Oct. 24-2020 Oct. 14) |
| 2 Y 10 M | | 1817 | 401.5 | | | 59.85 | *30 mg/kg qwk (M) |
| | | | | | | | (2019 Sep. 5-December 2023) |
| | | | | | | | *40 mg/kg qwk (M) |
| | | | | | | | (2019 Oct. 24-2020 Oct. 14) |
| 3 Y | 16.4 | 1393-1444 | | 41 | 42 | 32.58 | change to ATB200 20 mg/kg q2w |
| 3 Y 1 M | 16.7 | 1762 | | | | 25.49 | ATB200 20 mg/kg q2w |
| 3 Y 2 M | 17.8 | 1717 | 151.5 | | | 39.18 | ATB200 20 mg/kg q2w |
| 3 Y 3 M | 18.2 | 1674 | | 61.1 | 38 | 50.74 | Change ATB200 to 20 mg/kg QW |
| 3 Y 4 M | 18.3 | 1834 | 36.3 | | | 41.53 | ATB200 20 mg/kg QW |
| 3 Y 5 M | 18.3 | 1524 | 231.9 | | | 34.85 | Change ATB200 to 25 mg/kg QW |
| 3 Y 6 M | 18.8 | 922 | 33.24 | 60.3 | 43.6 | 47.66 | ATB200 25 mg/kg QW |
| 3 Y 7 M | 18.8 | 1687 | | | | 45.88 | ATB200 25 mg/kg QW |
| 3 Y 8 M | 18.8 | 1661 | 180.6 | | | 36.19 | Change ATB200 to 30 mg/kg QW |
| 3 Y 9 M | 19.1 | 1574 | 42 | 60.3 | 50.93 | 38.75 | ATB200 30 mg/kg QW |
| 3 Y 10 M | 19.1 | 1082 | | | | 25.44 | ATB200 30 mg/kg QW |
| 3 Y 11 M | 19.1 | 1304 | | | | 24.37 | ATB200 30 mg/kg QW |
| 4 Y 0 M | 19.5 | 1027 | 42.38 | 50 | 50.8 | 25.26 | ATB200 30 mg/kg QW |
| 4 Y 1 M | 19.5 | 1277 | | | | | Change AT2221 to 115 mg QW |
| 4 Y 2 M | 19.5 | 1187 | 44.29 | | | | AT2221 115 mg QW |
| 4 Y 3 M | 20.5 | 1120 | 45.52 | | | | ATB200 20 mg/kg, |
| | | | | | | | AT2221 130 mg |
| 4 Y 4 M | 20.5 | 1283 | | | | 29.08 | ATB200 20 mg/kg, |
| | | | | | | | AT2221 130 mg |
| 4 Y 5 M | 20.5 | 766 | | | | | ATB200 20 mg/kg, |
| | | | | | | | AT2221 130 mg |
| 4 Y 6 M | 20.6 | 1146 | 29.25 | 57 | 39.1 | | ATB200 30 mg/kg, |
| | | | | | | | AT2221 130 mg |

Example 19: Treatment with ATB200/ATB2221 in a Pediatric IOPD Patient Receiving Immunomodulation Pretreatment

A 3.5M old CRIM+ patient who screened positive for Pompe Disease is homozygous for the mutation c.1075C>A, p.Gly359Arg. At 3.5M of age, she started biweekly treatment with alglucosidase alfa (Myozyme®) at a dose of 25 mg/kg.

At 2 years of life, an immunomodulation protocol with rituximab (375 mg/m²) and IVIG (0.5 g/kg) was started. When the patient was 4Y3M methotrexate (375 mg/m², i.v.) was added to the immunomodulation protocol including rituximab and IVIG, and the patient remained on this treatment for 3 years. After a 1-year break, the patient was 8Y of age and began on the same immunomodulation protocol (methotrexate, rituximab, and IVIG) as a second round of immunomodulation treatment.

At 9Y1M of age, the patient began non-invasive ventilation treatment. She also had two paroxysmal episodes, likely epileptic seizures, that were well controlled with levetiracetam.

At 10Y4M of age, the patient began biweekly treatment with ATB200 (20 mg/kg i.v.) and AT2221 (195 mg) via the expanded access program (EAP). Three months later, the dose of AT2221 was adjusted to 130 mg p.o. and the treatment of the now 10Y7M patient changed to weekly administration. The patient at 11Y of age began premedication with ibuprofen and methylprednisolone before ATB200/AT2221 weekly treatment, as well as a single monthly immunomodulation protocol of rituximab and IVIG.

### Pharmacokinetic Data

At 10Y7M of age, the blood of the patient was collected to gather pharmacokinetic (PK) data for her treatment that included weekly administration of 20 mg/kg ATB200 and 130 mg AT2221. At baseline of her treatment, when she was 10Y7M, the patient demonstrated an AUC and Cₘₐₓ for ATB200 that were both lower than the adult 10^{th} percentile. For AT2221, the AUC was lower than the adult 10^{th} percentile and the Cₘₐₓ was within the adult 10^{th}-90^{th} percentile.

### Urinary Hex4 Levels

At treatment start, when the patient was 10Y4M, she presented with urinary Hex4 levels of 145.55 nmol/mol creatinine. After three months of treatment, when she was 10Y7M, her urinary Hex4 levels reduced more than half to 59.21 nmol/mol creatinine.

### Motor Scale Measurements

At the onset of ATB200/AT2221 treatment, when the patient was 10Y4M, her overall Gross Motor Function Measure-88 (GMFM-88) score was about 8, which steadily increased during 7 months of treatment to a score of about 15 when she turned 10Y11M of age.

At the start of her treatment, when she was 10Y4M, the patient was evaluated for GMFM. The patient received a score of 14 for her ability to complete lying and rolling. After 7 months of treatment, her score for lying and rolling increased 47% to 24 when she was 10Y11M of age.

At her treatment start, the 10Y4M patient received a score of 10 for her ability to sit. After 7 months of treatment, her score for sitting rose 28% to 17 when the patient was 10Y11M of age.

At 10Y4M of age, the patient could keep her arms in an antigravity position while lying down for about 70 seconds at the start of ATB200/AT2221 treatment. This time increased to 900 seconds after 1Y1M of treatment when the patient was 11Y5M. This value remained at 900 seconds after 1Y4M of treatment (patient at 11Y8M of age). The patient was unable to keep her legs in an antigravity position (Mingazzini, 45-degrees).

When accounting for breathing while the arms of the patient remained in an antigravity position while lying, the patient could breathe for 0 seconds after 6 months of TB200/AT2221 treatment when she was 10Y10M. At 11Y4M of age, this amount of time rose to 25 seconds after 1Y of treatment, and additionally increased to about 34 seconds after 1Y4M of ATB200/AT2221 treatment when the patient was 11Y8M of age.

At the start of ATB200/AT2221 treatment, the patient at 10Y4M was unable to ride a home bike. After 9M of treatment, the patient at 11Y1M of age was able to ride a home bike for 15 minutes. Following 10M of treatment, at 11Y2M of age, she was able to ride a home bike for 20 minutes. Her ability to ride a home bike for 20 minutes remained following receiving treatment for 1Y4M when she was 11Y8M of age.

### Ventilation

After 5 months of ATB200/AT2221 treatment, the patient at 10Y9M required non-invasive ventilation (NIV) for 19 hours per day. Her time on NIV was reduced to 13 hours after 11 months on treatment when the patient was 11Y3M and remained at 13 hours following 1Y5M treatment when the patient was 11Y9M.

After 11 months of treatment, the patient at 11Y3M was able to perform spirometry measurements of which she was unable to perform before.

### Follow-Up Parameters

The parents of the patient explained that she stabilized, and her general condition had improved on the weekly dosing regimen. She had stable cardiac ultrasounds, and no dynamics in laboratory results. She also managed to stay in school for 5 hours without NIV.

Example 20: Treatment with ATB200/ATB2221 in a Croatian Pediatric IOPD Patient

A 3M old CRIM-patient screened positive for Pompe Disease as having L552P and q757X mutations. At 4M of age, she was treated with ERT (alglucosidase alfa) at 20 mg/kg biweekly.

At 12Y6M, the patient began treatment with ATB200 (25 mg/kg) and AT2221 (175 mg) biweekly via the expanded access program (EAP). Her most recent regimen had been weekly treatment with ATB200 (25 mg/kg i.v.) and AT2221 (195 mg p.o.) which she switched to when she was 13 years old. She had not received premedication or immunomodulation therapy and she has had no adverse events related to treatment. She also does not need non-invasive ventilation (NIV).

### Pharmacokinetic Data

Blood samples were taken at the start of her ATB200/AT2221 treatment when she was 12Y6M of age to gather pharmacokinetic (PK) data for her treatment that included weekly administration of 25 mg/kg ATB200 and 125 mg AT2221. At the first administration, the patient at 12Y6M of age demonstrated an AUC and Cₘₐₓ for both ATB200 and AT2221 that were each within the adult 10^{th}-90^{th} percentile.

### Urinary Hex4 Levels

At treatment start, patient at 12Y6M presented with urinary Hex4 levels of 98.18 nmol/mol creatinine. After 1 month of biweekly treatment, at 12Y7M of age, her urinary Hex4 levels reduced 37% to 61.96 nmol/mol creatinine. Following 2 months of biweekly treatment, her urinary Hex4 levels had increased from the 1-month time point but were still 17% lower than her levels at treatment start, at 12Y6M of age.

### Motor Scale Measurements

At 12Y4M, the patient received an overall Gross Motor Function Measure-88 (GMFM-88) score of 40 two months before biweekly ATB200/AT2221 treatment. Two months after initiating biweekly treatment (at age 12Y6M), the GMFM-88 score of the patient at 12Y6M of age, increased to about 55-58 where it steadily remained after 6 months of biweekly treatment and 8 months of weekly treatment when the patient was 13Y8M.

Two months before her ATB200/AT2221 treatment, the patient at 12Y4M was evaluated for GMFM. The patient received a score of 51 for her ability to complete lying down and rolling. When she started biweekly treatment, as well as 6 months after followed by 8 months of weekly treatment, her GMFM score for this endpoint remained at 51 when the patient was between 12Y6M to 13Y8M of age.

Two months before the start of treatment, the patient at 12Y4M of age received a score of 60 for her ability to sit. On the onset of biweekly treatment, she was and remained at a score of 60 for her ability to sit. After 4 months of biweekly treatment, her score reduced 15% to 51 when she was 12Y10M. Her sitting score remained at 51 after 6 months of biweekly treatment followed by 8 months of weekly treatment when she turned 13Y8M of age.

At biweekly treatment start, the patient at 12Y6M of age was evaluated for GMFM. She received a score of 31 for her ability to crawl and kneel. After 4 months of biweekly treatment, this score increased to 35 as patient was 12Y10M. Following 6 months of biweekly treatment and 8 months of weekly treatment, her score for crawling and kneeling rose to 39 when the patient was 13Y8M.

At 12Y9M of age, the time that the patient could keep her arms in an antigravity position while lying down was about 2 minutes after 3 months of biweekly ATB200/AT2221 treatment. After 4 months of biweekly treatment, the patient at 12Y10M of age could keep her arms in an antigravity position for 3.5 minutes. After 5 months of biweekly treatment, the patient at 12Y11M of age could keep her arms in an antigravity position for 2 minutes and 40 seconds. Following 6 months of biweekly treatment followed by 4 months of weekly treatment, the patient at 13Y4M of age could keep her arms raised for 3 minutes and 17 seconds. Following 6 months of biweekly treatment followed by 5 months of weekly treatment, the patient at 13Y5M could keep her arms raised for 1 minutes and 40 seconds. After 6 months of biweekly treatment followed by 6 months of weekly treatment, the patient at 13Y6M of age could keep her arms raised for 5 minutes and 25 seconds. The amount of time that the patient at 13Y9M of age could keep her arms raised following 6 months of biweekly treatment followed by 9 months of weekly treatment was 3 minutes and 20 seconds.

After 3 months of biweekly treatment, the patient at 12Y9M could keep her legs in an antigravity position (Mingazzini, 45-degrees) for 10 seconds. The patient was able to increase this time to 14 seconds after 4 months of biweekly treatment when she was 12Y10M. Following 5 months of biweekly treatment, the patient at 12Y11M of age could keep her legs raised for 28 seconds. After 6 months of biweekly treatment followed by 2 months of weekly treatment, the patient at 13Y2M could keep her legs raised for 3 minutes and 20 seconds. The amount of time that the patient at 13Y4M could keep her legs in an antigravity position following 6 months of biweekly treatment followed by 4 months of weekly treatment was 5 minutes and 50 seconds. This time spiked to 14 minutes and 10 seconds following 6 months of biweekly treatment followed by 5 months of weekly treatment in the patient who was at the time 13Y5M of age. After 6 months of biweekly treatment followed by 7 months of weekly treatment 1Y1M of treatment, the amount of time that the patient at 13Y7M of age could keep her legs in the air was 22 minutes 30 seconds. After 6 months of biweekly treatment followed by 9 months of weekly treatment, this time reduced to 15 minutes 50 seconds in the patient at 13Y9M of age.

When accounting for breathing while the arms of the patient remained in an antigravity position while lying, the patient at 12Y9M could breathe for 10 seconds after 3 months of biweekly ATB200/AT2221 treatment. This doubled to 20 seconds after 4 months of biweekly treatment in the patient who was 12Y10M, and additionally increased to 32 seconds after 5 months of biweekly ATB200/AT2221 treatment when she was 12Y11M. After 6 months of biweekly treatment followed by 2 months of weekly treatment, the patient at 13Y2M could breathe for 48 seconds. After 6 months of biweekly treatment followed by 4 months of weekly treatment, the amount of time the patient at 13Y4M of age could breathe was reduced to 35 seconds. However, after 6 months of biweekly treatment followed by 6 months of weekly treatment, the patient at 13Y6M could breathe for 1 minute and 30 seconds. This time reduced to 40 seconds following 6 months of biweekly treatment and 9 months of weekly treatment in the patient who was 13Y9M of age.

After 4 months of biweekly ATB200/AT2221 treatment, the patient at 12Y10M was able to ride a home bike for 6 minutes. Following 5 months of biweekly treatment, her time to ride a home bike increased to 10 minutes in the patient at 12Y11M of age. The time that the patient at 13Y2M could ride a bike after 6 months of biweekly treatment followed by 2 months of weekly treatment spiked to 22 minutes. Another spike in time occurred following 6 months of biweekly treatment followed by 4 months of weekly treatment to 25 minutes for the patient at 13Y4M of age. Following 6 months of biweekly treatment and 8 months of weekly treatment, the patient at 13Y8M could ride a home bike for 27 minutes.

### Follow-Up Parameters

The parents of the patient explained that she stabilized, and her general condition had improved while on the weekly dosing regimen. Her cardiac ultrasounds were stable, and she had no dynamics in laboratory results. Her personal and parental observations disclosed that she has much more energy during the day on the weekly dosage.

Example 21: Treatment with ATB200/ATB2221 in an American Pediatric IOPD Female Patient

A 7M old CRIM+ female patient screened positive for Pompe Disease as having the following mutations: allele 1 c.1396delG, allele 2 c.1705dupT. At 8M of age, she initiated treatment with alglucosidase alfa (Myozyme®) at 20 mg/kg. At 3Y9M, the patient switched ERT therapy to Lumizyme at 40 mg/kg (950 mg) weekly.

At 12Y of age, the patient began treatment with ATB200 (25 mg/kg) and AT2221 (195 mg) biweekly via the expanded access program (EAP). She began home infusions at 13Y3M of age. As of January 2022, at the age of 13Y6M, the patient completed 39 doses. There continues to be discussion about adjusting the biweekly dosage to a weekly regimen.

### Pharmacokinetic Data

Blood sampling of the patient at 12Y of age was conducted to gather pharmacokinetic (PK) data for her treatment that included administration of 25 mg/kg ATB200 and 195 mg AT2221 biweekly. At baseline of treatment, the patient at 12Y of age demonstrated an AUC and Cₘₐₓ for ATB200 that were both lower than the adult 10^{th} percentile. For AT2221, the AUC and Cₘₐₓ were above the adult 90^{th} percentile.

Example 22: Treatment with ATB200/ATB2221 in a Pediatric Atypical IOPD Male Patient

A 2Y10M male patient at 47 kg screened positive for atypical IOPD having the mutation: c.1781G>A (p.R594H)/c.1935C>A (p.D645E). At 3Y1M of age, he initiated biweekly treatment with alglucosidase alfa (Myozyme®) at 20 mg/kg.

At 7Y6M of age, he switched to weekly administration of Myozyme continuing with the same dose (20 mg/kg). The dose of Myozyme administered to the patient reduced to 18.5 mg/kg weekly when he was 8Y5M of age. When the patient was of the age of 9Y4M, his Myozyme treatment was again reduced to 16.3 mg/kg for weekly treatment. Four months later, when the patient was 9Y8M, his weekly treatment of Myozyme increased to a dose of 20 mg/kg.

At 11Y5M of age, the patient began treatment with ATB200 (20 mg/kg) and AT2221 (195 mg) biweekly.

At the start of ATB200/AT2221 treatment, and infrequently for the subsequent year, titer levels of Anti-rhGAA antibody levels were measured in the patient. These levels generally increased over the course of the year. Exact titer levels of Anti-rhGAA antibody are provided in Table 24.

TABLE 24
Anti-rhGAA Antibody Titer Levels for
the Male Patient with Atypical IOPD.

| | | | Titer levels of |
|---|---|---|---|
| Patient | Time on ATB200/AT2221 | | Anti-rhGAA |
| Age | Treatment | | Ab |
| 11Y5M | 1 | day | 102400 |
| 11Y6M | 4 | weeks | 102400 |
| 11Y6M | 6 | weeks | 102400 |
| 11Y8M | 3 | months | 204800 |
| 11Y11M | 6 | months | 406900 |
| 12Y5M | 12 | months | 3276800 |

At 12Y5M of age, the patient has been receiving ATB200 (20 mg/kg) and AT2221 (195 mg) biweekly for 1 year. At this time, the patient weighed 46.5 kg. At this time, the PK levels of both ATB200 and AT2221 were taken from the patient. The AUC and Cₘₐₓ levels for ATB200 are within the adult 10^{th}-90^{th} percentile and near the adult median. The AUC for AT2221 was below the adult 10^{th} percentile but the Cₘₐₓ for AT2221 was just within the adult 10^{th} percentile minimum. AUC and Cₘₐₓ levels of ATB200 and AT2221 for the patient are provided in Table 25.

TABLE 25
AUC and Cₘₐₓ levels for ATB200 and AT2221 after 1 year
of treatment in the male patient with atypical IPOD.

| | ATB200 AUC | ATB200 Cₘₐₓ | AT2221 AUC | AT2221 Cₘₐₓ |
|---|---|---|---|---|
| Subject | (μg*h/mL) | (μg/mL) | (ng*hr/mL) | (ng/mL) |
| Patient with Atypical | 1257 | 245 | 14501 | 1811 |
| IOPD | | | | |
| Adult (median) | 1415 | 294 | 20475 | 2377 |
| Adult (10^{th}-90^{th} | 1001-1924 | 223-363 | 16097-24628 | 1801-3132 |
| percentile) | | | | |

### Example 23: Miglustat Liquid Formulations

Four different formulations of miglustat oral solution 40 mg/mL were prepared. Human study participants were randomized into one of three test groups. Two prototype batches (Prototype 1 and Prototype 4) were manufactured using the same excipients with different concentrations of sodium citrate dihydrate and citric acid monohydrate. The remaining 2 prototype batches (Prototype 2 and Prototype 3) contained additional excipients such as hypromellose and mixed berry flavor.

The formulations for the prototype lab-scale batches are outlined in Table 26.

TABLE 26
Miglustat Liquid Formulations

| | | Prototype 1 | | Prototype 2 | | Prototype 3 | | Prototype 4 | |
|---|---|---|---|---|---|---|---|---|---|
| | | | % | | % | | % | | % |
| Component | Functionality | mg/mL | (w/v) | mg/mL | (w/v) | mg/mL | (w/v) | mg/mL | (w/v) |
| Miglustat | Drug | 40 | 4.00 | 40 | 4.00 | 40 | 4.00 | 40 | 4.00 |
| | substance | | | | | | | | |
| Sodium Citrate | Buffering | 6.524 | 0.65 | 6.524 | 0.65 | 6.524 | 0.65 | 8.388 | 0.84 |
| USP/Ph. Eur. | agent | | | | | | | | |
| (Dihydrate) | | | | | | | | | |
| Citric Acid | Buffering | 2.693 | 0.27 | 2.693 | 0.27 | 2.693 | 0.27 | 3.463 | 0.35 |
| Monohydrate | agent | | | | | | | | |
| USP/Ph. Eur. | | | | | | | | | |
| Hypromellose | Thickening | — | — | 15 | 1.5 | 15 | 1.5 | — | — |
| USP/EP/JP | agent | | | | | | | | |
| Sucralose | Sweetener | 2 | 0.20 | 2 | 0.20 | 2 | 0.20 | 2 | 0.20 |
| NF/Ph. Eur. | | | | | | | | | |
| Potassium Sorbate | Preservative | 2 | 0.20 | 2 | 0.20 | 2 | 0.20 | 2 | 0.20 |
| NF | | | | | | | | | |
| Flavor (Mixed | Flavor | — | — | — | — | 2 | 0.20 | — | — |
| Berry) | | | | | | | | | |
| Purified Water | Vehicle | q.s. | — | q.s. | — | q.s. | — | q.s. | — |
| USP/Ph. Eur. | | | | | | | | | |

| | | |
|---|---|---|
| Hydrochloric Acid | pH modifier | For pH adjustment to 5.0 ± 0.5 |
| Sodium Hydroxide | pH modifier | For pH adjustment to 5.0 ± 0.5 |

PK Results from Oral Solutions of 4 Prototype Formulations Relative to Capsule Contents Tested in Rabbits

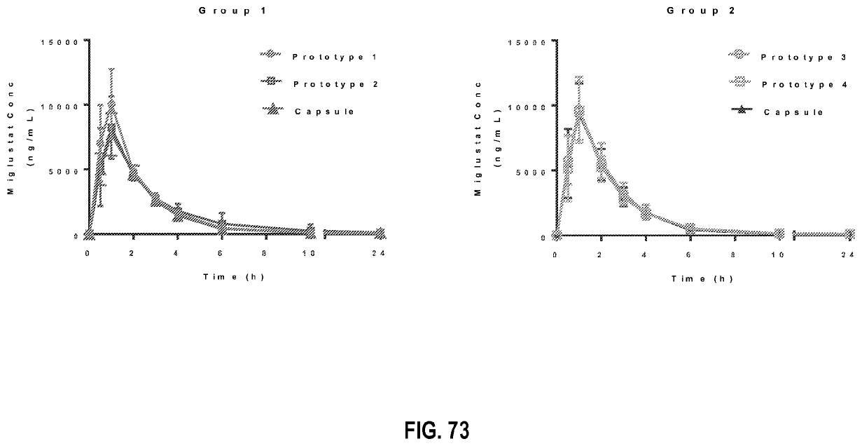

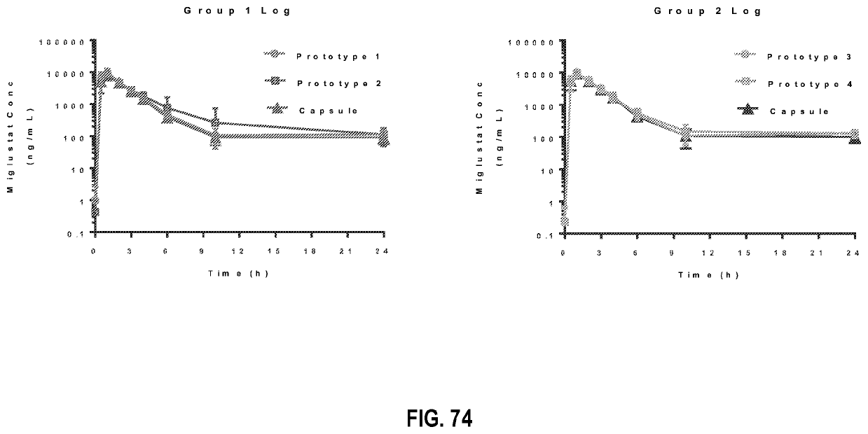

Previous studies were conducted for the selection of pediatric prototype formulations in the rabbit for the selection and advancement to humans. Two groups of 8 male rabbits each received AT221 (65 mg) via oral gavage in a fasted condition with a 4-day washout window between dosing. Group 1 was administered capsules of either prototype 1 or prototype 2 whereas group 2 was administered capsules of either prototype 3 or prototype 4. Blood samples were taken from each study animal to determine plasma concentrations of miglustat at various timepoints: Predose (time 0), 0.5 hours (h), 1 h, 2 h, 3 h, 4 h, 6 h, 10 h, and 24 h post-dose. Analyzing for the concentration (mean and SD) of miglustat in the rabbits over the course of 24 hours, our data suggests that prototypes 3 and 4 are more similar to the capsule than prototypes 1 and 2. This is shown in FIG. 73 and as log-transformed data in FIG. 74. As outlined in Table 27, although the rate of absorption between prototypes 1 and 2 were similar, neither prototype was bioequivalent to the Cₘₐₓ or AUC of the capsule after 1 hour of exposure. Also shown in Table 27, the rate of absorption between prototypes 3 and 4 were similar between formulations and both prototypes were bioequivalent to the Cₘₐₓ or AUC of the capsule after 1 hour of exposure, especially prototype 4.

TABLE 27
Miglustat Pediatric Prototype Formulation Evaluation in Rabbits

| | Cₘₐₓ | AUC₀₋ₜ | AUC_{0-∞} |
|---|---|---|---|
| Treatment | (ng/mL) | (ng*h/mL) | (ng*h/mL) |
| Capsule (Group 1) | 7574 (21.4) | 19589 (9.6) | 19898 (9.3) |
| Prototype 1 | 9769 (27.4) | 23043 (14.2) | 23497 (13.9) |
| Prototype 2 | 7902 (29.2) | 22538 (18.1) | 22918 (17.7) |
| Capsule (Group 2) | 9254 (23) | 22656 (15.9) | 23102 (16.8) |
| Prototype 3 | 9702 (22.8) | 23829 (17.8) | 24258 (17.7) |
| Prototype 4 | 9525 (22.5) | 23263 (16.3) | 23845 (18) |

The stability data for the AT2221 (40 mg/mL) oral solution is outlined in Table 28. Acceptable stability was found for all 4 prototypes with apparent greater stabilities for prototype 3 and 4.

TABLE 28
Miglustat Oral Solution Formulation Stability Data
AT2221 Oral Solution, 40 mg/mL

| | | 1 M | | 3 M | |
|---|---|---|---|---|---|
| | T₀ | Assay | Assay | Assay | Assay |
| Prototype | Assay | 25° C./60% RH | 40° C./75% RH | 25° C./60% RH | 40° C./75% RH |
| #1 | 91.2 | 92.9 | 93.1 | 88.4 | 87.9 |
| (35 nm Na Citrate) | | | | | |
| #2 | 89.9 | 91.2 | 92.2 | 86.9 | 85.7 |
| (thickener) | | | | | |
| #3 | 93.1 | 88.9 | 89.1 | 90 | 89.3 |
| (thickener + flavor) | | | | | |
| #4 | 93 | 89.4 | 90 | 91.1 | 90.9 |
| (45 nm Na Citrate) | | | | | |

### Example 24: PK and BE Study of 260 mg Capsule and Liquid Formulations

Human study participants were randomized into one of three test groups. Group 1 (n=36) received 4× capsules (total dose 260 mg).). These capsules were similar to those used in the previously described ATB200 clinical studies. Group 2 (n=36) and Group 3 (n=36) received a 260 mg dose of miglustat with prototypes 3 and 4, respectively (40 mg/mL×65 mL=260 mg). Participants were dosed on days 1, 4 and 7 with a 72-hour washout period. Blood samples were taken periodically.

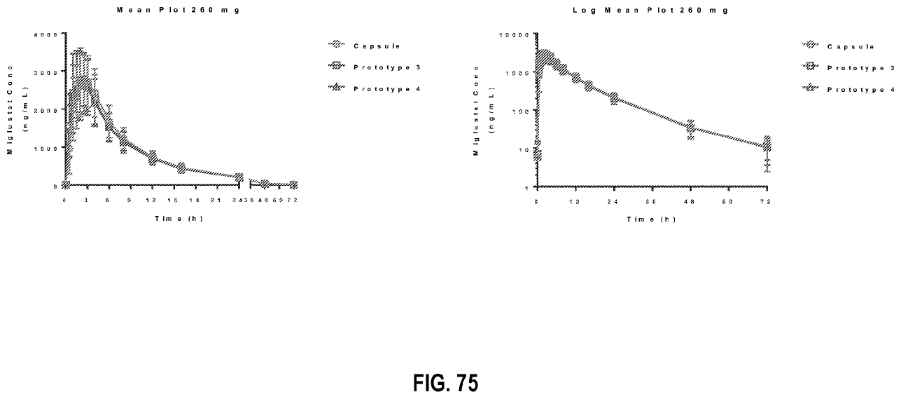

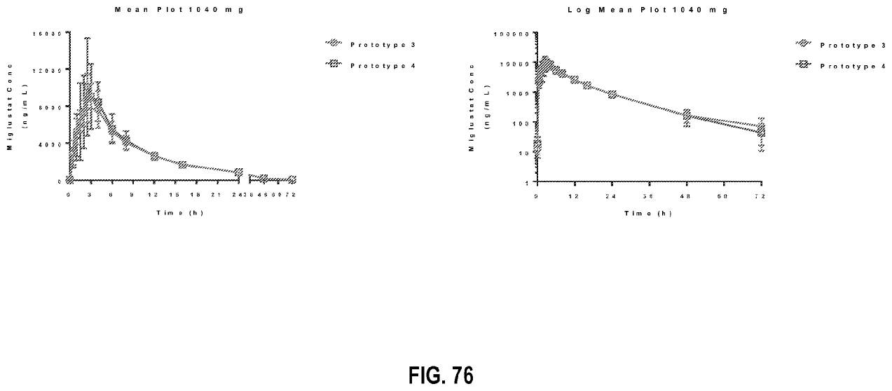

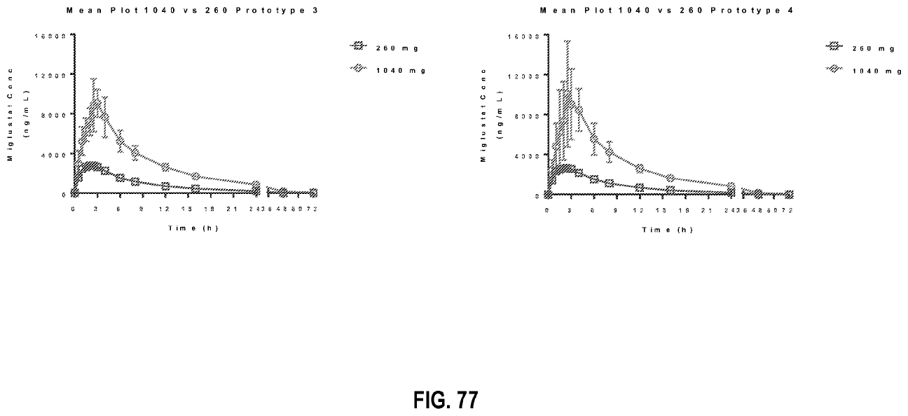

PK summary plots of the mean±SD and log-transformed mean±SD concentrations of miglustat (ng/mL) in humans for prototypes 3 or 4 are shown in FIG. 75 for the dose of 260 mg; data for the 1040 mg dose is shown in FIG. 76. A dose proportionality PK summary plot is shown in FIG. 77 for comparing between the mean concentration of miglustat (ng/ml) over 72 hours in humans following administration of either prototype 3 or 4 at a dose of either 260 mg or 1040 mg. PK data is shown in Table 29. An ANOVA analysis between the capsule and prototype formulations 3 or 4 for the 260 mg dose is shown in Table 30. Both doses from prototypes 3 and 4 are shown to have similar PK profiles to the reference capsule dose. Cₘₐₓ, AUC₀₋ₜ, and AUC_{0-∞} all show bioequivalence between doses from prototypes 3 and 4 and the reference capsule dose. Based on the ANOVA results, prototype 3 shows better bioequivalence with respect to the reference capsule dose.

TABLE 29
PK Summary Data

| | | | | | | | |
|---|---|---|---|---|---|---|---|
| | Cₘₐₓ | Tₘₐₓ | AUC₀₋ₜ | AUC_{0-∞} | T_{1/2} | CL/F | V_{z}/F |
| Formulation | (ng/mL) | (h) | (ng*h/mL) | (ng*h/mL) | (h) | (L/h) | (L) |

260 mg PK Summary

| | | | | | | | |
|---|---|---|---|---|---|---|---|
| Capsule | 2947 | 2.8 | 26610 | 26763 | 9.8 | 9.9 | 141 |
| | (26.6) | (1-4) | (18.8) | (18.7) | (18.6) | (19.6) | (32.2) |
| Prototype 3 | 3081 | 2 | 26252 | 26401 | 9.6 | 10 | 140 |
| | (27.4) | (1-4) | (19.8) | (19.7) | (20.9) | (20.5) | (31.7) |
| Prototype 4 | 2971 | 2 | 25421 | 25566 | 10.1 | 10.4 | 140 |
| | (30.2) | (1-4) | (22.5) | (22.4) | (15.8) | (24.9) | (31.7) |

1040 mg PK Summary

| | | | | | | | |
|---|---|---|---|---|---|---|---|
| Prototype 3 | 9824 | 3 | 91264 | 92271 | 10.7 | 11.3 | 176 |
| | (22.5) | (2.5-4) | (11.4) | (10.6) | (25.2) | (10.7) | (33.6) |
| Prototype 4 | 10329 | 2.5 | 92187 | 92846 | 10 | 11.3 | 165 |
| | (41.3) | (2.5-4) | (17.5) | (17.2) | (22.4) | (16.6) | (32.9) |

Ratio of 1040 mg vs 260 mg PK Summary

| | | | |
|---|---|---|---|
| Prototype 3 | 3.6 | 3.7 | 3.7 |
| Prototype 4 | 3.8 | 3.8 | 3.8 |

TABLE 30
260 mg ANOVA Results

| | Ref GeoLSM | | | Point Estimate | |
|---|---|---|---|---|---|
| PK Parameter | (Capsule) | Prototype | Test GeoLSM | (Ratio % Ref) | 90% CI |
| Cₘₐₓ | 2947 | 3 | 3041 | 103.2 | (98, 108.7) |
| Cₘₐₓ | 2947 | 4 | 2971 | 100.8 | (95.8, 106.1) |
| AUC₀₋ₜ | 26610 | 3 | 26020 | 97.8 | (95.1, 100.5) |
| AUC₀₋ₜ | 26610 | 4 | 25421 | 95.5 | (93, 98.2) |
| AUC_{0-∞} | 26763 | 3 | 26166 | 97.8 | (95.2, 100.5) |
| AUC_{0-∞} | 26763 | 4 | 25566 | 95.5 | (93, 98.1) |

What is claimed is:

1. A method of treating Pompe disease in a subject less than 18 years old, the method comprising administering to the subject a population of recombinant human acid α-glucosidase (rhGAA) molecules, concurrently or sequentially with an enzyme stabilizer,
wherein the dose of the population of recombinant human acid α-glucosidase (rhGAA) molecules is in a range of about 15 mg/kg to about 40 mg/kg, and the dose of the enzyme stabilizer is in a range of about 15 mg to about 300 mg.

2. The method of claim 1, wherein the subject has infantile-onset Pompe disease (IOPD).

3. The method of claim 1 or 2, wherein the subject is an enzyme replacement therapy (ERT)-experienced subject.

4. The method of claim 1 or 2, wherein the subject is an enzyme replacement therapy (ERT)-naïve subject.

5. The method of any one of claims 1-4, wherein the enzyme stabilizer comprises miglustat.

6. method of any one of claims 1-5, wherein the dose of the population of rhGAA is about 20 mg/kg to about 30 mg/kg.

7. The method of any one of claims 1-6, wherein the dose of the population of rhGAA is about 25 mg/kg.

8. The method of any one of claims 1-7, wherein the dose of the population of rhGAA is about 30 mg/kg.

9. The method of any one of claims 1-8, wherein the subject is less than 30 days old.

10. The method of any one of claims 1-9, wherein the dose of miglustat is about 15 mg to about 25 mg.

11. The method of any one of claims 1-10, wherein the dose of miglustat is about 20 mg.

12. The method of any one of claims 1-11, wherein the subject is one month to less than 6 months old.

13. The method of any one of claims 1-12, wherein the dose of miglustat is about 30 mg to about 50 mg.

14. The method of any one of claims 1-13, wherein the dose of miglustat is about 40 mg.

15. The method of any one of claims 1-14, wherein the subject is 6 months to less than 2 years old.

16. The method of any one of claims 1-15, wherein the subject weighs less than 10 kg and the dose of miglustat is about 50 mg to about 80 mg.

17. The method of any one of claims 1-16, wherein the subject weighs less than 10 kg and the dose of miglustat is about 65 mg.

18. The method of any one of claims 1-17, wherein the subject weighs 10 kg to less than 20 kg and the dose of miglustat is about 70 mg to about 100 mg.

19. The method of any one of claims 1-18, wherein the subject weighs 10 kg to less than 20 kg and the dose of miglustat is about 85 mg.

20. The method of any one of claims 1-19, wherein the subject is 2 years to less than 6 years old.

21. The method of any one of claims 1-20, wherein the subject weighs less than 10 kg and the dose of miglustat is about 70 mg to about 100 mg.

22. The method of any one of claims 1-21, wherein the subject weighs less than 10 kg and the dose of miglustat is about 85 mg.

23. The method of any one of claims 1-22, wherein the subject weighs 10 kg to less than 20 kg and the dose of miglustat is about 100 mg to about 130 mg.

24. The method of any one of claims 1-23, wherein the subject weighs 10 kg to less than 20 kg and the dose of miglustat is about 115 mg.

25. The method of any one of claims 1-24, wherein the subject weighs 20 kg to less than 30 kg and the dose of miglustat is about 110 mg to about 150 mg.

26. The method of any one of claims 1-25, wherein the subject weighs 20 kg to less than 30 kg and the dose of miglustat is about 130 mg.

27. The method of any one of claims 1-26, wherein the subject weighs 30 kg to less than 40 kg and the dose of miglustat is about 150 mg to about 200 mg.

28. The method of any one of claims 1-27, wherein the subject weighs 30 kg to less than 40 kg and the dose of miglustat is about 175 mg.

29. The method of any one of claims 1-28, wherein the subject is 6 years to less than 12 years old.

30. The method of any one of claims 1-29, wherein the subject weighs 10 kg to less than 20 kg and the dose of miglustat is about 100 mg to about 130 mg.

31. The method of any one of claims 1-30, wherein the subject weighs 10 kg to less than 20 kg and the dose of miglustat is about 115 mg.

32. The method of any one of claims 1-31, wherein the subject weighs 20 kg to less than 30 kg and the dose of miglustat is about 110 mg to about 150 mg.

33. The method of any one of claims 1-32, wherein the subject weighs 20 kg to less than 30 kg and the dose of miglustat is about 130 mg.

34. The method of any one of claims 1-33, wherein the subject weighs 30 kg to less than 40 kg and the dose of miglustat is about 150 mg to about 200 mg.

35. The method of any one of claims 1-34, wherein the subject weighs 30 kg to less than 40 kg and the dose of miglustat is about 175 mg.

36. The method of any one of claims 1-35, wherein the subject weighs 40 kg to less than 50 kg and the dose of miglustat is about 170 mg to about 220 mg.

37. The method of any one of claims 1-36, wherein the subject weighs 40 kg to less than 50 kg and the dose of miglustat is about 195 mg.

38. The method of any one of claims 1-37, wherein the subject weighs at least 50 kg and the dose of miglustat is about 200 mg to about 300 mg.

39. The method of any one of claims 1-38, wherein the subject weighs at least 50 kg and the dose of miglustat is about 260 mg.

40. The method of any one of claims 1-39, wherein the subject is 12 years to less than 18 years old.

41. The method of any one of claims 1-40, wherein the subject weighs 30 kg to less than 40 kg and the dose of miglustat is about 150 mg to about 200 mg.

42. The method of any one of claims 1-41, wherein the subject weighs 30 kg to less than 40 kg and the dose of miglustat is about 175 mg.

43. The method of any one of claims 1-42, wherein the subject weighs 40 kg to less than 50 kg and the dose of miglustat is about 170 mg to about 220 mg.

44. The method of any one of claims 1-43, wherein the subject weighs 40 kg to less than 50 kg and the dose of miglustat is about 195 mg.

45. The method of any one of claims 1-44, wherein the subject weighs at least 50 kg and the dose of miglustat is about 200 mg to about 300 mg.

46. The method of any one of claims 1-45, wherein the subject weighs at least 50 kg and the dose of miglustat is about 260 mg.

47. The method of any one of claims 1-46, wherein each rhGAA molecule comprises seven potential N-glycosylation sites;
wherein 40%-60% of the N-glycans on the rhGAA molecules are complex type N-glycans;
wherein the rhGAA molecules comprise at least 0.5 mol bis-mannose-6-phosphate (bis-M6P) per mol of rhGAA at the first potential N-glycosylation site as determined using liquid chromatography tandem mass spectrometry (LC-MS/MS).

48. The method of any one of claims 1-47, wherein the rhGAA and enzyme stabilizer are administered weekly.

49. The method of any one of claims 1-47, wherein the rhGAA and enzyme stabilizer are administered every other week.

50. A method of treating infantile-onset Pompe disease (IOPD) in a subject less than 18 years old, the method comprising administering to the subject a population of recombinant human acid α-glucosidase (rhGAA) molecules, concurrently or sequentially with an enzyme stabilizer,
wherein the dose of the population of recombinant human acid α-glucosidase (rhGAA) molecules is in a range of about 20 mg/kg to about 30 mg/kg, and the dose of the enzyme stabilizer is in a range of about 15 mg to about 300 mg.

51. The method of claim 50, wherein the enzyme stabilizer comprises miglustat.

52. The method of claim 50 or 51, wherein the dose of the population of rhGAA is about 30 mg/kg.

53. The method of any one of claims 50-52, wherein the subject is less than 30 days old.

54. The method of any one of claims 50-53, wherein the dose of miglustat is about 20 mg.

55. The method of any one of claims 50-54, wherein the subject is one month to less than 6 months old.

56. The method of any one of claims 50-55, wherein the dose of miglustat is about 40 mg.

57. The method of any one of claims 50-56, wherein the subject is 6 months to less than 2 years old.

58. The method of any one of claims 50-57, wherein the subject weighs less than 10 kg and the dose of miglustat is about 65 mg.

59. The method of any one of claims 50-58, wherein the subject weighs 10 kg to less than 20 kg and the dose of miglustat is about 85 mg.

60. The method of any one of claims 50-59, wherein the subject is 2 years to less than 6 years old.

61. The method of any one of claims 50-60, wherein the subject weighs less than 10 kg and the dose of miglustat is about 85 mg.

62. The method of any one of claims 50-61, wherein the subject weighs 10 kg to less than 20 kg and the dose of miglustat is about 115 mg.

63. The method of any one of claims 50-62, wherein the subject is 6 years to less than 12 years old.

64. The method of any one of claims 50-63, wherein the subject weighs 10 kg to less than 20 kg and the dose of miglustat is about 115 mg.

65. The method of any one of claims 50-64, wherein the subject is 12 years to less than 18 years old.

66. The method of any one of claims 50-65, wherein the subject weighs 20 kg to less than 30 kg and the dose of miglustat is about 130 mg.

67. The method of any one of claims 50-66, wherein the subject weighs 30 kg to less than 40 kg and the dose of miglustat is about 175 mg.

68. The method of any one of claims 50-67, wherein the subject weighs 40 kg to less than 50 kg and the dose of miglustat is about 195 mg.

69. The method of any one of claims 50-68, wherein the subject weighs at least 50 kg, and the dose of miglustat is about 260 mg.

70. The method of any one of claims 1-49, wherein the miglustat or pharmaceutically acceptable salt thereof is administered prior to administration of the population of rhGAA molecules, optionally one hour prior to administration of the population of rhGAA molecules.


SEQUENCE LISTING (6 sequences)

SEQ ID NO: 1 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - DNA, 3624 bp, Homo sapiens
  source 1..3624: mol_type = unassigned DNA
cagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgggcctgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggcctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgcttcccggctgcccagagggctggatgcctgccggtccccgagcaagcctgggaactcaggaaaattcacaggacttgggagattctaaatcttaagtgcaattattttaataaaaggggcatttggaatc

SEQ ID NO: 3 - protein, 952 aa, Homo sapiens; identical to SEQ ID NO: 1

SEQ ID NO: 4 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 5 - protein, 896 aa, Homo sapiens
QQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 6 - protein, 896 aa, Homo sapiens
QQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC